(19) United States
(12) Patent Application Publication
(10) Pub. No.: US 2009/0203764 A1
(43) Pub. Date: Aug. 13, 2009

(54) INHIBITORS OF SEMICARBAZIDE-SENSITIVE AMINE OXIDASE (SSAO) AND VAP-1 MEDIATED ADHESION USEFUL FOR TREATMENT AND PREVENTION OF DISEASES

(76) Inventors: Eric Yanjun WANG, San Diego, CA (US); David S. Jones, San Diego, CA (US); Anne M. O'Rourke, Encinitas, CA (US); Mary T. MacDonald, San Diego, CA (US); Hongfeng Gao, San Diego, CA (US); Huong-thu Ton-Nu, San Diego, CA (US); Christina A. Kessler, San Diego, CA (US); Matthew D. Linnik, Solana Beach, CA (US)

Correspondence Address: MORRISON & FOERSTER LLP, 755 PAGE MILL RD, PALO ALTO, CA, 94304-1018, US

(21) Appl. No.: 12/426,885

(22) Filed: Apr. 20, 2009

Related U.S. Application Data
(63) Continuation of application No. 11/731,819, filed on Mar. 30, 2007.
(60) Provisional application No. 60/787,751, filed on Mar. 31, 2006.
(60) Provisional application No. 60/834,016, filed on Jul. 28, 2006.
(60) Provisional application No. 60/855,481, filed on Oct. 30, 2006.

(51) Int. Cl. A61K 31/137; A61P 29/00; A61K 31/381

(52) U.S. Cl. 514/438; 514/649

(57) ABSTRACT

Compositions and methods of using compositions for treatment of inflammatory diseases and immune disorders are provided. Allylamino compounds are disclosed which are inhibitors of semicarbazide-sensitive amine oxidase (SSAO) and/or vascular adhesion protein 1 (VAP-1). The compounds have therapeutic utility in suppressing inflammation and inflammatory responses, and in treatment of several disorders, including multiple sclerosis and stroke.

### CROSS REFERENCE TO RELATED APPLICATIONS

This application is a continuation of U.S. patent application Ser. No. 11/731,819, filed Mar. 30, 2007, which claims the priority benefit of U.S. Provisional Patent Application No. 60/787,751 filed Mar. 31, 2006, U.S. Provisional Patent Application No. 60/834,016 filed Jul. 28, 2006, and U.S. Provisional Patent Application No. 60/855,481 filed Oct. 30, 2006. The contents of those applications are hereby incorporated herein by reference in their entirety.

### TECHNICAL FIELD

This application relates to compositions and methods for inhibiting semicarbazide-sensitive amine oxidase (SSAO), also known as vascular adhesion protein-1 (VAP-1), for treatment and prevention of inflammation, inflammatory diseases and autoimmune disorders.

### BACKGROUND

Human vascular adhesion protein-1 (VAP-1) is a type 2, 180 kD homodimeric endothelial cell adhesion molecule. Cloning and sequencing of VAP-1 revealed that the VAP-1 cDNA sequence is identical to that of the previously known protein semicarbazide-sensitive amine oxidase (SSAO), a copper-containing amine oxidase. The precise difference (if any) between the membrane-bound VAP-1 adhesion protein and the soluble SSAO enzyme has not yet been determined; one hypothesis indicates that proteolytic cleavage of the membrane-bound VAP-11 molecule results in the soluble SSAO enzyme. Both the membrane-bound VAP-1 protein and the soluble SSAO enzyme have amine oxidase enzymatic activity. Thus membrane-bound VAP-1 can function both as an amine oxidase and a cell adhesion molecule.

Semicarbazide-sensitive amine oxidase is a member of a group of enzymes; that group is referred to generically as semicarbazide-sensitive amine oxidases (SSAOs). SSAOs are mostly soluble enzymes that catalyze oxidative deamination of primary amines. The reaction results in the formation of the corresponding aldehyde and release of H₂O₂ and ammonium. These enzymes are different from monoamine oxidases A and B (MAO-A and MAO-B, respectively), in terms of their substrates, inhibitors, cofactors, subcellular localization and function. To date, no physiological function has been definitively associated with SSAOs, and even the nature of the physiological substrates is not firmly established (reviewed in Buffoni F. and Ignesti G. (2000) Mol. Genetics. Metabl. 71:559-564). However, they have been implicated in the metabolism of exogenous and endogenous amines and in the regulation of glucose transport.

SSAO molecules are highly conserved across species; the closest homologue to the human protein is the bovine serum amine oxidase (about 85% identity). Substrate specificity and tissue distribution vary considerably among different species. In humans, SSAO specific activity has been detected in most tissues but with marked differences (highest in aorta and lung). Human and rodent plasma have very low SSAO activity compared with ruminants. Depletion studies suggest that SSAO/VAP-1 accounts for ˜90% of cell and serum SSAO activity (Jaakkola K. et al. (1999) Am. J. Pathol. 155:1953).

Membrane-bound VAP-1 is primarily expressed in high endothelial cells (ECs) of lymphatic organs, sinusoidal ECs of the liver and small caliber venules of many other tissues. Moreover, SSAO/VAP-1 is also found in dendritic cells of germinal centers and is abundantly present in adipocytes, pericytes and smooth muscle cells. However, it is absent from capillaries, ECs of large blood vessels, epithelial cells, fibroblasts and leukocytes other than dendritic cells (Salmi M. et al. (2001) Trends Immunol. 22:211). Studies in clinical samples revealed that SSAO/VAP-1 is upregulated on vasculature at many sites of inflammation, such as synovitis, allergic and other skin inflammations, and inflammatory bowel disease (IBD). However, expression appears to be controlled by additional mechanisms. Animal studies indicate that the luminal SSAO/VAP-1 is induced only upon elicitation of inflammation. Thus, in ECs, SSAO/VAP-1 is stored in intracellular granules and is translocated onto the luminal surface only at sites of inflammation.

In the serum of healthy adults a soluble form of SSAO/VAP-1 is found at a concentration of 80 ng/ml. Soluble SSAO/VAP-1 levels increase in certain liver diseases and in diabetes, but remain normal in many other inflammatory conditions. Soluble SSAO/VAP-1 has an N-terminal amino acid sequence identical to the proximal extracellular sequence of the membrane bound form of SSAO/VAP-1. In addition, there is good evidence that at least a significant portion of the soluble molecule is produced in the liver by proteolytic cleavage of sinusoidal VAP-1 (Kurkijarvi R. et al. (2000) Gastroenterology 119:1096).

SSAO/VAP-1 regulates leukocyte adhesion to ECs. Studies show that SSAO/VAP-1 is involved in the adhesion cascade at sites where induction/activation of selectins, chemokines, immunoglobulin superfamily molecules, and integrins takes place. In the appropriate context, nevertheless, inactivation of SSAO/VAP-1 function has an independent and significant effect on the overall extravasion process. A recent study shows that both the direct adhesive and enzymatic functions of SSAO/VAP-1 are involved in the adhesion cascade (Salmi M. et al. (2001) Immunity 14:265). In this study, it was proposed that the SSAO activity of VAP-1 is directly involved in the pathway of leukocyte adhesion to endothelial cells by a novel mechanism involving direct interaction with an amine substrate presented on a VAP-1 ligand expressed on the surface of a leukocyte. Under physiological laminar shear, it seems that SSAO/VAP-1 first comes into play after tethering (which takes place via binding of selectins to their ligands) when lymphocytes start to roll on ECs. Accordingly, anti-VAP-1 monoclonal antibodies inhibit ˜50% of lymphocyte rolling and significantly reduce the number of firmly bound cells. In addition, inhibition of VAP-1 enzymatic activity by SSAO inhibitors, also results in a >40% reduction in the number of rolling and firmly bound lymphocytes. Thus, inhibitors of SSAO/VAP-1 enzymatic activity could reduce leukocyte adhesion in areas of inflammation and thereby reduce leukocyte trafficking into the inflamed region and, consequently, reduce the inflammatory process itself.

Increased SSAO activity has been found in the plasma and islets of Type I and Type II diabetes patients and animal models, as well as after congestive heart failure, and in an atherosclerosis mouse model (Salmi M., et al. (2002) Am. J. Pathol. 161:2255; Bono P. et al (1999) Am. J. Pathol. 155:1613; Boomsma F. et al (1999) Diabetologia 42:233; Gronvall-Nordquist J. et al (2001) J. Diabetes Complications 15:250; Ferre I. et al. (2002) Neurosci. Lett. 15; 321: 21; Conklin D. J. et al. (1998) Toxicological Sciences 46: 386; Yu P. H. and Deng Y. L. (1998) Atherosclerosis 140:357; Vidrio H. et al. (2002) General Pharmacology 35:195; Conklin D. J. (1999) Toxicology 138: 137). In addition to upregulation of expression of VAP-1 in the inflamed joints of rheumatoid arthritis (RA) patients and in the venules from lamina propria and Peyer's patches of IBD patients, increased synthesis of VAP-1 was also found in chronic skin inflammation and liver disease (Lalor P. F. et al. (2002) J. Immunol. 169:983; Jaakkola K. et al. (2000) Am. J. Pathol. 157:463; Salmi M. and Jalkanen S. (2001) J. Immunol. 166:4650; Lalr P. F. et al. (2002) Immunol Cell Biol 80:52; Salmi M et al. (1997) J. Clin. Invest. 99:2165; Kurkijarvi R. et al. (1998) J. Immunol. 161:1549).

In summary, SSAO/VAP-1 is an inducible endothelial enzyme that mediates the interaction between leukocytes and inflamed vessels. The fact that SSAO/VAP-1 has both enzymatic and adhesion activities together with the strong correlation between its upregulation in many inflammatory conditions, makes it a potential therapeutic target for all the above-mentioned disease conditions.

### DISCLOSURE OF THE INVENTION

SSAO inhibitors can block inflammation and autoimmune processes, as well as other pathological conditions associated with an increased level of the circulating amine substrates and/or products of SSAO. In one embodiment, the invention relates to a method of inhibiting an inflammatory response by administration of compounds to inhibit SSAO enzyme activity (where the enzyme activity is due either to soluble SSAO enzyme or membrane-bound VAP-1 protein, or due to both) and/or inhibit binding to VAP-1 protein. In another embodiment, the inflammatory response is an acute inflammatory response. In another embodiment, the invention relates to treating or preventing diseases mediated at least in part by SSAO or VAP-1, as generally indicated by one or more of abnormal levels of SSAO and/or VAP-1 or abnormal activity of SSAO and/or VAP-1 (where the abnormal activity of VAP-1 may affect its binding function, its amine oxidase function, or both), by administering a therapeutically effective amount of an SSAO inhibitor, or administering a therapeutically effective combination of SSAO inhibitors. In another embodiment, the invention relates to a method of treating or preventing immune disorders, by administering a therapeutically effective amount of an SSAO inhibitor, or administering a therapeutically effective combination of SSAO inhibitors. In another embodiment, the invention relates to a method of treating or preventing multiple sclerosis (including chronic multiple sclerosis), by administering a therapeutically effective amount of an SSAO inhibitor, or administering a therapeutically effective combination of SSAO inhibitors. In another embodiment, the invention relates to a method of treating or preventing ischemic diseases (for example, stroke) and/or the sequelae thereof (for example, an inflammatory response), by administering a therapeutically effective amount of an SSAO inhibitor, or administering a therapeutically effective combination of SSAO inhibitors. The SSAO inhibitors administered can inhibit the SSAO activity of soluble SSAO, the SSAO activity of membrane-bound VAP-1, binding to membrane-bound VAP-1, or any two of those activities, or all three of those activities. In another embodiment, the invention relates to a method of inhibiting SSAO activity or inhibiting binding to VAP-1 in vitro using the compounds provided herein. In another embodiment, the invention relates to a method of inhibiting SSAO activity or inhibiting binding to VAP-1 in vivo, that is, in a living organism, such as a vertebrate, mammal, or human, using the compounds provided herein.

In another embodiment, the present invention relates to various compounds which are useful for inhibiting SSAO enzyme activity (where the enzyme activity is due either to soluble SSAO enzyme or membrane-bound VAP-1 protein, or due to both) and/or inhibition of binding to membrane-bound VAP-1 protein. In another embodiment, the present invention relates to methods of using various compounds to inhibit SSAO enzyme activity (where the enzyme activity is due either to soluble SSAO enzyme or membrane-bound VAP-1 protein, or due to both). In another embodiment, the present invention relates to methods of inhibiting binding to VAP-1 protein.

In another embodiment, the present invention relates to methods of treating or preventing inflammation, by administering an SSAO inhibitor which has a specificity for inhibition of SSAO as compared to MAO-A and/or MAO-B, of about 10-fold, greater than about 10-fold, about 100-fold, greater than about 100-fold, about 500-fold, greater than about 500-fold, about 1,000-fold, greater than about 1000-fold, about 5,000-fold, or greater than about 5000-fold.

In another embodiment, the present invention relates to methods of treating or preventing an immune or autoimmune disorder, by administering an SSAO inhibitor which has a specificity for inhibition of SSAO as compared to MAO-A and/or MAO-B of about 10-fold, greater than about 10-fold, about 100-fold, greater than about 100-fold, about 500-fold, greater than about 500-fold, about 1,000-fold, greater than about 1000-fold, about 5,000-fold, or greater than about 5000-fold.

In another embodiment, the present invention relates to methods of treating or preventing inflammation, by administering an SSAO inhibitor which has a specificity for inhibition of SSAO as compared to diamine oxidase of about 10-fold, greater than about 10-fold, about 100-fold, greater than about 100-fold, about 500-fold, greater than about 500-fold, about 1,000-fold, greater than about 1000-fold, about 5,000-fold, or greater than about 5000-fold.

In another embodiment, the present invention relates to methods of treating or preventing an immune or autoimmune disorder, by administering an SSAO inhibitor which has a specificity for inhibition of SSAO as compared to diamine oxidase of about 10-fold, greater than about 10-fold, about 100-fold, greater than about 100-fold, about 500-fold, greater than about 500-fold, about 1,000-fold, greater than about 1000-fold, about 5,000-fold, or greater than about 5000-fold.

The inflammation or inflammatory disease or immune or autoimmune disorder to be treated by the SSAO inhibitors of the specificity indicated may be, or may be caused by, multiple sclerosis (including chronic multiple sclerosis); synovitis; systemic inflammatory sepsis; inflammatory bowel diseases; Crohn's disease; ulcerative colitis; Alzheimer's disease; vascular dementia; atherosclerosis; rheumatoid arthritis; juvenile rheumatoid arthritis; pulmonary inflammatory conditions; asthma; skin inflammatory conditions and diseases; contact dermatitis; liver inflammatory and autoimmune conditions; autoimmune hepatitis; primary biliary cirrhosis; sclerosing cholangitis; autoimmune cholangitis; alcoholic liver disease; Type I diabetes and/or complications thereof; Type II diabetes and/or complications thereof; atherosclerosis; chronic heart failure; congestive heart failure; ischemic diseases such as stroke and/or complications thereof; and myocardial infarction and/or complications thereof. In another embodiment, the inflammatory disease or immune disorder to be treated or prevented by the present invention is multiple sclerosis (including chronic multiple sclerosis). In another embodiment, the inflammatory disease or immune disorder to be treated or prevented by the present invention is stroke or the inflammatory complications resulting from stroke.

In another embodiment, the present invention relates to methods of treating or preventing inflammation, by administering one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 as described herein in a therapeutically effective amount, or in an amount sufficient to treat or prevent inflammation. In another embodiment, the present invention relates to methods of treating or preventing immune or autoimmune disorders, by administering one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 described herein in a therapeutically effective amount, or in an amount sufficient to treat or prevent an immune or autoimmune disorder.

In another embodiment, the present invention relates to methods of treating or preventing inflammation, by administering one or more of the compounds I-1, I-2, I-3, I-4, I-5, I-6, I-7, I-8, I-9, I-10, I-11, I-12, I-13, I-14, I-15, I-16, I-17, I-18, I-19, I-20, I-21, I-22, I-23, I-24, I-25, I-26, I-27, I-28, I-29, I-30, I-31, I-32, I-33, I-34, I-35, I-36, I-37, I-38, I-39, I-40, I-41, I-42, I-43, I-44, I-45, I-46, I-47, I-48, I-49, I-50, I-51, I-52, I-53, I-54, I-55, I-56, I-57, I-58, I-59, I-60, I-61, I-62, I-63, I-64, I-65, I-66, I-67, I-68, I-69, I-70, I-71, I-72, I-73, I-74, I-75, I-76, I-77, I-78, I-79, I-80, I-81, I-82, I-83, I-84, I-85, I-86, I-87, I-88, I-89, I-90, I-91, I-92, I-93, I-94, I-95, I-96, I-97, I-98, I-99, I-100, I-101, I-102, I-103, I-104, I-105, I-106, I-107, I-108, I-109, II-1, II-2, II-3, II-4, II-5, II-6, II-7, II-8, II-9, II-10, II-11, II-12, II-13, II-14, II-15, II-16, II-17, II-18, II-19, II-20, II-21, II-22, II-23, IV-1, IV-2, IV-3, IV-4, IV-5, IV-6, IV-7, IV-8, IV-9, or IV-10 described herein in a therapeutically effective amount, or in an amount sufficient to treat or prevent inflammation. In another embodiment, the present invention relates to methods of treating or preventing immune or autoimmune disorders, by administering one or more of the compounds I-1, I-2, I-3, I-4, I-5, I-6, I-7, I-8, I-9, I-10, I-11, I-12, I-13, I-14, I-15, I-16, I-17, I-18, I-19, I-20, I-21, I-22, I-23, I-24, I-25, I-26, I-27, I-28, I-29, I-30, I-31, I-32, I-33, I-34, I-35, I-36, I-37, I-38, I-39, I-40, I-41, I-42, I-43, I-44, I-45, I-46, I-47, I-48, I-49, I-50, I-51, I-52, I-53, I-54, I-55, I-56, I-57, I-58, I-59, I-60, I-61, I-62, I-63, I-64, I-65, I-66, I-67, I-68, I-69, I-70, I-71, I-72, I-73, I-74, I-75, I-76, I-77, I-78, I-79, I-80, I-81, I-82, I-83, I-84, I-85, I-86, I-87, I-88, I-89, I-90, I-91, I-92, I-93, I-94, I-95, I-96, I-97, I-98, I-99, I-100, I-101, I-102, I-103, I-104, I-105, I-106, I-107, I-108, I-109, II-1, II-2, II-3, II-4, II-5, II-6, II-7, II-8, II-9, II-10, II-11, II-12, II-13, II-14, II-15, II-16, II-17, II-18, II-19, II-20, II-21, II-22, II-23, IV-1, IV-2, IV-3, IV-4, IV-5, IV-6, IV-7, IV-8, IV-9, or IV-10 described herein in a therapeutically effective amount, or in an amount sufficient to treat or prevent an immune or autoimmune disorder.

In another embodiment, the invention relates to compounds of formula I:

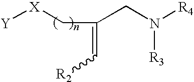

wherein Y is aryl or heteroaryl optionally substituted with one or more groups of the form R₁, wherein each R₁ is independently selected from C₁-C₈ alkyl, C₃-C₈ cycloalkyl, —O—C₁-C₈ alkyl, —O—C₃-C₈ cycloalkyl, —C₆-C₁₀ aryl, —O—C₁-C₄ alkyl-C₆-C₁₀ aryl, —S—C₁-C₈ alkyl, —CF₃, —S—CF₃, —OCF₃, —OCH₂CF₃, F, Cl, Br, I, —NO₂, —OH, —CN, —NR₅R₆, —NHR₇, and —S(O₂)—(C₁-C₈ alkyl); R₂ is selected from H, F, Cl, C₁-C₄ alkyl, and —CF₃; R₃ and R₄ are independently selected from H, —C₁-C₈ alkyl, —C₁-C₄ alkyl-C₆-C₁₀ aryl, or R₃ and R₄ together with the nitrogen to which they are attached form a nitrogen-containing ring (including, but not limited to, morpholino, piperidino, and piperazino); R₅ and R₆ are independently selected from H, —C₁-C₈ alkyl, —C₁-C₄ alkyl-C₆-C₁₀ aryl, or R₅ and R₆ together with the nitrogen to which they are attached form a nitrogen-containing ring (including, but not limited to, morpholino, piperidino, and piperazino); R₇ is selected from —C(═O)—(C₁-C₈ alkyl) and —C(═O)—(C₆-C₁₀ aryl); X is —CH₂—, —O—, or —S—; n is 0, 1, 2, or 3. In another embodiment, R₁ is selected from C₁-C₄ alkyl, —O—C₁-C₄ alkyl or —S—C₁-C₈ alkyl; and any stereoisomer, mixture of stereoisomers, prodrug, metabolite, crystalline form, non-crystalline form, hydrate, solvate, or salt thereof.

In another embodiment, formula I is subject to the proviso that when Y is phenyl, R₃ and R₄ are both H, X is CH₂, and n is 0, then there is at least one R₁ substituent. In another embodiment, formula I is subject to the proviso that when Y is phenyl, R₃ and R₄ are both H, X is CH₂, and n is 0, then if at least one R₁ substituent is —OCH₃, then there is at least one additional R₁ substituent which is not —OCH₃. In another embodiment, formula I is subject to the proviso that when Y is phenyl, R₃ and R₄ are both H, X is CH₂, and n is 0, then if at least one R₁ substituent is —OH, then there is at least one additional R₁ substituent which is not —OH.

In another embodiment, formula I is subject to the proviso that when Y is phenyl, R₃ and R₄ are both H, X is O or S, and n is 1, then there is at least one R₁ substituent. In another embodiment, formula I is subject to the proviso that when Y is phenyl, R₃ and R₄ are both H, X is O or S, and n is 1, then the phenyl substituents are not Cl, —CF₃, or F in the ortho or para position. In another embodiment, formula I is subject to the proviso that when Y is phenyl, R₃ and R₄ are both H, X is O or S, and n is 1, then the phenyl substituents are not 3-chloro-5-fluoro. In another embodiment, formula I is subject to the proviso that when Y is phenyl, R₃ and R₄ are both H, X is O or S, and n is 1, then if at least one R₁ substituent is —OCH₃, then there is at least one additional R₁ substituent is not —OCH₃. In another embodiment, formula I is subject to the proviso that when Y is phenyl, R₃ and R₄ are both H, X is O or S, and n is 1, if at least one R₁ substituent is —OH, then there is at least one additional R₁ substituent which is not —OH.

In another embodiment, formula I is subject to the proviso that when Y is phenyl, R₃ and R₄ are both H, X is CH₃, and n is 1, then there is at least one R₁ substituent. In another embodiment, formula I is subject to the proviso that when Y is phenyl, R₃ and R₄ are both H, X is CH₃, and n is 1, then the phenyl substituent is not F in the para position.

In another embodiment, formula I is subject to the proviso that when Y is phenyl, R₃ and R₄ are both H, X is CH₃, and n is 2, then the phenyl substituents are not 3,4-dimethoxy.

The compounds of formula I with provisos are designated as compounds of formula I-P.

In another embodiment, X is CH₂ and n is 0 or 1. In another embodiment, X is CH₂ and n is 0. In another embodiment, X is CH₂ and n is 1. In another embodiment of the compounds of formula I, Y is phenyl, optionally substituted with one or more R₁ substituents. In another embodiment R₃ and R₄ are both H. In another embodiment, R₂ is F. In another embodiment, R₂ is Cl. In another embodiment, X is O and n is 0.

In another embodiment, the compounds of formula I or I-P are in the E configuration of the double bond; those compounds are designated as compounds of formula I-E or I-P-E, respectively. In another embodiment, the compounds of formula I or I-P are in the Z configuration of the double bond; those compounds are designated as compounds of formula I-Z or I-P-Z, respectively.

In another embodiment, the invention relates to compounds of formula I-A:

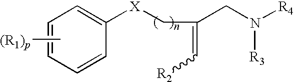

wherein each R₁ is independently selected from H, C₁-C₈ alkyl, C₃-C₈ cycloalkyl, —O—C₁-C₈ alkyl, —O—C₃-C₈ cycloalkyl, —C₆-C₁₀ aryl, —O—C₁-C₄ alkyl-C₆-C₁₀ aryl, —S—C₁-C₈ alkyl, —CF₃, —OCF₃, —S—CF₃, —OCH₂CF₃, F, Cl, Br, I, —NO₂, —OH, —CN, —NR₅R₆, —NHR₇, and —S(O₂)—(C₁-C₈ alkyl); R₂ is selected from H, F, Cl, C₁-C₄ alkyl, and —CF₃; R₃ and R₄ are independently selected from H, —C₁-C₈ alkyl, —C₁-C₄ alkyl-C₆-C₁₀ aryl, or R₃ and R₄ together with the nitrogen to which they are attached form a nitrogen-containing ring (including, but not limited to, morpholino, piperidino, and piperazino); R₅ and R₆ are independently selected from H, —C₁-C₈ alkyl, —C₁-C₄ alkyl-C₆-C₁₀ aryl, or R₅ and R₆ together with the nitrogen to which they are attached form a nitrogen-containing ring (including, but not limited to, morpholino, piperidino, and piperazino); R₇ is selected from —C(═O)—(C₁-C₈ alkyl) and —C(═O)—(C₆-C₁₀ aryl); X is —CH₂—, —O—, or —S—; n is 0, 1, 2, or 3; and p is 0, 1, 2, or 3; and any stereoisomer, mixture of stereoisomers, prodrug, metabolite, crystalline form, non-crystalline form, hydrate, solvate, or salt thereof.

In another embodiment, formula I-A is subject to the proviso that when R₃ and R₄ are both H, X is CH₂, and n is 0, then there is at least one R₁ substituent. In another embodiment, formula I-A is subject to the proviso that when R₃ and R₄ are both H, X is CH₂, and n is 0, then if at least one R₁ substituent is —OCH₃, then there is at least one additional R₁ substituent which is not —OCH₃. In another embodiment, formula I-A is subject to the proviso that when R₃ and R₄ are both H, X is CH₂, and n is 0, then if at least one R₁ substituent is —OH, then there is at least one additional R₁ substituent which is not —OH.

In another embodiment, formula I-A is subject to the proviso that when R₃ and R₄ are both H, X is O or S, and n is 1, then there is at least one R₁ substituent. In another embodiment, formula I-A is subject to the proviso that when R₃ and R₄ are both H, X is O or S, and n is 1, then the phenyl substituents are not Cl, —CF₃, or F in the ortho or para position. In another embodiment, formula I-A is subject to the proviso that when R₃ and R₄ are both H, X is O or S, and n is 1, then the phenyl substituents are not 3-chloro-5-fluoro. In another embodiment, formula I-A is subject to the proviso that when R₃ and R₄ are both H, X is O or S, and n is 1, then if at least one R₁ substituent is —OCH₃, then there is at least one additional R₁ substituent is not —OCH₃. In another embodiment, formula I-A is subject to the proviso that when R₃ and R₄ are both H, X is O or S, and n is 1, then if at least one R₁ substituent is —OH, then there is at least one additional R₁ substituent which is not —OH.

In another embodiment, formula I-A is subject to the proviso that when R₃ and R₄ are both H, X is CH₃, and n is 1, then there is at least one R₁ substituent. In another embodiment, formula I-A is subject to the proviso that when R₃ and R₄ are both H, X is CH₃, and n is 1, then the phenyl substituent is not F in the para position.

The compounds of formula I-A with provisos are designated as compounds of formula I-AP.

In another embodiment, the compounds of formula I-A or I-AP are in the E configuration of the double bond; those compounds are designated as compounds of formula I-A-E or I-AP-E, respectively. In another embodiment, the compounds of formula I-A or I-AP are in the Z configuration of the double bond; those compounds are designated as compounds of formula I-AZ or I-AP-Z, respectively.

In one embodiment of the compounds of formula I-A, X is CH₂ and n is 0 or 1. In another embodiment, X is CH₂ and n is 0. In another embodiment, X is CH₂ and n is 1. In another embodiment, R₃ and R₄ are both H. In another embodiment, R₂ is F. In another embodiment, R₂ is Cl. In another embodiment, X is O and n is 0.

In another embodiment, the invention relates to compounds of formula I-B:

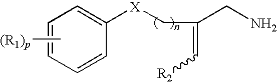

wherein each R₁ is independently selected from H, C₁-C₈ alkyl, C₃-C₈ cycloalkyl, —O—C₁-C₈ alkyl, —O—C₃-C₈ cycloalkyl, —C₆-C₁₀ aryl, —O—C₁-C₄ alkyl-C₆-C₁₀ aryl, —S—C₁-C₈ alkyl, —CF₃, —OCF₃, —S—CF₃, —OCH₂CF₃, F, Cl, Br, I, —NO₂, —OH, —CN, —NR₅R₆, —NHR₇, and —S(O₂)—(C₁-C₈ alkyl); R₂ is selected from H, F, Cl, C₁-C₄ alkyl, and —CF₃; R₅ and R₆ are independently selected from H, —C₁-C₈ alkyl, —C₁-C₄ alkyl-C₆-C₁₀ aryl, or R₅ and R₆ together with the nitrogen to which they are attached form a nitrogen-containing ring (including, but not limited to, morpholino, piperidino, and piperazino); R₇ is selected from —C(═O)—(C₁-C₈ alkyl) and —C(═O)—(C₆-C₁₀ aryl); X is —CH₂— or —O—; n is 0, 1, 2, or 3; and p is 0, 1, 2, or 3; and any stereoisomer, mixture of stereoisomers, prodrug, metabolite, crystalline form, non-crystalline form, hydrate, solvate, or salt thereof.

In another embodiment, formula I-B is subject to the proviso that when X is CH₂, and n is 0, then there is at least one R₁ substituent. In another embodiment, formula I-B is subject to the proviso that when X is CH₂, and n is 0, then if at least one R₁ substituent is —OCH₃, then there is at least one additional R₁ substituent which is not —OCH₃. In another embodiment, formula I-B is subject to the proviso that when X is CH₂, and n is 0, then if at least one R₁ substituent is —OH, then there is at least one additional R₁ substituent which is not —OH.

In another embodiment, formula I-B is subject to the proviso that when X is O or S, and n is 1, then there is at least one R₁ substituent. In another embodiment, formula I-B is subject to the proviso that when X is O or S, and n is 1, then the phenyl substituents are not Cl, —CF₃, or F in the ortho or para position. In another embodiment, formula I-B is subject to the proviso that when X is O or S, and n is 1, then phenyl substituents are not 3-chloro-5-fluoro. In another embodiment, formula I-B is subject to the proviso that when X is O or S, and n is 1, then if at least one R₁ substituent is —OCH₃, then there is at least one additional R₁ substituent which is not —OCH₃. In another embodiment, formula I-B is subject to the proviso that when X is O or S, and n is 1, then if at least one R₁ substituent is —OH, then there is at least one additional R₁ substituent which is not —OH.

In another embodiment, formula I-B is subject to the proviso that when X is CH₃, and n is 1, then there is at least one R₁ substituent. In another embodiment, formula I-B is subject to the proviso that when X is CH₃, and n is 1, then the phenyl substituent is not F in the para position.

The compounds of formula I-B with provisos are designated as compounds of formula I-BP.

In another embodiment, the compounds of formula I-B or I-BP are in the E configuration of the double bond; those compounds are designated as compounds of formula I-B-E or I-BP-E, respectively. In another embodiment, the compounds of formula I-B or I-BP are in the Z configuration of the double bond; those compounds are designated as compounds of formula I-BZ or I-BP-Z, respectively.

In another embodiment of the compounds of formula I-B, X is CH₂ and n is 0 or 1. In another embodiment, X is CH₂ and n is 0. In another embodiment, X is CH₂ and n is 1. In another embodiment, R₂ is F. In another embodiment, R₂ is Cl. In another embodiment, X is O and n is 0.

In one embodiment of the compounds of formula I, I-P, I-E, I-P-E, I-Z, I-P-Z, I-A, I-AP, I-A-E, I-AP-E, I-A-Z, I-AP-Z, I-B, I-BP, I-B-E, I-BP-E, I-B-Z, or I-BP-Z, n is 0. In another embodiment, R₁ is selected from C₁-C₄ alkyl or —O—C₁-C₄ alkyl. In another embodiment, n is 0 and X is —CH₂—. In another embodiment, n is 0, X is —CH₂—, and R₅ and R₆ are H or —C₁-C₈ alkyl. In another embodiment, n is 0, X is —CH₂—, R₅ and R₆ are H or —C₁-C₈ alkyl, and each R₁ is independently selected from H, C₁-C₄ alkyl, C₃-C₈ cycloalkyl, —O—C₁-C₄ alkyl, —S—C₁-C₄ alkyl, CF₃, —OCF₃, F, and Cl. In another embodiment, n is 0, X is —CH₂—, R₅ and R₆ are H or —C₁-C₈ alkyl, each R₁ is independently selected from H, C₁-C₄ alkyl, C₃-C₈ cycloalkyl, —O—C₁-C₄ alkyl, —S—C₁-C₈ alkyl, CF₃, —OCF₃, F, and Cl, and p is 1 or 2. In another embodiment, n is 0, X is —CH₂—, R₅ and R₆ are H or —C₁-C₈ alkyl, and each R₁ is independently selected from H, C₁-C₄ alkyl, —S—C₁-C₄ alkyl, and —O—C₁-C₄ alkyl. In another embodiment, n is 0, X is —CH₂—, R₅ and R₆ are H or —C₁-C₈ alkyl, each R₁ is independently selected from H, C₁-C₄ alkyl, —S—C₁-C₄ alkyl, and —O—C₁-C₄ alkyl, and p is 1 or 2. In another embodiment, n is 0, X is —CH₂—, R₅ and R₆ are H or —C₁-C₈ alkyl, each R₁ is independently selected from H, C₁-C₄ alkyl, —S—C₁-C₄ alkyl, and —O—C₁-C₄ alkyl, and p is 1.

In another embodiment, the present invention relates to any one of the compounds of general formula I of the formula:

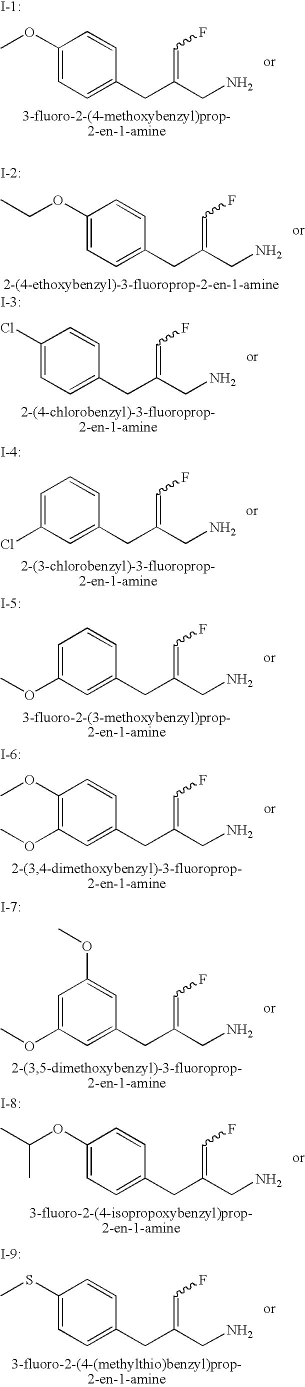

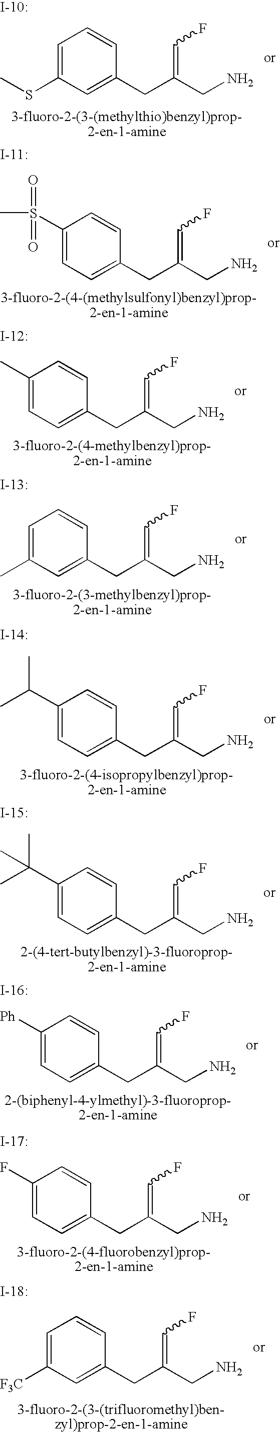

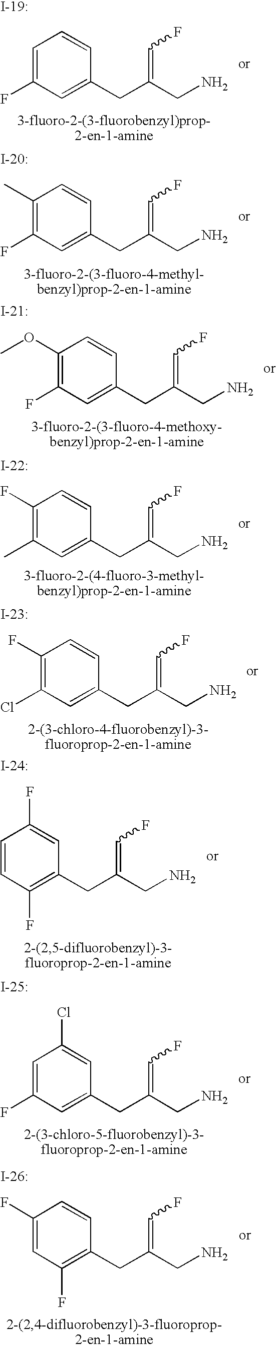

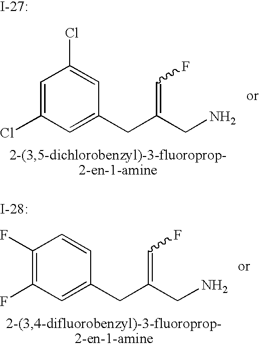

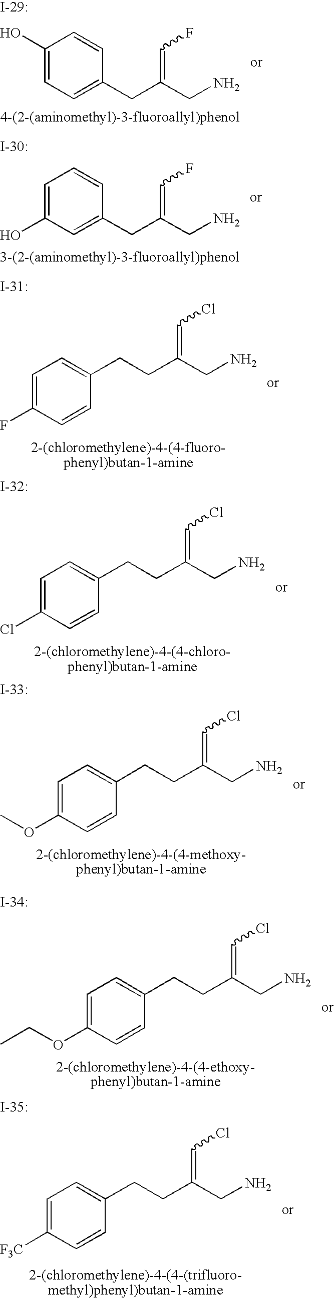

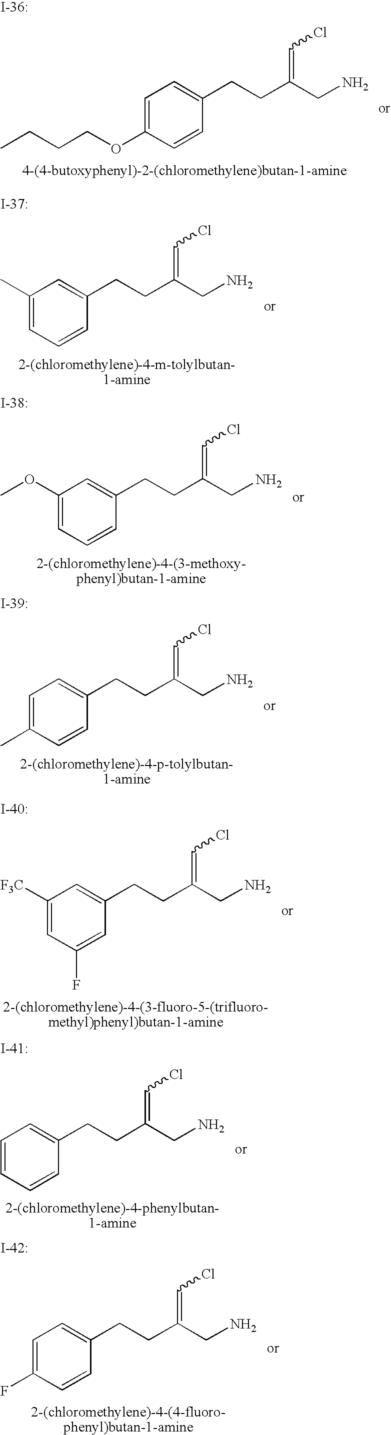

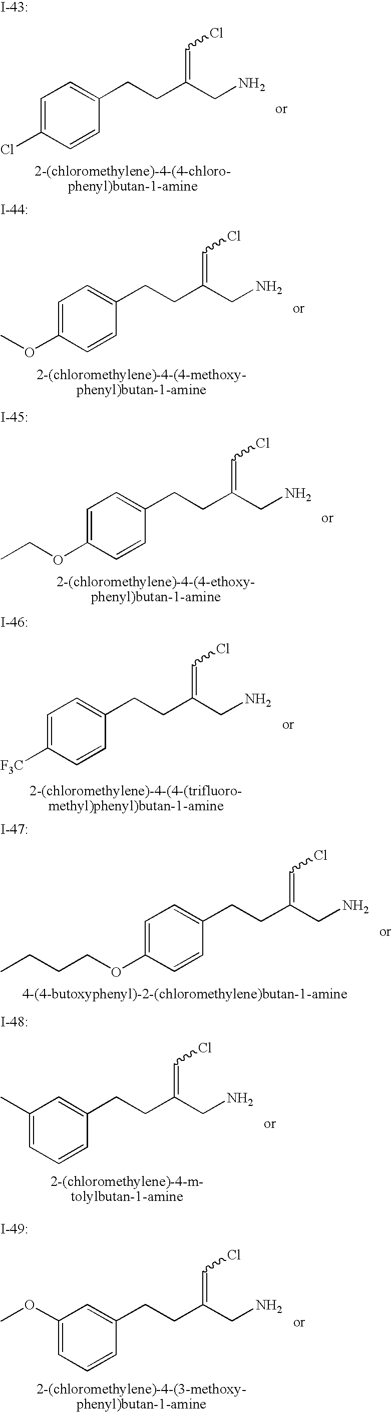

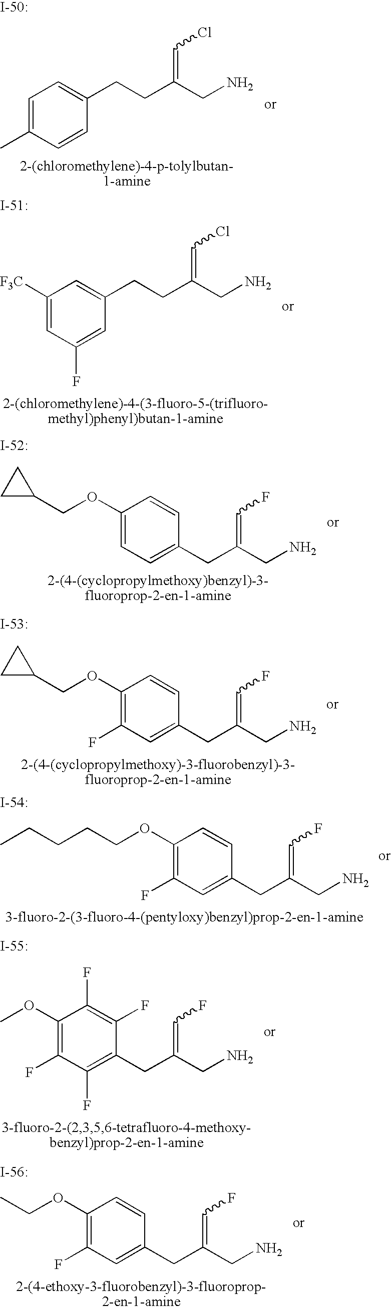

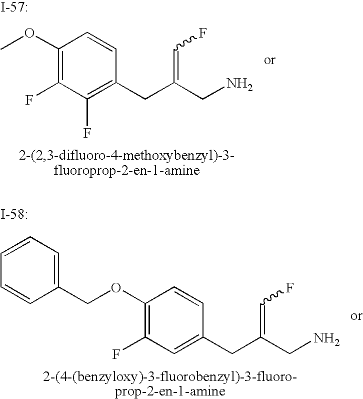

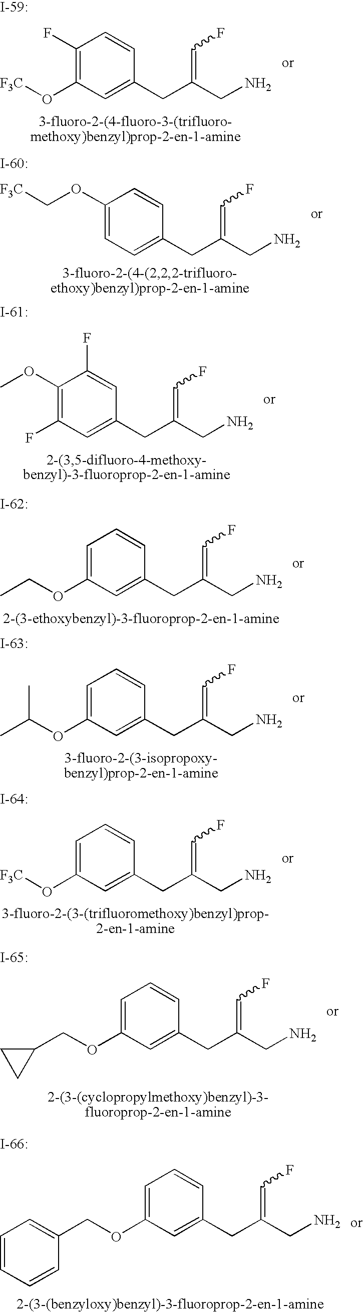

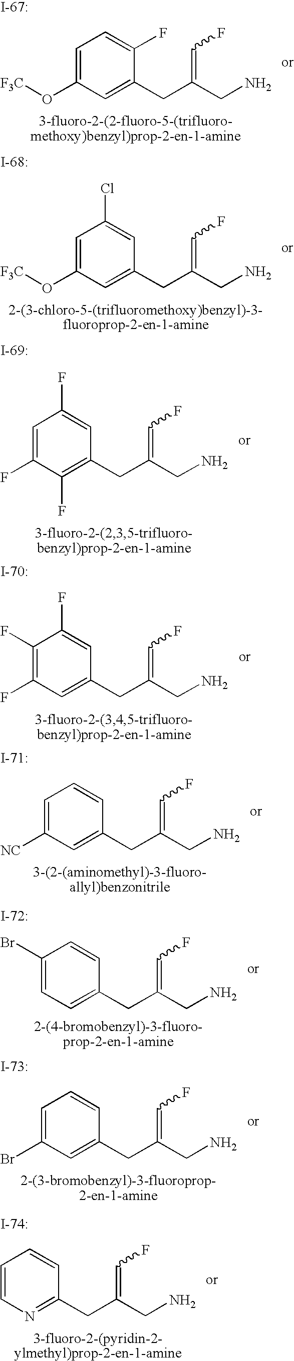

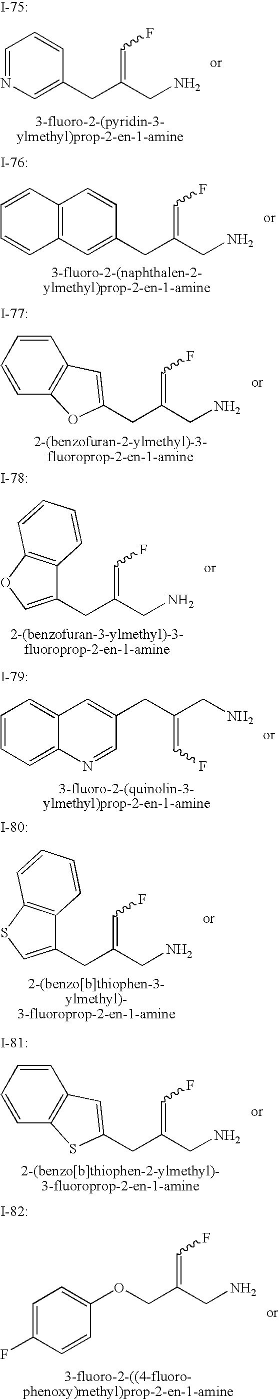

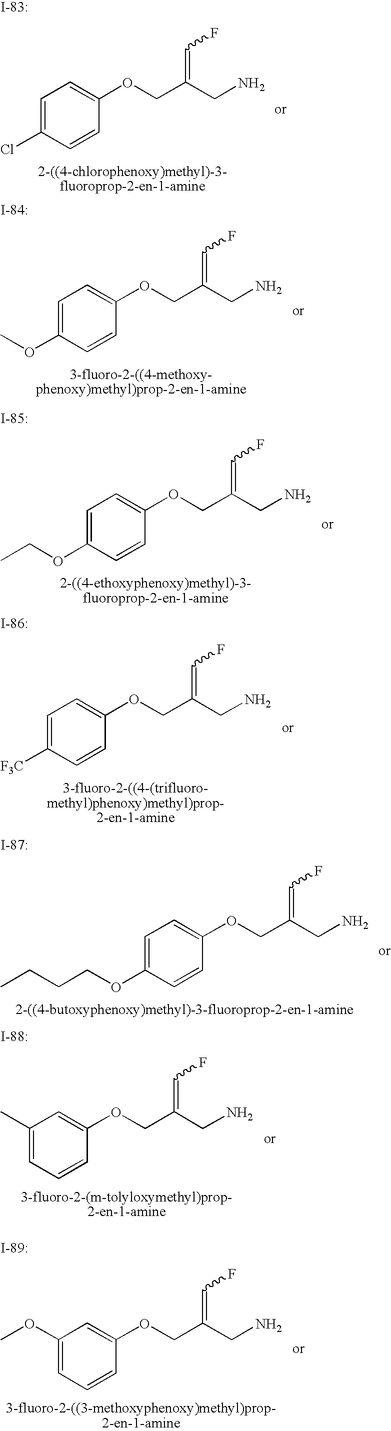

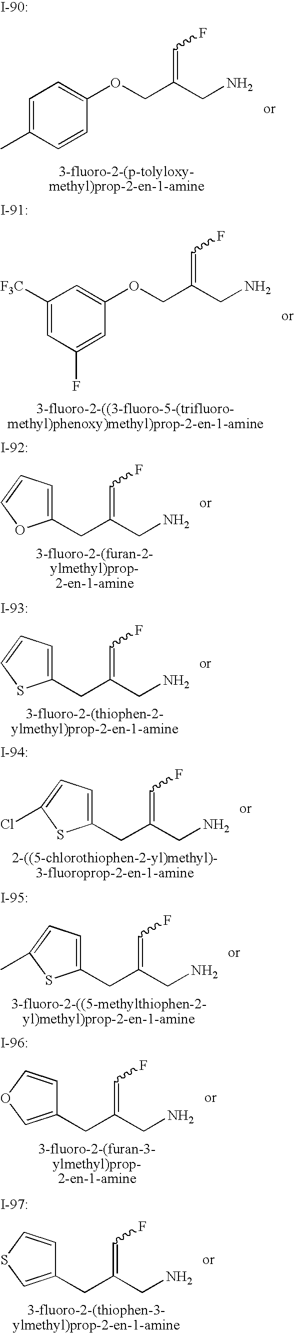

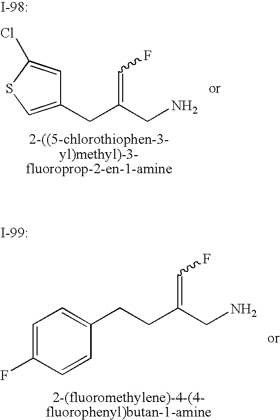

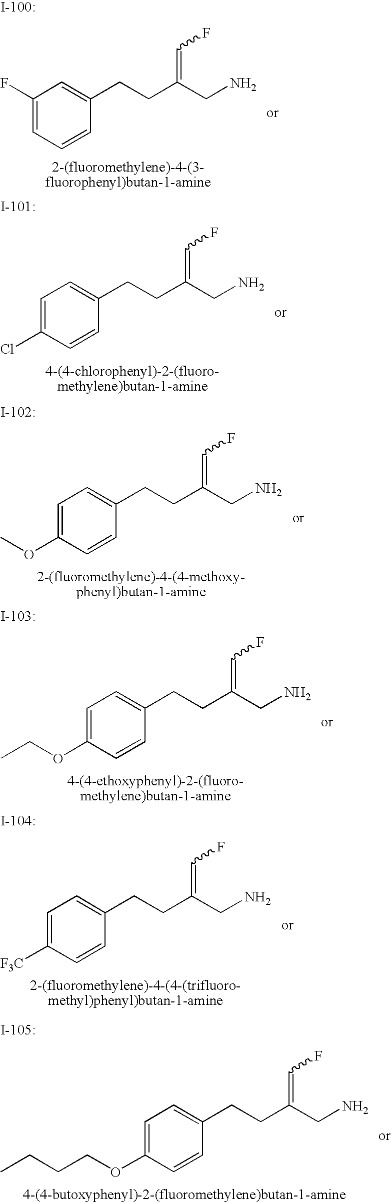

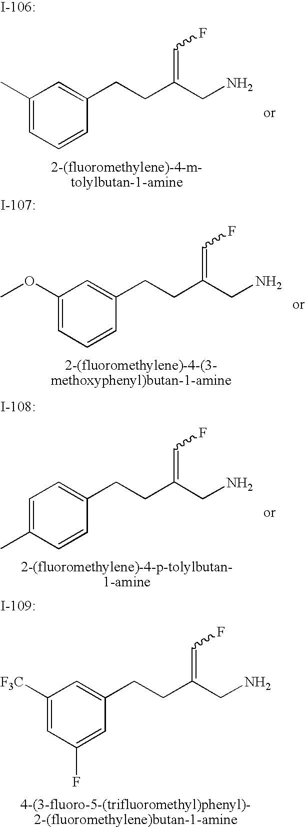

or any stereoisomer, mixture of stereoisomers, prodrug, metabolite, crystalline form, non-crystalline form, hydrate, solvate, or salt thereof.

In another embodiment, the present invention relates to any one of the compounds of general formula I:
I-1-Z: (Z)-3-fluoro-2-(4-methoxybenzyl)prop-2-en-1-amine,
I-2-Z: (Z)-2-(4-ethoxybenzyl)-3-fluoroprop-2-en-1-amine,
I-3-Z: (Z)-2-(4-chlorobenzyl)-3-fluoroprop-2-en-1-amine,
I-4-Z: (Z)-2-(3-chlorobenzyl)-3-fluoroprop-2-en-1-amine,
I-5-Z: (Z)-3-fluoro-2-(3-methoxybenzyl)prop-2-en-1-amine,
I-6-Z: (Z)-2-(3,4-dimethoxybenzyl)-3-fluoroprop-2-en-1-amine,
I-7-Z: (Z)-2-(3,5-dimethoxybenzyl)-3-fluoroprop-2-en-1-amine,
I-8-Z: (Z)-3-fluoro-2-(4-isopropoxybenzyl)prop-2-en-1-amine,
I-9-Z: (Z)-3-fluoro-2-(4-(methylthio)benzyl)prop-2-en-1-amine,
I-10-Z: (Z)-3-fluoro-2-(3-(methylthio)benzyl)prop-2-en-1-amine,
I-11-Z: (Z)-3-fluoro-2-(4-(methylsulfonyl)benzyl)prop-2-en-1-amine,
I-12-Z: (Z)-3-fluoro-2-(4-methylbenzyl)prop-2-en-1-amine,
I-13-Z: (Z)-3-fluoro-2-(3-methylbenzyl)prop-2-en-1-amine,
I-14-Z: (Z)-3-fluoro-2-(4-isopropylbenzyl)prop-2-en-1-amine,
I-15-Z: (Z)-2-(4-tert-butylbenzyl)-3-fluoroprop-2-en-1-amine,
I-16-Z: (Z)-2-(biphenyl-4-ylmethyl)-3-fluoroprop-2-en-1-amine,
I-17-Z: (Z)-3-fluoro-2-(4-fluorobenzyl)prop-2-en-1-amine,
I-18-Z: (Z)-3-fluoro-2-(3-(trifluoromethyl)benzyl)prop-2-en-1-amine,
I-19-Z: (Z)-3-fluoro-2-(3-fluorobenzyl)prop-2-en-1-amine,
I-20-Z: (Z)-3-fluoro-2-(3-fluoro-4-methylbenzyl)prop-2-en-1-amine,
I-21-Z: (Z)-3-fluoro-2-(3-fluoro-4-methoxybenzyl)prop-2-en-1-amine,
I-22-Z: (Z)-3-fluoro-2-(4-fluoro-3-methylbenzyl)prop-2-en-1-amine,
I-23-Z: (Z)-2-(3-chloro-4-fluorobenzyl)-3-fluoroprop-2-en-1-amine,
I-24-Z: (Z)-2-(2,5-difluorobenzyl)-3-fluoroprop-2-en-1-amine,
I-25-Z: (Z)-2-(3-chloro-5-fluorobenzyl)-3-fluoroprop-2-en-1-amine,
I-26-Z: (Z)-2-(2,4-difluorobenzyl)-3-fluoroprop-2-en-1-amine,
I-27-Z: (Z)-2-(3,5-dichlorobenzyl)-3-fluoroprop-2-en-1-amine,
I-28-Z: (Z)-2-(3,4-difluorobenzyl)-3-fluoroprop-2-en-1-amine,
I-29-Z: (Z)-4-(2-(aminomethyl)-3-fluoroallyl)phenol,
I-30-Z: (Z)-3-(2-(aminomethyl)-3-fluoroallyl)phenol,
I-31-Z: (Z)-2-(chloromethylene)-4-(4-fluorophenyl)butan-1-amine,
I-32-Z: (Z)-2-(chloromethylene)-4-(4-chlorophenyl)butan-1-amine,
I-33-Z: (Z)-2-(chloromethylene)-4-(4-methoxyphenyl)butan-1-amine,
I-34-Z: (Z)-2-(chloromethylene)-4-(4-ethoxyphenyl)butan-1-amine,
I-35-Z: (Z)-2-(chloromethylene)-4-(4-(trifluoromethyl)phenyl)butan-1-amine,
I-36-Z: (Z)-4-(4-butoxyphenyl)-2-(chloromethylene)butan-1-amine,
I-37-Z: (Z)-2-(chloromethylene)-4-m-tolylbutan-1-amine,
I-38-Z: (Z)-2-(chloromethylene)-4-(3-methoxyphenyl)butan-1-amine,
I-39-Z: (Z)-2-(chloromethylene)-4-p-tolylbutan-1-amine,
I-40-Z: (Z)-2-(chloromethylene)-4-(3-fluoro-5-(trifluoromethyl)phenyl)butan-1-amine,
I-41-Z: (Z)-2-(chloromethylene)-4-phenylbutan-1-amine,
I-42-Z: (Z)-2-(chloromethylene)-4-(4-fluorophenyl)butan-1-amine,
I-43-Z: (Z)-2-(chloromethylene)-4-(4-chlorophenyl)butan-1-amine,
I-44-Z: (Z)-2-(chloromethylene)-4-(4-methoxyphenyl)butan-1-amine,
I-45-Z: (Z)-2-(chloromethylene)-4-(4-ethoxyphenyl)butan-1-amine,
I-46-Z: (Z)-2-(chloromethylene)-4-(4-(trifluoromethyl)phenyl)butan-1-amine,
I-47-Z: (Z)-4-(4-butoxyphenyl)-2-(chloromethylene)butan-1-amine,
I-48-Z: (Z)-2-(chloromethylene)-4-m-tolylbutan-1-amine,
I-49-Z: (Z)-2-(chloromethylene)-4-(3-methoxyphenyl)butan-1-amine,
I-50-Z: (Z)-2-(chloromethylene)-4-p-tolylbutan-1-amine,
I-51-Z: (Z)-2-(chloromethylene)-4-(3-fluoro-5-(trifluoromethyl)phenyl)butan-1-amine,
I-52-Z: (Z)-2-(4-(cyclopropylmethoxy)benzyl)-3-fluoroprop-2-en-1-amine,
I-53-Z: (Z)-2-(4-(cyclopropylmethoxy)-3-fluorobenzyl)-3-fluoroprop-2-en-1-amine,
I-54-Z: (Z)-3-fluoro-2-(3-fluoro-4-(pentyloxy)benzyl)prop-2-en-1-amine,
I-55-Z: (Z)-3-fluoro-2-(2,3,5,6-tetrafluoro-4-methoxybenzyl)prop-2-en-1-amine,
I-56-Z: (Z)-2-(4-ethoxy-3-fluorobenzyl)-3-fluoroprop-2-en-1-amine,
I-57-Z: (Z)-2-(2,3-difluoro-4-methoxybenzyl)-3-fluoroprop-2-en-1-amine,
I-58-Z: (Z)-2-(4-(benzyloxy)-3-fluorobenzyl)-3-fluoroprop-2-en-1-amine,
I-59-Z: (Z)-3-fluoro-2-(4-fluoro-3-(trifluoromethoxy)benzyl)prop-2-en-1-amine,
I-60-Z: (Z)-3-fluoro-2-(4-(2,2,2-trifluoroethoxy)benzyl)prop-2-en-1-amine,
I-61-Z: (Z)-2-(3,5-difluoro-4-methoxybenzyl)-3-fluoroprop-2-en-1-amine,
I-62-Z: (Z)-2-(3-ethoxybenzyl)-3-fluoroprop-2-en-1-amine,
I-63-Z: (Z)-3-fluoro-2-(3-isopropoxybenzyl)prop-2-en-1-amine,
I-64-Z: (Z)-3-fluoro-2-(3-(trifluoromethoxy)benzyl)prop-2-en-1-amine,
I-65-Z: (Z)-2-(3-(cyclopropylmethoxy)benzyl)-3-fluoroprop-2-en-1-amine,
I-66-Z: (Z)-2-(3-(benzyloxy)benzyl)-3-fluoroprop-2-en-1-amine,
I-67-Z: (Z)-3-fluoro-2-(2-fluoro-5-(trifluoromethoxy)benzyl)prop-2-en-1-amine,
I-68-Z: (Z)-2-(3-chloro-5-(trifluoromethoxy)benzyl)-3-fluoroprop-2-en-1-amine,
I-69-Z: (Z)-3-fluoro-2-(2,3,5-trifluorobenzyl)prop-2-en-1-amine,
I-70-Z: (Z)-3-fluoro-2-(3,4,5-trifluorobenzyl)prop-2-en-1-amine,
I-71-Z: (Z)-3-(2-(aminomethyl)-3-fluoroallyl)benzonitrile,
I-72-Z: (Z)-2-(4-bromobenzyl)-3-fluoroprop-2-en-1-amine,
I-73-Z: (Z)-2-(3-bromobenzyl)-3-fluoroprop-2-en-1-amine,
I-74-Z: (Z)-3-fluoro-2-(pyridin-2-ylmethyl)prop-2-en-1-amine,
I-75-Z: (Z)-3-fluoro-2-(pyridin-3-ylmethyl)prop-2-en-1-amine,
I-76-Z: (Z)-3-fluoro-2-(naphthalen-2-ylmethyl)prop-2-en-1-amine,
I-77-Z: (Z)-2-(benzofuran-2-ylmethyl)-3-fluoroprop-2-en-1-amine,
I-78-Z: (Z)-2-(benzofuran-3-ylmethyl)-3-fluoroprop-2-en-1-amine,
I-79-Z: (Z)-3-fluoro-2-(quinolin-3-ylmethyl)prop-2-en-1-amine,
I-80-Z: (Z)-2-(benzo[b]thiophen-3-ylmethyl)-3-fluoroprop-2-en-1-amine,
I-81-Z: (Z)-2-(benzo[b]thiophen-2-ylmethyl)-3-fluoroprop-2-en-1-amine,
I-82-E: (E)-3-fluoro-2-((4-fluorophenoxy)methyl)prop-2-en-1-amine,
I-83-E: (E)-2-((4-chlorophenoxy)methyl)-3-fluoroprop-2-en-1-amine,
I-84-E: (E)-3-fluoro-2-((4-methoxyphenoxy)methyl)prop-2-en-1-amine,
I-85-E: (E)-2-((4-ethoxyphenoxy)methyl)-3-fluoroprop-2-en-1-amine,
I-86-E: (E)-3-fluoro-2-((4-(trifluoromethyl)phenoxy)methyl)prop-2-en-1-amine,
I-87-E: (E)-2-((4-butoxyphenoxy)methyl)-3-fluoroprop-2-en-1-amine,
I-88-E: (E)-3-fluoro-2-(m-tolyloxymethyl)prop-2-en-1-amine,
I-89-E: (E)-3-fluoro-2-((3-methoxyphenoxy)methyl)prop-2-en-1-amine,
I-90-E: (E)-3-fluoro-2-(p-tolyloxymethyl)prop-2-en-1-amine,
I-91-E: (E)-3-fluoro-2-((3-fluoro-5-(trifluoromethyl)phenoxy)methyl)prop-2-en-1-amine,
I-92-Z: (Z)-3-fluoro-2-(furan-2-ylmethyl)prop-2-en-1-amine,
I-93-Z: (Z)-3-fluoro-2-(thiophen-2-ylmethyl)prop-2-en-1-amine,
I-94-Z: (Z)-2-((5-chlorothiophen-2-yl)methyl)-3-fluoroprop-2-en-1-amine,
I-95-Z: (Z)-3-fluoro-2-((5-methylthiophen-2-yl)methyl)prop-2-en-1-amine,
I-96-Z: (Z)-3-fluoro-2-(furan-3-ylmethyl)prop-2-en-1-amine,
I-97-Z: (Z)-3-fluoro-2-(thiophen-3-ylmethyl)prop-2-en-1-amine,
I-98-Z: (Z)-2-((5-chlorothiophen-3-yl)methyl)-3-fluoroprop-2-en-1-amine,
I-99-Z: (Z)-2-(fluoromethylene)-4-(4-fluorophenyl)butan-1-amine,
I-100-Z: (Z)-2-(fluoromethylene)-4-(3-fluorophenyl)butan-1-amine,
I-101-Z: (Z)-4-(4-chlorophenyl)-2-(fluoromethylene)butan-1-amine,
I-102-Z: (Z)-2-(fluoromethylene)-4-(4-methoxyphenyl)butan-1-amine,
I-103-Z: (Z)-4-(4-ethoxyphenyl)-2-(fluoromethylene)butan-1-amine,
I-104-Z: (Z)-2-(fluoromethylene)-4-(4-(trifluoromethyl)phenyl)butan-1-amine,
I-105-Z: (Z)-4-(4-butoxyphenyl)-2-(fluoromethylene)butan-1-amine,
I-106-Z: (Z)-2-(fluoromethylene)-4-m-tolylbutan-1-amine,
I-107-Z: (Z)-2-(fluoromethylene)-4-(3-methoxyphenyl)butan-1-amine,
I-108-Z: (Z)-2-(fluoromethylene)-4-p-tolylbutan-1-amine,
I-109-Z: (Z)-4-(3-fluoro-5-(trifluoromethyl)phenyl)-2-(fluoromethylene)butan-1-amine,
   or any stereoisomer, mixture of stereoisomers, prodrug, metabolite, crystalline form, non-crystalline form, hydrate, solvate, or salt thereof.

In another embodiment, the present invention relates to any one of the compounds of general formula I:
I-1-E: (E)-3-fluoro-2-(4-methoxybenzyl)prop-2-en-1-amine,
I-2-E: (E)-2-(4-ethoxybenzyl)-3-fluoroprop-2-en-1-amine,
I-3-E: (E)-2-(4-chlorobenzyl)-3-fluoroprop-2-en-1-amine,
I-4-E: (E)-2-(3-chlorobenzyl)-3-fluoroprop-2-en-1-amine,
I-5-E: (E)-3-fluoro-2-(3-methoxybenzyl)prop-2-en-1-amine,
I-6-E: (E)-2-(3,4-dimethoxybenzyl)-3-fluoroprop-2-en-1-amine,
I-7-E: (E)-2-(3,5-dimethoxybenzyl)-3-fluoroprop-2-en-1-amine,
I-8-E: (E)-3-fluoro-2-(4-isopropoxybenzyl)prop-2-en-1-amine,
I-9-E: (E)-3-fluoro-2-(4-(methylthio)benzyl)prop-2-en-1-amine,
I-10-E: (E)-3-fluoro-2-(3-(methylthio)benzyl)prop-2-en-1-amine,
I-11-E: (E)-3-fluoro-2-(4-(methylsulfonyl)benzyl)prop-2-en-1-amine,
I-12-E: (E)-3-fluoro-2-(4-methylbenzyl)prop-2-en-1-amine,
I-13-E: (E)-3-fluoro-2-(3-methylbenzyl)prop-2-en-1-amine,
I-14-E: (E)-3-fluoro-2-(4-isopropylbenzyl)prop-2-en-1-amine,
I-15-E: (E)-2-(4-tert-butylbenzyl)-3-fluoroprop-2-en-1-amine,
I-16-E: (E)-2-(biphenyl-4-ylmethyl)-3-fluoroprop-2-en-1-amine,
I-17-E: (E)-3-fluoro-2-(4-fluorobenzyl)prop-2-en-1-amine,
I-18-E: (E)-3-fluoro-2-(3-(trifluoromethyl)benzyl)prop-2-en-1-amine,
I-19-E: (E)-3-fluoro-2-(3-fluorobenzyl)prop-2-en-1-amine,
I-20-E: (E)-3-fluoro-2-(3-fluoro-4-methylbenzyl)prop-2-en-1-amine,
I-21-E: (E)-3-fluoro-2-(3-fluoro-4-methoxybenzyl)prop-2-en-1-amine,
I-22-E: (E)-3-fluoro-2-(4-fluoro-3-methylbenzyl)prop-2-en-1-amine,
I-23-E: (E)-2-(3-chloro-4-fluorobenzyl)-3-fluoroprop-2-en-1-amine,
I-24-E: (E)-2-(2,5-difluorobenzyl)-3-fluoroprop-2-en-1-amine,
I-25-E: (E)-2-(3-chloro-5-fluorobenzyl)-3-fluoroprop-2-en-1-amine,
I-26-E: (E)-2-(2,4-difluorobenzyl)-3-fluoroprop-2-en-1-amine,
I-27-E: (E)-2-(3,5-dichlorobenzyl)-3-fluoroprop-2-en-1-amine,
I-28-E: (E)-2-(3,4-difluorobenzyl)-3-fluoroprop-2-en-1-amine,
I-29-E: (E)-4-(2-(aminomethyl)-3-fluoroallyl)phenol,
I-30-E: (E)-3-(2-(aminomethyl)-3-fluoroallyl)phenol,
I-31-E: (E)-2-(chloromethylene)-4-(4-fluorophenyl)butan-1-amine,
I-32-E: (E)-2-(chloromethylene)-4-(4-chlorophenyl)butan-1-amine,
I-33-E: (E)-2-(chloromethylene)-4-(4-methoxyphenyl)butan-1-amine,
I-34-E: (E)-2-(chloromethylene)-4-(4-ethoxyphenyl)butan-1-amine,
I-35-E: (E)-2-(chloromethylene)-4-(4-(trifluoromethyl)phenyl)butan-1-amine,
I-36-E: (E)-4-(4-butoxyphenyl)-2-(chloromethylene)butan-1-amine,
I-37-E: (E)-2-(chloromethylene)-4-m-tolylbutan-1-amine,
I-38-E: (E)-2-(chloromethylene)-4-(3-methoxyphenyl)butan-1-amine,
I-39-E: (E)-2-(chloromethylene)-4-p-tolylbutan-1-amine,
I-40-E: (E)-2-(chloromethylene)-4-(3-fluoro-5-(trifluoromethyl)phenyl)butan-1-amine,
I-41-E: (E)-2-(chloromethylene)-4-phenylbutan-1-amine,
I-42-E: (E)-2-(chloromethylene)-4-(4-fluorophenyl)butan-1-amine,
I-43-E: (E)-2-(chloromethylene)-4-(4-chlorophenyl)butan-1-amine,
I-44-E: (E)-2-(chloromethylene)-4-(4-methoxyphenyl)butan-1-amine,
I-45-E: (E)-2-(chloromethylene)-4-(4-ethoxyphenyl)butan-1-amine,
I-46-E: (E)-2-(chloromethylene)-4-(4-(trifluoromethyl)phenyl)butan-1-amine,
I-47-E: (E)-4-(4-butoxyphenyl)-2-(chloromethylene)butan-1-amine,
I-48-E: (E)-2-(chloromethylene)-4-m-tolylbutan-1-amine,
I-49-E: (E)-2-(chloromethylene)-4-(3-methoxyphenyl)butan-1-amine,
I-50-E: (E)-2-(chloromethylene)-4-p-tolylbutan-1-amine,
I-51-E: (E)-2-(chloromethylene)-4-(3-fluoro-5-(trifluoromethyl)phenyl)butan-1-amine,
I-52-E: (E)-2-(4-(cyclopropylmethoxy)benzyl)-3-fluoroprop-2-en-1-amine,
I-53-E: (E)-2-(4-(cyclopropylmethoxy)-3-fluorobenzyl)-3-fluoroprop-2-en-1-amine,
I-54-E: (E)-3-fluoro-2-(3-fluoro-4-(pentyloxy)benzyl)prop-2-en-1-amine,
I-55-E: (E)-3-fluoro-2-(2,3,5,6-tetrafluoro-4-methoxybenzyl)prop-2-en-1-amine,
I-56-E: (E)-2-(4-ethoxy-3-fluorobenzyl)-3-fluoroprop-2-en-1-amine,
I-57-E: (E)-2-(2,3-difluoro-4-methoxybenzyl)-3-fluoroprop-2-en-1-amine,
I-58-E: (E)-2-(4-(benzyloxy)-3-fluorobenzyl)-3-fluoroprop-2-en-1-amine,
I-59-E: (E)-3-fluoro-2-(4-fluoro-3-(trifluoromethoxy)benzyl)prop-2-en-1-amine,
I-60-E: (E)-3-fluoro-2-(4-(2,2,2-trifluoroethoxy)benzyl)prop-2-en-1-amine,
I-61-E: (E)-2-(3,5-difluoro-4-methoxybenzyl)-3-fluoroprop-2-en-1-amine,
I-62-E: (E)-2-(3-ethoxybenzyl)-3-fluoroprop-2-en-1-amine,
I-63-E: (E)-3-fluoro-2-(3-isopropoxybenzyl)prop-2-en-1-amine,
I-64-E: (E)-3-fluoro-2-(3-(trifluoromethoxy)benzyl)prop-2-en-1-amine,
I-65-E: (E)-2-(3-(cyclopropylmethoxy)benzyl)-3-fluoroprop-2-en-1-amine,
I-66-E: (E)-2-(3-(benzyloxy)benzyl)-3-fluoroprop-2-en-1-amine,
I-67-E: (E)-3-fluoro-2-(2-fluoro-5-(trifluoromethoxy)benzyl)prop-2-en-1-amine,
I-68-E: (E)-2-(3-chloro-5-(trifluoromethoxy)benzyl)-3-fluoroprop-2-en-1-amine,
I-69-E: (E)-3-fluoro-2-(2,3,5-trifluorobenzyl)prop-2-en-1-amine,
I-70-E: (E)-3-fluoro-2-(3,4,5-trifluorobenzyl)prop-2-en-1-amine,
I-71-E: (E)-3-(2-(aminomethyl)-3-fluoroallyl)benzonitrile,
I-72-E: (E)-2-(4-bromobenzyl)-3-fluoroprop-2-en-1-amine,
I-73-E: (E)-2-(3-bromobenzyl)-3-fluoroprop-2-en-1-amine,
I-74-E: (E)-3-fluoro-2-(pyridin-2-ylmethyl)prop-2-en-1-amine,
I-75-E: (E)-3-fluoro-2-(pyridin-3-ylmethyl)prop-2-en-1-amine,
I-76-E: (E)-3-fluoro-2-(naphthalen-2-ylmethyl)prop-2-en-1-amine,
I-77-E: (E)-2-(benzofuran-2-ylmethyl)-3-fluoroprop-2-en-1-amine,
I-78-E: (E)-2-(benzofuran-3-ylmethyl)-3-fluoroprop-2-en-1-amine,
I-79-E: (E)-3-fluoro-2-(quinolin-3-ylmethyl)prop-2-en-1-amine,
I-80-E: (E)-2-(benzo[b]thiophen-3-ylmethyl)-3-fluoroprop-2-en-1-amine,
I-81-E: (E)-2-(benzo[b]thiophen-2-ylmethyl)-3-fluoroprop-2-en-1-amine,
I-82-Z: (Z)-3-fluoro-2-((4-fluorophenoxy)methyl)prop-2-en-1-amine,
I-83-Z: (Z)-2-((4-chlorophenoxy)methyl)-3-fluoroprop-2-en-1-amine,
I-84-Z: (Z)-3-fluoro-2-((4-methoxyphenoxy)methyl)prop-2-en-1-amine,
I-85-Z: (Z)-2-((4-ethoxyphenoxy)methyl)-3-fluoroprop-2-en-1-amine,
I-86-Z: (Z)-3-fluoro-2-((4-(trifluoromethyl)phenoxy)methyl)prop-2-en-1-amine,
I-87-Z: (Z)-2-((4-butoxyphenoxy)methyl)-3-fluoroprop-2-en-1-amine,
I-88-Z: (Z)-3-fluoro-2-(m-tolyloxymethyl)prop-2-en-1-amine,
I-89-Z: (Z)-3-fluoro-2-((3-methoxyphenoxy)methyl)prop-2-en-1-amine,
I-90-Z: (Z)-3-fluoro-2-(p-tolyloxymethyl)prop-2-en-1-amine,
I-91-Z: (Z)-3-fluoro-2-((3-fluoro-5-(trifluoromethyl)phenoxy)methyl)prop-2-en-1-amine,
I-92-E: (E)-3-fluoro-2-(furan-2-ylmethyl)prop-2-en-1-amine,
I-93-E: (E)-3-fluoro-2-(thiophen-2-ylmethyl)prop-2-en-1-amine,
I-94-E: (E)-2-((5-chlorothiophen-2-yl)methyl)-3-fluoroprop-2-en-1-amine,
I-95-E: (E)-3-fluoro-2-((5-methylthiophen-2-yl)methyl)prop-2-en-1-amine,
I-96-E: (E)-3-fluoro-2-(furan-3-ylmethyl)prop-2-en-1-amine,
I-97-E: (E)-3-fluoro-2-(thiophen-3-ylmethyl)prop-2-en-1-amine,
I-98-E: (E)-2-((5-chlorothiophen-3-yl)methyl)-3-fluoroprop-2-en-1-amine,
I-99-E: (E)-2-(fluoromethylene)-4-(4-fluorophenyl)butan-1-amine,
I-100-E: (E)-2-(fluoromethylene)-4-(3-fluorophenyl)butan-1-amine,
I-101-E: (E)-4-(4-chlorophenyl)-2-(fluoromethylene)butan-1-amine,
I-102-E: (E)-2-(fluoromethylene)-4-(4-methoxyphenyl)butan-1-amine,
I-103-E: (E)-4-(4-ethoxyphenyl)-2-(fluoromethylene)butan-1-amine,
I-104-E: (E)-2-(fluoromethylene)-4-(4-(trifluoromethyl)phenyl)butan-1-amine,
I-105-E: (E)-4-(4-butoxyphenyl)-2-(fluoromethylene)butan-1-amine,
I-106-E: (E)-2-(fluoromethylene)-4-m-tolylbutan-1-amine,
I-107-E: (E)-2-(fluoromethylene)-4-(3-methoxyphenyl)butan-1-amine,
I-108-E: (E)-2-(fluoromethylene)-4-p-tolylbutan-1-amine,
I-109-E: (E)-4-(3-fluoro-5-(trifluoromethyl)phenyl)-2-(fluoromethylene)butan-1-amine,
   or any stereoisomer, mixture of stereoisomers, prodrug, metabolite, crystalline form, non-crystalline form, hydrate, solvate, or salt thereof.

In another embodiment, the present invention relates to methods of using one or more of the compounds of formula I-1, I-2, I-3, I-4, I-5, I-6, I-7, I-8, I-9, I-10, I-11, I-12, I-13, I-14, I-15, I-16, I-17, I-18, I-19, I-20, I-21, I-22, I-23, I-24, I-25, I-26, I-27, I-28, I-29, I-30, I-31, I-32, I-33, I-34, I-35, I-36, I-37, I-38, I-39, I-40, I-41, I-42, I-43, I-44, I-45, I-46, I-47, I-48, I-49, I-50, I-51, I-52, I-53, I-54, I-55, I-56, I-57, I-58, I-59, I-60, I-61, I-62, I-63, I-64, I-65, I-66, I-67, I-68, I-69, I-70, I-71, I-72, I-73, I-74, I-75, I-76, I-77, I-78, I-79, I-80, I-81, I-82, I-83, I-84, I-85, I-86, I-87, I-88, I-89, I-90, I-91, I-92, I-93, I-94, I-95, I-96, I-97, I-98, I-99, I-100, I-101, I-102, I-103, I-104, I-105, I-106, I-107, I-108, I-109 to inhibit SSAO enzyme activity (whether the enzyme activity is due either to soluble SSAO enzyme or membrane-bound VAP-1 protein, or due to both) and/or inhibit binding to VAP-1 protein. The compound(s) can be used for a method of inhibiting SSAO activity or inhibiting binding to VAP-1 in vitro, by supplying the compound(s) to the in vitro environment in an amount sufficient to inhibit SSAO activity or inhibit binding to VAP-1. The compound(s) can also be used for a method of inhibiting SSAO activity or inhibiting binding to VAP-1 in vivo, that is, in a living organism, such as a vertebrate, mammal, or human, by administering the compound(s) to the organism in an amount sufficient to inhibit SSAO activity or inhibit binding to VAP-1. In another embodiment, the present invention relates to methods of using one or more compounds of formula I-1, I-2, I-3, I-4, I-5, I-6, I-7, I-8, I-9, I-10, I-11, I-12, I-13, I-14, I-15, I-16, I-17, I-18, I-19, I-20, I-21, I-22, I-23, I-24, I-25, I-26, I-27, I-28, I-29, I-30, I-31, I-32, I-33, I-34, I-35, I-36, I-37, I-38, I-39, I-40, I-41, I-42, I-43, I-44, I-45, I-46, I-47, I-48, I-49, I-50, I-51, I-52, I-53, I-54, I-55, I-56, I-57, I-58, I-59, I-60, I-61, I-62, I-63, I-64, I-65, I-66, I-67, I-68, I-69, I-70, I-71, I-72, I-73, I-74, I-75, I-76, I-77, I-78, I-79, I-80, I-81, I-82, I-83, I-84, I-85, I-86, I-87, I-88, I-89, I-90, I-91, I-92, I-93, I-94, I-95, I-96, I-97, I-98, I-99, I-100, I-101, I-102, I-103, I-104, I-105, I-106, I-107, I-108, I-109 to treat or prevent inflammation or immune disorders. In another embodiment, the present invention relates to methods of using one or more compounds of formula I-1, I-2, I-3, I-4, I-5, I-6, I-7, I-8, I-9, I-10, I-11, I-12, I-13, I-14, I-15, I-16, I-17, I-18, I-19, I-20, I-21, I-22, I-23, I-24, I-25, I-26, I-27, I-28, I-29, I-30, I-31, I-32, I-33, I-34, I-35, I-36, I-37, I-38, I-39, I-40, I-41, I-42, I-43, I-44, I-45, I-46, I-47, I-48, I-49, I-50, I-51, I-52, I-53, I-54, I-55, I-56, I-57, I-58, I-59, I-60, I-61, I-62, I-63, I-64, I-65, I-66, I-67, I-68, I-69, I-70, I-71, I-72, I-73, I-74, I-75, I-76, I-77, I-78, I-79, I-80, I-81, I-82, I-83, I-84, I-85, I-86, I-87, I-88, I-89, I-90, I-91, I-92, I-93, I-94, I-95, I-96, I-97, I-98, I-99, I-100, I-101, I-102, I-103, I-104, I-105, I-106, I-107, I-108, I-109 to suppress or reduce inflammation, or to suppress or reduce an inflammatory response. In another embodiment, the present invention relates to methods of treating or preventing inflammation, by administering on or more compounds described in formula I-1, I-2, I-3, I-4, I-5, I-6, I-7, I-8, I-9, I-10, I-11, I-12, I-13, I-14, I-15, I-16, I-17, I-18, I-19, I-20, I-21, I-22, I-23, I-24, I-25, I-26, I-27, I-28, I-29, I-30, I-31, I-32, I-33, I-34, I-35, I-36, I-37, I-38, I-39, I-40, I-41, I-42, I-43, I-44, I-45, I-46, I-47, I-48, I-49, I-50, I-51, I-52, I-53, I-54, I-55, I-56, I-57, I-58, I-59, I-60, I-61, I-62, I-63, I-64, I-65, I-66, I-67, I-68, I-69, I-70, I-71, I-72, I-73, I-74, I-75, I-76, I-77, I-78, I-79, I-80, I-81, I-82, I-83, I-84, I-85, I-86, I-87, I-88, I-89, I-90, I-91, I-92, I-93, I-94, I-95, I-96, I-97, I-98, I-99, I-100, I-101, I-102, I-103, I-104, I-105, I-106, I-107, I-108, I-109 in a therapeutically effective amount, or in an amount sufficient to treat or prevent inflammation. In another embodiment, the present invention relates to methods of treating or preventing immune or autoimmune disorders, by administering one or more compounds of formula I-1, I-2, I-3, I-4, I-5, I-6, I-7, I-8, I-9, I-10, I-11, I-12, I-13, I-14, I-15, I-16, I-17, I-18, I-19, I-20, I-21, I-22, I-23, I-24, I-25, I-26, I-27, I-28, I-29, I-30, I-31, I-32, I-33, I-34, I-35, I-36, I-37, I-38, I-39, I-40, I-41, I-42, I-43, I-44, I-45, I-46, I-47, I-48, I-49, I-50, I-51, I-52, I-53, I-54, I-55, I-56, I-57, I-58, I-59, I-60, I-61, I-62, I-63, I-64, I-65, I-66, I-67, I-68, I-69, I-70, I-71, I-72, I-73, I-74, I-75, I-76, I-77, I-78, I-79, I-80, I-81, I-82, I-83, I-84, I-85, I-86, I-87, I-88, I-89, I-90, I-91, I-92, I-93, I-94, I-95, I-96, I-97, I-98, I-99, I-100, I-101, I-102, I-103, I-104, I-105, I-106, I-107, I-108, I-109 in a therapeutically effective amount, or in an amount sufficient to treat or prevent the immune or autoimmune disorder.

In another embodiment, the invention relates to compounds of formula II:

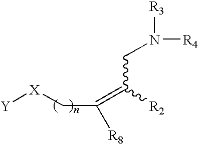

wherein Y is a phenyl, naphthyl, or pyridyl group optionally substituted with one or more groups of the form R₁, wherein each R₁ is independently selected from H, C₁-C₈ alkyl, C₃-C₈ cycloalkyl, —O—C₁-C₈ alkyl, —O—C₃-C₈ cycloalkyl, —C₆-C₁₀ aryl, —O—C₁-C₄ alkyl-C₆-C₁₀ aryl, —S—C₁-C₈ alkyl, —CF₃, —OCF₃, F, Cl, Br, I, —NO₂, —OH, —NR₅R₆, —NHR₇, and —S(O₂)—(C₁-C₈ alkyl); R₂ is selected from H, F, Cl, C₁-C₄ alkyl, and CF₃; R₃ and R₄ are independently selected from H, —C₁-C₈ alkyl, —C₁-C₄ alkyl-C₆-C₁₀ aryl, or R₃ and R₄ together with the nitrogen to which they are attached form a nitrogen-containing ring (including, but not limited to, morpholino, piperidino, and piperazino); R₅ and R₆ are independently selected from H, —C₁-C₈ alkyl, —C₁-C₄ alkyl-C₆-C₁₀ aryl, or R₅ and R₆ together with the nitrogen to which they are attached form a nitrogen-containing ring (including, but not limited to, morpholino, piperidino, and piperazino); R₇ is selected from —C(═O)—(C₁-C₈ alkyl) and —C(═O)—(C₆-C₁₀ aryl); R₈ is selected from H, C₁-C₈ alkyl, C₆-C₁₀ aryl, —C₁-C₄ alkyl-C₆-C₁₀ aryl, and —CF₃; X is —CH₂—, —O—, or —S—; n is 0, 1, 2, or 3; and p is 0, 1, 2, or 3; and any stereoisomer, mixture of stereoisomers, prodrug, metabolite, crystalline form, non-crystalline form, hydrate, solvate, or salt thereof. In another embodiment, X is CH₂ and n is 0 or 1. In another embodiment, X is CH₂ and n is 0. In another embodiment, X is CH₂ and n is 1. In another embodiment, X is O or S and n is 1. In another embodiment, R₁ is selected from C₁-C₄ alkyl, —S—C₁-C₄ alkyl, or —O—C₁-C₄ alkyl. In another embodiment, Y is phenyl, optionally substituted with one or more R₁ substituents. In another embodiment, R₃ and R₄ are both H. In another embodiment, the compounds of formula II are in the E configuration of the double bond; those compounds are designated as compounds of formula II-E. In another embodiment, the compounds of formula II are in the Z configuration of the double bond; those compounds are designated as compounds of formula II-Z.

In another embodiment, the invention relates to compounds of formula II-A:

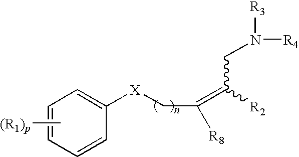

wherein each R₁ is independently selected from H, C₁-C₈ alkyl, C₃-C₈ cycloalkyl, —O—C₁-C₈ alkyl, —O—C₃-C₈ cycloalkyl, —C₆-C₁₀ aryl, —O—C₁-C₄ alkyl-C₆-C₁₀ aryl, —S-C₁-C₈ alkyl, —CF₃, —OCF₃, F, Cl, Br, I, —NO₂, —OH, —NR₅R₆, —NHR₇, and —S(O₂)—(C₁-C₈ alkyl); R₂ is selected from H, F, Cl, C₁-C₄ alkyl, and CF₃; R₃ and R₄ are independently selected from H, —C₁-C₈ alkyl, —C₁-C₄ alkyl-C₆-C₁₀ aryl, or R₃ and R₄ together with the nitrogen to which they are attached form a nitrogen-containing ring (including, but not limited to, morpholino, piperidino, and piperazino); R₅ and R₆ are independently selected from H, —C₁-C₈ alkyl, —C₁-C₄ alkyl-C₆-C₁₀ aryl, or R₅ and R₆ together with the nitrogen to which they are attached form a nitrogen-containing ring (including, but not limited to, morpholino, piperidino, and piperazino); R₇ is selected from —C(═O)—(C₁-C₈ alkyl) and —C(═O)—(C₆-C₁₀ aryl); R₈ is selected from H, C₁-C₈ alkyl, C₆-C₁₀ aryl, —C₁-C₄ alkyl-C₆-C₁₀ aryl, and —CF₃; X is —CH₂—, —O—, or —S—; n is 0, 1, 2, or 3; and p is 0, 1, 2, or 3; and any stereoisomer, mixture of stereoisomers, prodrug, metabolite, crystalline form, non-crystalline form, hydrate, solvate, or salt thereof. In another embodiment, X is CH₂ and n is 0 or 1. In another embodiment, X is CH₂ and n is 0. In another embodiment, X is CH₂ and n is 1. In another embodiment, X is O or S and n is 1. In another embodiment, R₁ is selected from C₁-C₄ alkyl, —S—C₁-C₄ alkyl, or —O—C₁-C₄ alkyl. In another embodiment, R₃ and R₄ are both H. In another embodiment, the compounds of formula II-A are in the E configuration of the double bond; those compounds are designated as compounds of formula II-A-E. In another embodiment, the compounds of formula II-A are in the Z configuration of the double bond; those compounds are designated as compounds of formula II-A-Z.

In one embodiment of the compounds of formula II, II-E, II-Z, II-A, II-A-Z, or II-A-E, n is 0. In another embodiment, R₁ is selected from C₁-C₄ alkyl or —O—C₁-C₄ alkyl. In another embodiment, n is 0 and X is —CH₂—. In another embodiment, n is 0, X is —CH₂—, and R₃ and R₄ are H. In another embodiment, n is 0, X is —CH₂—, R₃ and R₄ are H, and each R₁ is independently selected from H, C₁-C₄ alkyl, C₃-C₈ cycloalkyl, —O—C₁-C₄ alkyl, —S—C₁-C₄ alkyl, CF₃, —O—CF₃, F, and Cl. In another embodiment, n is 0, X is —CH₂—, R₃ and R₄ are H, each R₁ is independently selected from H, C₁-C₄ alkyl, C₃-C₈ cycloalkyl, —O—C₁-C₄ alkyl, —S—C₁-C₄ alkyl, CF₃, —O—CF₃, F, and Cl, and p is 1 or 2.

In another embodiment, the invention relates to compounds of formula II-B:

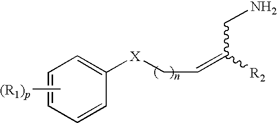

wherein each R₁ is independently selected from H, C₁-C₈ alkyl, C₃-C₈ cycloalkyl, —O—C₁-C₈ alkyl, —O—C₃-C₈ cycloalkyl, —C₆-C₁₀ aryl, —O—C₁-C₄ alkyl-C₆-C₁₀ aryl, —S—C₁-C₈ alkyl, —CF₃, —OCF₃, F, Cl, Br, I, —NO₂, —OH, —NR₅R₆, —NHR₇, and —S(O₂)—(C₁-C₈ alkyl); R₂ is selected from H, F, C₁-C₄ alkyl, and CF₃; R₅ and R₆ are independently selected from H, —C₁-C₈ alkyl, —C₁-C₄ alkyl-C₆-C₁₀ aryl, or R₅ and R₆ together with the nitrogen to which they are attached form a nitrogen-containing ring (including, but not limited to, morpholino, piperidino, and piperazino); R₇ is selected from —C(═O)—(C₁-C₈ alkyl) and —C(═O)—(C₆-C₁₀ aryl); X is —CH₂—, —O—, or —S—; n is 0, 1, 2, or 3; and p is 0, 1, 2, or 3; and any stereoisomer, mixture of stereoisomers, prodrug, metabolite, crystalline form, non-crystalline form, hydrate, solvate, or salt thereof. In another embodiment, the compounds of formula I′-B are in the E configuration of the double bond; those compounds are designated as compounds of formula I′-B-E. In another embodiment, the compounds of formula II-B are in the Z configuration of the double bond; those compounds are designated as compounds of formula II-B-Z.

In one embodiment of the compounds of formula II-B, II-B-Z, or II-B-E, n is 0. In another embodiment, R₁ is selected from C₁-C₄ alkyl, —S—C₁-C₄ alkyl, or —O—C₁-C₄ alkyl. In another embodiment, n is 0 and X is —CH₂—. In another embodiment, n is 0, X is —CH₂—, and R₂ is F. In another embodiment, n is 0, X is —CH₂—, R₂ is F, and each R₁ is independently selected from H, C₁-C₄ alkyl, C₃-C₈ cycloalkyl, —O—C₁-C₄ alkyl, —S—C₁-C₄ alkyl, CF₃, —O—CF₃, F, and Cl. In another embodiment, n is 0, X is —CH₂—, R₂ is F, each R₁ is independently selected from H, C₁-C₄ alkyl, C₃-C₈ cycloalkyl, —O—C₁-C₄ alkyl, —S—C₁-C₄ alkyl, —CF₃, —O—CF₃, F, and Cl, and p is 1 or 2. In another embodiment, n is 0, X is —CH₂—, R₂ is F, each R₁ is independently selected from H, C₁-C₄ alkyl, C₃-C₈ cycloalkyl, —O—C₁-C₄ alkyl, —S—C₁-C₄ alkyl, CF₃, —O—CF₃, F, and Cl, and p is 1. In another embodiment, n is 0, X is —CH₂—, R₂ is F, each R₁ is independently selected from H, C₁-C₄ alkyl, C₃-C₈ cycloalkyl, —O—C₁-C₄ alkyl, —S—C₁-C₄ alkyl, CF₃, —O—CF₃, F, and Cl, and p is 2. In another embodiment, n is 0, X is —CH₂—, R₂ is F, each R₁ is independently selected from CF₃, —O—CF₃, —S—CF₃, —O—CH₃, F, and Cl, and p is 1. In another embodiment, n is 0, X is —CH₂—, R₂ is F, each R₁ is independently selected from CF₃, —O—CF₃, —S—CF₃, —O—CH₃, F, and Cl, and p is 2.

In another embodiment, the present invention relates to any one of the compounds of general formula II of the formula:

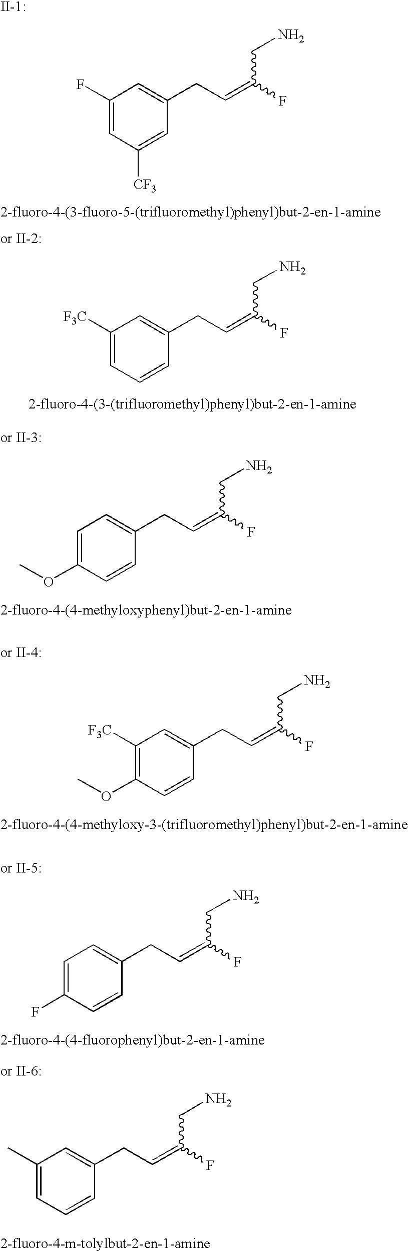

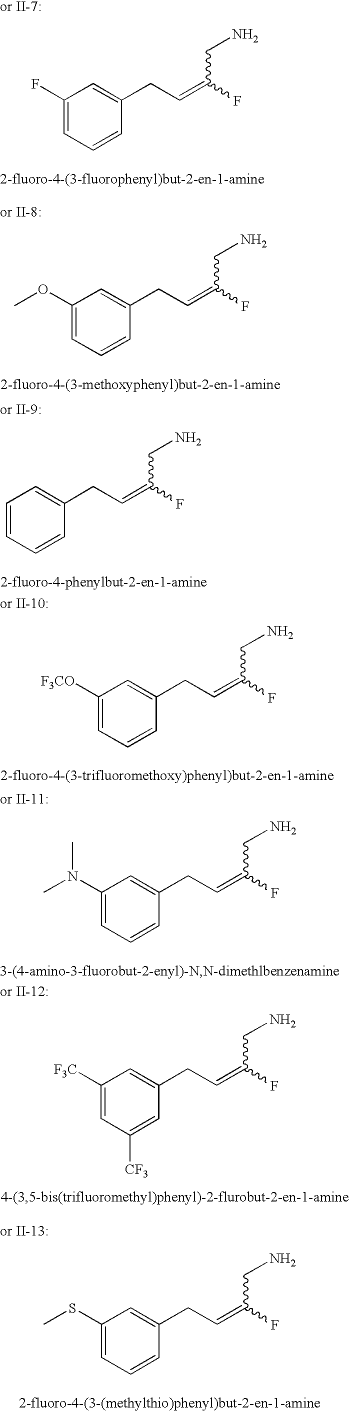

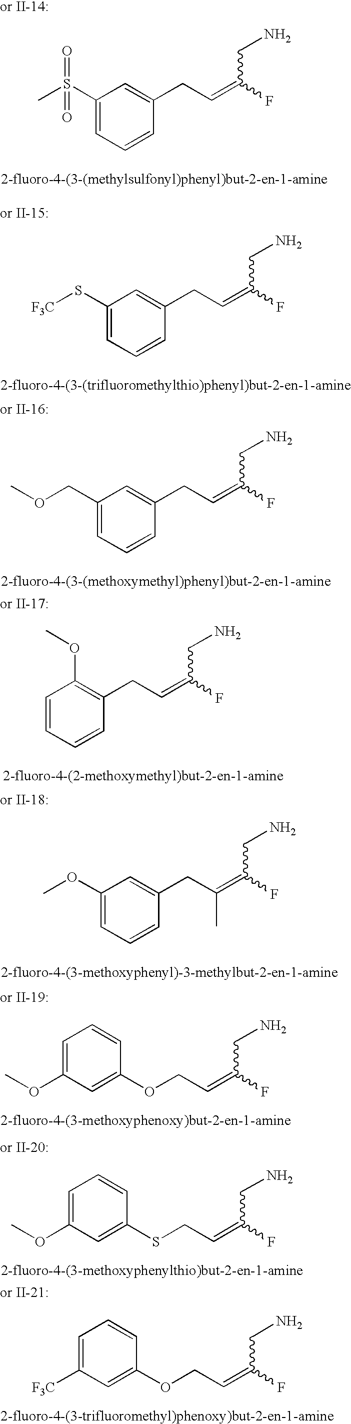

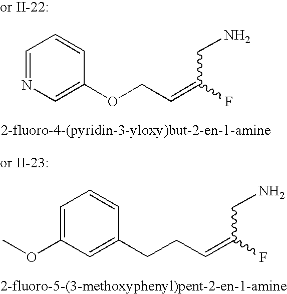

or any stereoisomer, mixture of stereoisomers, prodrug, metabolite, crystalline form, non-crystalline form, hydrate, solvate, or salt thereof.

In another embodiment, the present invention relates to any one of the compounds of general formula I:
II-1-E: (E)-2-fluoro-4-(3-fluoro-5-(trifluoromethyl)phenyl)but-2-en-1-amine,
II-2-E: (E)-2-fluoro-4-(3-(trifluoromethyl)phenyl)but-2-en-1-amine,
II-3-E: (E)-2-fluoro-4-(4-methoxyphenyl)but-2-en-1-amine,
II-4-E: (E)-2-fluoro-4-(4-methoxy-3-(trifluoromethyl)phenyl)but-2-en-1-amine,
II-5-E: (E)-2-fluoro-4-(4-fluorophenyl)but-2-en-1-amine,
II-6-E: (E)-2-fluoro-4-m-tolylbut-2-en-1-amine,
II-7-E: (E)-2-fluoro-4-(3-fluorophenyl)but-2-en-1-amine,
II-8-E: (E)-2-fluoro-4-(3-methoxyphenyl)but-2-en-1-amine,
II-9-E: (E)-2-fluoro-4-phenylbut-2-en-1-amine,
II-10-E: (E)-2-fluoro-4-(3-(trifluoromethoxy)phenyl)but-2-en-1-amine,
II-11-E: (E)-3-(4-amino-3-fluorobut-2-enyl)-N,N-dimethylbenzenamine,
II-12-E: (E)-4-(3,5-bis(trifluoromethyl)phenyl)-2-fluorobut-2-en-1-amine,
II-13-E: (E)-2-fluoro-4-(3-(methylthio)phenyl)but-2-en-1-amine,
II-14-E: (E)-2-fluoro-4-(3-(methylsulfonyl)phenyl)but-2-en-1-amine,
II-15-E: (E)-2-fluoro-4-(3-(trifluoromethylthio)phenyl)but-2-en-1-amine,
II-16-E: (E)-2-fluoro-4-(3-(methoxymethyl)phenyl)but-2-en-1-amine,
II-17-E: (E)-2-fluoro-4-(2-methoxyphenyl)but-2-en-1-amine,
II-18-E: (E)-2-fluoro-4-(3-methoxyphenyl)-3-methylbut-2-en-1-amine,
II-19-E: (E)-2-fluoro-4-(3-methoxyphenoxy)but-2-en-1-amine,
II-20-E: (E)-2-fluoro-4-(3-methoxyphenylthio)but-2-en-1-amine,
II-21-E: (E)-2-fluoro-4-(3-(trifluoromethyl)phenoxy)but-2-en-1-amine,
II-22-E: (E)-2-fluoro-4-(pyridin-3-yloxy)but-2-en-1-amine,
II-23-E: (E)-2-fluoro-5-(3-methoxyphenyl)pent-2-en-1-amine,
   or any stereoisomer, mixture of stereoisomers, prodrug, metabolite, crystalline form, non-crystalline form, hydrate, solvate, or salt thereof.

In another embodiment, the present invention relates to any one of the compounds of general formula I:
II-1-E: (Z)-2-fluoro-4-(3-fluoro-5-(trifluoromethyl)phenyl)but-2-en-1-amine,
II-2-E: (Z)-2-fluoro-4-(3-(trifluoromethyl)phenyl)but-2-en-1-amine,
II-3-Z: (Z)-2-fluoro-4-(4-methoxyphenyl)but-2-en-1-amine,
II-4-Z: (Z)-2-fluoro-4-(4-methoxy-3-(trifluoromethyl)phenyl)but-2-en-1-amine,
II-5-Z: (Z)-2-fluoro-4-(4-fluorophenyl)but-2-en-1-amine,
II-6-Z: (Z)-2-fluoro-4-m-tolylbut-2-en-1-amine,
II-7-Z: (Z)-2-fluoro-4-(3-fluorophenyl)but-2-en-1-amine,
II-8-Z: (Z)-2-fluoro-4-(3-methoxyphenyl)but-2-en-1-amine,
II-9-Z: (Z)-2-fluoro-4-phenylbut-2-en-1-amine,
II-10-Z: (Z)-2-fluoro-4-(3-(trifluoromethoxy)phenyl)but-2-en-1-amine,
II-11-Z: (Z)-3-(4-amino-3-fluorobut-2-enyl)-N,N-dimethylbenzenamine,
II-12-Z: (Z)-4-(3,5-bis(trifluoromethyl)phenyl)-2-fluorobut-2-en-1-amine,
II-13-Z: (Z)-2-fluoro-4-(3-(methylthio)phenyl)but-2-en-1-amine,
II-14-Z: (Z)-2-fluoro-4-(3-(methylsulfonyl)phenyl)but-2-en-1-amine,
II-15-Z: (Z)-2-fluoro-4-(3-(trifluoromethylthio)phenyl)but-2-en-1-amine,
II-16-Z: (Z)-2-fluoro-4-(3-(methoxymethyl)phenyl)but-2-en-1-amine,
II-17-Z: (Z)-2-fluoro-4-(2-methoxyphenyl)but-2-en-1-amine,
II-18-Z: (Z)-2-fluoro-4-(3-methoxyphenyl)-3-methylbut-2-en-1-amine,
II-19-Z: (Z)-2-fluoro-4-(3-methoxyphenoxy)but-2-en-1-amine,
II-20-Z: (Z)-2-fluoro-4-(3-methoxyphenylthio)but-2-en-1-amine,
II-21-Z: (Z)-2-fluoro-4-(3-(trifluoromethyl)phenoxy)but-2-en-1-amine,
II-22-Z: (Z)-2-fluoro-4-(pyridin-3-yloxy)but-2-en-1-amine,
II-23-Z: (Z)-2-fluoro-5-(3-methoxyphenyl)pent-2-en-1-amine,
   or any stereoisomer, mixture of stereoisomers, prodrug, metabolite, crystalline form, non-crystalline form, hydrate, solvate, or salt thereof.

In another embodiment, the present invention relates to methods of using one or more compounds of formula II-1, II-2, I′-3, II-4, II-5, II-6, II-7, II-8, II-9, II-10, II-11, II-12, II-13, II-14, II-15, II-16, II-17, II-18, II-19, II-20, II-21, II-22, or II-23 to inhibit SSAO enzyme activity (whether the enzyme activity is due either to soluble SSAO enzyme or membrane-bound VAP 1 protein, or due to both) and/or inhibit binding to VAP-1 protein. The compound(s) can be used for a method of inhibiting SSAO activity or inhibiting binding to VAP-1 in vitro, by supplying the compound(s) to the in vitro environment in an amount sufficient to inhibit SSAO activity or inhibit binding to VAP-1. The compound(s) can also be used for a method of inhibiting SSAO activity or inhibiting binding to VAP-1 in vivo, that is, in a living organism, such as a vertebrate, mammal, or human, by administering the compound(s) to the organism in an amount sufficient to inhibit SSAO activity or inhibit binding to VAP-1. In another embodiment, the present invention relates to methods of using one or more compounds of formula II-1, II-2, II-3, II-4, II-5, II-6, II-7, II-8, II-9, II-10, II-1, II-12, II-13, II-14, II-15, II-16, II-17, II-18, II-19, II-20, II-21, II-22, or II-23 to treat or prevent inflammation or immune disorders. In another embodiment, the present invention relates to methods of using one or more compounds of formula II-1, II-2, II-3, II-4, II-5, II-6, II-7, II-8, II-9, II-10, II-11, II-12, II-13, II-14, II-15, II-16, II-17, II-18, II-19, II-20, II-21, II-22, or II-23 to suppress or reduce inflammation, or to suppress or reduce an inflammatory response. In another embodiment, the present invention relates to methods of treating or preventing inflammation, by administering one or more compounds of formula II-1, II-2, II-3, II-4, II-5, II-6, II-7, II-8, II-9, II-10, II-11, II-12, II-13, II-14, II-15, II-16, II-17, II-18, II-19, II-20, II-21, II-22, or II-23 in a therapeutically effective amount, or in an amount sufficient to treat or prevent inflammation. In another embodiment, the present invention relates to methods of treating or preventing immune or autoimmune disorders, by administering one or more compounds of formula I-1, II-2, II-3, II-4, II-5, II-6, II-7, II-8, II-9, II-10, II-11, II-12, II-13, II-14, II-15, II-16, II-17, II-18, II-19, II-20, II-21, II-22, or II-23 in a therapeutically effective amount, or in an amount sufficient to treat or prevent the immune or autoimmune disorder.

In another embodiment, the present invention relates to compounds of the formula III:

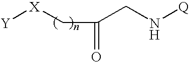

wherein Y is aryl, heteroaryl, or —C₁₋C₈ alkyl, optionally substituted with one or more groups from R₁, wherein R₁ is independently selected from —H, —C₁-C₈ alkyl, —C₃-C₈ cycloalkyl, —O—C₁-C₈ alkyl, —C₁-C₈ alkyl-O—C₁-C₈ alkyl, —O—C₃-C₈ cycloalkyl, —O—C₁-C₈ alkyl-C₃-C₈ cycloalkyl, —C₆-C₁₀ aryl, —O—C₁-C₄ alkyl-C₆-C₁₀ aryl, —S—C₁-C₈ alkyl, —CF₃, —S—CF₃, —OCF₃, —OCH₂CF₃, —CN, —F, —Cl, —Br, —I, —NO₂, —OH, —NHR₇, —NR₅R₆, and —S(O₂)—(C₁-C₈ alkyl); R₇ is selected from —C(═O)—(C₁-C₈ alkyl) and —C(═O)—(C₆-C₁₀ aryl); R₅ and R₆ are independently selected from —H, —C₁-C₈ alkyl, —C₁-C₄ alkyl-C₆-C₁₀ aryl, or R₅ and R₆ together with the nitrogen to which they are attached form a nitrogen-containing ring; X is selected from —CH₂—, —O—, and —S—; n is selected from 0, 1, 2, and 3; and Q is H or a suitable protecting group; and any stereoisomer, mixture of stereoisomers, prodrug, metabolite, crystalline form, non-crystalline form, hydrate, solvate, or salt thereof. In another embodiment, the compounds of formula III have the provisos that Y is not 4-pyridyl. In another embodiment, the compounds of formula III have the provisos that R₁ is not phenyl. The compounds of formula III with a proviso are designated as compounds of formula I′-P. In another embodiment, Q is a H. In another embodiment, Q is a Boc protecting group. In another embodiment, X is —CH₂—; and n is 0. In another embodiment, X is —CH₂—; and n is 0. In another embodiment, X is —O— or —S—; and n is 1 or 2.

In another embodiment, the present invention relates to compounds of the formula III-A:

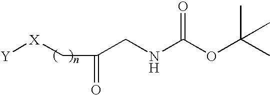

wherein Y is aryl or heteroaryl, optionally substituted with one or more groups from R₁, wherein R₁ is independently selected from —H, —C₁-C₈ alkyl, —C₃-C₈ cycloalkyl, —O—C₁-C₈ alkyl, —C₁-C₈ alkyl-O—C₁-C₈ alkyl, —O—C₃-C₈ cycloalkyl, —O—C₁-C₈ alkyl-C₃-C₈ cycloalkyl, —C₆-C₁₀ aryl, —O—C₁-C₄ alkyl-C₆-C₁₀ aryl, —S—C₁-C₈ alkyl, —CF₃, —OCF₃, —S—CF₃, —OCH₂CF₃, —CN, —F, —Cl, —Br, —I, —NO₂, —OH, —NHR₇, —NR₅R₆, and —S(O₂)—(C₁-C₈ alkyl); R₇ is selected from —C(═O)—(C₁-C₈ alkyl) and —C(═O)—(C₆-C₁₀ aryl); R₅ and R₆ are independently selected from —H, —C₁-C₈ alkyl, —C₁-C₄ alkyl-C₆-C₁₀ aryl, or R₅ and R₆ together with the nitrogen to which they are attached form a nitrogen-containing ring; X—CH₂—; n is selected from 0, 1, and 2; and PG is a suitable protecting group; any stereoisomer, mixture of stereoisomers, prodrug, metabolite, crystalline form, non-crystalline form, hydrate, solvate, or salt thereof. In another embodiment, the compounds of formula III-A have the provisos that Y is not 4-pyridyl. In another embodiment, the compounds of formula III-A have the provisos that R₁ is not phenyl. The compounds of formula III-A with a proviso are designated as compounds of formula II-AP. In another embodiment, X is —CH₂—; and n is 0. In another embodiment, X is —CH₂—; and n is 0. In another embodiment, X is —O— or —S—; and n is 1 or 2.

In another embodiment, the present invention relates to any one of the compounds of general formula III of the formula:

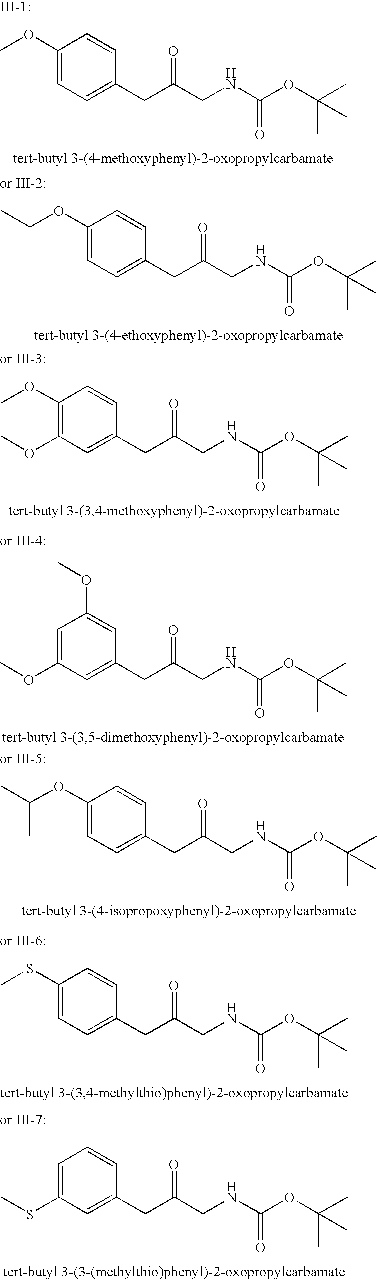

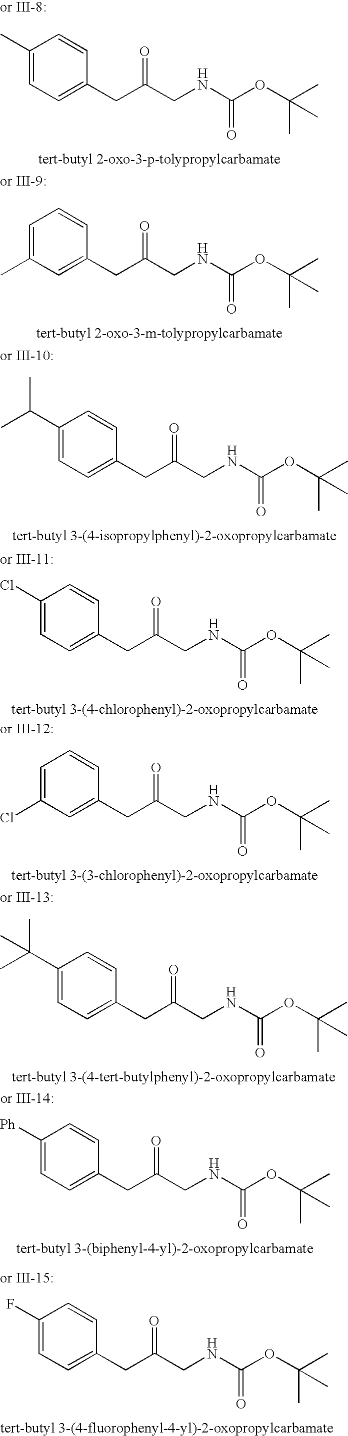

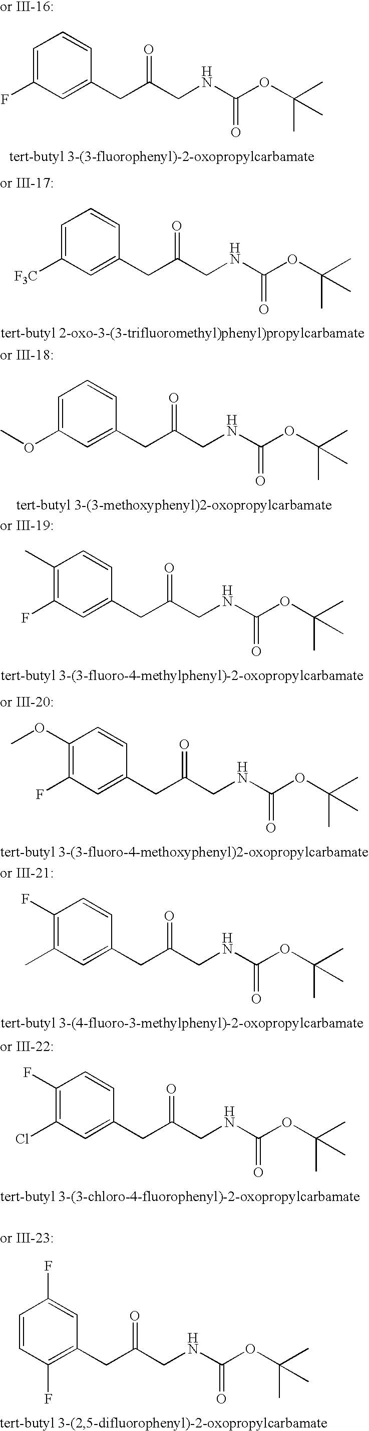

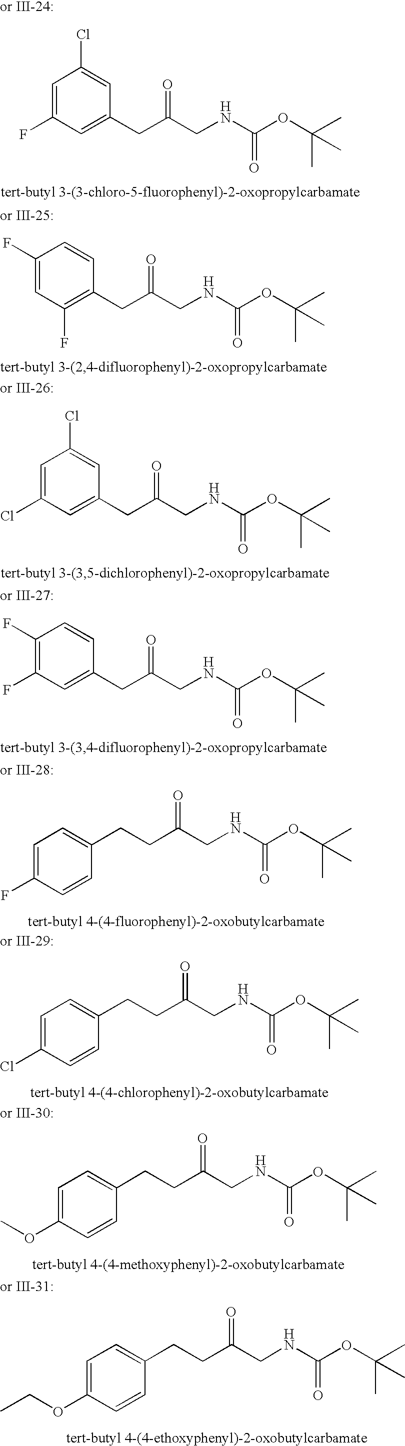

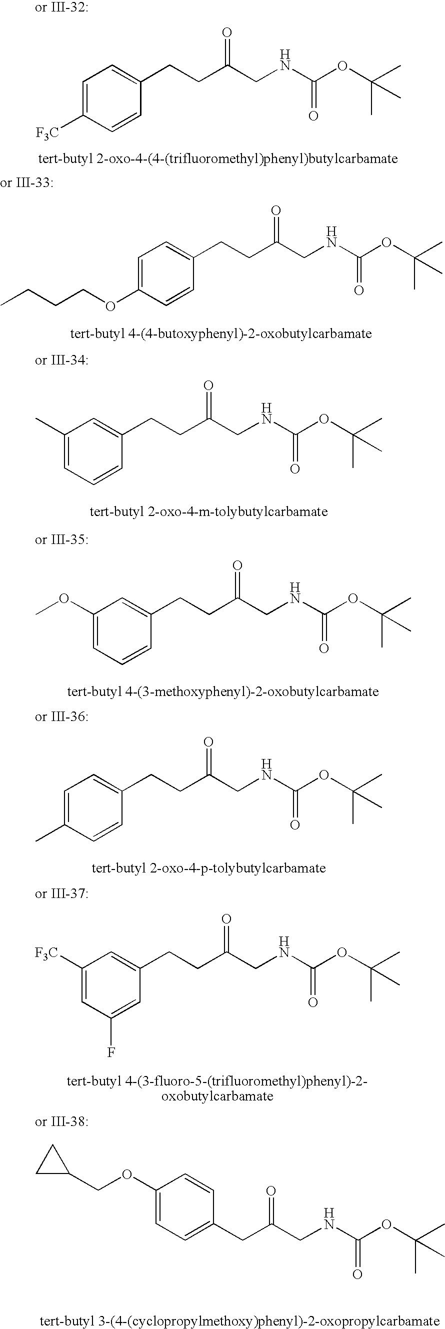

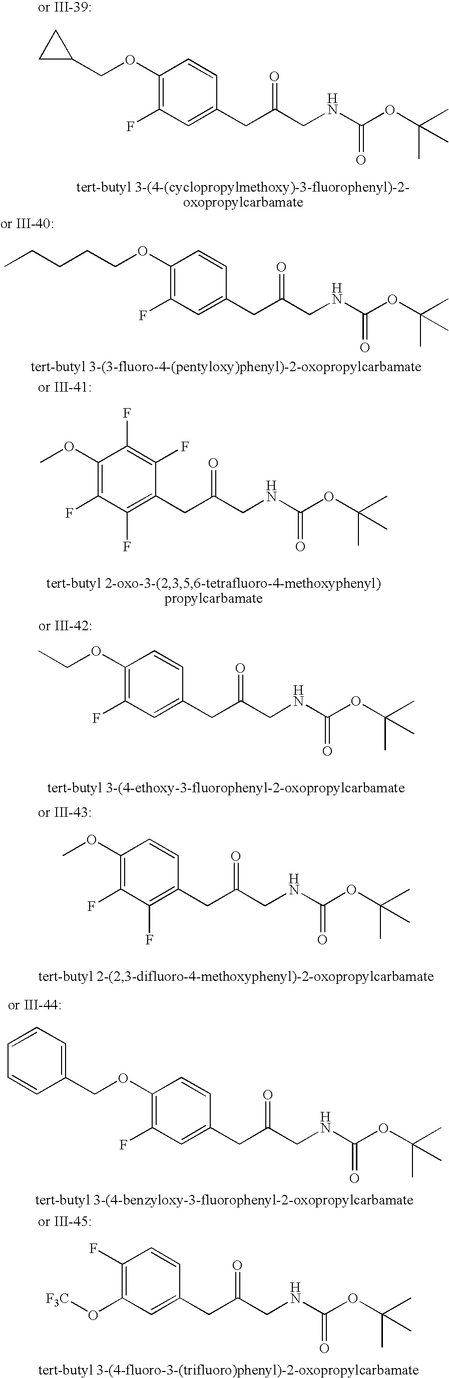

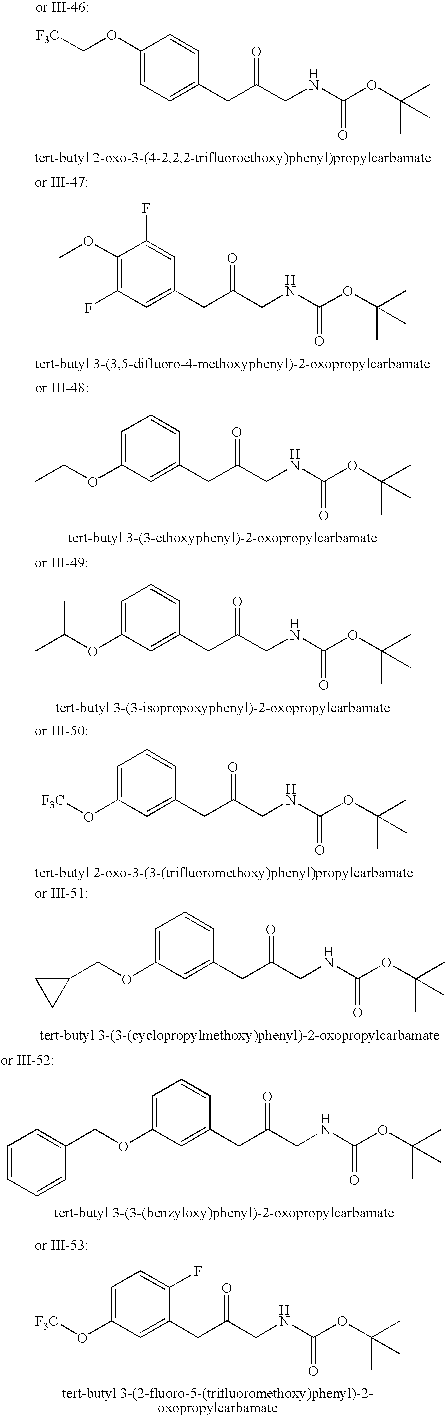

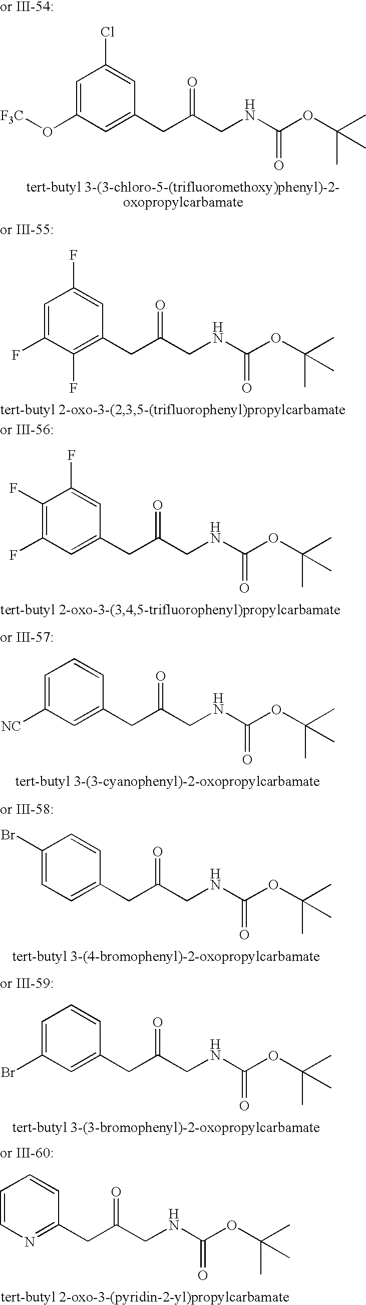

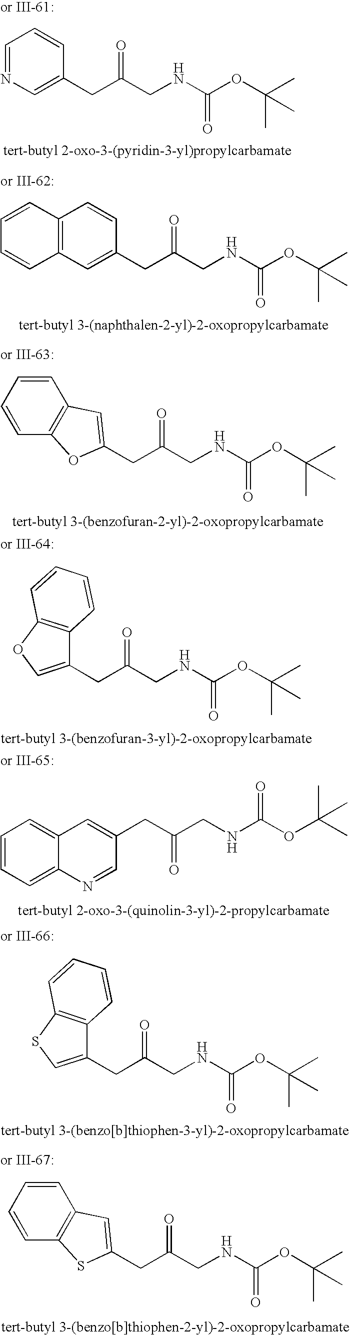

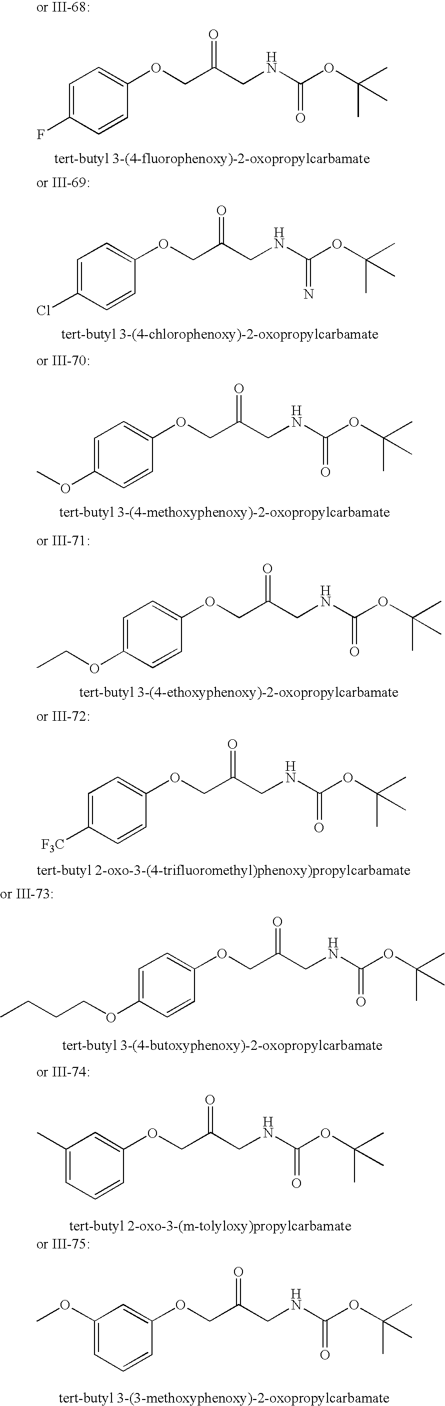

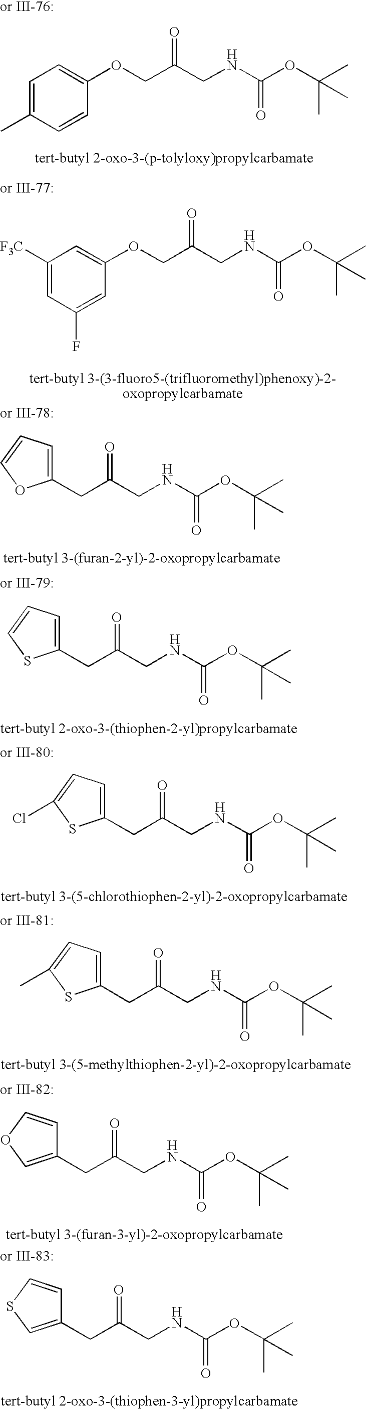

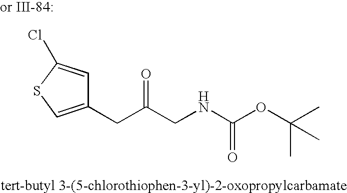

or any stereoisomer, mixture of stereoisomers, prodrug, metabolite, crystalline form, non-crystalline form, hydrate, solvate, or salt thereof.

In another embodiment, the present invention relates to compounds of the formula IV:

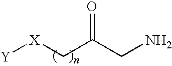

wherein Y is aryl, heteroaryl, or —C₁-C₈ alkyl, optionally substituted with one, two, three, four, or five groups from R₁, wherein R₁ is independently selected from —H, —C₁-C₈ alkyl, —C₃-C₈ cycloalkyl, —O—C₁-C₈ alkyl, —C₁-C₈ alkyl-O—C₁-C₈ alkyl, —O—C₃-C₈ cycloalkyl, —O—C₁-C₈ alkyl-C₃-C₈ cycloalkyl, —C₆-C₁₀ aryl, —O—C₁-C₄ alkyl-C₆-C₁₀ aryl, —S—C₁-C₈ alkyl, —CF₃, —S—CF₃, —OCF₃, —OCH₂CF₃, —CN, —F, —Cl, —Br, —I, —NO₂, —OH, —NHR₇, —NR₅R₆, and —S(O₂)—(C₁-C₈ alkyl); R₇ is selected from —C(═O)—(C₁-C₈ alkyl) and —C(═O)—(C₆-C₁₀ aryl); R₅ and R₆ are independently selected from —H, —C₁-C₈ alkyl, —C₁-C₄ alkyl-C₆-C₁₀ aryl, or R₅ and R₆ together with the nitrogen to which they are attached form a nitrogen-containing ring; X is selected from —CH₂—, —O—, and —S—; and n is selected from 0, 1, 2, and 3; and any stereoisomer, mixture of stereoisomers, prodrug, metabolite, crystalline form, non-crystalline form, hydrate, solvate, or salt thereof.

In another embodiment, formula IV is subject to the proviso that when Y is phenyl, R₃ and R₄ are both H, X is CH₂, and n is 0, then there is at least one R₁ substituent. In another embodiment, formula IV is subject to the proviso that when Y is phenyl, R₃ and R₄ are both H, X is CH₂, and n is 0, then if at least one R₁ substituent is —OCH₃, then there is at least one additional R₁ substituent which is not —OCH₃. In another embodiment, formula IV is subject to the proviso that when Y is phenyl, R₃ and R₄ are both H, X is CH₂, and n is 0, then if at least one R₁ substituent is —OH, then there is at least one additional R₁ substituent which is not —OH.

In another embodiment, formula IV is subject to the proviso that when Y is phenyl, R₃ and R₄ are both H, X is O or S, and n is 1, then there is at least one R₁ substituent. In another embodiment, formula IV is subject to the proviso that when Y is phenyl, R₃ and R₄ are both H, X is O or S, and n is 1, then the phenyl substituents are not Cl, —CF₃, or F in the ortho or para position. In another embodiment, formula IV is subject to the proviso that when Y is phenyl, R₃ and R₄ are both H, X is O or S, and n is 1, then the phenyl substituents are not 3-chloro-5-fluoro. In another embodiment, formula IV is subject to the proviso that when Y is phenyl, R₃ and R₄ are both H, X is O or S, and n is 1, then if at least one R₁ substituent is —OCH₃, then there is at least one additional R₁ substituent is not —OCH₃. In another embodiment, formula IV is subject to the proviso that when Y is phenyl, R₃ and R₄ are both H, X is O or S, and n is 1, if at least one R₁ substituent is —OH, then there is at least one additional R₁ substituent which is not —OH.

In another embodiment, formula IV is subject to the proviso that when Y is phenyl, R₃ and R₄ are both H, X is CH₃, and n is 1, then there is at least one R₁ substituent. In another embodiment, formula IV is subject to the proviso that when Y is phenyl, R₃ and R₄ are both H, X is CH₃, and n is 1, then the phenyl substituent is not F in the para position.

In another embodiment, formula IV is subject to the proviso that when Y is phenyl, R₃ and R₄ are both H, X is CH₃, and n is 2, then the phenyl substituents are not 3,4-dimethoxy.

In another embodiment, the present invention relates to any one of the compounds of general formula IV of the formula:

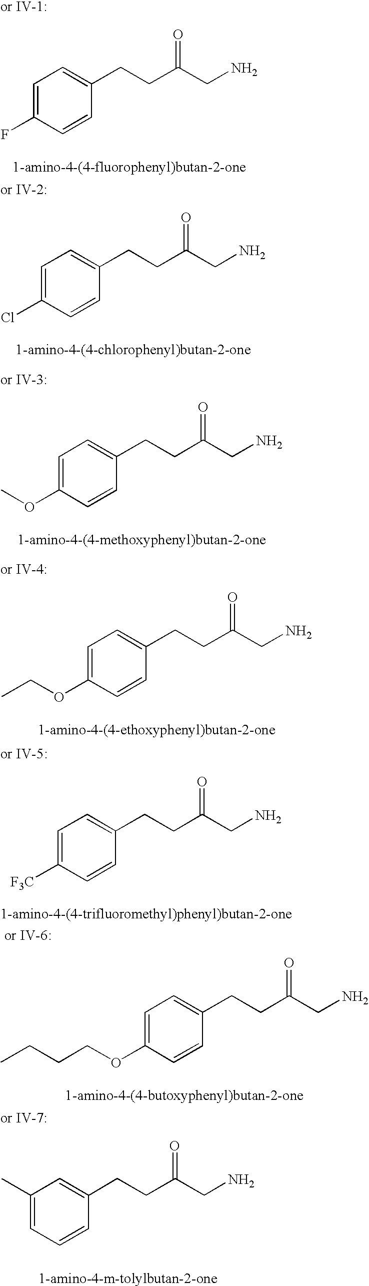

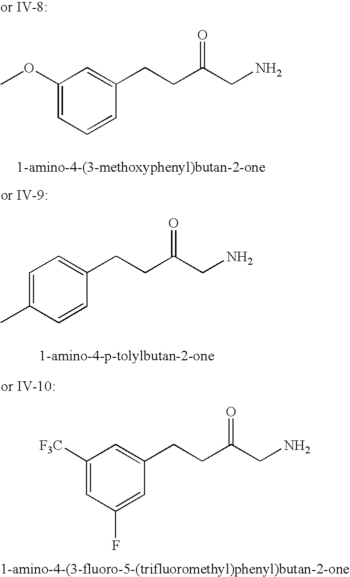

or any stereoisomer, mixture of stereoisomers, prodrug, metabolite, crystalline form, non-crystalline form, hydrate, solvate, or salt thereof.

In another embodiment, the present invention relates to methods of using one or more compounds of formula IV-1, IV-2, IV-3, IV-4, IV-5, IV-6, IV-7, IV-8, IV-9, or IV-10 to inhibit SSAO enzyme activity (whether the enzyme activity is due either to soluble SSAO enzyme or membrane-bound VAP 1 protein, or due to both) and/or inhibit binding to VAP-1 protein. The compound(s) can be used for a method of inhibiting SSAO activity or inhibiting binding to VAP-1 in vitro, by supplying the compound(s) to the in vitro environment in an amount sufficient to inhibit SSAO activity or inhibit binding to VAP-1. The compound(s) can also be used for a method of inhibiting SSAO activity or inhibiting binding to VAP-1 in vivo, that is, in a living organism, such as a vertebrate, mammal, or human, by administering the compound(s) to the organism in an amount sufficient to inhibit SSAO activity or inhibit binding to VAP-1. In another embodiment, the present invention relates to methods of using one or more compounds of formula IV-1, IV-2, IV-3, IV-4, IV-5, IV-6, IV-7, IV-8, IV-9, or IV-10 to treat or prevent inflammation or immune disorders. In another embodiment, the present invention relates to methods of using one or more compounds of formula IV-1, IV-2, IV-3, IV-4, IV-5, IV-6, IV-7, IV-8, IV-9, or IV-10 to suppress or reduce inflammation, or to suppress or reduce an inflammatory response. In another embodiment, the present invention relates to methods of treating or preventing inflammation, by administering one or more compounds of formula IV-1, IV-2, IV-3, IV-4, IV-5, IV-6, IV-7, IV-8, IV-9, or IV-10 in a therapeutically effective amount, or in an amount sufficient to treat or prevent inflammation. In another embodiment, the present invention relates to methods of treating or preventing immune or autoimmune disorders, by administering one or more compounds of formula IV-1, IV-2, IV-3, IV-4, IV-5, IV-6, IV-7, IV-8, IV-9, or IV-10 in a therapeutically effective amount, or in an amount sufficient to treat or prevent the immune or autoimmune disorder.

In another embodiment, the present invention relates to a process for preparing a compound of formula (SI):

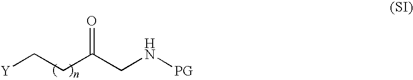

wherein Y is aryl, heteroaryl, or —C₁-C₈ alkyl, optionally substituted with one, two, three, four, or five groups from R₁, wherein each R₁ is independently selected from —C₁-C₈ alkyl, —C₃-C₈ cycloalkyl, —O—C₁-C₈ alkyl, —C₁-C₈ alkyl-O—C₁-C₈ alkyl, —O—C₃-C₈ cycloalkyl, —O—C₁-C₈ alkyl-C₃-C₈ cycloalkyl, —C₆-C₁₀ aryl, —O—C₁-C₄ alkyl-C₆-C₁₀ aryl, —S—C₁-C₈ alkyl, —S—CF₃—CF₃, —OCF₃, —OCH₂CF₃, —CN, —F, —Cl, —Br, —I, —NO₂, —OH, —NHR₄, —NR₅R₆, and —S(O₂)—(C₁-C₈ alkyl); R₄ is selected from —C(═O)—(C₁-C₈ alkyl) and —C(═O)—(C₆-C₁₀ aryl); R₅ and R₆ are independently selected from —H, —C₁-C₈ alkyl, —C₁-C₄ alkyl-C₆-C₁₀ aryl, or R₅ and R₆ together with the nitrogen to which they are attached form a nitrogen-containing ring; n is selected from 0, 1, 2, and 3; and PG is a suitable protecting group; and any stereoisomer, mixture of stereoisomers, prodrug, metabolite, crystalline form, non-crystalline form, hydrate, solvate, or salt thereof;
   comprising reacting a compound of formula SI-A:

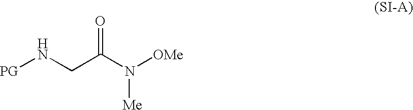

wherein PG is defined above; or any stereoisomer, mixture of stereoisomers, prodrug, metabolite, crystalline form, non-crystalline form, hydrate, solvate, or salt thereof;
   and a compound of formula SI-B:

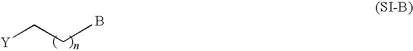

wherein Y and n are as defined above; B is selected from MgX and Li; and X is selected from —F, —Cl, —Br and —I; or any stereoisomer, mixture of stereoisomers, prodrug, metabolite, crystalline form, non-crystalline form, hydrate, solvate, or salt thereof;
   in a suitable solvent (e.g., THF) to form a compound of formula SI:

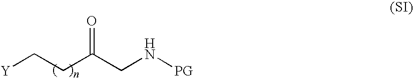

wherein Y, n, and PG are as defined above; any stereoisomer, mixture of stereoisomers, prodrug, metabolite, crystalline form, non-crystalline form, hydrate, solvate, or salt thereof.

In one embodiment of the process for preparing a compound of formula SI, n is 0 or 1. In another embodiment, n is 0. In another embodiment, n is 1. In another embodiment, Y is phenyl, optionally substituted with one or more R₁ substituents. In another embodiment Y is phenyl, optionally substituted with one or more R₁ substituents, and n is 0 or 1. In another embodiment, Y is phenyl, optionally substituted with one or more R₁ substituents, and n is 0. In another embodiment, Y is phenyl, optionally substituted with one or more R₁ substituents, and n is 1. In another embodiment, each R₁ is independently selected from —C₁-C₈ alkyl, —C₃-C₈ cycloalkyl, —O—C₁-C₈ alkyl, —O—C₃-C₈ cycloalkyl, —O—C₁-C₈ alkyl-C₃-C₈ cycloalkyl, —C₆-C₁₀ aryl, —O—C₁-C₄ alkyl-C₆-C₁₀ aryl, —S—C₁-C₈ alkyl, —CF₃, —OCF₃, —OCH₂CF₃, —CN, —F, —Cl, —Br. In another embodiment, Y is phenyl, optionally substituted with one or more R₁ substituents, n is 0 or 1 and each R₁ is independently selected from —C₁-C₈ alkyl, —C₃-C₈ cycloalkyl, —O—C₁-C₈ alkyl, —O—C₃-C₈ cycloalkyl, —O—C₁-C₈ alkyl-C₃-C₈ cycloalkyl, —C₆-C₁₀ aryl, —O—C₁-C₄ alkyl-C₆-C₁₀ aryl, —S—C₁-C₈ alkyl, —CF₃, —OCF₃, —OCH₂CF₃, —CN, —F, —Cl, —Br. In another embodiment, Y is phenyl, optionally substituted with one or more R₁ substituents, n is 0 and each R₁ is independently selected from —C₁-C₈ alkyl, —C₃-C₈ cycloalkyl, —O—C₁-C₈ alkyl, —O—C₃-C₈ cycloalkyl, —O—C₁-C₈ alkyl-C₃-C₈ cycloalkyl, —C₆-C₁₀ aryl, —O—C₁-C₄ alkyl-C₆-C₁₀ aryl, —S—C₁-C₈ alkyl, —CF₃, —OCF₃, —OCH₂CF₃, —CN, —F, —Cl, —Br. In another embodiment, Y is phenyl, optionally substituted with one or more R₁ substituents, n is 1 and each R₁ is independently selected from —C₁-C₈ alkyl, —C₃-C₈ cycloalkyl, —O—C₁-C₈ alkyl, —O—C₃-C₈ cycloalkyl, —O—C₁-C₈ alkyl-C₃-C₈ cycloalkyl, —C₆-C₁₀ aryl, —O—C₁-C₄ alkyl-C₆-C₁₀ aryl, —S—C₁-C₈ alkyl, —CF₃, —OCF₃, —OCH₂CF₃, —CN, —F, —Cl, —Br. In another embodiment, Y is phenyl with one R₁ group of the form —OCH₂CH₃. In another embodiment, Y is phenyl with two R₁ groups of the form —OMe. In another embodiment, Y is phenyl, n is 0 and Y has one R₁ group is of the form —OCH₂CH₃. In another embodiment, Y is phenyl, n is 0, and Y has two R₁ groups of the form —OMe. In another embodiment, Y is phenyl, n is 1 and Y has one R₁ group of the form —OCH₂CH₃. In another embodiment, Y is phenyl, n is 1 and Y has two R₁ groups of the form —OMe. In another embodiment, the protecting group (PG) is tert-butyloxycarbonyl (BOC). In another embodiment, Y is phenyl, optionally substituted with one or more R₁ substituents, and the protecting group (PG) is tert-butyloxycarbonyl (BOC). In another embodiment, Y is phenyl, optionally substituted with one or more R₁ substituents, n is 0 or 1 and the protecting group (PG) is tert-butyloxycarbonyl (BOC).

In one embodiment of the process for preparing a compound of formula SI, B is magnesium bromide.

In another embodiment, the present invention relates to a process for preparing a compound of formula SII:

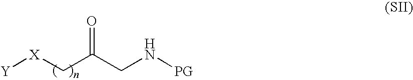

wherein Y is aryl, heteroaryl, or —C₁-C₈ alkyl, optionally substituted with one, two, three, four, or five groups from R₁, wherein each R₁ is independently selected from —H, —C₁-C₈ alkyl, —C₃-C₈ cycloalkyl, —O—C₁-C₈ alkyl, —C₁-C₈ alkyl-O—C₁-C₈ alkyl, —O—C₃-C₈ cycloalkyl, —O—C₁-C₈ alkyl-C₃-C₈ cycloalkyl, —C₆-C₁₀ aryl, —O—C₁-C₄ alkyl-C₆-C₁₀ aryl, —S—C₁-C₈ alkyl, —S—CF₃, —CF₃, —OCF₃, —OCH₂CF₃, —CN, —F, —Cl, —Br, —I, —NO₂, —OH, —NHR₄, —NR₅R₆, and —S(O₂)—(C₁-C₈ alkyl); R₄ is selected from —C(═O)—(C₁-C₈ alkyl) and —C(═O)—(C₆-C₁₀ aryl); R₅ and R₆ are independently selected from —H, —C₁-C₈ alkyl, —C₁-C₄ alkyl-C₆-C₁₀ aryl, or R₅ and R₆ together with the nitrogen to which they are attached form a nitrogen-containing ring; X is selected from —CH₂—, —O—, and —S—; n is selected from 0, 1, 2, and 3; and PG is a suitable protecting group; or any stereoisomer, mixture of stereoisomers, prodrug, metabolite, crystalline form, non-crystalline form, hydrate, solvate, or salt thereof.
   comprising the steps of:
   (a) reacting a compound of formula SII-A:

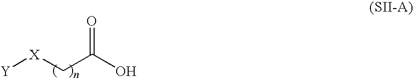

wherein X, Y, and n are as defined above; or any stereoisomer, mixture of stereoisomers, prodrug, metabolite, crystalline form, non-crystalline form, hydrate, solvate, or salt thereof;
   with a halogenating agent in a suitable solvent (e.g., DMF or dichloromethane) to form a compound of formula SII-B:

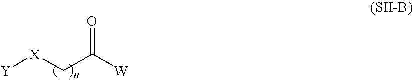

wherein W is selected from —F, —Cl, —Br or —I; and X, Y, and n are as defined above; or any stereoisomer, mixture of stereoisomers, prodrug, metabolite, crystalline form, non-crystalline form, hydrate, solvate, or salt thereof;
   (b) reacting the compound of formula SII-B with (trimethylsilyl)diazomethane (SiMe₃CHN₂) in a suitable solvent (e.g., dichloromethane) to form a compound of formula SII-C:

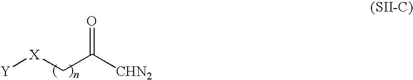

wherein X, Y, and n are as defined above; or any stereoisomer, mixture of stereoisomers, prodrug, metabolite, crystalline form, non-crystalline form, hydrate, solvate, or salt thereof;
   (c) reacting the compound of formula SII-C with HZ;
wherein Z is selected from —F, —Cl, —Br or —I;
   in a suitable solvent (e.g., dichloromethane) to form a compound of formula SII-D:

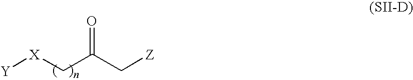

wherein and X, Y, n, and Z are as defined above; or any stereoisomer, mixture of stereoisomers, prodrug, metabolite, crystalline form, non-crystalline form, hydrate, solvate, or salt thereof;
   (d) reacting the compound of formula SII-D with sodium azide (NaN₃) in a suitable solvent (e.g., DMF) to form a compound of formula SII-E:

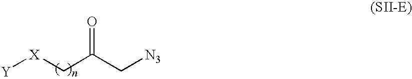

wherein X, Y, and n are as defined above; or any stereoisomer, mixture of stereoisomers, prodrug, metabolite, crystalline form, non-crystalline form, hydrate, solvate, or salt thereof;
   (e) reacting the compound of formula SII-E with an anhydride in the presence of a reducing agent in a suitable solvent (e.g., EtOH) to from a compound of formula SII:

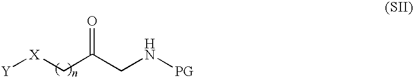

wherein X, Y, PG, and n are as defined above; or any stereoisomer, mixture of stereoisomers, prodrug, metabolite, crystalline form, non-crystalline form, hydrate, solvate, or salt thereof.

In one embodiment of the process for preparing a compound of formula SII, n is 0 or 1. In another embodiment, n is 0. In another embodiment, n is 1. In another embodiment, Y is phenyl, optionally substituted with one or more R₁ substituents. In another embodiment Y is phenyl, optionally substituted with one or more R₁ substituents, and n is 0 or 1. In another embodiment, Y is phenyl, optionally substituted with one or more R₁ substituents, and n is 0. In another embodiment, Y is phenyl, optionally substituted with one or more R₁ substituents, and n is 1. In another embodiment X is —CH₂—. In another embodiment X is —O—. In another embodiment X is —S—. In another embodiment X is selected from —CH₂—, —O—, and —S— and n is 1. In another embodiment, each R₁ is independently selected from —C₁-C₈ alkyl, —C₃-C₈ cycloalkyl, —O—C₁-C₈ alkyl, —O—C₃-C₈ cycloalkyl, —O—C₁-C₈ alkyl-C₃-C₈ cycloalkyl, —C₆-C₁₀ aryl, —O—C₁-C₄ alkyl-C₆-C₁₀ aryl, —S—C₁-C₈ alkyl, —CF₃, —OCF₃, —OCH₂CF₃, —CN, —F, —Cl, —Br. In another embodiment, Y is phenyl, optionally substituted with one or more R₁ substituents, n is 0 or 1 and each R₁ is independently selected from —C₁-C₈ alkyl, —C₃-C₈ cycloalkyl, —O—C₁-C₈ alkyl, —O—C₃-C₈ cycloalkyl, —O—C₁-C₈ alkyl-C₃-C₈ cycloalkyl, —C₆-C₁₀ aryl, —O—C₁-C₄ alkyl-C₆-C₁₀ aryl, —S—C₁-C₈ alkyl, —CF₃, —OCF₃, —OCH₂CF₃, —CN, —F, —Cl, —Br. In another embodiment, Y is phenyl, optionally substituted with one or more R₁ substituents, n is 0 and each R₁ is independently selected from —C₁-C₈ alkyl, —C₃-C₈ cycloalkyl, —O—C₁-C₈ alkyl, —O—C₃-C₈ cycloalkyl, —O—C₁-C₈ alkyl-C₃-C₈ cycloalkyl, —C₆-C₁₀ aryl, —O—C₁-C₄ alkyl-C₆-C₁₀ aryl, —S-C₁-C₈ alkyl, —CF₃, —OCF₃, —OCH₂CF₃, —CN, —F, —Cl, —Br. In another embodiment, Y is phenyl, optionally substituted with one or more R₁ substituents, n is 1 and each R₁ is independently selected from —C₁-C₈ alkyl, —C₃-C₈ cycloalkyl, —O—C₁-C₈ alkyl, —O—C₃-C₈ cycloalkyl, —O—C₁-C₈ alkyl-C₃-C₈ cycloalkyl, —C₆-C₁₀ aryl, —O—C₁-C₄ alkyl-C₆-C₁₀ aryl, —S—C₁-C₈ alkyl, —CF₃, —OCF₃, —OCH₂CF₃, —CN, —F, —Cl, —Br. In another embodiment, Y is phenyl, optionally substituted with one or more R₁ substituents, and one R₁ group is —OCH₂CH₃. In another embodiment, Y is phenyl, optionally substituted with one or more R₁ substituents, and two R₁ groups are —OMe. In another embodiment, Y is phenyl, optionally substituted with one or more R₁ substituents, n is 0 and one R₁ group is —OCH₂CH₃. In another embodiment, Y is phenyl, optionally substituted with one or more R₁ substituents, n is 0 two R₁ groups are —OMe.

In another embodiment, Y is phenyl, optionally substituted with one or more R₁ substituents, n is 1 and one R₁ group is —OCH₂CH₃. In another embodiment, Y is phenyl, optionally substituted with one or more R₁ substituents, n is 1 and two R₁ groups are —OMe. In another embodiment, Z is —Br. In another embodiment, the anhydride is tert-butyloxycarbonyl (Boc₂O) and the protection group (PG) is tert-butyloxycarbonyl (Boc).

In one embodiment of the process for preparing a compound of formula SII, the halogenating agent is oxalyl chloride (C₂Cl₂O₂) and W is —Cl. In another embodiment, step (a) is carried out in the presence of dimethylformamide (DMF). In another embodiment, the reducing agent is Pd(OH)₂/C. In another embodiment step (e) is carried out in the presence of Et₃SiH. In another embodiment Z is —Br.

In another embodiment, the present invention relates to a process for preparing a compound of formula (SIII):

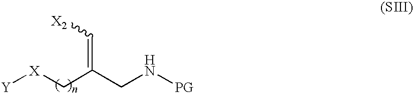

wherein Y is aryl, heteroaryl, or —C₁-C₈ alkyl, optionally substituted with one, two, three, four, or five groups from R₁, wherein each R₁ is independently selected from —C₁-C₈ alkyl, —C₃-C₈ cycloalkyl, —O—C₁-C₈ alkyl, —C₁-C₈ alkyl-O—C₁-C₈ alkyl, —O—C₃-C₈ cycloalkyl, —O—C₁-C₈ alkyl-C₃-C₈ cycloalkyl, —C₆-C₁₀ aryl, —O—C₁-C₄ alkyl-C₆-C₁₀ aryl, —S—C₁-C₈ alkyl, —S—CF₃, —CF₃, —OCF₃, —OCH₃CF₃, —CN, —F, —Cl, —Br, —I, —NO₂, —OH, —NHR₄, —NR₅R₆, and —S(O₂)—(C₁-C₈ alkyl); R₄ is selected from —C(═O)—(C₁-C₈ alkyl) and —C(═O)—(C₆-C₁₀ aryl); R₅ and R₆ are independently selected from —H, —C₁-C₈ alkyl, —C₁-C₄ alkyl-C₆-C₁₀ aryl, or R₅ and R₆ together with the nitrogen to which they are attached form a nitrogen-containing ring; X is selected from —CH₂—, —O—, and —S—; X₂ is selected from —F, —Cl, —Br, and —I; n is selected from 0, 1, 2 and 3; and PG is a suitable protecting group; or any stereoisomer, mixture of stereoisomers, prodrug, metabolite, crystalline form, non-crystalline form, hydrate, solvate, or salt thereof;
   comprising reacting compound SIII-A:

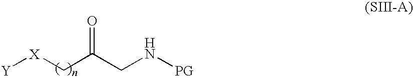

wherein X, Y, n, and PG are as defined above; or any stereoisomer, mixture of stereoisomers, prodrug, metabolite, crystalline form, non-crystalline form, hydrate, solvate, or salt thereof;
   with a phosphonium salt in a suitable solvent (e.g., THF) in the presence of a suitable base to from a mixture of E- and Z-isomers of formula SIII:

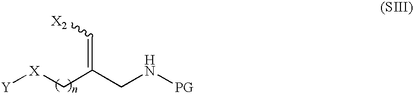

wherein X, X₂, Y, n, and PG are as defined above; or any stereoisomer, mixture of stereoisomers, prodrug, metabolite, crystalline form, non-crystalline form, hydrate, solvate, or salt thereof.

In one embodiment of the process for preparing a compound of formula SIII, n is 0 or 1. In another embodiment, n is 0. In another embodiment, n is 1. In another embodiment, Y is phenyl, optionally substituted with one or more R₁ substituents. In another embodiment Y is phenyl, optionally substituted with one or more R₁ substituents, and n is 0 or 1. In another embodiment, Y is phenyl, optionally substituted with one or more R₁ substituents, and n is 0. In another embodiment, Y is phenyl, optionally substituted with one or more R₁ substituents, and n is 1. In another embodiment X is —CH₂—. In another embodiment X is —O—. In another embodiment X is —S—. In another embodiment X is selected from —CH₂—, —O—, and —S— and n is 1. In another embodiment, each R₁ is independently selected from —C₁-C₈ alkyl, —C₃-C₈ cycloalkyl, —O—C₁-C₈ alkyl, —O—C₃-C₈ cycloalkyl, —O—C₁-C₈ alkyl-C₃-C₈ cycloalkyl, —C₆-C₁₀ aryl, —O—C₁-C₄ alkyl-C₆-C₁₀ aryl, —S—C₁-C₈ alkyl, —CF₃, —OCF₃, —OCH₂CF₃, —CN, —F, —Cl, —Br. In another embodiment, Y is phenyl, optionally substituted with one or more R₁ substituents, n is 0 or 1 and each R₁ is independently selected from —C₁-C₈ alkyl, —C₃-C₈ cycloalkyl, —O—C₁-C₈ alkyl, —O—C₃-C₈ cycloalkyl, —O—C₁-C₈ alkyl-C₃-C₈ cycloalkyl, —C₆-C₁₀ aryl, —O—C₁-C₄ alkyl-C₆-C₁₀ aryl, —S—C₁-C₈ alkyl, —CF₃, —OCF₃, —OCH₂CF₃, —CN, —F, —Cl, —Br. In another embodiment, Y is phenyl, optionally substituted with one or more R₁ substituents, n is 0 and each R₁ is independently selected from —C₁-C₈ alkyl, —C₃-C₈ cycloalkyl, —O—C₁-C₈ alkyl, —O—C₃-C₈ cycloalkyl, —O—C₁-C₈ alkyl-C₃-C₈ cycloalkyl, —C₆-C₁₀ aryl, —O—C₁-C₄ alkyl-C₆-C₁₀ aryl, —S—C₁-C₈ alkyl, —CF₃, —OCF₃, —OCH₂CF₃, —CN, —F, —Cl, —Br. In another embodiment, Y is phenyl, optionally substituted with one or more R₁ substituents, n is 1 and each R₁ is independently selected from —C₁-C₈ alkyl, —C₃-C₈ cycloalkyl, —O—C₁-C₈ alkyl, —O—C₃-C₈ cycloalkyl, —O—C₁-C₈ alkyl-C₃-C₈ cycloalkyl, —C₆-C₁₀ aryl, —O—C₁-C₄ alkyl-C₆-C₁₀ aryl, —S—C₁-C₈ alkyl, —CF₃, —OCF₃, —OCH₂CF₃, —CN, —F, —Cl, —Br. In another embodiment, Y is phenyl, optionally substituted with one or more R₁ substituents, and one R₁ group is —OCH₂CH₃. In another embodiment, Y is phenyl, optionally substituted with one or more R₁ substituents, and two R₁ groups are —OMe. In another embodiment, Y is phenyl, optionally substituted with one or more R₁ substituents, n is 0 and one R₁ group is —OCH₂CH₃. In another embodiment, Y is phenyl, optionally substituted with one or more R₁ substituents, n is 0, and two R₁ groups are —OMe. In another embodiment, Y is phenyl, optionally substituted with one or more R₁ substituents, n is 1 and one R₁ group is —OCH₂CH₃. In another embodiment, Y is phenyl, optionally substituted with one or more R₁ substituents, n is 1 and two R₁ groups are —OMe. In another embodiment, X₂ is F or Cl. In another embodiment, X₂ is F. In another embodiment, X₂ is Cl. In another embodiment, X₂ is F or Cl and n is 0 or 1. In another embodiment, X₂ is F or Cl and n is 0. In another embodiment, X₂ is F or Cl and n is 1. In another embodiment, X₂ is F or Cl, n is 0 or 1, and X is —CH₂—. In another embodiment, X₂ is F or Cl, n is 1, and X is —O—. In another embodiment, the protection group (PG) is tert-butyloxycarbonyl (Boc).

In one embodiment of the process for preparing a compound of formula SIII, the process for preparing a compound of formula SIII further comprises separation of the isomeric mixture. In another embodiment, the process comprises separation of the isomeric mixture by column chromatography. In another embodiment, the process further comprises the process for preparing SI for use as SIII-A. In another embodiment, the process further comprises the process for preparing SII for use as SIII-A. In another embodiment, the phosphonium salt is fluoromethyltriphenylphosphonium tetrafluoroborate (FCH₂PPh₃⁺BF₄⁻) and the base is sodium hydride (NaH).

In one embodiment of the process for preparing a compound of formula SIII, the Z enantiomer of SIII is produced in an amount greater than about 50%, greater than about 60%, greater than about 70%, greater than about 80%, greater than about 85%, greater than about 90%, greater than about 95%, greater than about 97%, greater than about 98%, or greater than about 99%.

In another embodiment, the process for preparing a compound of formula SIII further comprises removing the protecting group (PG) from SIII in a suitable solvent (e.g., dichloromethane) to form a compound of formula SIV:

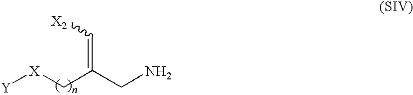

wherein X, Y, X₂, and n are as defined above; or any stereoisomer, mixture of stereoisomers, prodrug, metabolite, crystalline form, non-crystalline form, hydrate, solvate, or salt thereof.

In one embodiment of the process for preparing a compound of formula SIV, the protecting group (PG) is removed with a strong acid. In another embodiment, the protecting group (PG) is removed with trifluoroacetic acid (TFA).

In another embodiment, the present invention relates to a process for preparing a compound of formula SV:

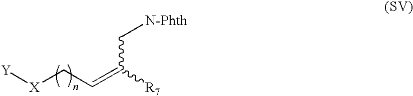

wherein Y is aryl, heteroaryl, or —C₁-C₈ alkyl, optionally substituted with one, two, three, four, or five groups from R₁, wherein each R₁ is independently selected from —C₁-C₈ alkyl, —C₃-C₈ cycloalkyl, —O—C₁-C₈ alkyl, —C₁-C₈ alkyl-O—C₁-C₈ alkyl, —O—C₃-C₈ cycloalkyl, —O—C₁-C₈ alkyl-C₃-C₈ cycloalkyl, —C₆-C₁₀ aryl, —O—C₁-C₄ alkyl-C₆-C₁₀ aryl, —S—C₁-C₈ alkyl, —S—CF₃, —CF₃, —OCF₃, —OCH₃CF₃, —CN, —F, —Cl, —Br, —I, —NO₂, —OH, —NHR₄, —NR₅R₆, and —S(O₂)—(C₁-C₈ alkyl); R₄ is selected from —C(═O)—(C₁-C₈ alkyl) and —C(═O)—(C₆-C₁₀ aryl); R₅ and R₆ are independently selected from —H, —C₁-C₈ alkyl, —C₁-C₄ alkyl-C₆-C₁₀ aryl, or R₅ and R₆ together with the nitrogen to which they are attached form a nitrogen-containing ring; X is selected from —CH₂—, —O—, and —S—; n is selected from 0, 1, 2 and 3; and R₇ is selected from —H, —F, —C₁-C₄ alkyl, and —CF₃; or any stereoisomer, mixture of stereoisomers, prodrug, metabolite, crystalline form, non-crystalline form, hydrate, solvate, or salt thereof,
   comprising the steps of:
   (a) reacting a compound of formula SV-A:

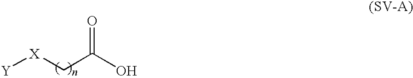

wherein X, Y, and n are as defined above; or any stereoisomer, mixture of stereoisomers, prodrug, metabolite, crystalline form, non-crystalline form, hydrate, solvate, or salt thereof;
   with R₈OH and an acid;
wherein R₈ is selected from —C₁-C₈ alkyl, —C₃-C₈ cycloalkyl, or —C₆-C₁₀ aryl; or any stereoisomer, mixture of stereoisomers, prodrug, metabolite, crystalline form, non-crystalline form, hydrate, solvate, or salt thereof,
   in a suitable solvent (e.g., MeOH) to form a compound of formula SV-B:

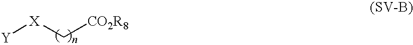

wherein X, Y, R₈, and n are as defined above; or any stereoisomer, mixture of stereoisomers, prodrug, metabolite, crystalline form, non-crystalline form, hydrate, solvate, or salt thereof;
   (b) reacting the compound of formula SV-B with a reducing agent in a suitable solvent (e.g., dichloromethane) to form a compound of formula SV-C:

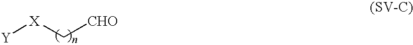

wherein X, Y, and n are as defined above; or any stereoisomer, mixture of stereoisomers, prodrug, metabolite, crystalline form, non-crystalline form, hydrate, solvate, or salt thereof;
   (c) reacting the compound of formula SV-C with a phosphonate ester in a suitable solvent (e.g., THF) in the presence of a suitable base to from a mixture of E- and Z-isomers of formula SV-D:

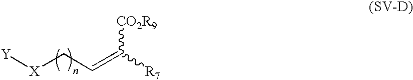

wherein X, Y, R₇ and n are as defined above; and
R₉ is selected from —C₁-C₈ alkyl, —C₃-C₈ cycloalkyl, and —C₆-C₁₀ aryl; or any stereoisomer, mixture of stereoisomers, prodrug, metabolite, crystalline form, non-crystalline form, hydrate, solvate, or salt thereof;
   (d) reacting the compound of formula SV-D with a reducing agent in a suitable solvent (e.g., hexane or toluene) to form a compound of formula SV-E:

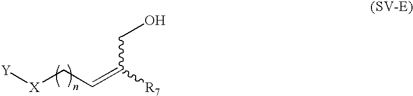

wherein X, Y, R₇ and n are defined above; or any stereoisomer, mixture of stereoisomers, prodrug, metabolite, crystalline form, non-crystalline form, hydrate, solvate, or salt thereof;
   (e) reacting the compound of formula SV-E with phthalimide in the presence of a phosphine and an azodicarboxylate reagent in a suitable solvent (e.g., THF) to form a compound of formula SV:

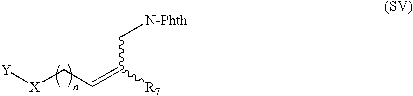

wherein X, Y, R₇ and n are defined above; or any stereoisomer, mixture of stereoisomers, prodrug, metabolite, crystalline form, non-crystalline form, hydrate, solvate, or salt thereof.

In one embodiment of the process for preparing a compound of formula SV, n is 0 or 1. In another embodiment, n is 0. In another embodiment, n is 1. In another embodiment, Y is phenyl, optionally substituted with one or more R₁ substituents. In another embodiment Y is phenyl, optionally substituted with one or more R₁ substituents, and n is 0 or 1. In another embodiment, Y is phenyl, optionally substituted with one or more R₁ substituents, and n is 0. In another embodiment, Y is phenyl, optionally substituted with one or more R₁ substituents, and n is 1. In another embodiment X is —CH₂—. In another embodiment X is —O—. In another embodiment X is —S—. In another embodiment X is selected from —CH₂—, —O—, and —S— and n is 1. In another embodiment, each R₁ is independently selected from —C₁-C₈ alkyl, —O—C₁-C₈ alkyl, —C₁-C₈ alkyl-O—C₁-C₈ alkyl, —S—C₁-C₈ alkyl, —CF₃, —OCF₃, —F, —S(O₂)—(C₁-C₈ alkyl), and —NR₅R₆. In another embodiment, Y is phenyl, optionally substituted with one or more R₁ substituents, n is 0 or 1 and each R₁ is independently selected from —C₁-C₈ alkyl, —O—C₁-C₈ alkyl, —C₁-C₈ alkyl-O—C₁-C₈ alkyl, —S—C₁-C₈ alkyl, —CF₃, —OCF₃, —F, —S(O₂)—(C₁-C₈ alkyl), and —NR₅R₆. In another embodiment, Y is phenyl, optionally substituted with one or more R₁ substituents, n is 0 and each R₁ is independently selected from —C₁-C₈ alkyl, —O—C₁-C₈ alkyl, —C₁-C₈ alkyl-O—C₁-C₈ alkyl, —S—C₁-C₈ alkyl, —CF₃, —OCF₃, —F, —S(O₂)—(C₁-C₈ alkyl), and —NR₅R₆. In another embodiment, Y is phenyl, optionally substituted with one or more R₁ substituents, n is 1 and each R₁ is independently selected from —C₁-C₈ alkyl, —O—C₁-C₈ alkyl, —C₁-C₈ alkyl-O—C₁-C₈ alkyl, —S—C₁-C₈ alkyl, —CF₃, —OCF₃, —F, —S(O₂)—(C₁-C₈ alkyl), and —NR₅R₆. In another embodiment, R₇ is —F. In another embodiment, Y is phenyl, optionally substituted with one or more R₁ substituents, and R₇ is —F. In another embodiment, Y is phenyl, optionally substituted with one or more R₁ substituents, n is O and R₇ is —F. In another embodiment, Y is phenyl, optionally substituted with one or more R₁ substituents, n is 0, X is —CH₃—, and R₇ is —F.

In one embodiment, the process for preparing a compound of formula SV further comprises separation of the isomeric mixture. In another embodiment, the process further comprises separation of the isomeric mixture by column chromatography. In another embodiment, R₈ is -Me. In another embodiment, the acid is H₂SO₄. In another embodiment, the reducing agent in step (b) is diisobutylaluminum hydride (DIBAL-H). In another embodiment, the reducing agent in step (d) is diisobutylaluminum hydride (DIBAL-H). In another embodiment, for step (c) the phosphonate ester is triethyl-2-fluoro-2-phosphoacetate, R₉ is —CH₂CH₃, and the base is isopropylmagnesium chloride (i-PrMgCl). In another embodiment, the phosphine is triphenyl phosphine (PPh₃) and the azodicarboxylate is diisopropyl azodicarboxylate (DIAD).

In one embodiment of the process for preparing a compound of formula SV, the Z enantiomer of SV is produced in an amount greater than about 50%, greater than about 60%, greater than about 70%, greater than about 80%, greater than about 85%, greater than about 90%, greater than about 95%, greater than about 97%, greater than about 98%, or greater than about 99%. In another embodiment, the E enantiomer of SV is produced in an amount greater than about 50%, greater than about 60%, greater than about 70%, greater than about 80%, greater than about 85%, greater than about 90%, greater than about 95%, greater than about 97%, greater than about 98%, or greater than about 99%.

In another embodiment, the process for preparing a compound of formula SV further comprises removing the N-phthalimido protecting group (PG) from SV in a suitable solvent (e.g., ethanol, followed by diethyl ether when forming and isolating the salt form) to form a compound of formula SVI:

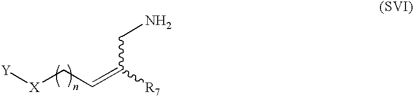

wherein Y, X, R₇, and n are defined above; or any stereoisomer, mixture of stereoisomers, prodrug, metabolite, crystalline form, non-crystalline form, hydrate, solvate, or salt thereof.

In one embodiment of the process for preparing a compound of formula SVI, the N-phthalimido protecting group (PG) is removed using hydrazine. In another embodiment, the N-phthalimido protecting group (PG) is removed using a primary amine. In another embodiment, the N-phthalimido protecting group (PG) is removed using a methyl amine.

In another embodiment, the present invention relates to a process for preparing a compound of formula SVII:

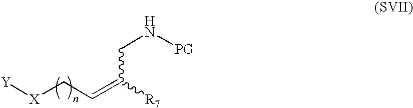

wherein Y is aryl, heteroaryl, or —C₁-C₈ alkyl, optionally substituted with one, two, three, four, or five groups from R₁, wherein each R₁ is independently selected from —C₁-C₈ alkyl, —C₃-C₈ cycloalkyl, —O—C₁-C₈ alkyl, —C₁-C₈ alkyl-O—C₁-C₈ alkyl, —O—C₃-C₈ cycloalkyl, —O—C₁-C₈ alkyl-C₃-C₈ cycloalkyl, —C₆-C₁₀ aryl, —O—C₁-C₄ alkyl-C₆-C₁₀ aryl, —S-C₁-C₈ alkyl, —S—CF₃, —CF₃, —OCF₃, —OCH₃CF₃, —CN, —F, —Cl, —Br, —I, —NO₂, —OH, —NHR₄, —NR₅R₆, and —S(O₂)—(C₁-C₈ alkyl); R₄ is selected from —C(═O)—(C₁-C₈ alkyl) and —C(═O)—(C₆-C₁₀ aryl); R₅ and R₆ are independently selected from —H, —C₁-C₈ alkyl, —C₁-C₄ alkyl-C₆-C₁₀ aryl, or R₅ and R₆ together with the nitrogen to which they are attached form a nitrogen-containing ring; X is selected from —O— and —S—; n is selected from 1, 2 and 3; and R₇ is selected from —H, —F, —C₁-C₄ alkyl, and —CF₃; and PG is a suitable protecting group; or any stereoisomer, mixture of stereoisomers, prodrug, metabolite, crystalline form, non-crystalline form, hydrate, solvate, or salt thereof; comprising:
   reacting a compound of formula SVII-A:

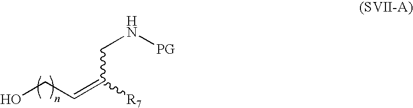

wherein R₇, n, and PG are as defined above; or any stereoisomer, mixture of stereoisomers, prodrug, metabolite, crystalline form, non-crystalline form, hydrate, solvate, or salt thereof;
   and a compound of formula SVII-B:

Y—X—H (SVII-B)
wherein Y and X are defined above; or any stereoisomer, mixture of stereoisomers, prodrug, metabolite, crystalline form, non-crystalline form, hydrate, solvate, or salt thereof;
   in the presence of a phosphine and an azodicarboxylate reagent in a suitable solvent (e.g., THF) to form a compound of formula SVII:

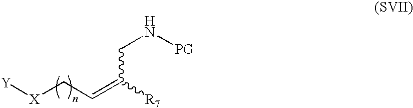

wherein A, Y, R₇, n, and PG are defined above; or any stereoisomer, mixture of stereoisomers, prodrug, metabolite, crystalline form, non-crystalline form, hydrate, solvate, or salt thereof.

In one embodiment of the process for preparing a compound of formula SVII, n is 1. In another embodiment, Y is phenyl, optionally substituted with one or more R₁ substituents. In another embodiment Y is phenyl, optionally substituted with one or more R₁ substituents, and n is 1. In another embodiment X is —O—. In another embodiment X is —S—. In another embodiment X is —O— and n is 1. In another embodiment X is —S— and n is 1. In another embodiment X is —O—, n is 1, and Y is phenyl, optionally substituted with one or more R₁ substituents. In another embodiment X is —S—, n is 1, and Y is phenyl, optionally substituted with one or more R₁ substituents. In another embodiment, each R₁ is independently selected from —C₁-C₈ alkyl, —O—C₁-C₈ alkyl, —C₁-C₈ alkyl-O—C₁-C₈ alkyl, —S—C₁-C₈ alkyl, —CF₃, —OCF₃, —F, —S(O₂)—(C₁-C₈ alkyl), and —NR₅R₆. In another embodiment, Y is phenyl, optionally substituted with one or more R₁ substituents, n is 0 or 1 and each R₁ is independently selected from —C₁-C₈ alkyl, —O—C₁-C₈ alkyl, —C₁-C₈ alkyl-O—C₁-C₈ alkyl, —S—C₁-C₈ alkyl, —CF₃, —OCF₃, —F, —S(O₂)—(C₁-C₈ alkyl), and —NR₅R₆. In another embodiment, Y is phenyl, optionally substituted with one or more R₁ substituents, n is 0 and each R₁ is independently selected from —C₁-C₈ alkyl, —O—C₁-C₈ alkyl, —C₁-C₈ alkyl-O—C₁-C₈ alkyl, —S—C₁-C₈ alkyl, —CF₃, —OCF₃, —F, —S(O₂)—(C₁-C₈ alkyl), and —NR₅R₆. In another embodiment, Y is phenyl, optionally substituted with one or more R₁ substituents, n is 1 and each R₁ is independently selected from —C₁-C₈ alkyl, —O—C₁-C₈ alkyl, —C₁-C₈ alkyl-O—C₁-C₈ alkyl, —S—C₁-C₈ alkyl, —CF₃, —OCF₃, —F, —S(O₂)—(C₁-C₈ alkyl), and —NR₅R₆. In another embodiment, R₇ is —F. In another embodiment, Y is phenyl, optionally substituted with one or more R₁ substituents, and R₇ is —F. In another embodiment, Y is phenyl, optionally substituted with one or more R₁ substituents, n is 1 and R₇ is —F. In another embodiment, the protection group (PG) is tert-butyloxycarbonyl (Boc).

In one embodiment, the process for preparing a compound of formula SVII further comprises separation of the isomeric mixture. In another embodiment, the process comprises separation of the isomeric mixture by column chromatography. In another embodiment, the phosphine is triphenyl phosphine (PPh₃) and the azodicarboxylate is diisopropyl azodicarboxylate (DIAD).

In one embodiment of the process for preparing a compound of formula SVII, the Z enantiomer of SVII is produced in an amount greater than about 50%, greater than about 60%, greater than about 70%, greater than about 80%, greater than about 85%, greater than about 90%, greater than about 95%, greater than about 97%, greater than about 98%, or greater than about 99%. In another embodiment, the E enantiomer of SVII is produced in an amount greater than about 50%, greater than about 60%, greater than about 70%, greater than about 80%, greater than about 85%, greater than about 90%, greater than about 95%, greater than about 97%, greater than about 98%, or greater than about 99%.

In another embodiment, the process for preparing a compound of formula SVII further comprises removing the protecting group (PG) from SVII in a suitable solvent (e.g., dichloromethane, followed by diethyl ether when forming and isolating the salt) to form a compound of formula SVIII:

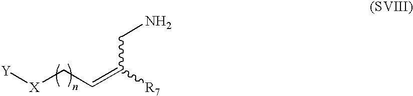

wherein X, Y, n, and R₇ are as defined above; or any stereoisomer, mixture of stereoisomers, prodrug, metabolite, crystalline form, non-crystalline form, hydrate, solvate, or salt thereof.

In one embodiment of the process for preparing a compound of formula SVIII, the protecting group (PG) is removed with a strong acid. In another embodiment, the protecting group (PG) is removed with trifluoroacetic acid (TFA).

In another embodiment, the present invention relates to a process for preparing a compound of formula SIX:

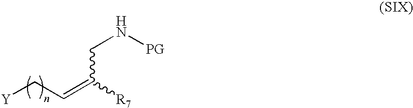

wherein Y is aryl, heteroaryl, or —C₁-C₈ alkyl, optionally substituted with one, two, three, four, or five groups from R₁, wherein each R₁ is independently selected from —C₁-C₈ alkyl, —C₃-C₈ cycloalkyl, —O—C₁-C₈ alkyl, —C₁-C₈ alkyl-O—C₁-C₈ alkyl, —O—C₃-C₈ cycloalkyl, —O—C₁-C₈ alkyl-C₃-C₈ cycloalkyl, —C₆-C₁₀ aryl, —O—C₁-C₄ alkyl-C₆-C₁₀ aryl, —S—C₁-C₈ alkyl, —S—CF₃, —CF₃, —OCF₃, —OCH₃CF₃, —CN, —F, —Cl, —Br, —I, —NO₂, —OH, —NHR₄, —NR₅R₆, and —S(O₂)—(C₁-C₈ alkyl); R₄ is selected from —C(═O)—(C₁-C₈ alkyl) and —C(═O)—(C₆-C₁₀ aryl); R₅ and R₆ are independently selected from —H, —C₁-C₈ alkyl, —C₁-C₄ alkyl-C₆-C₁₀ aryl, or R₅ and R₆ together with the nitrogen to which they are attached form a nitrogen-containing ring; n is selected from 1, 2 and 3; and R₇ is selected from —H, —F, —C₁-C₄ alkyl, and —CF₃; and PG is a suitable protecting group; or any stereoisomer, mixture of stereoisomers, prodrug, metabolite, crystalline form, non-crystalline form, hydrate, solvate, or salt thereof, comprising:
   reacting a compound of formula SIX-A:

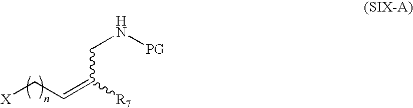

wherein X is selected from —F, —Cl, —Br, and —I; and R₇, PG, and n are as defined above; or any stereoisomer, mixture of stereoisomers, prodrug, metabolite, crystalline form, non-crystalline form, hydrate, solvate, or salt thereof;
   and a compound of formula SIX-B:

Y—B(OH)₂ (SIX-B)
wherein Y is defined above; or any stereoisomer, mixture of stereoisomers, prodrug, metabolite, crystalline form, non-crystalline form, hydrate, solvate, or salt thereof;
   in the presence of a metal complex in a suitable solvent (e.g., benzene) to form a compound of formula SIX:

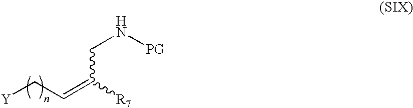

wherein Y, R₇, n, and PG are defined above; or any stereoisomer, mixture of stereoisomers, prodrug, metabolite, crystalline form, non-crystalline form, hydrate, solvate, or salt thereof.

In one embodiment of the process for preparing a compound of formula SIX, n is 1. In another embodiment, Y is phenyl, optionally substituted with one or more R₁ substituents. In another embodiment Y is phenyl, optionally substituted with one or more R₁ substituents, and n is 1. In another embodiment, each R₁ is independently selected from —C₁-C₈ alkyl, —O—C₁-C₈ alkyl, —C₁-C₈ alkyl-O—C₁-C₈ alkyl, —S—C₁-C₈ alkyl, —CF₃, —OCF₃, —F, —S(O₂)—(C₁-C₈ alkyl), and —NR₅R₆. In another embodiment, Y is phenyl, optionally substituted with one or more R₁ substituents, n is 1 and each R₁ is independently selected from —C₁-C₈ alkyl, —O—C₁-C₈ alkyl, —C₁-C₈ alkyl-O—C₁-C₈ alkyl, —S—C₁-C₈ alkyl, —CF₃, —OCF₃, —F, —S(O₂)—(C₁-C₈ alkyl), and —NR₅R₆. In another embodiment, R₇ is —F. In another embodiment, Y is phenyl, optionally substituted with one or more R₁ substituents, and R₇ is —F. In another embodiment, Y is phenyl, optionally substituted with one or more R₁ substituents, n is 1 and R₇ is —F.

In another embodiment, the process for preparing a compound of formula SIX further comprises separation of the isomeric mixture. In another embodiment, the process further comprises separation of the isomeric mixture by column chromatography. In another embodiment, the metal complex contains palladium. In another embodiment, the metal complex is Bis(dibenzylideneacetone)Pd(0).

In one embodiment of the process for preparing a compound of formula SIX, the Z enantiomer of SIX is produced in an amount greater than about 50%, greater than about 60%, greater than about 70%, greater than about 80%, greater than about 85%, greater than about 90%, greater than about 95%, greater than about 97%, greater than about 98%, or greater than about 99%. In another embodiment, the E enantiomer of SV is produced in an amount greater than about 50%, greater than about 60%, greater than about 70%, greater than about 80%, greater than about 85%, greater than about 90%, greater than about 95%, greater than about 97%, greater than about 98%, or greater than about 99%.

In another embodiment, the process for preparing a compound of formula SIX further comprises removing the protecting group (PG) from SIX in a suitable solvent (e.g., dichloromethane, followed by diethyl ether when forming and isolating the salt) to form a compound of formula SX:

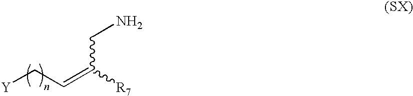

wherein Y, n, and R₇ are as defined above; or any stereoisomer, mixture of stereoisomers, prodrug, metabolite, crystalline form, non-crystalline form, hydrate, solvate, or salt thereof.

In one embodiment of the process for preparing a compound of formula SVIII, the protecting group (PG) is removed with a strong acid. In another embodiment, the protecting group (PG) is removed with trifluoroacetic acid (TFA).

In another embodiment, the present invention relates to methods of using one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 to inhibit SSAO enzyme activity (whether the enzyme activity is due either to soluble SSAO enzyme or membrane-bound VAP-1 protein, or due to both) and/or inhibit binding to VAP-1 protein. The compound(s) can be used for a method of inhibiting SSAO activity or inhibiting binding to VAP-1 in vitro, by supplying the compound to the in vitro environment in an amount sufficient to inhibit SSAO activity or inhibit binding to VAP-1. The compound(s) can also be used for a method of inhibiting SSAO activity or inhibiting binding to VAP-1 in vivo, that is, in a living organism, such as a vertebrate, mammal, or human, by administering the compound to the organism in an amount sufficient to inhibit SSAO activity or inhibit binding to VAP-1. In other embodiments of the invention, one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 is administered to a subject or patient in an amount sufficient to inhibit SSAO enzyme activity (whether the enzyme activity is due either to soluble SSAO enzyme or membrane-bound VAP-1 protein, or due to both) and/or inhibit binding to VAP-1 protein by at least about 50% or more than about 50%, while inhibiting MAO, MAO-A, MAO-B, or both MAO-A and MAO-B activity by no more than about 25%. In other embodiments of the invention, one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 is administered to a subject or patient in an amount sufficient to inhibit SSAO enzyme activity (whether the enzyme activity is due either to soluble SSAO enzyme or membrane-bound VAP-1 protein, or due to both) and/or inhibit binding to VAP-1 protein by at least about 75% or more than about 75%, while inhibiting MAO, MAO-A, MAO-B, or both MAO-A and MAO-B activity by no more than about 25%. In other embodiments of the invention, one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 is administered to a subject or patient in an amount sufficient to inhibit SSAO enzyme activity (whether the enzyme activity is due either to soluble SSAO enzyme or membrane-bound VAP-1 protein, or due to both) and/or inhibit binding to VAP-1 protein by at least about 75% or more than about 75%, while inhibiting MAO, MAO-A, MAO-B, or both MAO-A and MAO-B activity by no more than about 10%. In other embodiments of the invention, one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 is administered to a subject or patient in an amount sufficient to inhibit SSAO enzyme activity (whether the enzyme activity is due either to soluble SSAO enzyme or membrane-bound VAP-1 protein, or due to both) and/or inhibit binding to VAP-1 protein by at least about 90% or more than about 90%, while inhibiting MAO, MAO-A, MAO-B, or both MAO-A and MAO-B activity by no more than about 10%.

In other embodiments, the invention embraces unit dosage formulations of one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 which, when administered to a subject, is sufficient to inhibit SSAO enzyme activity (whether the enzyme activity is due either to soluble SSAO enzyme or membrane-bound VAP-1 protein, or due to both) and/or inhibit binding to VAP-1 protein by at least about 50% or more than about 50%, while inhibiting MAO, MAO-A, MAO-B, or both MAO-A and MAO-B activity by no more than about 25%; or is sufficient to inhibit SSAO enzyme activity (whether the enzyme activity is due either to soluble SSAO enzyme or membrane-bound VAP-1 protein, or due to both) and/or inhibit binding to VAP-1 protein by at least about 75% or more than about 75%, while inhibiting MAO, MAO-A, MAO-B, or both MAO-A and MAO-B activity by no more than about 25%; or is sufficient to inhibit SSAO enzyme activity (whether the enzyme activity is due either to soluble SSAO enzyme or membrane-bound VAP-1 protein, or due to both) and/or inhibit binding to VAP-1 protein by at least about 75% or more than about 75%, while inhibiting MAO, MAO-A, MAO-B, or both MAO-A and MAO-B activity by no more than about 10%; or is sufficient to inhibit SSAO enzyme activity (whether the enzyme activity is due either to soluble SSAO enzyme or membrane-bound VAP-1 protein, or due to both) and/or inhibit binding to VAP-1 protein by at least about 90% or more than about 90%, while inhibiting MAO, MAO-A, MAO-B, or both MAO-A and MAO-B activity by no more than about 10%.

In other embodiments of the invention, one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 is administered to a subject or patient in an amount sufficient to inhibit SSAO enzyme activity (whether the enzyme activity is due either to soluble SSAO enzyme or membrane-bound VAP-1 protein, or due to both) and/or inhibit binding to VAP-1 protein by at least about 50% or more than about 50%, while inhibiting diamine oxidase activity by no more than about 25%. In other embodiments of the invention, one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 is administered to a subject or patient in an amount sufficient to inhibit SSAO enzyme activity (whether the enzyme activity is due either to soluble SSAO enzyme or membrane-bound VAP-1 protein, or due to both) and/or inhibit binding to VAP-1 protein by at least about 75% or more than about 75%, while inhibiting diamine oxidase activity by no more than about 25%. In other embodiments of the invention, one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 is administered to a subject or patient in an amount sufficient to inhibit SSAO enzyme activity (whether the enzyme activity is due either to soluble SSAO enzyme or membrane-bound VAP-1 protein, or due to both) and/or inhibit binding to VAP-1 protein by at least about 75% or more than about 75%, while inhibiting diamine oxidase activity by no more than about 10%. In other embodiments of the invention, one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 is administered to a subject or patient in an amount sufficient to inhibit SSAO enzyme activity (whether the enzyme activity is due either to soluble SSAO enzyme or membrane-bound VAP-1 protein, or due to both) and/or inhibit binding to VAP-1 protein by at least about 90% or more than about 90%, while inhibiting diamine oxidase activity by no more than about 10%.

In other embodiments, the invention embraces unit dosage formulations of one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 which, when administered to a subject, is sufficient to inhibit SSAO enzyme activity (whether the enzyme activity is due either to soluble SSAO enzyme or membrane-bound VAP-1 protein, or due to both) and/or inhibit binding to VAP-1 protein by at least about 50% or more than about 50%, while inhibiting diamine oxidase activity by no more than about 25%; or is sufficient to inhibit SSAO enzyme activity (whether the enzyme activity is due either to soluble SSAO enzyme or membrane-bound VAP-1 protein, or due to both) and/or inhibit binding to VAP-1 protein by at least about 75% or more than about 75%, while inhibiting diamine oxidase activity by no more than about 25%; or is sufficient to inhibit SSAO enzyme activity (whether the enzyme activity is due either to soluble SSAO enzyme or membrane-bound VAP-1 protein, or due to both) and/or inhibit binding to VAP-1 protein by at least about 75% or more than about 75%, while inhibiting diamine oxidase activity by no more than about 10%; or is sufficient to inhibit SSAO enzyme activity (whether the enzyme activity is due either to soluble SSAO enzyme or membrane-bound VAP-1 protein, or due to both) and/or inhibit binding to VAP-1 protein by at least about 90% or more than about 90%, while inhibiting diamine oxidase activity by no more than about 10%.

In other embodiments of the invention, one or more compounds of formula I, I-P, I-E, I-P-E, I-P-Z, I-A, I-AP, I-A-E, I-AP-E, I-A-Z, I-AP-Z, I-B, I-BP, I-B-E, I-B P-E, I-B-Z, I-B P-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10, but excluding compound I-1-Z, is administered to a subject or patient in an amount sufficient to inhibit SSAO enzyme activity (whether the enzyme activity is due either to soluble SSAO enzyme or membrane-bound VAP-1 protein, or due to both) and/or inhibit binding to VAP-1 protein by at least about 50% or more than about 50%, while binding to a receptor or transporter (where the compound may inhibit, antagonize, activate, or agonize the receptor or transporter) listed in Table 2-A, Table 2-B, Table 2-C, or Table 2-D by no more than about 50%. In other embodiments of the invention, one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10, but excluding compound I-1-Z, is administered to a subject or patient in an amount sufficient to inhibit SSAO enzyme activity (whether the enzyme activity is due either to soluble SSAO enzyme or membrane-bound VAP-1 protein, or due to both) and/or inhibit binding to VAP-1 protein by at least about 75% or more than about 75%, while binding to a receptor or transporter (where the compound may inhibit, antagonize, activate, or agonize the receptor or transporter) listed in Table 2-A, Table 2-B, Table 2-C, or Table 2-D by no more than about 50%. In other embodiments of the invention, one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10, but excluding compound I-1-Z, is administered to a subject or patient in an amount sufficient to inhibit SSAO enzyme activity (whether the enzyme activity is due either to soluble SSAO enzyme or membrane-bound VAP-1 protein, or due to both) and/or inhibit binding to VAP-1 protein by at least about 50% or more than about 50%, while binding to a receptor or transporter (where the compound may inhibit, antagonize, activate, or agonize the receptor or transporter) listed in Table 2-A, Table 2-B, Table 2-C, or Table 2-D by no more than about 25%. In other embodiments of the invention, one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10, but excluding compound I-1-Z, is administered to a subject or patient in an amount sufficient to inhibit SSAO enzyme activity (whether the enzyme activity is due either to soluble SSAO enzyme or membrane-bound VAP-1 protein, or due to both) and/or inhibit binding to VAP-1 protein by at least about 75% or more than about 75%, while binding to a receptor or transporter (where the compound may inhibit, antagonize, activate, or agonize the receptor or transporter) listed in Table 2-A, Table 2-B, Table 2-C, or Table 2-D by no more than about 25%. In other embodiments of the invention, one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, I-B-E, II-B-Z, any one of II-1 through II-23, IV, and any one of IV-1 through IV-10, but excluding compound I-1-Z, is administered to a subject or patient in an amount sufficient to inhibit SSAO enzyme activity (whether the enzyme activity is due either to soluble SSAO enzyme or membrane-bound VAP-1 protein, or due to both) and/or inhibit binding to VAP-1 protein by at least about 75% or more than about 75%, while binding to a receptor or transporter (where the compound may inhibit, antagonize, activate, or agonize the receptor or transporter) listed in Table 2-A, Table 2-B, Table 2-C, or Table 2-D by no more than about 10%. In other embodiments of the invention, one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10, but excluding compound I-1-Z, is administered to a subject or patient in an amount sufficient to inhibit SSAO enzyme activity (whether the enzyme activity is due either to soluble SSAO enzyme or membrane-bound VAP-1 protein, or due to both) and/or inhibit binding to VAP-1 protein by at least about 90% or more than about 90%, while binding to a receptor or transporter (where the compound may inhibit, antagonize, activate, or agonize the receptor or transporter) listed in Table 2-A, Table 2-B, Table 2-C, or Table 2-D by no more than about 10%. In any of the foregoing embodiments, any one, any five, any ten, or all of the receptors and transporters listed may be selected as the receptor or transporter which falls at or below the level specified for binding, inhibiting, antagonizing, activating, or agonizing. In any of the foregoing embodiments, the binding can be measured by an assay such as a competitive binding assay. In any of the foregoing embodiments, the binding can be measured by the procedures listed in Table 2 and Table 3.

In other embodiments, the invention embraces unit dosage formulations of one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10, but excluding compound I-1-Z, which, when administered to a subject, is sufficient to inhibit SSAO enzyme activity (whether the enzyme activity is due either to soluble SSAO enzyme or membrane-bound VAP-1 protein, or due to both) and/or inhibit binding to VAP-1 protein by at least about 50% or more than about 50%, while binding to a receptor or transporter (where the compound may inhibit, antagonize, activate, or agonize the receptor or transporter) listed in Table 2-A, Table 2-B, Table 2-C, or Table 2-D by no more than about 50%; or is sufficient to inhibit SSAO enzyme activity (whether the enzyme activity is due either to soluble SSAO enzyme or membrane-bound VAP-1 protein, or due to both) and/or inhibit binding to VAP-1 protein by at least about 75% or more than about 75%, while binding to a receptor or transporter (where the compound may inhibit, antagonize, activate, or agonize the receptor or transporter) listed in Table 2-A, Table 2-B, Table 2-C, or Table 2-D by no more than about 50%; or is sufficient to inhibit SSAO enzyme activity (whether the enzyme activity is due either to soluble SSAO enzyme or membrane-bound VAP-1 protein, or due to both) and/or inhibit binding to VAP-1 protein by at least about 50% or more than about 50%, while binding to a receptor or transporter (where the compound may inhibit, antagonize, activate, or agonize the receptor or transporter) listed in Table 2-A, Table 2-B, Table 2-C, or Table 2-D by no more than about 25%; or is sufficient to inhibit SSAO enzyme activity (whether the enzyme activity is due either to soluble SSAO enzyme or membrane-bound VAP-1 protein, or due to both) and/or inhibit binding to VAP-1 protein by at least about 75% or more than about 75%, while binding to a receptor or transporter (where the compound may inhibit, antagonize, activate, or agonize the receptor or transporter) listed in Table 2-A, Table 2-B, Table 2-C, or Table 2-D by no more than about 25%; or is sufficient to inhibit SSAO enzyme activity (whether the enzyme activity is due either to soluble SSAO enzyme or membrane-bound VAP-1 protein, or due to both) and/or inhibit binding to VAP-1 protein by at least about 75% or more than about 75%, while binding to a receptor or transporter (where the compound may inhibit, antagonize, activate, or agonize the receptor or transporter) listed in Table 2-A, Table 2-B, Table 2-C, or Table 2-D by no more than about 10%; or is sufficient to inhibit SSAO enzyme activity (whether the enzyme activity is due either to soluble SSAO enzyme or membrane-bound VAP-1 protein, or due to both) and/or inhibit binding to VAP-1 protein by at least about 90% or more than about 90%, while binding to a receptor or transporter (where the compound may inhibit, antagonize, activate, or agonize the receptor or transporter) listed in Table 2-A, Table 2-B, Table 2-C, or Table 2-D by no more than about 10%. In any of the foregoing embodiments, any one, any five, any ten, or all of the receptors and transporters listed may be selected as the receptor or transporter which falls at or below the level specified for binding, inhibiting, antagonizing, activating, or agonizing. In any of the foregoing embodiments, the binding can be measured by an assay such as a competitive binding assay. In any of the foregoing embodiments, the binding can be measured by the procedures listed in Table 2 and Table 3.

In other embodiments of the invention, one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 is administered to a subject or patient in an amount sufficient to inhibit SSAO enzyme activity (whether the enzyme activity is due either to soluble SSAO enzyme or membrane-bound VAP-1 protein, or due to both) and/or inhibit binding to VAP-1 protein by at least about 50% or more than about 50%, while binding to a receptor (where the compound may inhibit, antagonize, activate, or agonize the receptor) listed in Table 2-A, Table 2-B, or Table 2-C (with the proviso that the 5-HT_{1A} receptor is excluded) by no more than about 50%. In other embodiments of the invention, one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 is administered to a subject or patient in an amount sufficient to inhibit SSAO enzyme activity (whether the enzyme activity is due either to soluble SSAO enzyme or membrane-bound VAP-1 protein, or due to both) and/or inhibit binding to VAP-1 protein by at least about 75% or more than about 75%, while binding to a receptor (where the compound may inhibit, antagonize, activate, or agonize the receptor) listed in Table 2-A, Table 2-B, or Table 2-C (with the proviso that the 5-HT_{1A} receptor is excluded) by no more than about 50%. In other embodiments of the invention, one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 is administered to a subject or patient in an amount sufficient to inhibit SSAO enzyme activity (whether the enzyme activity is due either to soluble SSAO enzyme or membrane-bound VAP-1 protein, or due to both) and/or inhibit binding to VAP-1 protein by at least about 90% or more than about 90%, while binding to a receptor (where the compound may inhibit, antagonize, activate, or agonize the receptor) listed in Table 2-A, Table 2-B, or Table 2-C (with the proviso that the 5-HT_{1A} receptor is excluded) by no more than about 50%. In other embodiments of the invention, one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 is administered to a subject or patient in an amount sufficient to inhibit SSAO enzyme activity (whether the enzyme activity is due either to soluble SSAO enzyme or membrane-bound VAP-1 protein, or due to both) and/or inhibit binding to VAP-1 protein by at least about 50% or more than about 50%, while binding to a receptor (where the compound may inhibit, antagonize, activate, or agonize the receptor) listed in Table 2-A, Table 2-B, or Table 2-C (with the proviso that the 5-HT_{1A} receptor is excluded) by no more than about 30%. In other embodiments of the invention, one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 is administered to a subject or patient in an amount sufficient to inhibit SSAO enzyme activity (whether the enzyme activity is due either to soluble SSAO enzyme or membrane-bound VAP-1 protein, or due to both) and/or inhibit binding to VAP-1 protein by at least about 75% or more than about 75%, while binding to a receptor (where the compound may inhibit, antagonize, activate, or agonize the receptor) listed in Table 2-A, Table 2-B, or Table 2-C (with the proviso that the 5-HT_{1A} receptor is excluded) by no more than about 30%. In other embodiments of the invention, one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 is administered to a subject or patient in an amount sufficient to inhibit SSAO enzyme activity (whether the enzyme activity is due either to soluble SSAO enzyme or membrane-bound VAP-1 protein, or due to both) and/or inhibit binding to VAP-1 protein by at least about 90% or more than about 90%, while binding to a receptor (where the compound may inhibit, antagonize, activate, or agonize the receptor) listed in Table 2-A, Table 2-B, or Table 2-C (with the proviso that the 5-HT_{1A} receptor is excluded) by no more than about 30%. In any of the foregoing embodiments, the compound can be compound I-1-Z. In any of the foregoing embodiments, any one, any five, any ten, or all of the receptors listed may be selected as the receptor which falls at or below the level specified for binding, inhibiting, antagonizing, activating, or agonizing. In any of the foregoing embodiments, the binding can be measured by an assay such as a competitive binding assay. In any of the foregoing embodiments, the binding can be measured by the procedures listed in Table 2 and Table 3.

In other embodiments, the invention embraces unit dosage formulations of one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 which, when administered to a subject, is sufficient to inhibit SSAO enzyme activity (whether the enzyme activity is due either to soluble SSAO enzyme or membrane-bound VAP-1 protein, or due to both) and/or inhibit binding to VAP-1 protein by at least about 50% or more than about 50%, while binding to a receptor (where the compound may inhibit, antagonize, activate, or agonize the receptor) listed in Table 2-A, Table 2-B, or Table 2-C (with the proviso that the 5-HT_{1A} receptor is excluded) by no more than about 50%. In other embodiments, the invention embraces unit dosage formulations of one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 which, when administered to a subject, is sufficient to inhibit SSAO enzyme activity (whether the enzyme activity is due either to soluble SSAO enzyme or membrane-bound VAP-1 protein, or due to both) and/or inhibit binding to VAP-1 protein by at least about 75% or more than about 75%, while binding to a receptor (where the compound may inhibit, antagonize, activate, or agonize the receptor) listed in Table 2-A, Table 2-B, or Table 2-C (with the proviso that the 5-HT_{1A} receptor is excluded) by no more than about 50%. In other embodiments, the invention embraces unit dosage formulations of one or more compounds of formula I, I-P, I-E, I-P-E, I-P-Z, I-A, I-A-P, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 which, when administered to a subject, is sufficient to inhibit SSAO enzyme activity (whether the enzyme activity is due either to soluble SSAO enzyme or membrane-bound VAP-1 protein, or due to both) and/or inhibit binding to VAP-1 protein by at least about 90% or more than about 90%, while binding to a receptor (where the compound may inhibit, antagonize, activate, or agonize the receptor) listed in Table 2-A, Table 2-B, or Table 2-C (with the proviso that the 5-HT_{1A} receptor is excluded) by no more than about 50%. In other embodiments, the invention embraces unit dosage formulations of one or more compounds of formula I, I-P, I-E, I-P-E, I-P-Z, I-A, I-AP, I-A-E, I-AP-E, I-A-Z, I-AP-Z, I-B, I-B P, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 which, when administered to a subject, is sufficient to inhibit SSAO enzyme activity (whether the enzyme activity is due either to soluble SSAO enzyme or membrane-bound VAP-1 protein, or due to both) and/or inhibit binding to VAP-1 protein by at least about 50% or more than about 50%, while binding to a receptor (where the compound may inhibit, antagonize, activate, or agonize the receptor) listed in Table 2-A, Table 2-B, or Table 2-C (with the proviso that the 5-HT_{1A} receptor is excluded) by no more than about 30%. In other embodiments, the invention embraces unit dosage formulations of one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 which, when administered to a subject, is sufficient to inhibit SSAO enzyme activity (whether the enzyme activity is due either to soluble SSAO enzyme or membrane-bound VAP-1 protein, or due to both) and/or inhibit binding to VAP-1 protein by at least about 75% or more than about 75%, while binding to a receptor (where the compound may inhibit, antagonize, activate, or agonize the receptor) listed in Table 2-A, Table 2-B, or Table 2-C (with the proviso that the 5-HT_{1A} receptor is excluded) by no more than about 30%. In other embodiments, the invention embraces unit dosage formulations of one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 which, when administered to a subject, is sufficient to inhibit SSAO enzyme activity (whether the enzyme activity is due either to soluble SSAO enzyme or membrane-bound VAP-1 protein, or due to both) and/or inhibit binding to VAP-1 protein by at least about 90% or more than about 90%, while binding to a receptor (where the compound may inhibit, antagonize, activate, or agonize the receptor) listed in Table 2-A, Table 2-B, or Table 2-C (with the proviso that the 5-HT_{1A} receptor is excluded) by no more than about 30%. In any of the foregoing embodiments, the compound can be compound I-1-Z. In any of the foregoing embodiments, any one, any five, any ten, or all of the receptors listed may be selected as the receptor which falls at or below the level specified for binding, inhibiting, antagonizing, activating, or agonizing. In any of the foregoing embodiments, the binding can be measured by an assay such as a competitive binding assay. In any of the foregoing embodiments, the binding can be measured by the procedures listed in Table 2 and Table 3.

In other embodiments of the invention, one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 is administered to a subject or patient in an amount sufficient to inhibit SSAO enzyme activity (whether the enzyme activity is due either to soluble SSAO enzyme or membrane-bound VAP-1 protein, or due to both) and/or inhibit binding to VAP-1 protein by at least about 50% or more than about 50%, while binding to the 5-HT_{1A} receptor, the dopamine transporter, or the serotonin transporter by no more than about 75%, preferably no more than about 50%, more preferably no more than about 30% (where the compound may inhibit, antagonize, activate, or agonize the receptor or transporter). In other embodiments of the invention, one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 is administered to a subject or patient in an amount sufficient to inhibit SSAO enzyme activity (whether the enzyme activity is due either to soluble SSAO enzyme or membrane-bound VAP-1 protein, or due to both) and/or inhibit binding to VAP-1 protein by at least about 75% or more than about 75%, while binding to the 5-HT_{1A} receptor, the dopamine transporter, or the serotonin transporter by no more than about 75%, preferably no more than about 50%, more preferably no more than about 30% (where the compound may inhibit, antagonize, activate, or agonize the receptor or transporter). In other embodiments of the invention, one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 is administered to a subject or patient in an amount sufficient to inhibit SSAO enzyme activity (whether the enzyme activity is due either to soluble SSAO enzyme or membrane-bound VAP-1 protein, or due to both) and/or inhibit binding to VAP-1 protein by at least about 90% or more than about 90%, while binding to the 5-HT_{1A} receptor, the dopamine transporter, or the serotonin transporter by no more than about 75%, preferably no more than about 50%, more preferably no more than about 30% (where the compound may inhibit, antagonize, activate, or agonize the receptor or transporter). In any of the foregoing embodiments, the compound can be compound I-1-Z. In any of the foregoing embodiments, any one, any two, any three, or all four of the receptors/transporters listed may be selected as the receptor/transporter which falls at or below the level specified for inhibiting, antagonizing, activating, or agonizing. In any of the foregoing embodiments, the binding can be measured by an assay such as a competitive binding assay. In any of the foregoing embodiments, the binding can be measured by the procedures listed in Table 2 and Table 3.

In other embodiments, the invention embraces unit dosage formulations of one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 which, when administered to a subject, is sufficient to inhibit SSAO enzyme activity (whether the enzyme activity is due either to soluble SSAO enzyme or membrane-bound VAP-1 protein, or due to both) and/or inhibit binding to VAP-1 protein by at least about 50% or more than about 50%, while binding to the 5-HT_{1A} receptor, the dopamine transporter, or the serotonin transporter by no more than about 75%, preferably no more than about 50%, more preferably no more than about 30% (where the compound may inhibit, antagonize, activate, or agonize the receptor or transporter). In other embodiments, the invention embraces unit dosage formulations of one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 which, when administered to a subject, is sufficient to inhibit SSAO enzyme activity (whether the enzyme activity is due either to soluble SSAO enzyme or membrane-bound VAP-1 protein, or due to both) and/or inhibit binding to VAP-1 protein by at least about 75% or more than about 75%, while binding to the 5-HT_{1A} receptor, the dopamine transporter, or the serotonin transporter by no more than about 75%, preferably no more than about 50%, more preferably no more than about 30% (where the compound may inhibit, antagonize, activate, or agonize the receptor or transporter). In other embodiments, the invention embraces unit dosage formulations of one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 which, when administered to a subject, is sufficient to inhibit SSAO enzyme activity (whether the enzyme activity is due either to soluble SSAO enzyme or membrane-bound VAP-1 protein, or due to both) and/or inhibit binding to VAP-1 protein by at least about 90% or more than about 90%, while binding to the 5-HT_{1A} receptor, the dopamine transporter, or the serotonin transporter by no more than about 75%, preferably no more than about 50%, more preferably no more than about 30% (where the compound may inhibit, antagonize, activate, or agonize the receptor or transporter). In any of the foregoing embodiments, the compound can be compound I-1-Z. In any of the foregoing embodiments, any one, any two, any three, or all four of the receptors/transporters listed may be selected as the receptor/transporter which falls at or below the level specified for inhibiting, antagonizing, activating, or agonizing. In any of the foregoing embodiments, the binding can be measured by an assay such as a competitive binding assay. In any of the foregoing embodiments, the binding can be measured by the procedures listed in Table 2 and Table 3.

In other embodiments of the invention, one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 is administered to a subject or patient in an amount sufficient to inhibit SSAO enzyme activity (whether the enzyme activity is due either to soluble SSAO enzyme or membrane-bound VAP-1 protein, or due to both) and/or inhibit binding to VAP-1 protein by at least about 50% or more than about 50%, while binding to the norepinephrine transporter by no more than about 75%, preferably no more than about 50%, more preferably no more than about 30% (where the compound may inhibit, antagonize, activate, or agonize the transporter). In other embodiments of the invention, one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 is administered to a subject or patient in an amount sufficient to inhibit SSAO enzyme activity (whether the enzyme activity is due either to soluble SSAO enzyme or membrane-bound VAP-1 protein, or due to both) and/or inhibit binding to VAP-1 protein by at least about 75% or more than about 75%, while binding to the norepinephrine transporter by no more than about 75%, preferably no more than about 50%, more preferably no more than about 30% (where the compound may inhibit, antagonize, activate, or agonize the transporter). In other embodiments of the invention, one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 is administered to a subject or patient in an amount sufficient to inhibit SSAO enzyme activity (whether the enzyme activity is due either to soluble SSAO enzyme or membrane-bound VAP-1 protein, or due to both) and/or inhibit binding to VAP-1 protein by at least about 90% or more than about 90%, while binding to the norepinephrine transporter by no more than about 75%, preferably no more than about 50%, more preferably no more than about 30% (where the compound may inhibit, antagonize, activate, or agonize the transporter). In any of the foregoing embodiments, the compound can be compound I-1-Z. In any of the foregoing embodiments, the binding can be measured by an assay such as a competitive binding assay. In any of the foregoing embodiments, the binding can be measured by the procedures listed in Table 2 and Table 3.

In other embodiments, the invention embraces unit dosage formulations of one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 which, when administered to a subject, is sufficient to inhibit SSAO enzyme activity (whether the enzyme activity is due either to soluble SSAO enzyme or membrane-bound VAP-1 protein, or due to both) and/or inhibit binding to VAP-1 protein by at least about 50% or more than about 50%, while binding to the norepinephrine transporter by no more than about 75%, preferably no more than about 50%, more preferably no more than about 30% (where the compound may inhibit, antagonize, activate, or agonize the transporter). In other embodiments, the invention embraces unit dosage formulations of one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 which, when administered to a subject, is sufficient to inhibit SSAO enzyme activity (whether the enzyme activity is due either to soluble SSAO enzyme or membrane-bound VAP-1 protein, or due to both) and/or inhibit binding to VAP-1 protein by at least about 75% or more than about 75%, while binding to the norepinephrine transporter by no more than about 75%, preferably no more than about 50%, more preferably no more than about 30% (where the compound may inhibit, antagonize, activate, or agonize the transporter). In other embodiments, the invention embraces unit dosage formulations of one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 which, when administered to a subject, is sufficient to inhibit SSAO enzyme activity (whether the enzyme activity is due either to soluble SSAO enzyme or membrane-bound VAP-1 protein, or due to both) and/or inhibit binding to VAP-1 protein by at least about 90% or more than about 90%, while binding to the norepinephrine transporter by no more than about 75%, preferably no more than about 50%, more preferably no more than about 30% (where the compound may inhibit, antagonize, activate, or agonize the transporter). In any of the foregoing embodiments, the compound can be compound I-1-Z. In any of the foregoing embodiments, the binding can be measured by an assay such as a competitive binding assay. In any of the foregoing embodiments, the binding can be measured by the procedures listed in Table 2 and Table 3.

In another embodiment, the present invention relates to methods of using one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 to treat or prevent inflammation or immune disorders. In another embodiment, the present invention relates to methods of using one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 to suppress or reduce inflammation, or to suppress or reduce an inflammatory response. In another embodiment, the present invention relates to methods of treating or preventing inflammation, by administering one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 in a therapeutically effective amount, or in an amount sufficient to treat or prevent inflammation. In another embodiment, the present invention relates to methods of treating or preventing immune or autoimmune disorders, by administering one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 in a therapeutically effective amount, or in an amount sufficient to treat or prevent the immune or autoimmune disorder.

In another embodiment, the inflammatory disease or immune disorder to be treated or prevented by one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 of the present invention is selected from the group consisting of multiple sclerosis (including chronic multiple sclerosis); synovitis; systemic inflammatory sepsis; inflammatory bowel diseases; Crohn's disease; ulcerative colitis; Alzheimer's disease; vascular dementia; atherosclerosis; rheumatoid arthritis; juvenile rheumatoid arthritis; pulmonary inflammatory conditions; asthma; skin inflammatory conditions and diseases; contact dermatitis; liver inflammatory and autoimmune conditions; autoimmune hepatitis; primary biliary cirrhosis; sclerosing cholangitis; autoimmune cholangitis; alcoholic liver disease; Type I diabetes and/or complications thereof; Type II diabetes and/or complications thereof; atherosclerosis; chronic heart failure; congestive heart failure; ischemic diseases such as stroke and/or complications thereof; and myocardial infarction and/or complications thereof. In another embodiment, the inflammatory disease or immune disorder to be treated or prevented by the present invention is multiple sclerosis (including chronic multiple sclerosis). In another embodiment, the inflammatory disease or immune disorder to be treated or prevented by the present invention is stroke or the inflammatory complications resulting from stroke.

A compound of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, I-B-E, II-B-Z, any one of II-1 through II-23, IV, and any one of IV-1 through IV-10 as described above can be administered singly in a therapeutically effective amount. A compound of formula compound of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, I-B-E, II-B-Z, any one of II-1 through II-23, IV, and any one of IV-1 through IV-10 as described above can be administered with one or more additional compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10, in a therapeutically effective amount. When administered in combination, the compounds can be administered in amounts that would be therapeutically effective were the compounds to be administered singly. Alternatively, when administered in combination, any or all of compounds can be administered in amounts that would not be therapeutically effective were the compounds to be administered singly, but which are therapeutically effective in combination. One or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 can also be administered with other compounds not included in formulas I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10; the compounds can be administered in amounts that are therapeutically effective when used as single drugs, or in amounts which are not therapeutically effective as single drugs, but which are therapeutically effective in combination. Also provided are pharmaceutically acceptable compositions comprising a therapeutically effective amount of one or more of the compounds disclosed herein or a therapeutically effective combination of two or more of the compounds disclosed herein, including the compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 above, and a pharmaceutically acceptable carrier; and human unit dosages thereof.

A compound of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 as described above can be prepared as an isolated pharmaceutical composition, and administered as an isolated pharmaceutical composition in conjunction with vehicles or other isolated compounds. That is, a compound of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 as described above can be isolated from other compounds (e.g., a compound which is discovered in a library screening assay can be purified out of the library, or synthesized de novo as a single compound). The degree of purification can be 90%, 95%, 99%, or whatever percentage of purity is required for pharmaceutical use of the compound. The isolated compound can then be combined with pharmaceutically acceptable vehicles, or can be combined with one or more isolated compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10, or with another therapeutic substance. A compound of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 as described above can be administered orally, in a pharmaceutical human unit dosage formulation. The pharmaceutical human unit dosage formulation can contain a therapeutically effective amount of a compound of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, and any one of IV-1 through IV-10 for treatment or prevention of any disease disclosed herein.

In another embodiment, the invention embraces one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, or any one of I-1 through I-109, such as I-1-Z, I-2-Z, or I-2-E, for use in therapy. In another embodiment, the invention embraces one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, I or any one of I-1 through I-109, such as I-1-Z, I-2-Z, or I-2-E, for manufacture of a medicament for treatment or prevention of inflammatory diseases. In another embodiment, the invention embraces one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, or any one of I-1 through I-109, such as I-1-Z, I-2-Z, or I-2-E, for manufacture of a medicament for treatment or prevention of immune or autoimmune diseases. In another embodiment, the invention embraces one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, or any one of I-1 through I-109, such as I-1-Z, I-2-Z, or I-2-E, for manufacture of a medicament for treatment or prevention of multiple sclerosis or chronic multiple sclerosis. In another embodiment, the invention embraces one or more compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, I or any one of I-1 through I-109, such as I-1-Z, I-2-Z, or I-2-E, for manufacture of a medicament for treatment or prevention of ischemic diseases (such as stroke) or the sequelae of ischemic diseases.

In another embodiment, the invention embraces one or more compounds of formula II, II-E, II-Z, II-A, II-A-E, II-A-Z, II-B, II-B-E, II-B-Z, II-1, II-2, II-3, II-4, II-5, II-6, II-7, II-8, II-9, II-10, II-11, II-12, II-13, II-14, II-15, II-16, II-17, II-18, II-19, II-20, II-21, II-22, or II-23, such as II-1-E, for use in therapy. In another embodiment, the invention embraces one or more compounds of formula II, II-E, II-Z, II-A, II-A-E, II-A-Z, II-B, II-B-E, II-B-Z, II-1, II-2, II-3, II-4, II-5, II-6, II-7, II-8, II-9, II-10, II-11, II-12, II-13, II-14, II-15, II-16, II-17, II-18, II-19, II-20, II-21, II-22, or II-23, such as II-1-E, for manufacture of a medicament for treatment or prevention of inflammatory diseases.

In another embodiment, the invention embraces one or more compounds of formula II, II-E, II-Z, II-A, II-A-E, II-A-Z, II-B, II-B-E, II-B-Z, II-1, II-2, II-3, II-4, II-5, II-6, II-7, II-8, II-9, II-10, II-11, II-12, II-13, II-14, II-15, II-16, II-17, II-18, II-19, II-20, II-21, II-22, or II-23, such as II-1-E, for manufacture of a medicament for treatment or prevention of immune or autoimmune diseases. In another embodiment, the invention embraces one or more compounds of formula II, II-E, II-Z, II-A, II-A-E, II-A-Z, II-B, II-B-E, II-B-Z, II-1, II-2, II-3, II-4, II-5, II-6, II-7, II-8, II-9, II-10, II-11, II-12, II-13, II-14, II-15, II-16, II-17, II-18, II-19, II-20, II-21, II-22, or II-23, such as II-1-E, for manufacture of a medicament for treatment or prevention of multiple sclerosis or chronic multiple sclerosis. In another embodiment, the invention embraces one or more compounds of formula II, II-E, II-Z, II-A, II-A-E, II-A-Z, II-B, II-B-E, II-B-Z, II-1, II-2, II-3, II-4, II-5, II-6, II-7, II-8, II-9, II-10, II-11, II-12, II-13, II-14, II-15, II-16, II-17, II-18, II-19, II-20, II-21, II-22, II-23, or II-24, such as II-1-E, for manufacture of a medicament for treatment or prevention of ischemic diseases (such as stroke) or the sequelae of ischemic diseases.

In another embodiment, the invention embraces one or more compounds of formula IV-1, IV-2, IV-3, IV-4, IV-5, IV-6, IV-7, IV-8, IV-9, or IV-10, for use in therapy. In another embodiment, the invention embraces one or more compounds of formula IV-1, IV-2, IV-3, IV-4, IV-5, IV-6, IV-7, IV-8, IV-9, or IV-10, for manufacture of a medicament for treatment or prevention of inflammatory diseases. In another embodiment, the invention embraces one or more compounds of formula IV-1, IV-2, IV-3, IV-4, IV-5, IV-6, IV-7, IV-8, IV-9, or IV-10, for manufacture of a medicament for treatment or prevention of immune or autoimmune diseases. In another embodiment, the invention embraces one or more compounds of formula IV-1, IV-2, IV-3, IV-4, IV-5, IV-6, IV-7, IV-8, IV-9, or IV-10, for manufacture of a medicament for treatment or prevention of multiple sclerosis or chronic multiple sclerosis. In another embodiment, the invention embraces one or more compounds of formula IV-1, IV-2, IV-3, IV-4, IV-5, IV-6, IV-7, IV-8, IV-9, or IV-10, for manufacture of a medicament for treatment or prevention of ischemic diseases (such as stroke) or the sequelae of ischemic diseases.

For all of the compounds, and methods using the compounds, disclosed herein, the compounds can be admixed with a pharmaceutically acceptable excipient or pharmaceutically acceptable carrier.

### BRIEF DESCRIPTION OF THE DRAWINGS

FIG. 1 depicts the effect of Compound II-1-E on carrageenan-induced rat paw edema.

FIG. 2 depicts the effect of Compound II-1-E on body weight (FIG. 2A) and survival (FIG. 2B) of mice with ulcerative colitis.

FIG. 3 depicts the effect of Compound II-1-E on development of acute experimental autoimmune encephalomyelitis.

FIG. 4 depicts the effect of Compound I-1-Z on development of murine anti-collagen-induced arthritis.

FIG. 5 depicts the effect of Compound I-1-Z on carrageenan-induced rat paw edema.

FIG. 6 depicts the effect of Compound I-2-Z on carrageenan-induced rat paw edema.

FIG. 7 depicts the dose responsive effect of Compound I-1-Z on carrageenan-induced rat paw edema.

FIG. 8 depicts the effect of therapeutic dosing with Compound I-1-Z on carrageenan-induced rat paw edema.

FIG. 9 depicts the effect of therapeutic dosing with Compound I-1-Z on anti-collagen antibody-induced arthritis.

FIG. 10 depicts the effect of Compound I-1-Z on cell trafficking.

FIG. 11 depicts the determination of ED50 of Compound I-1-Z for rat lung SSAO.

FIG. 12 depicts the effect of low doses of Compound I-1-Z on anti-collagen antibody-induced arthritis.

FIG. 13 depicts the effect of low doses of Compound I-1-Z on carrageenan-induced rat paw edema.

FIG. 14 depicts the effect of doses of Compound I-1-Z on LPS-induced lung inflammation.

FIG. 15 depicts the effect of doses of Compound I-1-Z on collagen-induced arthritis. Panel A shows clinical arthritis scores, while Panel B shows percent incidence.

FIG. 16 depicts the effect of route of administration of Compound I-1-Z on cell trafficking.

### MODES FOR CARRYING OUT THE INVENTION

The present invention relates to various compounds which are useful for inhibiting SSAO enzyme activity (where the enzyme activity is due either to soluble SSAO enzyme or membrane-bound VAP-1 protein, or due to both) and/or inhibition of binding to membrane-bound VAP-1 protein. The present invention also relates to methods of using various compounds to inhibit SSAO enzyme activity (where the enzyme activity is due either to soluble SSAO enzyme or membrane-bound VAP-1 protein, or due to both) and/or inhibit binding to VAP-1 protein. The present invention also relates to methods of using various compounds to treat or prevent inflammation or immune disorders, and to reduce or suppress inflammation or inflammatory responses.

Compounds for use in the invention can be assayed for SSAO inhibitory activity by the protocol in the examples below. It is preferable to use compounds which specifically inhibit SSAO over monoamine oxidase. The specificity of the compounds for SSAO inhibitory activity versus MAO-A and MAO-B inhibitory activity can be assayed by the protocol in the examples below. Compounds for use in the invention have an inhibitory activity (IC₅₀) against SSAO of about <1 μM, more preferably of about 100 nM, and more preferably of about 10 nM. Preferably, compounds for use in the invention also have a specificity for SSAO versus MAO-A of about 10-fold, greater than about 10-fold, about 100-fold, greater than about 100-fold, about 500-fold, greater than about 500-fold, about 1,000-fold, greater than about 1000-fold, about 5,000-fold, or greater than about 5000-fold (where specificity for SSAO versus MAO-A is defined as the ratio of the IC₅₀ of a compound for MAO-A to the IC₅₀ of the same compound for SSAO; that is, a compound with an IC₅₀ of 10 μM for MAO-A and an IC₅₀ of 20 nM for SSAO has a specificity of 500 for SSAO versus MAO-A). Compounds for use in the invention also have a specificity for SSAO versus MAO-B of about 10-fold, greater than about 10-fold, about 100-fold, greater than about 100-fold, about 500-fold, greater than about 500-fold, about 1,000-fold, greater than about 1000-fold, about 5,000-fold, or greater than about 5000-fold (where specificity for SSAO versus MAO-B is defined as the ratio of the IC₅₀ of a compound for MAO-B to the IC₅₀ of the same compound for SSAO). Table 1 below provides experimental values for several of the compounds for use in the invention.

The term “inhibit binding to VAP-1 protein” is meant to indicate inhibition (which can include partial to complete inhibition) of binding between, for example, a cell expressing the SSAO/VAP-1 protein on its surface, and a binding partner of SSAO/VAP-1 protein. Such binding occurs, for example, when a cell expressing the SSAO/VAP-1 protein on its surface, such as a high endothelial cell (HEC) interacts with another cell expressing a binding partner of SSAO/VAP-1 protein, such as a leukocyte. Thus “inhibit binding to VAP-1 protein” embraces inhibition of adhesion between a cell expressing the SSAO/VAP-1 protein on its surface, and another cell expressing a binding partner of SSAO/VAP-1 protein. Such adhesion events include, for example, cell rolling. As this disclosure (including the examples) clearly indicates, such inhibition can occur either in vitro or in vivo. Binding can be inhibited by about 5% or by greater than about 5%, about 10% or by greater than about 10%, about 20% or by greater than about 20%, about 30% or by greater than about 30%, about 40% or by greater than about 40%, about 50% or by greater than about 50%, about 60% or by greater than about 60%, about 70% or by greater than about 70%, about 80% or by greater than about 80%, about 90% or by greater than about 90%, or about 95% or by greater than about 95%.

The invention includes all salts of the compounds described herein, as well as methods of using such salts of the compounds. The invention also includes all non-salt forms of any salt of a compound named herein, as well as other salts of any salt of a compound named herein. In one embodiment, the salts of the compounds comprise pharmaceutically acceptable salts. Pharmaceutically acceptable salts are those salts which retain the biological activity of the free compounds and which can be administered as drugs or pharmaceuticals to humans and/or animals. The desired salt of a basic compound may be prepared by methods known to those of skill in the art by treating the compound with an acid. Examples of inorganic acids include, but are not limited to, hydrochloric acid, hydrobromic acid, sulfuric acid, nitric acid, and phosphoric acid. Examples of organic acids include, but are not limited to, formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid. Salts of basic compounds with amino acids, such as aspartate salts and glutamate salts, can also be prepared. The desired salt of an acidic compound can be prepared by methods known to those of skill in the art by treating the compound with a base. Examples of inorganic salts of acid compounds include, but are not limited to, alkali metal and alkaline earth salts, such as sodium salts, potassium salts, magnesium salts, and calcium salts; ammonium salts; and aluminum salts. Examples of organic salts of acid compounds include, but are not limited to, procaine, dibenzylamine, N-ethylpiperidine, N,N′-dibenzylethylenediamine, and triethylamine salts. Salts of acidic compounds with amino acids, such as lysine salts, can also be prepared.

The invention includes all stereoisomers of the compounds referred to in the above formulas, including enantiomers and diastereomers. The invention includes all enantiomers of any chiral compound disclosed, in either substantially pure levorotatory or dextrorotatory form, or in a racemic mixture, or in any ratio of enantiomers. The invention includes any diastereomers of the compounds referred to in the above formulas in diastereomerically pure form and in the form of mixtures in all ratios. For compounds disclosed as an E isomer, the invention also includes the Z isomer; for compounds disclosed as the Z isomer, the invention also includes the E isomer. The invention also includes all solvates of the compounds referred to in the above formulas, including all hydrates of the compounds referred to in the above formulas. The invention also includes all polymorphs, including crystalline and non-crystalline forms of the compounds referred to in the above formulas. The invention also includes all salts of the compounds referred to in the above formulas, particularly pharmaceutically-acceptable salts. Metabolites and prodrugs of the compounds referred to in the above formulas are also embraced by the invention. In all uses of the compounds of the above formulas disclosed herein, the invention also includes use of any or all of the stereochemical, enantiomeric, diastereomeric, E or Z forms, solvates, hydrates, polymorphic, crystalline, non-crystalline, salt, pharmaceutically acceptable salt, metabolite and prodrug variations of the compounds as described.

Unless stereochemistry is explicitly indicated in a chemical structure or chemical name, the chemical structure or chemical name is intended to embrace all possible stereoisomers of the compound depicted. For example, the compound I-1 is intended to embrace compounds I-1-E and I-1-Z.

The term “alkyl” refers to saturated aliphatic and alicyclic groups including straight-chain, branched-chain, cyclic groups, and combinations thereof, having the number of carbon atoms specified, or if no number is specified, having up to 12 carbon atoms. “Straight-chain alkyl” or “linear alkyl” groups refers to alkyl groups that are neither cyclic nor branched, commonly designated as “n-alkyl” groups. Examples of alkyl groups include, but are not limited to, groups such as methyl, ethyl, n-propyl, isopropyl, butyl, n-butyl, isobutyl, sec-butyl, t-butyl, pentyl, n-pentyl, hexyl, heptyl, octyl, nonyl, decyl, undecyl, dodecyl, neopentyl, cyclopropyl, cyclobutyl, cyclopentyl, cyclohexyl, and adamantyl. Cycloalkyl groups can consist of one ring, including, but not limited to, groups such as cycloheptyl, or multiple fused rings, including, but not limited to, groups such as adamantyl or norbornyl.

“Substituted alkyl” refers to alkyl groups substituted with one or more substituents including, but not limited to, groups such as halogen (fluoro, chloro, bromo, and iodo), alkoxy, acyloxy, amino, hydroxyl, mercapto, carboxy, benzyloxy, phenyl, benzyl, cyano, nitro, thioalkoxy, carboxaldehyde, carboalkoxy and carboxamide, or a functionality that can be suitably blocked, if necessary for purposes of the invention, with a protecting group. Examples of substituted alkyl groups include, but are not limited to, —CF₃, —CF₂—CF₃, and other perfluoro and perhalo groups; —CH₂—OH; —CH₂CH₂CH(NH₂)CH₃, etc.

The term “alkenyl” refers to unsaturated aliphatic and alicyclic groups including straight-chain (linear), branched-chain, cyclic groups, and combinations thereof, having the number of carbon atoms specified, or if no number is specified, having up to 12 carbon atoms, which contain at least one double bond (—C═C—). Examples of alkenyl groups include, but are not limited to, —CH₂—CH═CH—CH₃; and —CH₂—CH₂-cyclohexenyl, where the ethyl group can be attached to the cyclohexenyl moiety at any available carbon valence. The term “alkynyl” refers to unsaturated aliphatic and alicyclic groups including straight-chain (linear), branched-chain, cyclic groups, and combinations thereof, having the number of carbon atoms specified, or if no number is specified, having up to 12 carbon atoms, which contain at least one triple bond (—C≡C—). “Hydrocarbon chain” or “hydrocarbyl” refers to any combination of straight-chain, branched-chain, or cyclic alkyl, alkenyl, or alkynyl groups, and any combination thereof. “Substituted alkenyl,” “substituted alkynyl,” and “substituted hydrocarbon chain” or “substituted hydrocarbyl” refer to the respective group substituted with one or more substituents, including, but not limited to, groups such as halogen, alkoxy, acyloxy, amino, hydroxyl, mercapto, carboxy, benzyloxy, phenyl, benzyl, cyano, nitro, thioalkoxy, carboxaldehyde, carboalkoxy and carboxamide, or a functionality that can be suitably blocked, if necessary for purposes of the invention, with a protecting group.

“Aryl” or “Ar” refers to an aromatic carbocyclic group having a single ring (including, but not limited to, groups such as phenyl) or two or more condensed rings (including, but not limited to, groups such as naphthyl or anthryl), and includes both unsubstituted and substituted aryl groups. Aryls, unless otherwise specified, contain from 6 to 12 carbon atoms in the ring portion. A preferred range for aryls is from 6 to 10 carbon atoms in the ring portion. “Substituted aryls” refers to aryls substituted with one or more substituents, including, but not limited to, groups such as alkyl, alkenyl, alkynyl, hydrocarbon chains, halogen, alkoxy, acyloxy, amino, hydroxyl, mercapto, carboxy, benzyloxy, phenyl, benzyl, cyano, nitro, thioalkoxy, carboxaldehyde, carboalkoxy and carboxamide, or a functionality that can be suitably blocked, if necessary for purposes of the invention, with a protecting group. “Aralkyl” designates an alkyl-substituted aryl group, where any aryl can attached to the alkyl; the alkyl portion is a straight or branched chain of 1 to 6 carbon atoms, preferably the alkyl chain contains 1 to 3 carbon atoms. When an aralkyl group is indicated as a substituent, the aralkyl group can be connected to the remainder of the molecule at any available valence on either its alkyl moiety or aryl moiety; e.g., the tolyl aralkyl group can be connected to the remainder of the molecule by replacing any of the five hydrogens on the aromatic ring moiety with the remainder of the molecule, or by replacing one of the alpha-hydrogens on the methyl moiety with the remainder of the molecule. Preferably, the aralkyl group is connected to the remainder of the molecule via the alkyl moiety.

A preferred aryl group is phenyl, which can be substituted or unsubstituted. Preferred substituents for substituted phenyl groups are lower alkyl (—C₁-C₄ alkyl), or a halogen (chlorine (—Cl), bromine (—Br), iodine (—I), or fluorine (—F); preferred halogen substituents for phenyl groups are chlorine and fluorine), hydroxy (—OH), or lower alkoxy (—C₁-C₄ alkoxy), such as methoxy, ethoxy, propyloxy (propoxy) (either n-propoxy or i-propoxy), and butoxy (either n-butoxy, i-butoxy, sec-butoxy, or tert-butoxy); a preferred alkoxy substituent is methoxy. Substituted phenyl groups preferably have one or two substituents; more preferably, one substituent.

“Heteroalkyl,” “heteroalkenyl,” and “heteroalkynyl” refer to alkyl, alkenyl, and alkynyl groups, respectively, that contain the number of carbon atoms specified (or if no number is specified, having up to 12 carbon atoms) which contain one or more heteroatoms as part of the main, branched, or cyclic chains in the group. Heteroatoms include, but are not limited to, N, S, O, and P; N and O are preferred. Heteroalkyl, heteroalkenyl, and heteroalkynyl groups may be attached to the remainder of the molecule either at a heteroatom (if a valence is available) or at a carbon atom. Examples of heteroalkyl groups include, but are not limited to, groups such as —O—CH₃, —CH₂—O—CH₃, —CH₂—CH₂—O—CH₃, —S—CH₂—CH₂—CH₃, —CH₂—CH(CH₃)—S—CH₃, —CH₂—CH₂—NH—CH₂—CH₂—, 1-ethyl-6-propylpiperidino, and morpholino. Examples of heteroalkenyl groups include, but are not limited to, groups such as —CH═CH—NH—CH(CH₃)—CH₂—. “Heteroaryl” or “HetAr” refers to an aromatic carbocyclic group having a single ring (including, but not limited to, examples such as pyridyl, imidazolyl, thiophene, or furyl) or two or more condensed rings (including, but not limited to, examples such as indolizinyl or benzothienyl) and having at least one hetero atom, including, but not limited to, heteroatoms such as N, O, P, or S, within the ring. Unless otherwise specified, heteroalkyl, heteroalkenyl, heteroalkynyl, and heteroaryl groups have between one and five heteroatoms and between one and twelve carbon atoms. “Substituted heteroalkyl,” “substituted heteroalkenyl,” “substituted heteroalkynyl,” and “substituted heteroaryl” groups refer to heteroalkyl, heteroalkenyl, heteroalkynyl, and heteroaryl groups substituted with one or more substituents, including, but not limited to, groups such as alkyl, alkenyl, alkynyl, benzyl, hydrocarbon chains, halogen, alkoxy, acyloxy, amino, hydroxyl, mercapto, carboxy, benzyloxy, phenyl, benzyl, cyano, nitro, thioalkoxy, carboxaldehyde, carboalkoxy and carboxamide, or a functionality that can be suitably blocked, if necessary for purposes of the invention, with a protecting group. Examples of such substituted heteroalkyl groups include, but are not limited to, piperazine, substituted at a nitrogen or carbon by a phenyl or benzyl group, and attached to the remainder of the molecule by any available valence on a carbon or nitrogen, —NH—SO₂-phenyl, —NH—(C═O)O-alkyl, —NH—(C═O)O-alkyl-aryl, and —NH—(C═O)-alkyl. If chemically possible, the heteroatom(s) and/or the carbon atoms of the group can be substituted. The heteroatom(s) can also be in oxidized form, if chemically possible.

The term “alkoxy” as used herein refers to an alkyl, alkenyl, alkynyl, or hydrocarbon chain linked to an oxygen atom and having the number of carbon atoms specified, or if no number is specified, having up to 12 carbon atoms. Examples of alkoxy groups include, but are not limited to, groups such as methoxy, ethoxy, propyloxy (propoxy) (either n-propoxy or i-propoxy), and butoxy (either n-butoxy, i-butoxy, sec-butoxy, or tert-butoxy). The groups listed in the preceding sentence are preferred alkoxy groups; a particularly preferred alkoxy substituent is methoxy.

The terms “halo” and “halogen” as used herein refer to the Group VIIa elements (Group 17 elements in the 1990 IUPAC Periodic Table, IUPAC Nomenclature of Inorganic Chemistry, Recommendations 1990) and include Cl, Br, F and I substituents. Preferred halogen substituents are Cl and F.

“Protecting group” refers to a chemical group that exhibits the following characteristics: 1) reacts selectively with the desired functionality in good yield to give a protected substrate that is stable to the projected reactions for which protection is desired; 2) is selectively removable from the protected substrate to yield the desired functionality; and 3) is removable in good yield by reagents compatible with the other functional group(s) present or generated in such projected reactions. Examples of suitable protecting groups can be found in Greene et al. (1991) Protective Groups in Organic Synthesis, 3rd Ed. (John Wiley & Sons, Inc., New York). Amino protecting groups include, but are not limited to, mesitylenesulfonyl (Mts), benzyloxycarbonyl (CBz or Z), t-butyloxycarbonyl (Boc), t-butyldimethylsilyl (TBS or TBDMS), 9-fluorenylmethyloxycarbonyl (Fmoc), tosyl, benzenesulfonyl, 2-pyridyl sulfonyl, or suitable photolabile protecting groups such as 6-nitroveratryloxy carbonyl (Nvoc), nitropiperonyl, pyrenylmethoxycarbonyl, nitrobenzyl, α-,α-dimethyl-dimethoxybenzyloxycarbonyl (DDZ), 5-bromo-7-nitroindolinyl, and the like. Hydroxyl protecting groups include, but are not limited to, Fmoc, TBS, photolabile protecting groups (such as nitroveratryl oxymethyl ether (Nvom)), Mom (methoxy methyl ether), and Mem (methoxy ethoxy methyl ether), NPEOC (4-nitrophenethyloxycarbonyl) and NPEOM (4-nitrophenethyloxymethyloxycarbonyl).

### General Synthetic Methods

A method of synthesizing compounds of formula I, I-P, I-E, I-Z, I-P-E, I-P-Z, I-A, I-AP, I-A-E, I-AP-E, I-A-Z, I-AP-Z, I-B, I-BP, I-B-E, I-BP-E, I-B-Z, and/or I-BP-Z, is by adapting the synthesis for compounds I-1-E and I-1-Z:

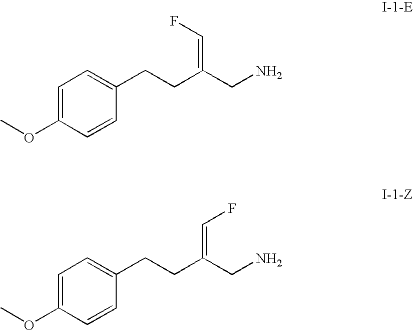

which is shown below in Scheme 1:

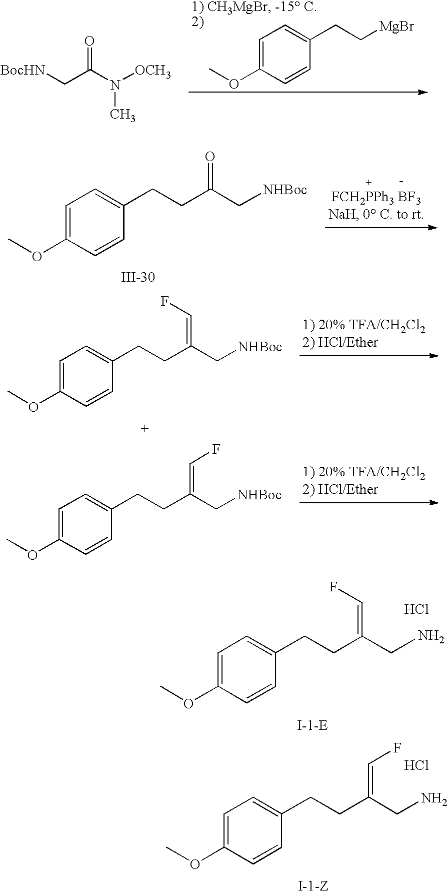

The ω-phenyl alkyl bromide compound was used to form the Grignard reagent based on conditions well known in the art by reacting ω-phenyl alkyl bromide with Mg metal. The Grignard reagent was coupled with Boc-protected glycine Weinreb amide in the presence of MeMgBr to give ketone derivative III-30. Wittig reaction of the ketone with the appropriate reagent provided Z and E isomers, which were then separated using column chromatography. Upon removing the Boc protecting group under acidic conditions, the final compounds are obtained as the TFA salts, which were easily converted to the HCl salts of I-1-E and I-1-Z.

Intermediate compound III-30 is of formulas III, III-A, and III-B, and the synthesis shown in Scheme I for III-30 exemplifies one method of synthesizing compounds of any one of formulas III, III-A, and III-B.

If the appropriate ω-phenyl alkyl bromide is not commercially available, the bromide can be synthesized from the corresponding acid as exemplified in the synthesis of 4-trifluoromethylphenylethyl bromide:

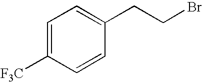

which is shown below in Scheme 2:

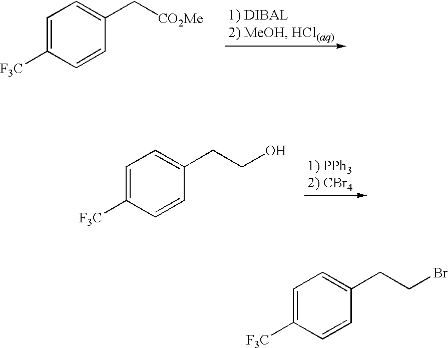

Thus, ω-phenyl alkyl acid was first converted to its corresponding methyl ester under conditions well known in the art. The methyl ester was then be reduced, either at room temperature with lithium aluminum hydride, or at −78° C. with DIBAL, to provide the corresponding alcohol. The alcohol was then converted to the desired bromide in the presence of CBr₄ and PPh₃.

Another method of synthesizing compounds of formula I, I-P, I-E, I-Z, I-P-E, I-P-Z, I-A, I-AP, I-A-E, I-AP-E, I-A-Z, I-AP-Z, I-B, I-BP, I-B-E, I-BP-E, I-B-Z, and/or I-BP-Z is by an alternate synthesis to the protected amino ketone intermediate, as exemplified by the synthesis of compound III-17:

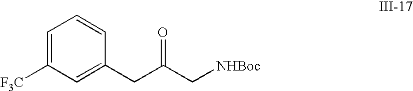

which is shown below in Scheme 3:

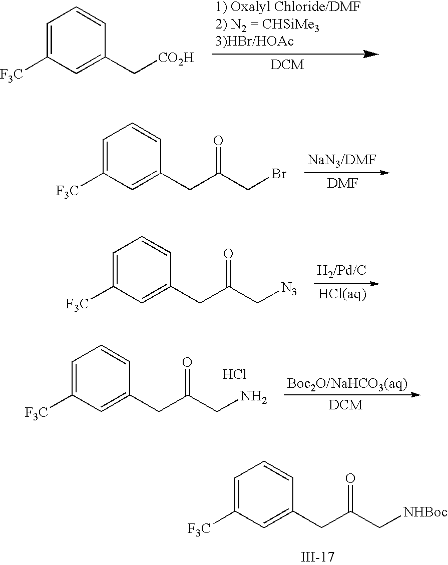

A solution of carboxylate starting material was treated with oxalyl chloride and DMF to form an acid chloride intermediate. The crude product was then treated with trimethylsilyldiazomethane followed by HBr in acetic acid to yield 3-(3-Trifluoromethylphenyl)-2-oxopropyl bromide. The α-bromo ketone was then subjected to sodium azide to yield 3-(3-Trifluoromethylphenyl)-2-oxopropylazide, followed by hydrogenation to generate 3-(3-Trifluoromethylphenyl)-2-oxopropylamine hydrochloride. The amino ketone was then treated with Boc anhydride under basic conditions to yield the protected amino ketone III-17. The intermediate III-17 was then subjected the Wittig conditions and deprotection described in Scheme 1 to generate the desired product.

Intermediate compound III-17 is of formula III, III-A, and III-B, and its synthesis shown in Scheme I exemplifies one method of synthesizing compounds of any one of formulas III, III-A, and III-B.

Synthesizing compounds of formula I, I-P, I-E, I-Z, I-P-E, I-P-Z, I-A, I-AP, I-A-E, I-AP-E, I-A-Z, I-AP-Z, I-B, I-BP, I-B-E, I-BP-E, I-B-Z, and/or I-BP-Z where X is O or S can be accomplished by adapting the synthesis described above wherein starting material carboxylate is properly substituted with an oxygen or sulfur atom. Preparation of carboxylate starting material where X is O or S is described below:

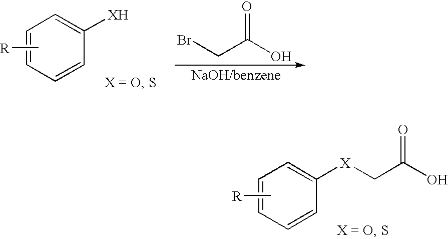

Thus a properly substituted phenol or thiophenol is treated with base, followed by addition of bromoacetic acid. The reaction mixture is acidified and the product extracted with EtOAc and recrystallized from EtOAc/hexane. The O/S substituted carboxylate product is then used to generate the appropriately protected amino ketone under conditions described in Scheme 3 followed by subsequent Wittig treatment and deprotection described in Scheme 1 to yield the desired product.

A method of synthesizing compounds of formula II, II-E, II-Z, II-A, II-A-E, II-A-Z, II-B, II-B-E, and/or II-B-Z, is by adapting the synthesis for compounds II-1-E and II-1-Z:

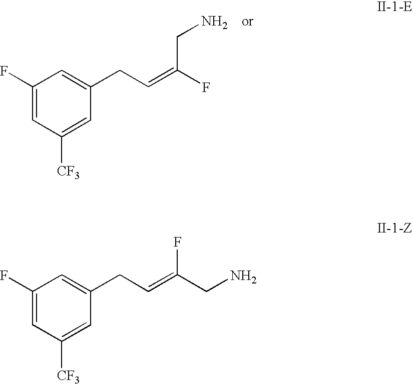

which is shown below in Scheme 4:

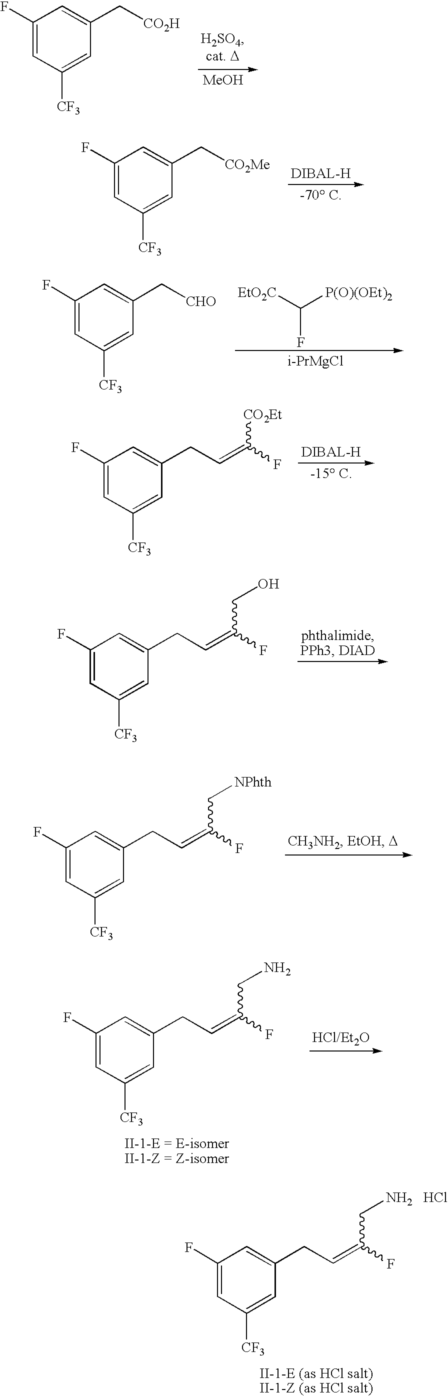

The carboxylate starting material was esterified under acidic conditions to generate the methyl ester, which was then reduced with DIBAL-H to the ω-phenyl alkyl aldehyde. The aldehyde was reacted with the ylid generated from the reaction of triethyl 2-fluoro-2-phosphonoacetate and NaH resulting in the ethyl ester E & Z isomers. The cis and trans isomers were separated using column chromatography and each isomer individually reduced using DIBAL to yield. The resulting alcohol produced was then coupled with phthalimide under Mitsunobu conditions to give the phthalimide derivative. The phthalimide protecting group was removed with MeNH₂ to generate II-1-E and II-1-Z followed by acidification to give the final compound as the HCl salt. Note that the final compounds in Scheme 4 are the hydrochloride salts of II-1-E and II-1-Z.

Removal of the phthalimide protecting group in Scheme 4 may also be immediately followed by treatment with HCl to form the salt form of the desired product without isolation of the free amine intermediate.

For compounds where the substituent R₈ is other than hydrogen, the corresponding ketone can be used as the starting material. Thus, for example, in the synthesis depicted in Scheme 4, the compound R—CH₂—CHO (where R is 3-fluoro-5-trifluoromethyl) would be replaced by a compound of the formula R—CH₂—C(═O)—R₈. If the corresponding ketone is not commercially available, it can be produced by reacting the corresponding carboxylic acid with two equivalents of the appropriate alkyllithium reagent (R₈—Li) or via other methods known to the skilled artisan.

A method of synthesizing compounds of formula II, II-E, II-Z, II-A, II-A-E, II-A-Z, II-B, II-B-E, and/or II-B-Z, where X is O or S can be accomplished by adapting the synthesis of compound II-19:

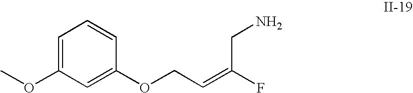

which is shown below in Scheme 5:

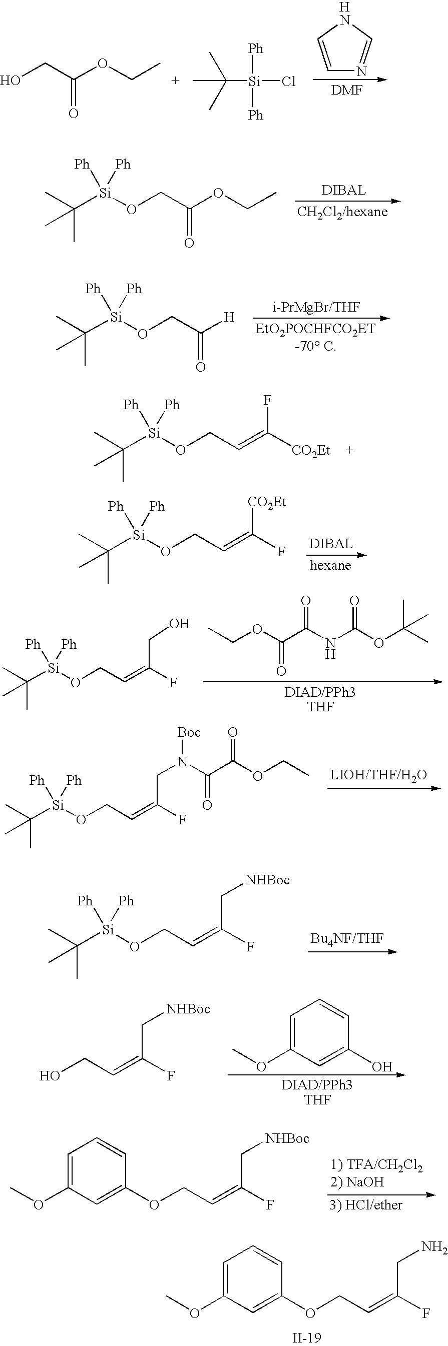

A solution of ethyl glycolate and imidazole in DMF was cooled and treated with tert-butyldiphenyl silane chloride to generate the acetate followed by reduction to the aldehyde with DIBAL. The aldehyde was exposed to triethyl 2-fluoro-2-phosphonacetate and isopropylmagnesium chloride in THF to form the fluoro substituted intermediate, then reduced to the primary alcohol by treatment with DIBAL and coupled with (E)-N-t-Butoxycarbonyl-N-(ethoxyoxoacetyl)-4-(tert-butyldiphenylsilanyloxy)-2-fluoro-2-butenylamine. The product was as treated with tetrabutylammonium fluoride trihydrate to form alcohol (E)-tert-butyl 2-fluoro-4-hydroxybut-2-enylcarbamate. The intermediate was coupled to the desired substituted phenol with triphenyl phosphine and DIAD in THF to generate the Boc-protected precursor, followed by standard acid treatment to remove the Boc protecting group forming the final product II-19.

Intermediate (E)-tert-butyl 2-fluoro-4-hydroxybut-2-enylcarbamate may be coupled to any number of substituted phenols, thiophenols, heterocyclic hydroxyls, or heterocyclic thiols using the methods of Scheme 5 to synthesize compounds of formula II, II-E, II-Z, II-A, II-A-E, II-A-Z, II-B, II-B-E, and/or II-B-Z, where X is O or S.

### Methods of Use

The compounds discussed herein can be used in a variety of manners. One such use is in treatment or prevention of inflammation, inflammatory diseases, inflammatory responses, and certain other diseases, as described in more detail below under “Treatment and Prevention of Diseases.” Other uses include inhibiting SSAO enzyme activity and/or VAP-1 binding activity or VAP-1 amine oxidase activity, both in vivo and in vitro. An example of in vitro use of the compounds is use in assays, such as conventional assays or high-throughput screening assays. Compounds containing nitro (NO₂), bromo (Br), and/or iodo (I) groups can be used for treatment and prevention, but should be evaluated carefully for toxicity due to the presence of the nitro, bromo, and/or iodo groups. These compounds can also be useful intermediate compounds (e.g., the nitro group can be reduced to an amino group in a synthetic pathway).

### Treatment and Prevention of Diseases

Compounds discussed herein are useful for treating or preventing inflammation and inflammatory conditions, and for treating or preventing immune and autoimmune disorders. The compounds are also useful for treating or preventing one or more of a variety of diseases caused by or characterized by inflammation or immune disorders. Thus the compounds can be used to treat or prevent diseases caused by inflammation, and can also be used to treat or prevent diseases which cause inflammation. The compounds are used for treatment or prevention in mammals, preferably humans. “Treating” a disease with the compounds discussed herein is defined as administering one or more of the compounds discussed herein, with or without additional therapeutic agents, in order to palliate, ameliorate, stabilize, reverse, slow, delay, reduce, or eliminate either the disease or one or more symptoms of the disease, or to retard or stop the progression of the disease or of one or more symptoms of the disease. To “prevent” a disease means to suppress the occurrence of a disease or symptoms of a disease before its clinical manifestation. Prevention or suppression can be partial or total. It should be noted that the use of the compounds and/or methods for treatment and the use of the compounds and/or methods for prevention need not be mutually exclusive. “Therapeutic use” of the compounds discussed herein is defined as using one or more of the compounds discussed herein to treat or prevent a disease, as defined above. A “therapeutically effective amount” of a compound is an amount of the compound, which, when administered to a subject, is sufficient to treat, prevent, reduce, or eliminate either the disease or one or more symptoms of the disease, or to retard the progression of the disease or of one or more symptoms of the disease, or to reduce the severity of the disease or of one or more symptoms of the disease. A “therapeutically effective amount” can be given in one or more administrations.

The subjects undergoing treatment or preventive therapy with the compounds and methods of the invention include vertebrates, preferably mammals, more preferably humans.

Diseases which can be treated or prevented with the compound and methods of the invention include inflammation, inflammatory responses, inflammatory diseases and immune disorders. It should be noted that inflammatory diseases can be caused by immune disorders, and that immune disorders are often accompanied by inflammation, and therefore both inflammation and immune disorders may be treated or prevented simultaneously by the compounds and methods of the invention. Diseases which can be treated or prevented with the compounds and methods of the invention include, but are not limited to, multiple sclerosis (including chronic multiple sclerosis); synovitis; systemic inflammatory sepsis; inflammatory bowel diseases; Crohn's disease; ulcerative colitis; Alzheimer's disease; atherosclerosis; rheumatoid arthritis; juvenile rheumatoid arthritis; pulmonary inflammatory conditions; asthma; skin inflammatory conditions and diseases; contact dermatitis; liver inflammatory and autoimmune conditions; autoimmune hepatitis; primary biliary cirrhosis; sclerosing cholangitis; autoimmune cholangitis; alcoholic liver disease; Type I diabetes and/or complications thereof; Type II diabetes and/or complications thereof; atherosclerosis; ischemic diseases such as stroke and/or complications thereof; and myocardial infarction. In another embodiment, the inflammatory disease or immune disorder to be treated or prevented by the present invention is multiple sclerosis. In another embodiment, the inflammatory disease or immune disorder to be treated or prevented by the present invention is chronic multiple sclerosis. In another embodiment, the inflammatory disease or immune disorder to be treated or prevented by the present invention is the inflammatory complications resulting from stroke.

### Modes of Administration

The compounds described for use in the present invention can be administered to a mammalian, preferably human, subject via any route known in the art, including, but not limited to, those disclosed herein. Methods of administration include but are not limited to, intravenous, oral, intraarterial, intramuscular, topical, via inhalation (e.g. as mists or sprays), via nasal mucosa, subcutaneous, transdermal, intraperitoneal, gastrointestinal, and directly to a specific or affected organ. Oral administration is a preferred route of administration. The compounds described for use herein can be administered in the form, of tablets, pills, powder mixtures, capsules, granules, injectables, creams, solutions, suppositories, emulsions, dispersions, food premixes, and in other suitable forms. The compounds can also be administered in liposome formulations. Additional methods of administration are known in the art.

The compounds can be administered in prodrug form. Prodrugs are derivatives of the compounds which are themselves relatively inactive, but which convert into the active compound when introduced into the subject in which they are used, by a chemical or biological process in vivo, such as an enzymatic conversion. Suitable prodrug formulations include, but are not limited to, peptide conjugates of the compounds of the invention and esters of compounds of the inventions. Further discussion of suitable prodrugs is provided in H. Bundgaard, Design of Prodrugs, New York: Elsevier, 1985; in R. Silverman, The Organic Chemistry of Drug Design and Drug Action, Boston: Elsevier, 2004; in R. L. Juliano (ed.), Biological Approaches to the Controlled Delivery of Drugs (Annals of the New York Academy of Sciences, v. 507), New York: New York Academy of Sciences, 1987; and in E. B. Roche (ed.), Design of Biopharmaceutical Properties Through Prodrugs and Analogs (Symposium sponsored by Medicinal Chemistry Section, APhA Academy of Pharmaceutical Sciences, November 1976 national meeting, Orlando, Fla.), Washington: The Academy, 1977. It should be noted that in all of the synthetic schemes presented, the penultimate compounds can be used as prodrugs. That is, the Boc-protected, and similarly protected or derivatized compounds (which appear in the synthetic pathway prior to the desired active compound and/or salt of the desired compound) can be used as prodrugs.

The compounds of the present invention may be administered in an effective amount within the dosage range of about 0.1 μg/kg to about 300 mg/kg, or within about 1.0 μg/kg to about 40 mg/kg body weight, or within about 1.0 μg/kg to about 20 mg/kg body weight, preferably between about 1.0 μg/kg to about 10 mg/kg body weight. Compounds of the present invention may be administered in a single daily dose, or the total daily dosage may be administered in divided dosage of two, three or four times daily.

The pharmaceutical dosage form which contains the compounds described herein is conveniently admixed with a non-toxic pharmaceutical organic carrier or a non-toxic pharmaceutical inorganic carrier; that is, with a pharmaceutically acceptable excipient or pharmaceutically acceptable carrier. Typical pharmaceutically-acceptable carriers include, for example, mannitol, urea, dextrans, lactose, potato and maize starches, magnesium stearate, talc, vegetable oils, polyalkylene glycols, ethyl cellulose, poly(vinylpyrrolidone), calcium carbonate, ethyl oleate, isopropyl myristate, benzyl benzoate, sodium carbonate, gelatin, potassium carbonate, silicic acid, and other conventionally employed acceptable carriers. The pharmaceutical dosage form can also contain non-toxic auxiliary substances such as emulsifying, preserving, or wetting agents, and the like. A suitable carrier is one which does not cause an intolerable side effect, but which allows the compound(s) to retain its pharmacological activity in the body. Formulations for parenteral and nonparenteral drug delivery are known in the art and are set forth in Remington. The Science and Practice of Pharmacy, 20th Edition, Lippincott, Williams & Wilkins (2000). Solid forms, such as tablets, capsules and powders, can be fabricated using conventional tableting and capsule-filling machinery, which is well known in the art. Solid dosage forms, including tablets and capsules for oral administration in unit dose presentation form, can contain any number of additional non-active ingredients known to the art, including such conventional additives as excipients; desiccants; colorants; binding agents, for example syrup, acacia, gelatin, sorbitol, tragacanth, or polyvinylpyrollidone; fillers, for example lactose, sugar, maize-starch, calcium phosphate, sorbitol or glycine; tableting lubricants, for example magnesium stearate, talc, polyethylene glycol or silica; disintegrants, for example potato starch; or acceptable wetting agents such as sodium lauryl sulfate. The tablets can be coated according to methods well known in standard pharmaceutical practice. Liquid forms for ingestion can be formulated using known liquid carriers, including aqueous and non-aqueous carriers such as sterile water, sterile saline, suspensions, oil-in-water and/or water-in-oil emulsions, and the like. Liquid formulations can also contain any number of additional non-active ingredients, including colorants, fragrance, flavorings, viscosity modifiers, preservatives, stabilizers, and the like. For parenteral administration, the compounds for use in the invention can be administered as injectable dosages of a solution or suspension of the compound in a physiologically acceptable diluent or sterile liquid carrier such as water, saline, or oil, with or without additional surfactants or adjuvants. An illustrative list of carrier oils would include animal and vegetable oils (e.g., peanut oil, soy bean oil), petroleum-derived oils (e.g., mineral oil), and synthetic oils. In general, for injectable unit doses, sterile liquids such as water, saline, aqueous dextrose and related sugar solutions, and ethanol and glycol solutions such as propylene glycol or polyethylene glycol are preferred liquid carriers.

The pharmaceutical unit dosage chosen is preferably fabricated and administered to provide a concentration of drug in the blood, tissues, organs, or other targeted region of the body which is therapeutically effective for use in treatment of one or more of the diseases described herein. The optimal effective concentration of the compounds of the invention can be determined empirically and will depend on the type and severity of the disease, route of administration, disease progression and health, mass and body area of the patient. Such determinations are within the skill of one in the art. The compounds for use in the invention can be administered as the sole active ingredient, or can be administered in combination with another active ingredient.

### Kits

The invention also provides articles of manufacture and kits containing materials useful for treating or preventing diseases such as inflammatory diseases, autoimmune diseases, multiple sclerosis (including chronic multiple sclerosis); synovitis; systemic inflammatory sepsis; inflammatory bowel diseases; Crohn's disease; ulcerative colitis; Alzheimer's disease; atherosclerosis; rheumatoid arthritis; juvenile rheumatoid arthritis; pulmonary inflammatory conditions; asthma; skin inflammatory conditions and diseases; contact dermatitis; liver inflammatory and autoimmune conditions; autoimmune hepatitis; primary biliary cirrhosis; sclerosing cholangitis; autoimmune cholangitis; alcoholic liver disease; Type I diabetes and/or complications thereof; Type II diabetes and/or complications thereof; atherosclerosis; ischemic diseases such as stroke and/or complications thereof; and myocardial infarction; or for inhibiting SSAO enzyme activity (whether the enzyme activity is due either to soluble SSAO enzyme or membrane-bound VAP-1 protein, or due to both) and/or inhibiting binding to VAP-1 protein. The article of manufacture comprises a container with a label. Suitable containers include, for example, bottles, vials, and test tubes. The containers may be formed from a variety of materials such as glass or plastic. The container holds a composition having an active agent which is effective for treating or preventing diseases or for inhibiting SSAO or VAP-1 enzyme activity or binding to VAP-1 protein. The active agent in the composition is one or more of the compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, or any one of IV-1 through IV-10. The label on the container indicates that the composition is used for treating or preventing diseases such as inflammatory or autoimmune diseases, or for inhibiting SSAO or VAP-1 enzyme activity or binding to VAP-1 protein, and may also indicate directions for either in vivo or in vitro use, such as those described above.

The invention also provides kits comprising any one or more of the compounds of formula I, I-P, I-E, I-P-E, I-P-Z, I-A, I-AP, I-A-E, I-AP-E, I-A-Z, I-AP-Z, I-B, I-BP, I-B-E, I-BP-E, I-B-Z, I-BP-Z, any one of I-1 through I-109, II, II-E, II-Z, II-A, II-A-E, II-A-Z, II-B, II-B-E, II-B-Z, any one of II-1 through II-23, IV, or any one of IV-1 through IV-10. In some embodiments, the kit of the invention comprises the container described above. In other embodiments, the kit of the invention comprises the container described above and a second container comprising a buffer. It may further include other materials desirable from a commercial and user standpoint, including other buffers, diluents, filters, needles, syringes, and package inserts with instructions for performing any methods described herein (such as methods for treating or preventing autoimmune or inflammatory diseases, and methods for inhibiting SSAO or VAP-1 enzyme activity or binding to VAP-1 protein).

In other aspects, the kits may be used for any of the methods described herein, including, for example, to treat an individual with autoimmune or inflammatory disease, such as multiple sclerosis or ischemic disease (such as stroke) and the sequelae thereof.

The disclosures of all publications, patents, patent applications and published patent applications referred to herein by an identifying citation are hereby incorporated herein by reference in their entirety.

The invention will be further understood by the following nonlimiting examples. It should be noted that, while the compounds are typically described as salts, the disclosure expressly includes the non-salt forms of the compounds, as well as any other salt of the compound.

### EXAMPLES

The syntheses of the compounds are depicted in Schemes I, II, III and IV above, as well as in the following examples.

### Example 1

### Synthesis of Compounds of Formula III

### tert-butyl 4-(4-methoxyphenyl)-2-oxobutylcarbamate (III-30)

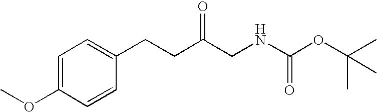

To a cooled suspension of N-(tert-butoxycarbonyl) glycine N′-methoxy-N′-methylamide (1.86 g, 8.53 mmol) in THF (15 mL) at −15° C. under N₂ was added dropwise a solution of MeMgBr in toluene/THF (1.4M, 5.97 mL, 8.36 mmol, 0.98 eq.). To the resulting clear solution was added a solution of 4-methoxyphenylethyl magnesium bromide Grignard reagent in THF (0.5 M, 21.5 mL, 10.7 mmol, 1.26 eq). The resulting mixture was stirred at −15° C. and allowed to warm gradually to room temperature for a 4 hours period, at which time TLC showed the reaction was completed. The reaction mixture was cooled in an ice-bath, and a solution of aqueous HCl (1N, 20 mL) was added, followed by ethyl acetate (60 mL). The layers were separated, and the aqueous layer was extracted with ethyl acetate (2×30 mL). The combined organic layers were washed with brine (30 mL), dried (Na₂SO₄), filtered, and concentrated in vacuo to give an oil, which was then purified by flash column chromatography (silica gel, 10% EtOAc/hexane) to give the ketone product. (2.28 g, 91%) ¹H NMR (CDCl₃, 300 MHz) δ 1.44 (s, 9H), 2.72 (t, J=7.5 Hz, 2H), 2.88 (t, J=7.5 Hz, 2H), 3.78 (s, 3H), 3.97 (d, J=5.1 Hz, 2H), 5.20 (s, 1H), 6.82 (d, J=8.4 Hz, 2H), 7.09 (d, J=8.4 Hz, 2H).

The compounds in the remainder of this example were synthesized according to the procedure for III-30 described above using the appropriate Grignard reagent.

tert-butyl 3-(4-methoxyphenyl)-2-oxopropylcarbamate (III-1): ¹H NMR (CDCl₃, 300 MHz) δ 1.43 (s, 9H), 3.66 (s, 2H), 3.80 (s, 3H), 3.95 (d, J=5.1 Hz, 2H), 6.48 (br s, 1H), 6.87 (d, J=8.4 Hz, 2H), 7.13 (d, J=8.4 Hz, 2H).

tert-butyl 3-(4-ethoxyphenyl)-2-oxopropylcarbamate (III-2): mp 58-59° C. ¹H NMR (CDCl₃, 300 MHz) δ 1.41 (t, J=6.6 Hz, 3H), 1.43 (s, 9H), 3.65 (s, 2H), 4.01 (q, J=6.6 Hz, 2H), 4.05 (s, 2H), 5.18 (br s, 1H), 6.85 (d, J=8.4 Hz, 2H), 7.11 (d, J=8.4 Hz, 2H).

tert-butyl 3-(3,4-dimethoxyphenyl)-2-oxopropylcarbamate (III-3): ¹H NMR (CDCl₃, 300 MHz) δ 1.43 (s, 9H), 3.66 (s, 2H), 3.87 (s, 6H), 4.07 (br s, 2H), 5.18 (br s, 1H), 6.70-6.86 (m, 3H).

tert-butyl 3-(3,5-dimethoxyphenyl)-2-oxopropylcarbamate (III-4): (0.51 g, 18%). ¹H NMR (CDCl₃, 300 MHz) δ 1.43 (s, 9H), 3.65 (s, 2H), 3.78 (s, 6H), 4.06 (br s, 2H), 6.34-6.41 (m, 3H).

tert-butyl 3-(4-isopropoxyphenyl)-2-oxopropylcarbamate (III-5): (8.2 g, 90%). ¹H NMR (CDCl₃, 300 MHz) δ 1.33 (d, J=6 Hz, 6H), 1.43 (s, 9H), 3.42 (br s, 2H), 4.04 (d, J=4.2 Hz, 2H), 4.52 (quintet, J=6.0 Hz, 1H), 5.19 (br s, 1H), 6.84 (d, J=8.4 Hz, 2H), 7.10 (d, J=8.4 Hz, 2H).

tert-butyl 3-(4-methylthiophenyl)-2-oxopropylcarbamate (III-6): (1.99 g, 73%): ¹H NMR (CDCl₃, 300 MHz) δ 1.43 (s, 9H), 2.48 (s, 3H), 3.68 (s, 2H), 4.05 (d, J=4.2 Hz, 2H), 5.30 (br s, 1H), 7.13 (d, J=8.4 Hz, 2H), 7.22 (d, J=8.4 Hz, 2H).

tert-butyl 3-(4-methylphenyl)-2-oxopropylcarbamate (III-8): (3.0 g, 60%). Mp 64-65° C. ¹H NMR (CDCl₃, 300 MHz) δ 1.43 (s, 9H), 2.33 (s, 3H), 3.68 (s, 2H), 4.04 (br s, 2H), 7.01-7.22 (m, 4H).

tert-butyl 3-(3-methylphenyl)-2-oxopropylcarbamate (III-9): (2.17 g, 68%). ¹H NMR (CDCl₃, 300 MHz) δ 1.43 (s, 9H), 3.69 (s, 2H), 3.80 (s, 3H), 3.82 (s, 2H), 6.70-6.91 (m, 3H), 7.19-7.32 (m, 1H).

tert-butyl 3-(4-isopropylphenyl)-2-oxopropylcarbamate (III-10): (7.45 g, 86%): ¹H NMR (CDCl₃, 300 MHz) δ 1.24 (d, J=6.9 Hz, 6H), 1.43 (s, 9H), 2.89 (quintet, J=6.9 Hz, 1H), 3.69 (s, 2H), 4.06 (d, J=4.8 Hz, 2H), 5.20 (br s, 1H), 7.13 (d, J=8.4 Hz, 2H), 7.20 (d, J=8.4 Hz, 2H).

tert-butyl 3-(4-chlorophenyl)-2-oxopropylcarbamate (II-11): (1.83 g, 43%): ¹H NMR (CDCl₃, 300 MHz) δ 1.43 (s, 9H), 3.70 (s, 2H), 4.06 (d, J=4.8 Hz, 2H), 5.18 (br s, 1H), 7.14 (d, J=7.8 Hz, 2H), 7.31 (d, J=7.8 Hz, 2H).

tert-butyl 3-(3-chlorophenyl)-2-oxopropylcarbamate (III-12): (2.45 g, 94%). ¹H NMR (CDCl₃, 300 MHz) δ 1.44 (s, 9H), 3.71 (s, 2H), 4.08 (br s, 2H), 7.06-7.31 (m, 4H).

tert-butyl 3-(4-tert-butylphenyl)-2-oxopropylcarbamate (III-13): (1.0 g, 34%): ¹H NMR (CDCl₃, 300 MHz) δ 1.31 (s, 9H), 1.32 (s, 9H), 3.68 (s, 2H), 4.05 (d, J=4.2 Hz, 2H), 5.21 (br s, 1H), 7.13 (d, J=8.4 Hz, 2H), 7.32 (d, J=8.4 Hz, 2H).

tert-butyl 3-(4-phenylphenyl)-2-oxopropylcarbamate (III-14): (3.45 g, 71%). ¹H NMR (CDCl₃, 300 MHz) δ 1.43 (s, 9H), 3.77 (s, 2H), 4.11 (s, 2H), 7.25-7.64 (m, 9H).

tert-butyl 3-(4-fluorophenyl)-2-oxopropylcarbamate (III-15): (2.24 g, 56%). Mp: 80-81° C. ¹H NMR (CDCl₃, 300 MHz) δ 1.43 (s, 9H), 3.70 (s, 2H), 4.06 (br s, 2H), 7.02 (t, J=8.4 Hz, 2H), 7.18 (q, J=8.7, 6.6 Hz, 2H).

tert-butyl 3-(3-fluorophenyl)-2-oxopropylcarbamate (III-16): (0.6 g, 24%): ¹H NMR (CDCl₃, 300 MHz) δ 1.44 (s, 9H), 3.73 (s, 2H), 4.07 (d, J=4.8 Hz, 2H), 5.18 (br s, 1H), 6.90-7.16 (m, 3H), 7.28-7.36 (m, 1H).

tert-butyl 3-(3-methoxyphenyl)-2-oxopropylcarbamate (III-18): (2.18 g, 68%): ¹H NMR (CDCl₃, 300 MHz) δ 1.43 (s, 9H), 3.69 (s, 2H), 3.80 (s, 3H), 4.06 (d, J=4.8 Hz, 2H), 5.18 (br s, 1H), 6.73-6.90 (m, 3H), 7.21-7.30 (m, 1H).

tert-butyl 3-(3-fluoro-4-methylphenyl)-2-oxopropylcarbamate (III-19): (1.94 g, 55%). ¹H NMR (CDCl₃, 300 MHz) δ 1.43 (s, 9H), 2.27 (s, 3H), 3.68 (s, 2H), 4.02 (br s, 2H), 6.81-7.20 (m, 3H).

tert-butyl 3-(3-fluoro-4-methoxyphenyl)-2-oxopropylcarbamate (III-20): (1.22 g, 39%). ¹H NMR (CDCl₃, 300 MHz) δ 1.43 (s, 9H), 3.65 (s, 2H), 3.81 (s, 3H), 3.84 (s, 2H), 6.91-7.15 (m, 3H)

tert-butyl 3-(4-fluoro-3-methylphenyl)-2-oxopropylcarbamate (III-21): (1.5 g, 56%). ¹H NMR (CDCl₃, 300 MHz) δ 1.43 (s, 9H), 2.26 (s, 3H), 3.65 (s, 2H), 4.05 (br s, 2H), 6.90-7.14 (m, 3H).

tert-butyl 3-(3-chloro-4-fluorophenyl)-2-oxopropylcarbamate (III-22): (2.51 g, 47%). ¹H NMR (CDCl₃, 300 MHz) δ 1.44 (s, 9H), 3.69 (s, 2H), 4.07 (brs, 2H), 7.04-7.18 (m, 2H), 7.24-7.28 (m, 1H).

tert-butyl 3-(2,5-difluorophenyl)-2-oxopropylcarbamate (III-23): (2.7 g, 53%). Mp: 73-74° C. ¹H NMR (CDCl₃, 300 MHz) δ 1.43 (s, 9H), 3.75 (s, 2H), 4.09 (br s, 2H), 6.86-7.11 (m, 3H).

tert-butyl 3-(3-chloro-5-fluorophenyl)-2-oxopropylcarbamate (III-24): (2.56 g, 93%). ¹H NMR (CDCl₃, 300 MHz) δ 1.44 (s, 9H), 3.71 (s, 2H), 4.08 (s, 2H), 6.81-6.89 (m, 1H), 6.97-7.07 (m, 2H).

tert-butyl 3-(2,4-difluorophenyl)-2-oxopropylcarbamate (III-25): (1.57 g, 37%). ¹H NMR (CDCl₃, 300 MHz) δ 1.46 9s, 9H0, 3.73 (s, 2H), 4.09 (br s, 2H), 6.69-6.96 (m, 2H), 7.09-7.24 (m, 1H).

tert-butyl 3-(3,5-dichlorophenyl)-2-oxopropylcarbamate (III-26): (2.64 g, 90%). ¹H NMR (CDCl₃, 300 MHz) δ 1.43 (s, 9H), 3.65 (s, 2H), 4.06 (br s, 2H), 6.34-6.41 (m, 3H).

tert-butyl 3-(3,4-difluorophenyl)-2-oxopropylcarbamate (III-27): (2.13 g, 50%). Mp: 50-51° C. ¹H NMR (CDCl₃, 300 MHz) δ 1.43 (s, 9H), 3.69 (s, 2H), 4.06 (br s, 2H), 6.85-7.23 (m, 3H).

tert-butyl 4-(4-fluorophenyl)-2-oxobutylcarbamate (III-28): (2.29 g, 71%). ¹H NMR (CDCl₃, 300 MHz) δ 1.44 (s, 9H), 2.73 (t, J=7.2 Hz, 2H), 2.91 (t, J=7.2 Hz, 2H), 3.97 (d, J=5.4 Hz, 2H), 6.96 (t, J=8.4 Hz, 2H), 7.10-7.18 (m, 2H).

tert-butyl 4-(4-chlorophenyl)-2-oxobutylcarbamate (III-29): (2.05 g, 55%). ¹H NMR (CDCl₃, 300 MHz) δ 1.44 (s, 9H), 2.74 (t, J=7.2 Hz, 2H), 2.90 (t, J=7.2 Hz, 2H), 3.97 (d, J=2.7 Hz, 2H), 7.10 (d, J=8.9 Hz, 2H), 7.25 (d, J=8.9 Hz, 2H).

tert-butyl 4-(4-ethoxyphenyl)-2-oxobutylcarbamate (III-31): (3.79 g, 57%). ¹H NMR (CDCl₃, 300 MHz) δ 1.40 (t, J=8.4 Hz, 3H), 1.44 (s, 9H), 2.71 (t, J=7.2 Hz, 2H), 2.89 (t, J=7.2 Hz, 2H), 3.96 (d, J=4.8 Hz, 2H), 4.00 (q, J=6.6 Hz, 2H), 6.81 (d, J=8.7 Hz, 2H), 7.07 (d, J=8.7 Hz, 2H).

tert-butyl 4-(4-trifluoromethylphenyl)-2-oxobutylcarbamate (III-32): (0.97 g, 20%). ¹H NMR (CDCl₃, 300 MHz) δ 1.44 (s, 9H), 2.78 (t, J=7.2 Hz, 2H), 3.00 (t, J=7.2 Hz, 2H), 3.99 (d, J=4.8 Hz, 2H), 7.29 (d, J=8.1 Hz, 2H), 7.54 (d, J=8.1 Hz, 2H).

tert-butyl 4-(4-n-butoxyphenyl)-2-oxobutylcarbamate (III-33): (1.24 g, 13%). ¹H NMR (CDCl₃, 300 MHz) δ 0.97 (t, J=7.2 Hz, 3H), 1.44 (s, 9H), 1.46-1.54 (m, 2H) 1.69-1.81 (m, 2H), 2.72 (t, J=7.8 Hz, 2H), 2.87 (t, J=7.8 Hz, 2H), 3.87-7.01 (m, 4H), 6.81 (d, J=8.7 Hz, 2H), 7.07 (d, J=8.7 Hz, 2H).

tert-butyl 4-(3-methylphenyl)-2-oxobutylcarbamate (III-34): (6.13 g, 97%). ¹H NMR (CDCl₃, 300 MHz) δ 1.44 (s, 9H), 2.32 (s, 3H), 2.74 (t, J=7.2 Hz, 2H), 2.90 (t, J=7.2 Hz, 2H), 3.98 (d, J=4.8 Hz, 2H), 6.90-7.06 (m, 3H), 7.17 (t, J=8.1 Hz, 1H).

tert-butyl 4-(3-methoxyphenyl)-2-oxobutylcarbamate (III-35): (6.63 g, 99%). ¹H NMR (CDCl₃, 300 MHz) δ 1.44 (s, 9H), 2.76 (t, J=7.2 Hz, 2H), 2.90 (t, J=7.2 Hz, 2H), 3.79 (s, 3H), 3.98 (d, J=4.8 Hz, 2H), 6.68-6.84 (m, 3H), 7.20 (t, J=7.8 Hz, 1H).

tert-butyl 4-(4-methylphenyl)-2-oxobutylcarbamate (III-36): (3.04 g, 80%). Mp: 62-63° C. ¹H NMR (CDCl₃, 300 MHz) δ 1.44 (s, 9H), 2.31 (s, 3H), 2.73 (t, J=7.2 Hz, 2H), 2.90 (t, J=7.2 Hz, 2H), 3.97 (d, J=4.2 Hz, 2H), 7.02-7.14 (m, 4H).

tert-butyl 4-(3-fluoro-5-trifluoromethylphenyl)-2-oxobutylcarbamate (III-37): (0.19 g, 33%). ¹H NMR (CDCl₃, 300 MHz) δ 1.44 (s, 9H), 2.79 (t, J=7.2 Hz, 2H), 3.00 (t, J=7.2 Hz, 2H), 3.99 (d, J=4.8 Hz, 2H), 7.06-7.31 (m, 3H).

### Example 2

### Alternate Synthesis of Compounds of Formula III

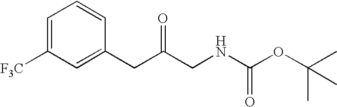

3-(3-Trifluoromethylphenyl)-2-oxopropyl bromide: To a cooled solution of 3-trifluoromethylphenyl acetic acid (1.0 g, 4.9 mmol) in dichloromethane (15 mL) was added dropwise oxalyl chloride (0.73 mL, 8.2 mmol, 1.7 eq). The mixture was stirred at 5° C. for 5 min, then DMF (10 drops) was added. The resulting mixture was stirred at ice-cooled bath for 30 min, then concentrated in vacuo. The residue was used directly in the next step without further purification. It was dissolved in dichloromethane (15 mL). The mixture was cooled in an ice-bath. To this cooled solution was added dropwise a solution of trimethylsilyldiazomethane in hexane (2.0 M, 2.5 mL, 5.0 mmol). The resulting mixture was stirred in an ice bath for 45 min, concentrated in vacuo. The residue was used directly in the nest step without any further purification. To a cooled solution of product from previous step in dichloromethane (15 mL) was added 30% HBr in HOAc (0.61 mL). The resulting mixture was heated at 55° C. for 45 min, cooled to room temperature. Saturated NaHCO₃ solution (˜5 mL) was added. The organic layer was separated, washed with H₂O (20 mL) and brine (20 mL), dried (MgSO₄), filtered, and concentrated to give a brownish oil. MS: 281 (M+H)⁺.

3-(3-Trifluoromethylphenyl)-2-oxopropylazide: The oil was dissolved in DMF (10 mL). The solution was cooled in an ice bath, and NaN₃ (0.64 g, 9.8 mmol) was added. The resulting reaction mixture was stirred at 5° C. for 2 hours, then poured into H₂O (30 mL). The mixture was extracted with EtOAc (2×20 mL). The combined organic layers were washed with brine (20 mL), dried (MgSO₄), filtered, and concentrated. The residue was purified on flash column chromatography (silica gel, 5-10% EtOAc/hexane) to give a yellow oil (0.23 g, 19%). ¹H NMR (CDCl₃, 300 MHz) δ 3.82 (s, 2H), 4.02 (s, 2H), 7.36-7.59 (m, 4H). MS: 244 (M+H)⁺.

3-(3-Trifluoromethylphenyl)-2-oxopropylamine hydrochloride: A mixture of azide (0.47 g, 193 mmol), 12N HCl (0.5 mL), and 10% Pd/C (50% wet, 0.47 g) in MeOH (30 mL) was hydrogenated at 5 psi for 2 hours. The mixture was filtered through a pad of Celite. The filtrate was concentrated in vacuo to give a light brow semisolid (0.49 g). MS: 218 (M+H)⁺.

N-t-Butoxycarbonyl-3-(3-trifluoromethylphenyl)-2-oxopropylamine (III-17): To a stirred suspension of amine hydrochloride (0.49 g) in dichloromethane (10 mL) was added successively a solution of NaHCO₃ (0.16 g) in H₂O (3 mL), NaCl (0.39 g), and a solution of Boc₂O (0.422 g) in dichloromethane (5 mL). The resulting mixture was heated to reflux for 90 min, cooled to room temperature, and diluted with H₂O (10 mL), and extracted with dichloromethane (2×10 mL), dried (MgSO₄), filtered, and concentrated. The residue was purified on flash column chromatography (silica gel, 10 EtOAc/hexane) to give an oil (0.54 g). ¹H NMR (CDCl₃, 300 MHz) δ 1.44 (s, 9H), 3.80 (s, 2H), 4.08 (br s, 2H), 7.36-7.58 (m, 4H).

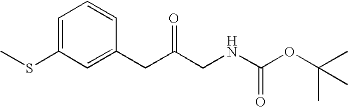

3-(3-Methylthiophenyl)-2-oxopropyl bromide: To a cooled solution of 3-methylthiophenylacetic acid (4.2 g, 23 mmol) in dichloromethane (68 mL) was added dropwise oxalyl chloride (3.48 mL, 39.2 mmol, 1.7 eq). The mixture was stirred at 5° C. for 5 min, then DMF (335 μL) was added. The resulting mixture was stirred at ice-cooled bath for 30 min, then concentrated in vacuo. The residue was used directly in the next step without further purification. It was dissolved in dichloromethane (68 mL). The mixture was cooled in an ice-bath. To this cooled solution was added dropwise a solution of trimethylsilyldiazomethane in hexane (2.0 M, 12.1 mL, 24.2 mmol). The resulting mixture was stirred in an ice bath for 45 min, concentrated in vacuo. The residue was used directly in the nest step without any further purification. To a cooled solution of product from previous step in dichloromethane (70 mL) was added 45% HBr in HOAc (3.1 mL). The resulting mixture was heated at 55° C. for 45 min, cooled to room temperature. Saturated NaHCO₃ solution (23 mL) was added. The organic layer was separated, washed with H₂O (20 mL) and brine (20 mL), dried (MgSO₄), filtered, and concentrated to give a brownish oil (5.02 g). MS: 275 (M+H)⁺.

3-(3-Methylthiophenyl)-2-oxopropylazide: The oil was dissolved in DMF (46 mL). The solution was cooled in an ice bath, and NaN₃ (2.99 g, 46 mmol) was added. The resulting reaction mixture was stirred at 5° C. for 2 hours, then poured into H₂O (30 mL). The mixture was extracted with EtOAc (2×20 mL). The combined organic layers were washed with brine (20 mL), dried (MgSO₄), filtered, and concentrated. The residue was purified on flash column chromatography (silica gel, 5-10% EtOAc/hexane) to give a yellow oil (0.78 g, 50%). ¹H NMR (CDCl₃, 300 MHz) δ 2.48 (s, 3H), 3.71 (s, 2H), 3.99 (s, 2H), 6.98 (d, J=7.2 Hz, 1H), 7.09 (br s, 1H), 7.18 (dt, J=8.7, 2.1 Hz, 1H), 7.25-7.31 (m, 1H) MS: 222 (M+H)⁺.

3-(3-Methylthiophenyl)-2-oxopropylamine hydrochloride: To a solution of 3-(3-Methylthiophenyl)-2-oxopropylazide (2.26 g, 10.3 mmol) in EtOH (82 mL) was added SnCl₂ (5.84 g, 30.8 mmol). The resulting mixture was refluxed under N2 for 30 min, and then concentrated in vacuo. The residue was purified on flash column chromatography (silica gel, 10-12% MeOH/CH₂Cl₂ with 0.1% NH₄OH) to give a semisolid (2.01 g, 100%). ¹H NMR (CDCl₃, 300 MHz) δ 2.47 (s, 3H), 3.86 (s, 2H), 4.03 (s, 2H), 7.03 (d, J=7.2 Hz, 1H), 7.17 (s, 1H), 7.19-7.22 (m, 1H), 7.24-7.31 (m, 1H). MS: 196 (M+H)⁺.

N-t-Butoxycarbonyl-3-(3-methylthiophenyl)-2-oxopropylamine (II′-7): To a stirred suspension of amine hydrochloride (0.443 g) in dichloromethane (10 mL) was added successively a solution of NaHCO₃ (0.16 g) in H₂O (3 mL), NaCl (0.39 g), and a solution of Boc₂O (0.422 g) in dichloromethane (5 mL). The resulting mixture was heated to reflux for 90 min, cooled to room temperature, and diluted with H₂O (10 mL), and extracted with dichloromethane (2×10 mL), dried (MgSO₄), filtered, and concentrated. The residue was purified on flash column chromatography (silica gel, 10 EtOAc/hexane) to give an oil (0.17 g, 25%). ¹H NMR (CDCl₃, 300 MHz) δ 1.44 (s, 9H), 2.48 (s, 3H), 3.69 (s, 2H), 4.06 (br s, 2H), 6.98 (d, J=7.2 Hz, 1H), 7.09 (br s, 1H), 7.13-7.20 (m, 1H), 7.24-7.31 (m, 1H).

### Example 3

### Synthesis of phenyl alkyl bromides

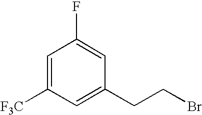

3-Fluoro-5-trifluoromethylphenyl ethanol: To cooled solution of methyl 3-fluoro-5-trifluoromethylphenyl acetate (6.54 g, 27.7 mmol) in hexane (30 mL) was added a solution of DIBAL in toluene (1.5 M, 40 mL, 60 mmol). The cooling bath was removed. The reaction mixture was stirred at room temperature for 90 min. The reaction mixture was cooled in an ice bath, and MeOH (55 mL) was added, followed by aqueous HCl (6 N, 11 mL). The resulting mixture was extracted with ether (2×30 mL). The combined ether layers were washed with brine (20 mL), dried (Na₂SO₄), filtered, and concentrated to give the product (5.7 g, 100%). ¹H NMR (CDCl₃, 300 MHz) δ 2.93 (t, J=6.4 Hz, 2H), 3.91 (t, J=6.4 Hz, 2H), 7.12-7.35 (m, 3H).

3-Fluoro-5-trifluoromethylphenylethylbromide: To a stirred solution of 3-fluoro-5-trifluoromethylphenyl ethanol (2.82 g, 13.5 mmol) in dichloromethane (90 mL) was added PPh₃ (4.26 g, 16.2 mmol), followed by CBr₄ (6.74 g, 20.3 mmol). The reaction mixture was stirred at room temperature under N₂ and monitored by TLC. After stirring at room temperature for 3 hours, TLC showed that the reaction was completed. The reaction mixture was poured into a solution of saturated NaHCO₃ (30 mL). The layers were separated. The aqueous layer was extracted with dichloromethane (2×30 mL). The combined organic layers were washed with brine (30 mL), dried (Na₂SO₄), filtered, and concentrated in vacuo. The residue was purified on flash column chromatography (silica gel, 2% EtOAc/hexane) to the product (2.28 g, 62%). ¹H NMR (CDCl₃, 300 MHz) δ 3.23 (t, J=7.2 Hz, 2H), 3.59 (t, J=7.2 Hz, 2H), 7.10-7.35 (m, 3H).

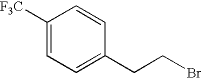

Methyl 4-Trifluoromethylphenyl acetate: (5.97 g, 100%). ¹H NMR (CDCl₃, 300 MHz) δ 3.70 (s, 2H), 3.71 (s, 3H), 7.41 (d, J=8.1 Hz, 2H), 7.59 (d, J=8.1 Hz, 2H).

4-Trifluoromethylphenyl ethanol: To a cooled solution of LAH in THF (1 M, 28 mL, 28 mmol) was added dropwise a solution of methyl 4-trifluoromethylphenyl acetate (5.97 g, 27 mmol) in THF (10 mL). The resulting mixture was stirred at room temperature for 2 hours, and then cooled in an ice bath. To this cooled mixture was added a saturated solution of NH₄Cl (excess). The resulting mixture was filtered. The solid was washed with EtOAc. The combined filtrate was washed with brine (20 mL), dried (MgSO₄), filtered, and concentrated in vacuo to give a oil (4.46 g, 87%). ¹H NMR (CDCl₃, 300 MHz) δ2.94 (t, J=7.2 Hz, 2H), 3.90 (t, J=7.2 Hz, 2H), 7.36 (d, J=8.1 Hz, 2H), 7.58 (d, J=8.1 Hz, 2H).

### Example 4

### Synthesis of Fluoro-Substituted Boc-Protected Precursors of Formula I

### (E)- and (Z)-N-t-Butoxycarbonyl-3-fluoro-2-{2-(4-methoxyphenyl)ethyl}allylamine

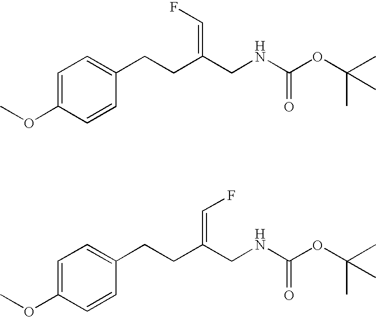

To a cooled suspension of fluoromethyltriphenylphosphonium tetrafluoroborate (ref: J. Fluorine Chem., 1985, 27, 85-89) (1.76 g, 4.61 mmol, 2 eq) in THF at 0° C. was added NaH (0.114 g, 4.75 mmol, 2.06 eq). The resulting mixture was stirred at 0° C. for one hour, at which time a solution of N-t-Butoxycarbonyl-4-(4-methoxyphenyl)-2-oxobutylamine (0.677 g, 2.31 mmol, 1 eq) in THF (10 mL) was added slowly. Upon completion of the addition, the ice-bath was then removed and the reaction was allowed to stir at room temperature overnight. The reaction mixture was poured into a beaker containing ice water (80 mL). The aqueous layer was extracted with ethyl acetate (2×70 mL), and the combined organic layers were washed with brine, dried (Na₂SO₄), filtered, and concentrated in vacuo to give an oil. It was then purified on flash column chromatography (silica gel, 2% EtOAc/hexane) to give the (E)-isomer as a white solid (0.244 g, 34%) m.p.: 40-41° C., ¹H NMR (CDCl₃, 300 MHz) δ 1.45 (s, 9H), 2.38 (t, J=8.1 Hz, 2H), 2.71 (t, J=6.6 Hz, 2H), 3.60 (s, 2H), 4.40 (s, 1H), 6.52 (d, J=84 Hz, 1H), 6.83 (d, J=8.4 Hz, 2H), 7.13 (d, J=8.4 Hz, 1H). (Z)-isomer as a white solid (0.181 g, 25%). m.p.: 32-33° C. ¹H NMR (CDCl₃, 300 MHz) δ 1.46 (s, 9H), 2.20 (m, 2H), 2.68 (t, J=8.1 Hz, 2H), 3.85 (s, 3H), 3.91 (d, J=5.1 Hz, 1H), 4.52 (s, 1H), 6.36 (d, J=85 Hz, 1H), 6.82 (d, J=8.4 Hz, 2H), 7.07 (d, J=8.4 Hz, 2H). The compounds in the remainder of this example were synthesized according to the procedure for (E)- and (Z)-N-t-Butoxycarbonyl-3-fluoro-2-{2-(4-methoxyphenyl)ethyl}allylamine described above using the appropriate starting material synthesized from Examples 1 or 2.

((E)- and (Z)-N-t-Butoxycarbonyl-3-fluoro-2-(4-methoxybenzyl)allylamine: (E)-isomer: 52-53° C. ¹H NMR (CDCl₃, 300 MHz) δ 1.43 (s, 9H), 3.39 (s, 2H), 3.54 (br s, 2H), 3.79 (s, 3H), 4.38 (br s, 1H), 6.64 (d, J=84.3 Hz, 1H), 6.83 (d, J=8.4 Hz, 2H), 7.14 (d, J=8.4 Hz, 2H). (Z)-isomer: 37-38° C. ¹H NMR (CDCl₃, 300 MHz) δ 1.44 (s, 9H), 3.18 (s, 2H), 3.79 (br s, 5H), 4.50 (br s, 1H), 6.45 (d, J=84.3 Hz, 1H), 6.84 (d, J=8.4 Hz, 2H), 7.11 (d, J=8.4 Hz, 2H).

(E)- and (Z)-N-t-Butoxycarbonyl-3-fluoro-2-(4-ethoxybenzyl)allylamine: (E)-isomer: mp 48-49° C. ¹H NMR (CDCl₃, 300 MHz) δ 1.40 (t, J=6.6 Hz, 3H), 1.43 (s, 9H), 3.39 (br s, 2H), 3.53 (br s, 2H), 4.00 (q, J=6.6 Hz, 2H), 4.39 (br s, 1H), 6.63 (d, J=84.9 Hz, 1H), 6.82 (d, J=8.4 Hz, 2H), 7.12 (d, J=8.4 Hz, 2H). (Z)-isomer: ¹H NMR (CDCl₃, 300 MHz) δ 1.40 (t, J=6.6 Hz, 3H), 1.44 (s, 9H), 3.18 (br s, 2H), 3.78 (br s, 2H), 4.01 (q, J=6.6 Hz, 2H), 4.50 (br s, 1H), 6.44 (d, J=84.9 Hz, 1H), 6.82 (d, J=8.4 Hz, 2H), 7.10 (d, J=8.4 Hz, 2H).

(E)- and (Z)-N-t-Butoxycarbonyl-3-fluoro-2-(3,4-dimethoxybenzyl)allylamine: (E)-isomer: ¹H NMR (CDCl₃, 300 MHz) δ 1.43 (s, 9H), 3.39 (s, 2H), 3.56 (br s, 2H), 3.85 (s, 3H), 3.87 (s, 3H), 4.45 (br s, 1H), 6.64 (d, J=84.3 Hz, 1H), 6.71-6.82 (m, 3H). (Z)-isomer: ¹H NMR (CDCl₃, 300 MHz) δ 1.44 (s, 9H), 3.19 (s, 2H), 3.80 (s, 2H), 3.86 (s, 3H), 3.88 (s, 3H), 4.50 (br s, 1H), 6.47 (d, J=84.6 Hz, 1H), 6.68-6.90 (m, 3H).

(E)-N-t-Butoxycarbonyl-3-fluoro-2-(4-isopropoxybenzyl)allylamine: ¹H NMR (CDCl₃, 300 MHz) δ 1.32 (d, J=6.0 Hz, 6H), 1.43 (s, 9H), 3.38 (s, 2H), 3.54 (br s, 2H), 4.39 (br s, 1H), 4.50 (quintet, J=6.0 Hz, 1H), 6.64 (d, J=84.9 Hz, 1H), 6.81 (d, J=8.7 Hz, 2H), 7.11 (d, J=8.7 Hz, 2H).

(E)-N-t-Butoxycarbonyl-3-fluoro-2-(4-isopropylbenzyl)allylamine: (0.3 g, 16%): ¹H NMR (CDCl₃, 300 MHz) δ 1.23 (d, J=6.6 Hz, 6H), 1.42 (s, 9H), 2.87 (quintet, J=6.6 Hz, 1H), 3.42 (s, 2H), 3.54 (br s, 2H), 4.43 (br s, 1H), 6.64 (d, J=84.3 Hz, 1H), 6.95 (d, J=6.6 Hz, 2H), 7.33 (d, J=6.6 Hz, 2H).

(E)-N-t-Butoxycarbonyl-3-fluoro-2-(3-fluoro-5-trifluoromethylphenylethyl)-allylamine: (0.29 g, 41%). Mp: 60-61° C. ¹H NMR (CDCl₃, 300 MHz) δ 1.46 (s, 9H), 2.33-2.52 (m, 2H), 2.76-2.91 (m, 2H), 3.66 (br s, 2H), 6.53 (d, J=83.7 Hz, 1H), 7.09-7.23-7.09 (m, 2H), 7.26-7.30 (m, 1H).

(Z)-N-t-Butoxycarbonyl-3-fluoro-2-(3-fluorophenylethyl)allylamine: (0.08 g, 21%). Mp: 67-68° C. ¹H NMR (CDCl₃, 300 MHz) δ 1.46 (s, 9H), 2.17-2.28 (m, 2H), 2.70-2.81 (m, 2H), 3.92 (br s, 2H), 6.36 (d, J=83.7 Hz, 1H), 6.83-6.99 (m, 3H), 7.17-7.31 (m, 1H).

(E)-N-t-Butoxycarbonyl-3-fluoro-2-(3-fluorophenylethyl)allylamine: (0.15 g, 41%). ¹H NMR (CDCl₃, 300 MHz) δ 1.45 (s, 9H), 2.33-2.47 (m, 2H), 2.68-2.82 (m, 2H), 3.62 (br s, 2H), 6.52 (d, J=83.7 Hz, 1H), 6.78-7.03 (m, 3H), 7.12-7.31 (m, 1H).

### Example 5

### Alternate Synthesis of Fluoro-Substituted Boc-Protected Precursors of Formula I

### (Z)-N-t-Butoxycarbonyl-3-fluoro-2-(4-isopropoxybenzyl)allylamine

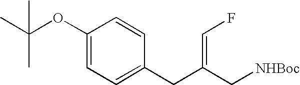

To a suspension of FCH₂P⁺Ph₃BF₄⁻ (4.98 g, 13.02 mmol) in THF (80 mL) at −78° C. under nitrogen was added a solution of sodium bis(trimethylsilyl)amide (NaHMDS) in THF (1.0M, 13.67 mL, 13.67 mmol). The reaction mixture was stirred at −78° C. under nitrogen for 1 h and then a solution of N-t-butoxycarbonyl-3-(4-isopropoxyphenyl)-2-oxopropylamine (2.00 g, 6.51 mmol) in THF (60 mL) was slowly added. The resulting mixture was stirred at −78° C. for 4.5 h and then allowed to warm gradually to room temperature. The mixture was stirred at room temperature overnight and then poured into cold water (50 mL). The layers were separated. The aqueous layer was extracted with EtOAc (3×50 mL). The combined organic layers were dried (Na₂SO₄), filtered, and concentrated in vacuo. The residue was purified on flash column chromatography (silica gel, 0-5% EtOAc/Hexane) to give the desired product as a colorless oil (0.95 g, 45%). ¹H NMR (CDCl₃, 300 MHz) δ 1.32 (d, J=6.0 Hz, 6H), 1.43 (s, 9H), 3.17 (d, J=3.9 Hz, 2H), 3.79 (br s, 2H), 4.51 (quintet, J=6.0 Hz, 1H), 6.45 (d, J=84.3 Hz, 1H), 6.82 (d, J=8.7 Hz, 2H), 7.09 (d, J=8.4 Hz, 2H).

The compounds in the remainder of this example were synthesized according to the procedure for (Z)-N-t-Butoxycarbonyl-3-fluoro-2-(4-isopropoxybenzyl)allylamine described above using the appropriate starting material synthesized from Examples 1 or 2.

(Z)-N-t-Butoxycarbonyl-3-fluoro-2-(4-isopropylbenzyl) allylamine: (1.05 g, 66%): ¹H NMR (CDCl₃, 300 MHz) δ 1.23 (d, J=6.9 Hz, 6H), 1.43 (s, 9H), 2.88 (quintet, J=6.9 Hz, 1H), 3.21 (d, J=3.0 Hz, 2H), 3.79 (br s, 2H), 4.48 (br s, 1H), 6.46 (d, J=84.9 Hz, 1H), 7.11 (d, J=8.9 Hz, 2H), 7.16 (d, J=8.9 Hz, 2H).

(Z)-N-t-Butoxycarbonyl-2-(4-chlorobenzyl)-3-fluoroallylamine: (0.32 g, 33%): ¹H NMR (CDCl₃, 300 MHz) δ 1.43 (s, 9H), 3.21 (d, J=3.0 Hz, 2H), 3.78 (br s, 2H), 4.50 (br s, 1H), 6.46 (d, J=84.3 Hz, 1H), 7.14 (d, J=7.8 Hz, 2H), 7.26 (d, J=7.8 Hz, 2H).

(Z)-N-t-Butoxycarbonyl-3-fluoro-2-(4-methylthiobenzyl)allylamine: (0.56 g, 56%): ¹H NMR (CDCl₃, 300 MHz) δ 1.43 (s, 9H), 2.47 (s, 3H), 3.20 (d, J=3.6 Hz, 2H), 3.79 (br s, 2H), 4.49 (br s, 1H), 6.46 (d, J=84.3 Hz, 1H), 7.13 (d, J=7.8 Hz, 2H), 7.24 (d, J=7.8 Hz, 2H).

(Z)-N-t-Butoxycarbonyl-2-(4-tert-butylbenzyl)-3-fluoroallylamine: (0.25 g, 25%): ¹H NMR (CDCl₃, 300 MHz) δ 1.31 (s, 9H), 1.43 (s, 9H), 3.21 (d, J=3.0 Hz, 2H), 3.79 (br s, 2H), 4.47 (br s, 1H), 6.47 (d, J=83.4 Hz, 1H), 7.13 (d, J=8.4 Hz, 2H), 7.32 (d, J=8.4 Hz, 2H).

(Z)-N-t-Butoxycarbonyl-3-fluoro-2-(3-fluorobenzyl)allylamine: (0.129 g, 23%): ¹H NMR (CDCl₃, 300 MHz) δ 1.43 (s, 9H), 3.24 (d, J=3.3 Hz, 2H), 3.79 (br s, 2H), 4.55 (br s, 1H), 6.48 (d, J=84.0 Hz, 1H), 6.84-7.06 (m, 3H), 7.19-7.32 (m, 1H).

(Z)-N-t-Butoxycarbonyl-3-fluoro-2-(3-methoxybenzyl)allylamine: (0.18 g, 17%): ¹H NMR (CDCl₃, 300 MHz) δ 1.45 (s, 9H), 3.23 (s, 2H), 3.76-3.85 (m, 5H), 4.49 (br s, 1H), 6.48 (d, J=84.0 Hz, 1H), 6.71-6.88 (m, 3H), 7.18-7.32 (m, 1H).

(Z)-N-t-Butoxycarbonyl-3-fluoro-2-(4-methylbenzyl)allylamine: (0.86 g, 81%). Mp 68-69° C. ¹H NMR (CDCl₃, 300 MHz) δ 1.43 (s, 9H), 2.32 (s, 3H), 3.21 (d, J=3.0 Hz, 2H), 3.78 (br s, 2H), 6.46 (d, J=84.9 Hz, 2H), 7.05-7.15 (m, 4H).

(Z)-N-t-Butoxycarbonyl-3-fluoro-2-(4-phenylbenzyl)allylamine: (0.27 g, 20%). Mp: 102-103° C. ¹H NMR (CDCl₃, 300 MHz) δ 1.44 (s, 9H), 3.29 (d, J=3.6 Hz, 2H), 3.82 (br s, 2H), 6.51 (d, J=84.3 Hz, 1H), 7.25-7.61 (m, 9H).

(Z)-N-t-Butoxycarbonyl-3-fluoro-2-(3-fluoro-4-methylbenzyl)allylamine: (0.4 g, 42%). ¹H NMR (CDCl₃, 300 MHz) δ 1.43 (s, 9H), 2.24 (s, 3H), 3.21 (d, J=3.6 Hz, 2H), 3.68 (s, 2H), 3.78 (br s, 2H), 6.47 (d, J=84 Hz, 1H), 6.80-7.15 (m, 3H).

(Z)-N-t-Butoxycarbonyl-3-fluoro-2-(3-fluoro-4-methoxybenzyl)allylamine: (0.26 g, 30%). ¹H NMR (CDCl₃, 300 MHz) δ 1.43 (s, 9H), 3.18 (d, J=3.9 Hz, 2H), 3.78 (br s, 2H), 3.89 (s, 3H), 6.47 (d, J=84 Hz, 1H), 6.84-6.99 (m, 3H).

(Z)-N-t-Butoxycarbonyl-3-fluoro-2-(4-fluoro-3-methylbenzyl)allylamine: (0.39 g, 46%). ¹H NMR (CDCl₃, 300 MHz) δ 1.43 (s, 9H), 2.25 (s, 3H), 3.18 (d, J=3.3 Hz, 2H), 3.77 (br s, 2H), 6.46 (d, J=84.3 Hz, 1H), 6.87-7.05 (m, 3H).

(Z)-N-t-Butoxycarbonyl-3-fluoro-2-(3-chloro-4-fluorobenzyl)allylamine: (0.24 g, 20%). ¹H NMR (CDCl₃, 300 MHz) δ 1.43 (s, 9H), 3.21 (d, J=3.0 Hz, 2H), 3.77 (br s, 2H), 6.48 (d, J=83.4 Hz, 1H), 7.03-7.12 (m, 2H), 7.19-7.28 (m, 1H).

(Z)-N-t-Butoxycarbonyl-3-fluoro-2-(2,5-difluorobenzyl)allylamine: (0.1 g, 12%). Mp: 82-83° C. ¹H NMR (CDCl₃, 300 MHz) δ 1.41 (s, 9H), 3.24 (d, J=3.9 Hz, 2H), 3.79 (br s, 2H), 6.45 (d, J=84.3 Hz, 2H), 6.79-7.02 (m, 3H).

(Z)-N-t-Butoxycarbonyl-3-fluoro-2-(3-chloro-5-fluorobenzyl)allylamine: (0.15 g, 25%). ¹H NMR (CDCl₃, 300 MHz) δ 1.43 (s, 9H), 3.22 (d, J=3.0 Hz, 2H), 3.79 (br s, 2H), 6.50 (d, J=83.7 Hz, 2H), 6.80-7.04 (m, 3H).

(Z)-N-t-Butoxycarbonyl-3-fluoro-2-(2,4-difluorobenzyl)allylamine: (0.3 g, 27%). ¹H NMR (CDCl₃, 300 MHz) δ 1.44 (s, 9H), 3.25 (d, J=3.0 Hz, 2H), 3.81 (br s, 2H), 6.45 (d, J=84.3 Hz, 1H), 6.74-6.89 (m, 2H), 7.14-7.28 (m, 1H).

(Z)-N-t-Butoxycarbonyl-3-fluoro-2-(3,5-dichlorobenzyl)allylamine: (0.17 g, 16%). ¹H NMR (CDCl₃, 300 MHz) δ 1.43 (s, 9H), 3.12 (d, J=3.0 Hz, 2H), 3.80 (br s, 2H), 6.51 (d, J=84.3 Hz, 1H), 7.10 (br s, 2H), 7.22-7.26 (m, 1H).

(Z)-N-t-Butoxycarbonyl-3-fluoro-2-(3,4-difluorobenzyl)allylamine: (0.2 g, 18%). ¹H NMR (CDCl₃, 300 MHz) δ 1.36 (s, 9H), 3.14 (d, J=3.0 Hz, 2H), 3.71 (br s, 2H), 6.42 (d, J=83.4 Hz, 1H), 6.82-7.07 (m, 3H).

(Z)-N-t-Butoxycarbonyl-3-fluoro-2-(3-fluoro-5-trifluoromethylphenylethyl)-allylamine: (0.16 g, 26%). Mp: 62-63° C. ¹H NMR (CDCl₃, 300 MHz) δ 1.47 (s, 9H), 2.16-2.29 (m, 2H), 2.76-2.90 (m, 2H), 3.95 (br s, 2H), 6.38 (d, J=83.7 Hz, 1H), 7.05-7.34 (m, 3H).

(Z)-N-t-Butoxycarbonyl-3-fluoro-2-(3-methylbenzyl)allylamine: (1.35 g, 23%). ¹H NMR (CDCl₃, 300 MHz) δ 1.44 (s, 9H), 2.33 (s, 3H), 3.21 (d, J=3.6 Hz, 2H), 3.79 (s, 2H), 6.46 (d, J=84.3 Hz, 1H), 6.95-7.10 (m, 3H), 7.19-7.29 (m, 1H).

(Z)-N-t-Butoxycarbonyl-3-fluoro-2-(3-trifluoromethylbenzyl)allylamine: (0.13 g, 23%). ¹H NMR (CDCl₃, 300 MHz) δ 1.42 (s, 9H), 3.29 (br s, 2H), 3.81 (br s, 2H), 6.46 (d, J=84.3 Hz, 2H), 6.79-7.56 (m, 4H).

(Z)-N-t-Butoxycarbonyl-3-fluoro-2-(3-methylthiobenzyl)allylamine: (0.2 g, 45%). ¹H NMR (CDCl₃, 300 MHz) δ 1.44 (s, 9H), 2.48 (s, 3H), 3.22 (d, J=3.0 Hz, 2H), 3.79 (br s, 2H), 6.47 (d, J=84 Hz, 1H), 6.94-7.01 (m, 1H), 7.07-7.15 (m, 2H), 7.18-7.26 (m, 1H).

(Z)-N-t-Butoxycarbonyl-3-fluoro-2-(4-fluorobenzyl)allylamine: (0.24 g, 36%). ¹H NMR (CDCl₃, 300 MHz) δ 1.43 (s, 9H), 3.22 (d, J=3.6 Hz, 2H), 3.77 (br s, 2H), 6.46 (d, J=84.3 Hz, 1H), 6.98 (t, J=8.7 Hz, 2H), 7.11-7.22 (m, 2H).

(Z)-N-t-Butoxycarbonyl-3-fluoro-2-(3-chlorobenzyl)allylamine: (0.08 g, 19%). ¹H NMR (CDCl₃, 300 MHz) δ 1.43 (s, 9H), 3.23 (d, J=3.0 Hz, 2H), 3.79 (br s, 2H), 6.48 (d, J=83.4 Hz, 1H), 7.06-7.35 (m, 4H).

(Z)-N-t-Butoxycarbonyl-3-fluoro-2-(3,5-dimethoxybenzyl)allylamine: (0.12 g, 20%). ¹H NMR (CDCl₃, 300 MHz) δ 1.43 (s, 9H), 3.18 (d, J=3.9 Hz, 2H), 3.78 (br s, 8H), 6.30-6.40 (m, 3H), 6.48 (d, J=83.4 Hz, 1H).

### Example 6

### Synthesis of Chloro-Substituted Boc-Protected Precursors of Formula I

General procedures for the preparation of (E) and (Z)-N-t-butoxycarbonyl-3-chloro-2-(arylethyl)allylamine: To a cooled suspension of NaH (1.5 eq) in THF (30 mL) was added (chloromethyl)triphenylphosphonium chloride (1.1 eq). The resulting mixture was stirred at 0° C. for 1 hour, and then a solution of a N-t-butoxycarbonyl-4-substituted-2-oxobutylamine (1.0 eq, prepared from Examples 1 or 2) was added during a period of 10-15 min. The resulting mixture was stirred at room temperature overnight, and then poured into cold water (40 mL). The layers were separated. The aqueous layer was extracted with EtOAc (2×20 mL). The combined organic layers were washed with brine (20 mL), dried (MgSO₄), filtered, and concentrated in vacuo. The residue was purified on flash column chromatography (silica gel, 5-10% EtOAc/hexane) to E and Z isomers.

(E)-N-t-Butoxycarbonyl-3-chloro-2-(4-fluorophenylethyl)allylamine: (0.42 g, 67%). ¹H NMR (CDCl₃, 300 MHz) δ 1.45 (s, 9H), 2.43-2.52 (m, 2H), 2.70-2.80 (m, 2H), 3.70-3.78 (m, 2H), 6.03 (s, 1H), 6.97 (t, J=8.7 Hz, 2H), 7.18 (q, J=8.7 5.4 Hz, 2H).

(E)-N-t-Butoxycarbonyl-3-chloro-2-(4-chlorophenylethyl)allylamine: (0.15 g, 13%). Mp: 73-74° C. ¹H NMR (CDCl₃, 300 MHz) δ 1.45 (s, 9H), 2.41-2.52 (m, 2H), 2.68-2.79 (m, 2H), 3.73 (d, J=5.4 Hz, 2H), 6.02 (s, 1H), 7.15 (d, J=8.4 Hz, 2H), 7.25 (d, J=8.4 Hz, 2H).

(E)-N-t-Butoxycarbonyl-3-chloro-2-(4-methoxyphenylethyl)allylamine: (0.45 g, 40%). ¹H NMR (CDCl₃, 300 MHz) δ 1.45 (s, 9H), 2.28-2.47 (m, 2H), 2.60-2.81 (m, 2H), 3.68-3.76 (m, 2H), 3.79 (s, 3H), 6.01 (s, 1H), 6.83 (d, J=8.7 Hz, 2H), 7.15 (d, J=8.7 Hz, 2H).

(E)-N-t-Butoxycarbonyl-3-chloro-2-(4-ethoxyphenylethyl)allylamine: (1.18 g, 44%). Mp: 78-79° C. ¹H NMR (CDCl₃, 300 MHz) δ 1.39 (t, J=6.6 Hz, 3H), 1.44 (s, 9H), 2.36-2.52 (m, 2H), 2.63-2.76 (m, 2H), 3.61-3.79 (m, 2H), 3.99 (q, J=6.9 Hz, 2H), 6.00 (s, 1H), 6.81 (d, J=8.4 Hz, 2H), 7.12 (d, J=8.4 Hz, 2H).

(E)-N-t-Butoxycarbonyl-3-chloro-2-(4-trifluoromethylphenylethyl)allylamine: (0.2 g, 27%). 1.44 (s, 9H), 2.46-2.56 (m, 2H), 2.79-2.89 (m, 2H), 3.73-3.82 (m, 2H), 6.04 (s, 1H), 7.34 (d, J=8.7 Hz, 2H), 7.55 (d, J=8.7 Hz, 2H).

(E)-N-t-Butoxycarbonyl-3-chloro-2-(4-n-butoxyphenylethyl)allylamine: (0.26 g, 19%). ¹H NMR (CDCl₃, 300 MHz) δ 0.97 (t, J=7.2 Hz, 3H), 1.45 (s, 9H), 1.46-1.54 (m, 2H) 1.70-1.82 (m, 2H), 2.42-1.51 (m, 2H), 2.66-2.75 (m, 2H), 3.72 (d, J=5.1 Hz, 2H), 3.94 (t, J=6.6 Hz, 2H), 6.02 (s, 1H), 6.83 (d, J=8.7 Hz, 2H), 7.13 (d, J=8.7 Hz, 2H).

(E)-N-t-Butoxycarbonyl-3-chloro-2-(3-methylphenylethyl)allylamine: (0.61 g, 36%). Mp: 73-74° C. ¹H NMR (CDCl₃, 300 MHz) δ 1.44 (s, 9H), 2.32 (s, 3H), 2.43-2.52 (m, 2H), 2.67-2.76 (m, 2H), 3.73 (d, J=5.4 Hz, 2H), 6.01 (s, 1H), 6.94-7.09 (m, 3H), 7.17 (t, J=7.8 Hz, 1H).

(E)-N-t-Butoxycarbonyl-3-chloro-2-(3-methoxyphenylethyl)allylamine: (0.8 g, 36%). Mp: 66-67° C. ¹H NMR (CDCl₃, 300 MHz) δ 1.45 (s, 9H), 2.45-2.54 (m, 2H), 2.70-2.79 (m, 2H), 3.74 (d, J=5.4 Hz, 2H), 3.80 (s, 3H), 6.02 (s, 1H), 6.68-6.89 (m, 3H), 7.20 (t, J=7.2 Hz, 1H).

(E)-N-t-Butoxycarbonyl-3-chloro-2-(methylphenylethyl)allylamine: (0.99 g, 60%). Mp: 53-54° C. ¹H NMR (CDCl₃, 300 MHz) δ 1.45 (s, 9H), 2.32 (s, 3H), 2.43-2.52 (m, 2H), 2.68-2.76 (m, 2H), 3.68-3.77 (m, 2H), 6.02 (s, 1H), 7.04-7.20 (m, 4H).

(E)-N-t-Butoxycarbonyl-3-chloro-2-(3-fluoro-5-trifluoromethylphenylethyl)-allylamine: (0.19 g, 46%). Mp: 59-60° C. ¹H NMR (CDCl₃, 300 MHz) δ 1.46 (s, 9H), 2.46-2.56 (m, 2H), 2.79-2.88 (m, 2H), 3.79 (d, J=6.3 Hz, 2H), 6.06 (s, 1H), 7.09-7.38 (m, 3H).

(Z)-N-t-Butoxycarbonyl-3-chloro-2-(4-fluorophenylethyl)allylamine: (0.095 g, 15%). ¹H NMR (CDCl₃, 300 MHz) δ 1.46 (s, 9H), 2.39 (t, J=8.7 Hz, 2H), 2.74 (t, J=8.7 Hz, 2H), 4.0 (d, J=6.6 Hz, 2H), 5.86 (s, 1H), 6.96 (t, J=8.7 Hz, 2H), 7.12 (t, J=8.7 5.4 Hz, 2H).

(Z)-N-t-Butoxycarbonyl-3-chloro-2-(4-chlorophenylethyl)allylamine: (0.05 g, 5%). Mp: 76-77° C. ¹H NMR (CDCl₃, 300 MHz) δ 1.46 (s, 9H), 2.38 (t, J=8.7 Hz, 2H), 2.74 (t, J=8.7 Hz, 2H), 4.0 (d, J=6.6 Hz, 2H), 5.86 (s, 1H), 7.09 (d, J=8.4 Hz, 2H), 7.24 (q, J=8.4 Hz, 2H).

(Z)-N-t-Butoxycarbonyl-3-chloro-2-(4-methoxyphenylethyl)allylamine: (0.15 g, 14%). ¹H NMR (CDCl₃, 300 MHz) δ 1.46 (s, 9H), 2.38 (t, J=7.8 Hz, 2H), 2.70 (t, J=8.4 Hz, 2H), 3.79 (s, 3H), 3.98 (d, J=6.0 Hz, 2H), 5.86 (s, 1H), 6.82 (d, J=8.4 Hz, 2H), 7.08 (d, J=8.4 Hz, 2H).

(Z)-N-t-Butoxycarbonyl-3-chloro-2-(4-ethoxyphenylethyl)allylamine: (0.29 g, 11%). Mp: 70-72° C. ¹H NMR (CDCl₃, 300 MHz) δ 1.40 (t, J=6.6 Hz, 3H), 1.46 (s, 9H), 2.38 (t, J=8.7 Hz, 2H), 2.70 (t, J=8.7 Hz, 2H), 3.92-4.06 (m, 4H), 5.86 (s, 1H), 6.81 (d, J=8.7 Hz, 2H), 7.06 (d, J=8.7 Hz, 2H).

(Z)-N-t-Butoxycarbonyl-3-chloro-2-(4-n-butoxyphenylethyl)allylamine: (0.08 g, 6%). ¹H NMR (CDCl₃, 300 MHz) δ 0.97 (t, J=7.2 Hz, 3H), 1.46 (s, 9H), 1.47-1.56 (m, 2H) 1.65-1.82 (m, 2H), 2.73 (t, J=8.7 Hz, 2H), 2.70 (t, J=8.7 Hz, 2H), 3.93 (t, J=6.6 Hz, 2H), 3.99 (d, J=6.0 Hz, 2H), 5.86 (s, 1H), 6.82 (d, J=8.7 Hz, 2H), 7.06 (d, J=8.7 Hz, 2H).

(Z)-N-t-Butoxycarbonyl-3-chloro-2-(3-methylphenylethyl)allylamine: (0.18 g, 11%). Mp: 60-61° C. ¹H NMR (CDCl₃, 300 MHz) δ 1.45 (s, 9H), 2.31 (s, 3H), 2.38 (t, J=7.8 Hz, 2H), 2.71 (t, J=7.8 Hz, 2H), 3.99 (d, J=6.0 Hz, 2H), 5.88 (s, 1H), 6.92-7.02 (m, 3H), 7.16 (t, J=7.2 Hz, 1H).

(Z)-N-t-Butoxycarbonyl-3-chloro-2-(3-methoxyphenylethyl)allylamine: (0.28 g, 13%). Mp: 69-70° C. ¹H NMR (CDCl₃, 300 MHz) δ 1.46 (s, 9H), 2.41 (t, J=8.4 Hz, 2H), 2.74 (t, J=8.4 Hz, 2H), 3.79 (s, 3H), 4.00 (d, J=6.0 Hz, 2H), 5.88 (s, 1H), 6.67-6.83 (m, 3H), 7.20 (t, J=7.8 Hz, 1H).

(Z)-N-t-Butoxycarbonyl-3-chloro-2-(4-methylphenylethyl)allylamine: (0.31 g, 20%). Mp: 68-69° C. ¹H NMR (CDCl₃, 300 MHz) δ 1.46 (s, 9H), 2.32 (s, 3H), 2.39 (t, J=8.4 Hz, 2H), 2.73 (t, J=8.4 Hz, 2H), 3.99 (d, J=5.4 Hz, 2H), 5.88 (s, 1H), 7.05 (d, J=7.2 Hz, 2H), 7.09 (d, J=7.2 Hz, 2H).

(Z)-N-t-Butoxycarbonyl-3-chloro-2-(3-fluoro-5-trifluoromethylphenylethyl)-allylamine: (0.08 g, 18%). Mp: 71-72° C. ¹H NMR (CDCl₃, 300 MHz) δ 1.46 (s, 9H), 2.42 (t, J=7.8 Hz, 2H), 2.84 (t, J=7.8 Hz, 2H), 4.03 (d, J=6.3 Hz, 2H), 5.90 (s, 1H), 7.03-7.31 (m, 3H).

(E)-N-t-Butoxycarbonyl-3-chloro-2-(phenylethyl)allylamine: (0.25 g, 23%). ¹H NMR (CDCl₃, 300 MHz) δ 1.45 (s, 9H), 2.45-2.54 (m, 2H), 2.71-2.82 (m, 2H), 3.72 (d, J=5.4 Hz, 2H), 6.02 (s, 1H), 7.11-7.41 (m, 5H).

### Example 7

### Synthesis of Compounds of Formula I

(E)-3-Fluoro-2-[2-(4-methoxyphenyl)-ethyl]-allylamine hydrochloride (I-102-E):

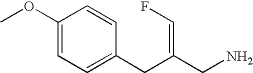

A mixture of (E)-isomer (E)-N-t-Butoxycarbonyl-3-fluoro-2-{2-(4-methoxyphenyl)ethyl}allylamine (0.235 g) in 20% TFA in CH₂Cl₂ (6 mL) was stirred at room temperature for 30 min. Then the TFA was evaporated to dryness, and the residue was dissolved in water (20 mL) and washed with ether (2×10 mL). The aqueous layer was basified to pH 10 by adding NaOH solution (5N). The resulting solution was saturated with NaCl and extracted with ether (2×20 mL). The combined ether layers were then washed with brine, dried (Na₂SO₄), filtered, and concentrated to give an oil. The oil was then dissolved in ether (5 mL), and a solution of HCl in ether (1.0M, 2 eq) was added. A white precipitate was formed. The solid was collected by filtration and washed with ether to give the final (E)-allylamine I-102-E as HCl salt (0.186 g). m.p.: 154-155° C. ¹H NMR (CD₃OD, 300 MHz) δ 2.51 (t, J=8.4 Hz, 2H), 2.72 (t, J=6.6 Hz, 2H), 3.41 (d, J=3 Hz, 1H), 3.76 (s, 3H), 6.84 (dd, J=8.4 Hz, 2H), 6.85 (d, J=82 Hz, 1H), 7.14 (d, J=8.4 Hz, 1H). Cacld for C₁₂H₁₇ClFNO: C, 58.65; H, 6.97; N, 5.70. Found: C, 58.29; H, 7.22; N, 6.07.

The compounds in the remainder of this example were synthesized according to the procedure for I-102-E described above using the appropriate starting material synthesized from Examples 4 or 5.

(Z)-3-Fluoro-2-[2-(4-methoxyphenyl)-ethyl]-allylamine hydrochloride (I-102-Z) HCl salt was synthesized using the similar procedure and obtained as a white solid (0.140 g). m.p.: 94-95° C. ¹H NMR (CD₃OD, 300 MHz) δ 2.33 (m, 2H), 2.70 (t, J=7.8 Hz, 2H), 3.62 (d, J=1.8 Hz, 2H), 3.75 (s, 3H), 6.7 (d, J=84 Hz, 1H), 6.83 (d, J=8.4 Hz, 2H), 7.11 (d, J=8.4 Hz, 2H). Cacld for Cl₂H₁₇ClFNO*0.02H₂O: C, 58.57; H, 6.98; N, 5.69. Found: C, 58.13; H, 7.22; N, 6.01.

(E)-3-Fluoro-2-(4-methoxybenzyl)-allylamine hydrochloride (I-1-E): 177-178° C. ¹H NMR (CD₃OD, 300 MHz) δ 3.43 (d, J=2.4 Hz, 2H), 3.52 (br s, 2H), 3.79 (s, 3H), 6.95 (d, J=82.2 Hz, 1H), 6.97 (d, J=8.4 Hz, 2H), 7.23 (d, J=8.4 Hz, 2H). Cacld for C₁₁H₁₅ClFNO: C, 57.02; H, 6.52; N, 6.04. Found: C, 57.22; H, 6.42; N, 6.33.

(Z)-3-Fluoro-2-(4-methoxybenzyl)-allylamine hydrochloride (I-1-Z): 160-161° C. ¹H NMR (CD₃OD, 300 MHz) δ 3.31 (d, J=3.6 Hz, 2H), 3.52 (d, J=2.4 Hz, 2H), 3.80 (s, 3H), 6.82 (d, J=82.2 Hz, 1H), 6.96 (d, J=8.4 Hz, 2H), 7.23 (d, J=8.4 Hz, 2H). Cacld for C₁₁H₁₅ClFNO: C, 57.02; H, 6.52; N, 6.04. Found: C, 57.18; H, 6.36; N, 6.30.

(E)-3-Fluoro-2-(4-ethoxybenzyl)-allylamine hydrochloride (I-2-E): mp 167-168° C. ¹H NMR (CD₃OD, 300 MHz) δ 1.37 (t, J=6.6 Hz, 3H), 3.33 (d, J=2.4 Hz, 2H), 3.50 (d, J=2.4 Hz, 2H), 4.00 (q, J=6.6 Hz, 2H), 6.86 (d, J=8.4 Hz, 2H), 7.01 (d, J=84.9 Hz, 1H), 7.14 (d, J=8.4 Hz, 2H). Cacld for Cl₂H₁₇ClFNO: C, 58.66; H, 6.97; N, 5.70. Found: C, 58.42; H, 6.91; N, 5.74.

(Z)-3-Fluoro-2-(4-ethoxybenzyl)-allylamine hydrochloride (I-2-Z): mp 153-154° C. ¹H NMR (CD₃OD, 300 MHz) δ 1.37 (t, J=6.6 Hz, 3H), 3.33 (br s, 2H), 3.49 (d, J=2.4 Hz, 2H), 4.00 (q, J=6.6 Hz, 2H), 6.83 (d, J=83.4 Hz, 1H), 6.88 (d, J=8.4 Hz, 2H), 7.15 (d, J=8.4 Hz, 2H). Cacld for C₁₂H₁₇ClFNO: C, 58.66; H, 6.97; N, 5.70. Found: C, 58.32; H, 6.93; N, 5.81.

(E)-3-Fluoro-2-(3,4-dimethoxybenzyl)-allylamine hydrochloride (I-6-E): mp 204-205° C. ¹H NMR (CD₃OD, 300 MHz) δ 3.31 (d, J=3.3 Hz, 2H), 3.41 (br s, 2H), 3.70 (s, 6H), 6.84 (d, J=82.2 Hz, 1H), 6.69-7.01 (m, 3H). Cacld for C₁₂H₁₇ClFNO₂*0.05H₂O: C, 54.88; H, 6.56; N, 5.33. Found: C, 54.42; H, 6.03; N, 5.48.

(Z)-3-Fluoro-2-(3,4-dimethoxybenzyl)-allylamine hydrochloride (I-6-Z): mp 220-221° C. ¹H NMR (CD₃OD, 300 MHz) δ 3.20 (s 2H), 3.44 (s 2H), 3.70 (s, 6H), 6.70 (d, J=82.2 Hz, 1H), 6.72-6.93 (m, 3H). ESMS m/z 226 (M+H)⁺.

### Example 8

### Alternate Synthesis of Compounds of Formula I

### (E)-3-Fluoro-2-(4-isopropoxybenzyl)allylamine hydrochloride (I-8-E)

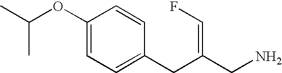

To a solution of E-N-t-butoxycarbonyl-3-fluoro-2-[2-(4-isopropoxyphenyl)ethyl]-allylamine (0.32 g, 0.99 mmol) in CH₂Cl₂ (5.0 mL) was added dropwise trifluoroacetic acid (2.0 mL). The resulting mixture was stirred at room temperature for 20 min and then concentrated in vacuo to give a semisolid. This semisolid was dissolved in H₂O (20 mL) and washed with ether (2×20 mL). The aqueous layer was basified with 2.0M NaOH solution to pH 12 and then extracted with ether (3×30 mL). The combined organic layers were dried over Na₂SO₄, filtered, and concentrated. The residue was dissolved in ether (10 mL). To this solution was added a solution of HCl in ether (2M, 2.0 mL). The solution was stirred at room temperature for 20 min. The precipitate was collected by filtration, washed with ether 4-5 times, and then dried in vacuo. A white solid (0.24 g, 93%) was obtained. mp: 156-157° C. ¹H NMR (MeOH-d4, 300 MHz) δ 1.28 (d, J=6.0 Hz, 6H), 3.35 (d, J=3.0 Hz, 2H), 3.50 (d, J=2.4 Hz, 2H), 4.55 (quintet, J=6.0 Hz, 1H), 6.85 (d, J=8.4 Hz, 2H), 7.00 (d, J=83.1 Hz, 1H), 7.14 (d, J=8.4 Hz, 2H). Calcd for C₁₃H₁₉ClFNO: C, 60.11; H, 7.37; N, 5.39. Found: C, 60.37; H, 7.28; N, 5.60.

The compounds in the remainder of this example were synthesized according to the procedure for I-8-E described above using the appropriate starting material synthesized from Examples 4, 5, or 6.

(Z)-2-(4-Chlorobenzyl)-3-fluoro-allylamine hydrochloride (I-3: (0.21 g, 86%): mp: 145-146° C. ¹H NMR (MeOH-d₄, 300 MHz) δ 3.38 (d, J=2.4 Hz, 2H), 3.49 (d, J=1.8 Hz, 2H), 6.87 (d, J=82.8 Hz, 1H), 7.24 (d, J=8.4 Hz, 2H), 7.34 (d, J=8.4 Hz, 2H). Calcd for C₁₀H₁₂Cl₂FN: C, 50.87; H, 5.12; N, 5.93. Found: C, 51.27; H, 5.41; N, 5.91.

(Z)-3-Fluoro-2-(3-chlorobenzyl)allylamine hydrochloride (I-4): (0.21 g, 86%). Mp: 145-146° C. ¹H NMR (MeOD-d₃, 300 MHz) δ 3.39 (d, J=2.4 Hz, 2H), 3.49 (d, J=1.8 Hz, 2H), 6.87 (d, J=82.8 Hz, 1H), 7.20-7.37 (m, 4H). Calcd for C₁₀H₁₂Cl₂FN: C, 50.87; H, 5.12; N, 5.93. Found: C, 51.27; H, 5.41; N, 5.91.

(Z)-3-Fluoro-2-(3-methoxybenzyl)allylamine hydrochloride (I-5-Z): (0.048 g, 47%): mp: 119.0-120.5° C. ¹H NMR (MeOH-d₄, 300 MHz) δ 3.37 (d, J=3.6 Hz, 2H), 3.5 (br s, 2H), 3.79 (s, 3H), 6.79-6.86 (m, 3H), 6.88 (d, J=83.7 Hz, 1H), 7.22-7.29 (m, 1H). HRMS (ESI-TOF) Calcd for C₁₁H₁₄FNOH: 196.1059. Found: 196.1049.

(Z)-3-Fluoro-2-(3,5-dimethoxybenzyl)allylamine hydrochloride (I-7): (0.05 g, 70%). Mp: 194-195° C. ¹H NMR (MeOD-d₃, 300 MHz) δ 3.31 (br s, 2H), 3.50 (br s, 2H), 3.76 (s, 6H), 6.38-6.44 (m, 3H), 6.90 (d, J=83.4 Hz, 1H). Calcd for C₁₂H₁₇ClFNO₂: C, 55.07; H, 6.55; N, 5.35. Found: C, 55.29; H, 6.35; N, 5.54.

(Z)-3-Fluoro-2-(4-isopropoxybenzyl)allylamine hydrochloride (I-8-Z): (0.71 g, 98%) was obtained. mp: 147-148° C. ¹H NMR (MeOH-d4, 300 MHz) δ 1.29 (d, J=6.0 Hz, 6H), 3.29-3.34 (m, 2H), 3.49 (d, J=2.4 Hz, 2H), 4.56 (quintet, J=6.3 Hz, 1H), 6.84 (d, J=83.4 Hz, 1H), 6.87 (d, J=8.7 Hz, 2H), 7.14 (d, J=8.4 Hz, 2H). Calcd for C₁₃H₁₉ClFNO: C, 60.11; H, 7.86; N, 5.39. Found: C, 60.22; H, 7.46; N, 5.50.

(Z)-3-Fluoro-2-(4-methylthiobenzyl)allylamine hydrochloride (I-9): (0.046 g, 50%): mp: 144-146° C. ¹H NMR (MeOH-d₄, 300 MHz) δ 2.46 (s, 3H), 3.36 (dd, J=3.6, 1.2 Hz, 2H), 3.49 (br s, 2H), 6.86 (d, J=83.7 Hz, 1H), 7.19 (d, J=8.4 Hz, 2H), 7.25 (d, J=8.4 Hz, 2H). Calcd for C₁₁H₁₅ClFNS*0.6H₂O: C, 51.10; H, 6.31; N, 5.42. Found: C, 51.16; H, 6.33; N, 5.68.

(Z)-3-Fluoro-2-(3-methylthiobenzyl)allylamine hydrochloride (I-10): (0.04 g, 45%). Mp: 145-146° C. ¹H NMR (MeOD-d₃, 300 MHz) δ 2.47 (s, 3H), 3.37 (d, J=4.5 Hz, 2H), 3.50 (d, J=2.4 Hz, 2H), 6.89 (d, J=84.9 Hz, 1H), 7.13-7.33 (m, 4H). Calcd for C₁₁H₁₅ClFNS: C, 53.33; H, 6.10; N, 5.65. Found: C, 53.39; H, 6.45; N, 5.97.

(Z)-3-Fluoro-2-(4-methylbenzyl)allylamine hydrochloride (I-12): (0.62 g, 96%). Mp 150-151° C. ¹H NMR (MeOD-d₃, 300 MHz) δ 2.31 (s, 3H), 3.35 (d, J=3.0 Hz, 2H), 3.48 (d, J=2.7 Hz, 2H), 6.84 (d, J=83.4 Hz, 2H), 7.10-7.19 (m, 4H). Calcd for C₁₁H₁₅CFN: C, 61.25; H, 7.01; N, 6.49. Found: C, 61.57; H, 7.27; N, 6.69.

(Z)-3-Fluoro-2-(3-methylbenzyl)allylamine hydrochloride (I-13): (0.03 g, 42%). Mp: 118-119° C. ¹H NMR (MeODE-d₃, 300 MHz) δ 2.32 (s, 3H), 3.37 (d, J=3.0 Hz, 2H), 3.49 (br s, 2H), 6.85 (d, J=83.1 Hz, 1H), 7.02-7.12 (m, 3H), 7.20 (t, J=8.4 Hz, 1H). Calcd for C₁₁H₁₅ClFN: C, 61.25; H, 7.01; N, 6.49. Found: C, 61.25; H, 6.76; N, 6.49.

(E)-3-Fluoro-2-(4-isopropylbenzyl)allylamine hydrochloride (I-14-E): (0.19 g, 96%): mp: 175-176° C. ¹H NMR (MeOH-d4, 300 MHz) δ 1.22 (d, J=7.4 Hz, 6H), 2.87 (quintet, J=6.9 Hz, 1H), 3.35 (d, J=3.0 Hz, 2H), 3.54 (d, J=2.4 Hz, 2H), 7.00 (d, J=81.3 Hz, 1H), 7.15 (d, J=8.4 Hz, 2H), 7.20 (d, J=8.4 Hz, 2H). Calcd for C₁₃H₁₉ClFN: C, 64.06; H, 7.86; N, 5.75. Found: C, 64.44; H, 7.96; N, 5.83.

(Z)-3-Fluoro-2-(4-isopropylbenzyl)allylamine hydrochloride (I-14-Z): (0.19 g, 96%): mp: 175-176° C. ¹H NMR (MeOH-d4, 300 MHz) δ 1.23 (d, J=7.2 Hz, 6H), 2.88 (quintet, J=6.6 Hz, 1H), 3.35 (d, J=3.0 Hz, 2H), 3.48 (d, J=2.7 Hz, 2H), 6.85 (d, J=83.7 Hz, 1H), 7.16 (d, J=8.1 Hz, 2H), 7.21 (d, J=8.1 Hz, 2H). Calcd for C₁₃H₁₉ClFN: C, 64.06; H, 7.86; N, 5.75. Found: C, 63.65; H, 7.92; N, 5.85.

(Z)-3-Fluoro-2-(4-tert-butylbenzyl)allylamine hydrochloride (I-15): (0.03 g, 20%): mp: 155-156° C. ¹H NMR (D₂O, 300 MHz) δ 1.12 (s, 9H), 3.19 (d, J=3.0 Hz, 2H), 3.40 (d, J=2.4 Hz, 2H), 6.69 (d, J=83.4 Hz, 1H), 7.10 (d, J=8.4 Hz, 2H), 7.32 (d, J=8.4 Hz, 2H). Calcd for C₁₄H₂₁ClFN: C, 65.23; H, 8.21; N, 5.43. Found: C, 64.94; H, 8.12; N, 5.60.

(Z)-3-Fluoro-2-(4-phenylbenzyl)allylamine hydrochloride (I-16): (0.21 g, 96%). Mp: 219-220° C. ¹H NMR (MeOD-d₃, 300 MHz) δ 3.45 (d, J=3.0 Hz, 2H), 3.54 (d, J=2.4 Hz, 2H), 6.92 (d, J=83.7 Hz, 1H), 7.29-7.61 (m, 9H). Calcd for C₁₆H₁₇ClFN: C, 69.19; H, 6.17; N, 5.04. Found: C, 69.00; H, 5.83; N, 5.24.

(Z)-3-Fluoro-2-(4-fluorobenzyl)allylamine hydrochloride (I-17): (0.15 g, 84%). Mp: 139-140° C. ¹H NMR (MeOD-d₃, 300 MHz) δ 3.38 (d, J=3.6 Hz, 2H), 3.49 (d, J=2.4 Hz, 2H), 6.86 (d, J=82.8 Hz, 1H), 7.06 (t, J=8.4 Hz, 2H), 7.22-7.34 (m, 2H). Calcd for C₁₀H₁₂ClF₂N: C, 54.68; H, 5.51; N, 6.38. Found: C, 54.23; H, 5.39; N, 6.61.

(Z)-3-Fluoro-2-(3-trifluoromethylbenzyl)allylamine hydrochloride (I-18): (0.08 g, 80%). Mp: 161-162° C. ¹H NMR (MeOD-d₃, 300 MHz) δ 3.31 (d, J=3.6 Hz, 2H), 3.42 (d, J=2.4 Hz, 2H), 6.69 (d, J=82.2 Hz, 2H), 7.35-7.55 (m, 4H). Calcd for C₁₁H₁₂ClF₄N: C, 48.99; H, 4.49; N, 5.19. Found: C, 49.18; H, 4.61; N, 5.31.

(Z)-3-Fluoro-2-(3-fluorobenzyl)allylamine hydrochloride (I-19-Z): (0.045 g, 63%): mp: 120.5-121.5° C. ¹H NMR (MeOH-d₄, 300 MHz) δ 3.45 (d, J=3.0 Hz, 2H), 3.51 (d, J=2.4 Hz, 2H), 6.90 (d, J=83.0 Hz, 1H), 6.94-7.15 (m, 3H), 7.30-7.41 (m, 1H). Calcd for C₁₀H₁₂ClF₂N*0.2H₂O: C, 53.80; H, 5.84; N, 6.27. Found: C, 53.82; H, 5.84; N, 6.52.

(Z)-3-Fluoro-2-(3-fluoro-4-methylbenzyl)allylamine hydrochloride (I-20): (0.15 g, 49%). Mp: 160-161° C. ¹H NMR (MeOD-d₃, 300 MHz) δ 2.24 (s, 3H), 3.37 (d, J=3.9 Hz, 2H), 3.49 (d, J=2.4 Hz, 2H), 6.88 (d, J=83.1 Hz, 1H), 6.91-6.99 (m, 2H), 7.20 (t, J=8.7 Hz, 1H). Calcd for C₁₁H₁₄ClF₂N: C, 56.54; H, 6.04; N, 5.99. Found: C, 56.43; H, 6.22; N, 6.19.

(Z)-3-Fluoro-2-(3-fluoro-4-methoxybenzyl)allylamine hydrochloride (I-21): (0.14 g, 67%). Mp: 170° C. (decompose). ¹H NMR (D₂O, 300 MHz) δ 3.15 (br s, 2H), 3.40 (br s, 2H), 3.71 (s, 3H), 6.67 (d, J=83.1 Hz, 1H), 6.85-7.02 (m, 3H). Calcd for C₁₁H₁₄ClF₂NO: C, 52.91; H, 5.65; N, 5.61. Found: C, 53.14; H, 5.58; N, 5.71.

(Z)-3-Fluoro-2-(4-fluoro-3-methylbenzyl)allylamine hydrochloride (I-22): (0.02 g, 42%). Mp: 158-159° C. ¹H NMR (MeOD-d₃, 300 MHz) δ 2.25 (s, 3H), 3.37 (d, J=3.3 Hz, 2H), 3.50 (br s, 2H), 6.85 (d, J=83.1 Hz, 1H), 6.94-7.18 (m, 3H). Calcd for C₁₁H₁₄ClF₂N*0.4H₂O: C, 51.34; H, 5.80; N, 5.44. Found: C, 51.01; H, 5.67; N, 5.59.

(Z)-3-Fluoro-2-(3-chloro-4-fluorobenzyl)allylamine hydrochloride (I-23): (0.08 g, 45%). Mp: 163-164° C. ¹H NMR (MeOD-d₃, 300 MHz) δ 3.39 (d, J=3.0 Hz, 2H), 3.50 (d, J=2.4 Hz, 2H), 6.91 (d, J=82.8 Hz, 1H), 7.19-7.27 (m, 2H), 7.37-7.43 (m, 1H). Calcd for C₁₀H₁₁Cl₂F₂N: C, 47.27; H, 4.36; N, 5.51. Found: C, 47.77; H, 4.50; N, 5.67.

(Z)-3-Fluoro-2-(2,5-difluorobenzyl)allylamine hydrochloride (I-24): (0.02 g, 51%). Mp: 105-106° C. ¹H NMR (MeOD-d₃, 300 MHz) δ 3.45 (d, J=3.6 Hz, 2H), 3.57 (d, J=2.4 Hz, 2H), 6.85 (d, J=82.8 Hz, 2H), 7.00-7.24 (m, 3H). Calcd for C₁₀H₁₁ClF₃N: C, 50.54; H, 4.67; N, 5.89. Found: C, 50.45; H, 4.92; N, 5.97.

(Z)-3-Fluoro-2-(3-chloro-5-fluorobenzyl)allylamine hydrochloride (I-25): (0.09 g, 45%). Mp: 158-159° C. ¹H NMR (MeOD-d₃, 300 MHz) δ 3.43 (d, J=3.0 Hz, 2H), 3.51 (d, J=1.8 Hz, 2H), 6.94 (d, J=83.1 Hz, 1H), 6.99-7.21 (m, 3H). ESMS m/z 218 (M+H)⁺.

(Z)-3-Fluoro-2-(2,4-difluorobenzyl)allylamine hydrochloride (I-26): (0.3 g, 27%). Mp: 130-131° C. ¹H NMR (MeOD-d₃, 300 MHz) δ 3.43 (d, J=3.0 Hz, 2H), 3.56 (d, J=2.4 Hz, 2H), 6.80 (d, J=84.3 Hz, 1H), 6.95-7.04 (m, 2H), 7.26-7.40 (m, 1H). Calcd for C₁₀H₁₁ClF₃N: C, 50.54; H, 4.67; N, 5.89. Found: C, 50.17; H, 5.06; N, 6.08.

(Z)-3-Fluoro-2-(3,5-dichlorobenzyl)allylamine hydrochloride (I-27): (0.06 g, 93%). Mp: 184-185° C. ¹H NMR (MeOD-d₃, 300 MHz) δ 3.40 (d, J=3.6 Hz, 2H), 3.51 (d, J=2.4 Hz, 2H), 6.94 (d, J=82.5 Hz, 1H), 7.24-7.29 (m, 2H), 7.36-7.39 (m, 1H). Calcd for C₁₀H₁₁Cl₃FN*0.4H₂O: C, 43.24; H, 4.28; N, 5.04. Found: C, 43.23; H, 4.21; N, 5.26.

(Z)-3-Fluoro-2-(3,4-difluorobenzyl)allylamine hydrochloride (I-28): (0.2 g, 18%). Mp: 141-142° C. ¹H NMR (MeOD-d₃, 300 MHz) δ 3.38 (d, J=3.0 Hz, 2H), 3.51 (d, J=2.4 Hz, 2H), 6.90 (d, J=82.8 Hz, 1H), 7.02-7.31 (m, 3H). Calcd for C₁₀H₁₁ClF₃N: C, 50.54; H, 4.67; N, 5.89. Found: C, 50.35; H, 5.00; N, 6.07.

(Z)-3-Chloro-2-(4-fluorophenylethyl)allylamine trifluoroacetate (I-31): (0.09 g, 90%). Mp: 69-70° C. ¹H NMR (MeOD-d₃, 300 MHz) δ 2.53 (t, J=7.8 Hz, 2H), 2.79 (t, J=7.8 Hz, 2H), 3.77 (br s, 2H), 6.34 (s, 1H), 7.01 (t, J=8.4 Hz, 2H), 7.25 (q, J=8.4, 3.0 Hz, 2H). Calcd for C₁₃H₁₄ClF₄NO₂: C, 47.65; H, 4.31; N, 4.27. Found: C, 48.09; H, 4.33; N, 4.47.

(Z)-3-Chloro-2-(4-chlorophenylethyl)allylamine trifluoroacetate (I-32): (0.03 g, 60%). Mp: 89-90° C. ¹H NMR (MeOD-d₃, 300 MHz) δ 2.53 (t, J=8.1 Hz, 2H), 2.79 (t, J=8.1 Hz, 2H), 3.77 (br s, 2H), 6.33 (s, 1H), 7.19 (d, J=8.7 Hz, 2H), 7.28 (d, J=8.7 Hz, 2H). Calcd for C₁₃H₁₄Cl₂F₃NO₂: C, 45.34; H, 4.10; N, 4.07. Found: C, 45.67; H, 4.11; N, 4.11.

(Z)-3-Chloro-2-(4-methoxyphenylethyl)allylamine hydrochloride (I-33): (0.02 g, 40%). Mp: 123-124° C. ¹H NMR (D₂O, 300 MHz) δ 2.27-2.40 (m, 2H), 2.54-2.65 (m, 2H), 3.61 (br s, 2H), 3.64 (s, 3H), 6.06 (s, 1H), 6.79 (d, J=8.4 Hz, 2H), 7.05 (d, J=8.4 Hz, 2H). Calcd for Cl₂H₁₇Cl₂NO: C, 54.97; H, 6.54; N, 5.34. Found: C, 54.84; H, 6.41; N, 5.37.

(Z)-3-Chloro-2-(4-ethoxyphenylethyl)allylamine hydrochloride (I-34): (0.2 g, 85%). Mp: 160-162° C. ¹H NMR (MeOD-d₃, 300 MHz) δ 1.36 (t, J=7.2 Hz, 3H), 2.46-2.55 (m, 2H), 2.68-2.82 (m, 2H), 3.74 (br s, 2H), 3.98 (q, J=7.2 Hz, 2H), 6.32 (s, 1H), 6.82 (d, J=8.4 Hz, 2H), 7.10 (d, J=8.4 Hz, 2H). Calcd for C₁₃H₁₉Cl₂NO: C, 56.53; H, 6.93; N, 5.07. Found: C, 56.49; H, 6.80; N, 5.14.

(Z)-3-Chloro-2-(4-n-butoxyphenylethyl)allylamine hydrochloride (I-36): (0.06 g, 94%). Mp: 133-134° C. ¹H NMR (MeOD-d₃, 300 MHz) δ 0.97 (t, J=7.2 Hz, 3H), 1.41-1.57 (m 2H), 1.65-1.79 (m, 2H), 2.45-2.58 (m, 2H), 2.67-2.80 (m, 2H), 3.73 (br s, 2H), 3.93 (t, J=6.0 Hz, 2H), 6.33 (s, 1H), 6.83 (d, J=8.4 Hz, 2H), 7.10 (d, J=8.4 Hz, 2H). Calcd for C₁₅H₂₃Cl₂NO: C, 59.21; H, 7.62; N, 4.60. Found: C, 59.24; H, 7.55; N, 4.52.

(Z)-3-Chloro-2-(3-methylphenylethyl)allylamine hydrochloride (I-37): (0.06 g, 48%). Mp: 102-103° C. ¹H NMR (MeOD-d₃, 300 MHz) δ 2.30 (s, 3H), 2.49-2.57 (m, 2H), 2.72-2.80 (m, 2H), 3.75 (br s, 2H), 6.34 (s, 1H), 6.97-7.06 (m, 3H), 7.16 (t, J=7.2 Hz, 1H). Calcd for C₁₂H₁₇Cl₂N: C, 58.55; H, 6.96; N, 5.69. Found: C, 58.46; H, 6.64; N, 5.57.

(Z)-3-Chloro-2-(3-methoxyphenylethyl)allylamine hydrochloride (I-38): (0.14 g, 69%). Mp: 139-140° C. ¹H NMR (MeOD-d₃, 300 MHz) δ 2.50-2.58 (m, 2H), 2.73-2.82 (m, 2H), 3.75 (br s, 2H), 3.77 (s, 3H), 6.35 (s, 1H), 6.73-6.82 (m, 3H), 7.19 (t, J=8.7 Hz, 1H). Calcd for C₁₂H₁₇Cl₂NO: C, 54.97; H, 6.54; N, 5.34. Found: C, 55.06; H, 6.29; N, 5.39.

(Z)-3-Chloro-2-(4-methylphenylethyl)allylamine hydrochloride (I-39): (0.01 g, 40%). Mp: 135° C. (decompose). ¹H NMR (D₂O, 300 MHz) δ 2.12 (s, 3H), 2.30-2.40 (m, 2H), 2.56-2.66 (m, 2H), 3.62 (br s, 2H), 6.06 (s, 1H), 6.95-7.10 (m, 4H). Calcd for C₁₂H₁₇Cl₂N: C, 58.55; H, 6.96; N, 5.69. Found: C, 58.75; H, 6.77; N, 5.38.

(Z)-3-Chloro-2-(3-fluoro-5-trifluoromethylphenylethyl)allylamine trifluoroacetate (I-40): (0.07 g, 93%). Mp: 51-52° C. ¹H NMR (MeOD-d₃, 300 MHz) δ 2.53-2.61 (m, 2H), 2.87-2.95 (m, 2H), 3.82 (s, 2H), 6.42 (s, 1H), 7.27-7.35 (m, 2H), 7.42 (s, 1H). Calcd for C₁₄H₁₃ClF₇NO₂: C, 42.49; H, 3.31; N, 3.54. Found: C, 42.59; H, 3.35; N, 3.58.

(E)-3-Chloro-2-(phenylethyl)allylamine hydrochloride (I-41): (0.15 g, 80%). Mp: 97-98° C. ¹H NMR (D₂O, 300 MHz) δ 2.47 (t, J=8.1 Hz, 2H), 2.67 (t, J=8.1 Hz, 2H), 3.75 (br s, 2H), 6.20 (s, 1H), 7.05-7.29 (m, 5H). Calcd for C₁₁H₁₅Cl₂N: C, 56.91; H, 6.51; N, 6.03. Found: C, 57.12; H, 6.62; N, 5.87.

(E)-3-Chloro-2-(4-fluorophenylethyl)allylamine trifluoroacetate (I-42): (0.34 g, 90%). Mp: 52-53° C. ¹H NMR (MeOD-d₃, 300 MHz) δ 2.57-2.64 (m, 2H), 2.74-2.83 (m, 2H), 3.55 (br s, 2H), 6.45 (s, 1H), 7.01 (t, J=8.4 Hz, 2H), 7.25 (q, J=8.4, 3.0 Hz, 2H). Calcd for C₁₃H₁₄ClF₄NO₂: C, 47.65; H, 4.31; N, 4.27. Found: C, 47.81; H, 4.12; N, 4.37.

(E)-3-Chloro-2-(4-chlorophenylethyl)allylamine trifluoroacetate (I-43): (0.13 g, 90%). Mp: 68-70° C. ¹H NMR (MeOD-d₃, 300 MHz) δ 2.56-2.66 (m, 2H), 2.72-2.81 (m, 2H), 3.55 (br s, 2H), 6.44 (s, 1H), 7.22 (d, J=8.7 Hz, 2H), 7.28 (d, J=8.7 Hz, 2H). Calcd for C₁₃H₁₄Cl₂F₃NO₂: C, 45.34; H, 4.10; N, 4.07. Found: C, 45.56; H, 4.12; N, 4.11.

(E)-3-Chloro-2-(4-methoxyphenylethyl)allylamine hydrochloride (I-44): (0.2 g, 80%). Mp: 138° C. (decompose). ¹H NMR (D₂O, 300 MHz) δ 2.40-2.49 (m, 2H), 2.58-2.67 (m, 2H), 3.38 (br s, 2H), 3.66 (s, 3H), 6.21 (s, 1H), 6.80 (d, J=8.4 Hz, 2H), 7.11 (d, J=8.4 Hz, 2H). Calcd for C₁₂H₁₇Cl₂NO: C, 54.97; H, 6.54; N, 5.34. Found: C, 55.14; H, 6.51; N, 5.67.

(E)-3-Chloro-2-(4-ethoxyphenylethyl)allylamine hydrochloride (I-45): (0.9 g, 82%). Mp: 104-106° C. ¹H NMR (MeOD-d₃, 300 MHz) δ 1.36 (t, J=7.2 Hz, 3H), 2.54-2.62 (m, 2H), 2.68-2.77 (m, 2H), 3.49 (br s, 2H), 3.99 (q, J=7.2 Hz, 2H), 6.43 (s, 1H), 6.82 (d, J=8.4 Hz, 2H), 7.13 (d, J=8.4 Hz, 2H). Calcd for C₁₃H₁₉Cl₂NO: C, 56.53; H, 6.93; N, 5.07. Found: C, 56.62; H, 6.94; N, 5.29.

(E)-3-Chloro-2-(4-trifluoromethylphenylethyl)allylamine hydrochloride (I-46): (0.1 g, 70%). Mp: 89-90° C. ¹H NMR (D₂O, 300 MHz) δ 2.49 (t, J=8.1 Hz, 2H), 2.73 (t, J=8.1 Hz, 2H), 3.40 (br s, 2H), 6.21 (s, 1H), 7.27 (d, J=7.8 Hz, 2H), 7.48 (d, J=7.8 Hz, 2H). Calcd for C₁₂H₁₄Cl₂F₃N: C, 48.02; H, 4.70; N, 4.67. Found: C, 47.84; H, 4.51; N, 4.70.

(E)-3-Chloro-2-(4-n-butoxyphenylethyl)allylamine hydrochloride (I-47): (0.9 g, 82%). Mp: 132-33° C. ¹H NMR (MeOD-d₃, 300 MHz) δ 0.97 (t, J=7.2 Hz, 3H), 1.44-1.56 (m 2H), 1.67-1.79 (m, 2H), 2.54-2.62 (m, 2H), 2.68-2.77 (m, 2H), 3.48 (br s, 2H), 3.93 (t, J=6.9 Hz, 2H), 6.43 (s, 1H), 6.83 (d, J=8.4 Hz, 2H), 7.15 (d, J=8.4 Hz, 2H). Calcd for C₁₅H₂₃Cl₂NO: C, 59.21; H, 7.62; N, 4.60. Found: C, 59.54; H, 7.45; N, 4.42.

(E)-3-Chloro-2-(3-methylphenylethyl)allylamine hydrochloride (I-48): (0.23 g, 48%). Mp: 95-97° C. ¹H NMR (MeOD-d₃, 300 MHz) δ 2.31 (s, 3H), 2.57-2.66 (m, 2H), 2.71-2.79 (m, 2H), 3.51 (br s, 2H), 6.45 (s, 1H), 6.98-7.10 (m, 3H), 7.16 (t, J=7.2 Hz, 1H). Calcd for C₁₂H₁₇Cl₂N: C, 58.55; H, 6.96; N, 5.69. Found: C, 58.65; H, 6.87; N, 5.58.

(E)-3-Chloro-2-(3-methoxyphenylethyl)allylamine hydrochloride (I-49): (0.36 g, 62%). Mp: 85-87° C. ¹H NMR (MeOD-d₃, 300 MHz) δ 2.58-2.67 (m, 2H), 2.73-2.81 (m, 2H), 3.52 (br s, 2H), 3.77 (s, 3H), 6.46 (s, 1H), 6.72-6.88 (m, 3H), 7.19 (d, J=8.1 Hz, 1H). Calcd for C₁₂H₁₇Cl₂NO: C, 54.97; H, 6.54; N, 5.34. Found: C, 54.89; H, 6.17; N, 5.33.

(E)-3-Chloro-2-(4-methylphenylethyl)allylamine hydrochloride (I-50): (0.04 g, 60%). Mp: 124-125° C. ¹H NMR (D₂O, 300 MHz) δ 2.13 (s, 3H), 2.39-2.49 (m, 2H), 2.58-2.68 (m, 2H), 3.38 (br s, 2H), 3.66 (s, 3H), 6.20 (s, 1H), 6.95-7.20 (m, 4H). Calcd for C₁₂H₁₇Cl₂N: C, 58.55; H, 6.96; N, 5.69. Found: C, 58.65; H, 6.87; N, 5.58.

(E)-3-Chloro-2-(3-fluoro-5-trifluoromethylphenylethyl)allylamine hydrochloride (I-51): (0.13 g, 96%). Mp: 96-97° C. ¹H NMR (MeOD-d₃, 300 MHz) δ 2.67 (t, J=8.7 Hz, 2H), 2.91 (t, J=8.7 Hz, 2H), 3.65 (s, 2H), 6.50 (s, 1H), 7.27-7.39 (m, 2H), 7.43 (s, 1H). Calcd for C₁₂H₁₃Cl₂F₄N: C, 45.30; H, 4.12; N, 4.40. Found: C, 45.70; H, 4.01; N, 4.44.

(Z)-3-Fluoro-2-(3-fluorophenylethyl)allylamine trifluoroacetate (I-100-E): (0.08 g, 92%). Mp: 47-48° C. ¹H NMR (MeOD-d₃, 300 MHz) δ 2.32-2.42 (m, 2H), 2.73-2.83 (m, 2H), 3.65 (d, J=1.8 Hz, 2H), 6.74 (d, J=83.7 Hz, 1H), 6.89-7.08 (m, 3H), 7.24-7.36 (m, 1H). Calcd for C₁₃H₁₄F₅NO₂: C, 50.15; H, 4.53; N, 4.5. Found: C, 49.86; H, 4.64; N, 4.46.

(E)-3-Fluoro-2-(3-fluorophenylethyl)allylamine trifluoroacetate (I-100-Z): (0.13 g, 98%). Mp: 72-73° C. ¹H NMR (MeOD-d₃, 300 MHz) δ 2.47-2.56 (m, 2H), 2.75-2.83 (m, 2H), 3.45 (d, J=2.7 Hz, 2H), 6.85 (d, J=82.4 Hz, 1H), 6.87-7.03 (m, 3H), 7.24-7.34 (m, 1H). Calcd for C₁₃H₁₄F₅NO₂: C, 50.15; H, 4.53; N, 4.5. Found: C, 50.04; H, 4.76; N, 4.92.

(E)-3-Fluoro-2-(3-fluoro-5-trifluoromethylphenylethyl)allylamine trifluoroacetate (I-109-E): (0.29 g, 96%). Mp: 58-59° C. ¹H NMR (MeOD-d₃, 300 MHz) δ 2.51-2.59 (m, 2H), 2.84-2.93 (m, 2H), 3.52 (br s, 2H), 6.87 (d, J=82.2 Hz, 1H), 7.25-7.34 (m, 2H), 7.40 (s, 1H). Calcd for C₁₄H₁₃F₈NO₂: C, 44.34; H, 3.45; N, 3.69. Found: C, 44.32; H, 3.30; N, 3.88.

(Z)-3-Fluoro-2-(3-fluoro-5-trifluoromethylphenylethyl)allylamine trifluoroacetate (I-109-Z): (0.15 g, 94%). Mp: 56-57° C. ¹H NMR (MeOD-d₃, 300 MHz) δ 2.35-2.46 (m, 2H), 2.82-2.91 (m, 2H), 3.70 (br s, 2H), 6.78 (d, J=83.7 Hz, 1H), 7.26-7.36 (m, 3H), 7.41 (s, 1H). Calcd for C₁₄H₁₃F₈NO₂: C, 44.34; H, 3.45; N, 3.69. Found: C, 43.98; H, 3.16; N, 3.71.

### Example 9

### Synthesis of Compound I-11

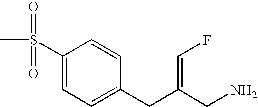

(Z)-N-t-Butoxycarbonyl-3-fluoro-2-(4-methanesulfonylbenzyl)allylamine: To a cooled solution of (Z)-N-t-Butoxycarbonyl-3-fluoro-2-(3-methylthiobenzyl)allylamine (0.36 g, 1.2 mmol) in EtOAc (5 mL) was added a solution of H₅IO₆/CrO₃ in acetonitrile (0.2 mL). The resulting mixture was stirred at −35° C. for 1 hour, quenched with saturated Na₂SO₃ solution (2 mL) and filtered. The solid was washed with EtOAc and filtered. The filtrate was washed with saturated Na₂SO₃ solution (2×10 mL), brine (20 mL), dried (MgSO₄), filtered, and concentrated. The residue was purified on column chromatography (silica gel, 40% EtOAc/hexane) to give an oil (0.34 g, 77%). ¹H NMR (CDCl₃, 300 MHz) δ 1.43 (s, 9H), 3.05 (s, 3H), 3.35 (d, J=3.0 Hz, 2H), 3.78 (br s, 2H), 6.53 (d, J=82.8 Hz, 1H), 7.45 (d, J=8.4 Hz, 2H), 7.90 (d, J=8.4 Hz, 1H).

(Z)-3-fluoro-2-(4-methanesulfonylbenzyl)allylamine trifluoroacetate (I-11): A mixture of (Z)-N-t-Butoxycarbonyl-3-fluoro-2-(3-methanesulfonylbenzyl)allylamine (0.34 g, 0.62 mmol) in 20% TFA/CH₂Cl₂ (5 mL) was stirred at room temperature for 30 min, concentrated. The residue was washed with ether. The solid was collected by filtration (0.02 g, 45%). Mp: 49-50° C. ¹H NMR (MeOD-d₃, 300 MHz) δ 3.11 (s, 3H), 3.51 (br s, 2H), 3.54 (d, J=3.6 Hz, 2H), 6.94 (d, J=83.1 Hz, 1H), 7.56 (d, J=8.1 Hz, 2H), 7.95 (d, J=8.1 Hz, 2H). ESMS m/z 244 (M+H)⁺.

### Example 10

### Methoxy Deprotections

### (Z)-3-Fluoro-2-(4-hydroxylbenzyl)allylamine hydrobromide (I-29)

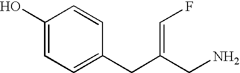

To a solution of (Z)-N-t-Butoxycarbonyl-3-fluoro-2-(4-methoxybenzyl)allylamine (0.4 g, 1.35 mmol) in dichloromethane (20 mL) was added a solution of BBr₃ in dichloromethane (1.0 M, 3.37 mL, 3.37 mmol). The reaction mixture was stirred at room temperature for 45 min, and concentrated in vacuo. The residue was purified by flash column chromatography (silica gel, 10-12% MeOH/CH₂Cl₂ with 0.1% NH₄OH) to give a solid (0.15 g, 62%). Mp: 176-177° C. ¹H NMR (MeOD-d₃, 300 MHz) δ 3.17 (d, J=3.6 Hz, 2H), 3.43 (d, J=2.7 Hz, 2H), 6.69 (d, J=83.7 Hz, 1H), 6.75 (d, J=8.4 Hz, 2H), 7.04 (d, J=8.4 Hz, 2H). Calcd for C₁₀H₁₃BrFNO*0.18H₂O: C, 45.26; H, 5.07; N, 5.28. Found: C, 44.88; H, 4.85; N, 5.52.

(Z)-3-Fluoro-2-(3-hydroxylbenzyl)allylamine citrate (I-30) was obtained using the same procedure as described above: (0.04 g, 45%). Mp: 145-146° C. ¹H NMR (MeOD-d₃, 300 MHz) δ 3.17-3.41 (m, 6H), 3.50 (br s, 2H), 6.62-6.77 (m, 3H), 6.87 (d, J=83.1 Hz, 1H), 7.09-7.21 (m, 1H).

### Example 11

### Aminoketone Deprotections

The compounds in this example were deprotected according to the procedure of either Example 7 or 8.

4-(4-Fluorophenyl)-2-oxobutylamine trifluoroacetate (IV-1): (0.2 g, 89%). Mp: 103-104° C. ¹H NMR (MeOD-d₃, 300 MHz) δ 2.78-2.99 (m, 4H), 3.92 (s, 2H), 6.99 (t, J=8.7 Hz, 2H), 7.19-7.28 (m, 2H). Calcd for C₁₂H₁₃F₄NO₃: C, 48.82; H, 4.44; N, 4.74. Found: C, 49.13; H, 4.35; N, 4.80.

4-(4-Chlorophenyl)-2-oxobutylamine trifluoroacetate (IV-2): (0.73 g, 87%). Mp: 125-126° C. ¹H NMR (MeOD-d₃, 300 MHz) δ 2.83-2.95 (m, 4H), 3.91 (s, 2H), 7.20 (d, J=8.7 Hz, 2H), 7.26 (d, J=8.7 Hz, 2H). Calcd for C₁₂H₁₃IF₃NO₃: C, 46.24; H, 4.20; N, 4.49. Found: C, 46.22; H, 4.32; N, 4.49.

4-(4-Methoxyphenyl)-2-oxobutylamine trifluoroacetate (IV-3): (1.02 g, 97%). Mp: 108-109° C. ¹H NMR (MeOD-d₃, 300 MHz) δ 2.77-2.90 (m, 4H), 3.74 (s, 3H), 3.89 (s, 2H), 6.82 (d, J=8.4 Hz, 2H), 7.12 (d, J=8.4 Hz, 2H). Calcd for C₁₃H₁₆ClF₃NO₄: C, 46.24; H, 4.20; N, 4.49. Found: C, 46.22; H, 4.32; N, 4.49.

4-(3-Methoxyphenyl)-2-oxobutylamine hydrochloride (IV-7): (0.35 g, 21%). Mp: 120-122° C. ¹H NMR (MeOD-d₃, 300 MHz) δ 2.68-2.91 (m, 4H), 3.73 (s, 3H), 3.91 (s, 2H), 6.68-6.82 (m, 3H), 7.12-7.21 (m, 1H). Calcd for C₁₁H₁₆ClFNO₂: C, 46.24; H, 4.20; N, 4.49. Found: C, 46.22; H, 4.32; N, 4.49.

4-(3-Fluoro-5-trifluoromethylphenyl)-2-oxobutylamine trifluoroacetate (IV-10): (0.5 g, 64%). Mp: 95-96° C. ¹H NMR (MeOD-d₃, 300 MHz) δ 2.83-2.95 (m, 4H), 3.91 (s, 2H), 7.20 (d, J=8.7 Hz, 2H), 7.26 (d, J=8.7 Hz, 2H). Calcd for C₁₃H₁₂F₇NO₃: C, 42.98; H, 3.33; N, 3.86. Found: C, 43.25; H, 3.22; N, 3.85.

### Example 12

### Synthesis of Ester Precursors to Compounds of Formula II

### Methyl 3-fluoro-5-trifluorophenylacetate

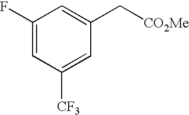

A mixture of 3-Fluoro-5-trifluoromethyl-phenylacetic acid (4.72 g, 21.2 mmol) and concentrated HCl (1.2 mL) in MeOH (50 mL) was refluxed for 2.5 h, then concentrated in vacuo. The residue was partitioned between 100 mL of EtOAc and washed sequentially with 0.5N aqueous NaOH (30 mL), 2% NaHCO₃ solution (30 mL), and brine (30 mL). The EtOAc layer was dried (Na₂SO₄), filtered, and concentrated to provide 5.01 g (quantitative) of methyl 3-fluoro-5-trifluoromethyl-phenylacetate as a colorless oil: ¹H NMR (CDCl₃, 300 MHz) δ 3.69 (s, 2H), 3.73 (s, 3H), 7.21-7.73 (m, 3H).

The compounds in the remainder of this example were synthesized according to the procedure for Methyl 3-fluoro-5-trifluorophenylacetate described above using the appropriate starting material.

Methyl 3-trifluoromethylphenylacetate: (5.26 g, 99%). ¹H NMR (CDCl₃, 300 MHz) δ 3.70 (s, 2H), 3.72 (s, 3H), 7.56-7.73 (m, 4H).

Methyl 4-methoxyphenylacetate: (5.38 g, 99%). ¹H NMR (CDCl₃, 300 MHz) δ 3.57 (s, 2H), 3.69 (s, 3H), 3.80 (s, 3H), 6.87 (d, J=8.1 Hz, 2H), 7.20 (d, J=8.1 Hz, 2H).

Methyl 4-fluorophenylacetate: (4.8 g, 88%). ¹H NMR (CDCl₃, 300 MHz) δ 3.60 (s, 2H), 3.70 (s, 3H), 7.01 (t, J=8.4 Hz, 2H), 7.26 (d, J=8.4 Hz, 2H).

Methyl 3-methylphenylacetate: (5.52 g, 98%). ¹H NMR (CDCl₃, 300 MHz) δ 2.34 (s, 3H), 3.59 (s, 2H), 3.69 (s, 3H), 6.96-7.32 (m, 4H).

Methyl 3-fluorophenylacetate: (5.24 g, 96%). ¹H NMR (CDCl₃, 300 MHz) δ 3.63 (s, 2H), 3.71 (s, 3H), 6.91-7.11 (m, 3H), 7.22-7.37 (m, 1H).

Methyl 3-methoxyphenylacetate: (5.38 g, 99%). ¹H NMR (CDCl₃, 300 MHz) δ 3.61 (s, 2H), 3.70 (s, 3H), 3.81 (s, 3H), 6.77-6.92 (m, 3H), 7.19-7.31 (m, 1H).

Methyl 3-trifluoromethoxyphenylacetate: (5.07 g, 96%). ¹H NMR (CDCl₃, 300 MHz) δ 3.65 (s, 2H), 3.71 (s, 3H), 7.10-7.29 (m, 3H), 7.35 (t, J=7.8 Hz, 1H).

Methyl 3,5-ditrifluoromethylphenylacetate: (5.07 g, 96%). ¹H NMR (CDCl₃, 300 MHz) δ 3.70 (s, 2H), 3.72 (s, 3H), 7.68 (s, 2H), 7.82 (s, 1H).

Methyl 3-methylthiophenylacetate: (4.1 g, 95%). ¹H NMR (CDCl₃, 300 MHz) δ 2.48 (s, 3H), 3.60 (s, 2H), 3.70 (s, 3H), 7.02-7.08 (m, 1H), 7.13-7.21 (m, 2H), 7.21-7.30 (m, 1H).

Methyl 3-trifluoromethylthiophenylacetate: (5.1 g, 95%). ¹H NMR (CDCl₃, 300 MHz) δ 3.67 (s, 2H), 3.71 (s, 3H), 7.37-7.46 (m, 2H), 7.54-7.63 (m, 2H).

Methyl 2-methoxyphenylacetate: (5.35 g, 99%). ¹H NMR (CDCl₃, 300 MHz) δ 3.64 (s, 2H), 3.69 (s, 3H), 3.82 (s, 3H), 6.83-6.99 (m, 2H), 7.14-7.33 (m, 2H).

Methyl 3-(3-methoxyphenyl)propionate: (5.17 g, 99%). ¹H NMR (CDCl₃, 300 MHz) δ 2.63 (t, J=8.7 Hz, 2H), 2.93 (t, J=8.7 Hz, 2H), 3.68 (s, 3H), 3.80 (s, 3H), 6.72-6.87 (m, 3H), 7.16-7.31 (m, 1H).

### Example 13

### Synthesis of Aldehyde Precursors to Compounds of Formula II

### 3-Fluoro-5-trifluoromethyl-phenylacetaldehyde

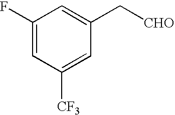

To a solution of 5.0 g (21.2 mmol) of methyl-3-fluoro-5-trifluorophenylacetate in 21.2 mL of CH₂Cl₂ and 10.6 mL of hexane, cooled to −70° C. in an argon atmosphere, was added 17.0 mL (25.4 mmol) of a 1.5M solution of DIBAL-H in toluene over approximately two minutes. The mixture was allowed to warm to −50° C. over 1 h, and 21.2 mL of MeOH was added, followed by 14 mL of 6 N HCl and 21.2 mL of H₂O. The mixture was extracted with EtOAc, and the EtOAc layer was washed sequentially with H₂O and brine, dried (MgSO₄), filtered, and concentrated. The residue was purified by silica gel chromatography (1/4 EtOAc/hexane) to provide 4.10 g (94%) of product as a colorless oil: ¹H NMR (CDCl₃) δ 3.82 (s, 2H), 7.11-7.31 (m, 3H), 9.80 (s, 1H).

The compounds in the remainder of this example were synthesized according to the procedure for 3-Fluoro-5-trifluoromethyl-phenylacetaldehyde described above using the appropriate starting material from Example 12.

3-Trifluormethylphenylacetaldehyde: (3.38 g, 75%). ¹H NMR (CDCl₃, 300 MHz) δ 3.80 (s, 2H), 7.37-7.65 (m, 4H), 9.80 (s, 1H).

4-Methoxyphenylacetaldehyde: (1.44 g, 67%). ¹H NMR (CDCl₃, 300 MHz) δ 3.63 (s, 2H), 3.80 (s, 3H), 6.91 (d, J=8.4 Hz, 2H) 7.14 (d, J=8.4 Hz, 2H), 9.73 (s, 1H).

4-Fluorophenylacetaldehyde: (1.23 g, 75%). ¹H NMR (CDCl₃, 300 MHz) δ 3.69 (s, 2H), 7.06 (t, J=8.4 Hz, 2H) 7.15-7.24 (m, 2H), 9.75 (s, 1H).

3-Methylphenylacetaldehyde: (1.1 g, 65%). ¹H NMR (CDCl₃, 300 MHz) δ 2.36 (s, 3H), 3.65 (s, 2H), 6.97-7.39 (m, 4H), 9.74 (s, 1H).

3-Fluorophenylacetaldehyde: (1.4 g, 73%). ¹H NMR (CDCl₃, 300 MHz) δ 3.71 (s, 2H), 6.89-7.10 (m, 3H), 7.28-7.41 (m, 1H), 9.76 (s, 1H).

3-Methoxyphenylacetaldehyde: (1.14 g, 58%). ¹H NMR (CDCl₃, 300 MHz) δ 3.35 (s, 2H), 3.81 (s, 3H), 6.73-6.92 (m, 3H) 7.24-7.36 (m, 1H), 9.74 (s, 1H).

3-Trifluoromethoxyphenylacetaldehyde: (1.49 g, 67%). ¹H NMR (CDCl₃, 300 MHz) δ 3.75 (s, 2H), 7.08-7.19 (m, 3H), 7.41 (t, J=7.8 Hz, 1H)., 9.78 (s, 1H).

3,5-Ditrifluoromethylphenylacetaldehyde: (1.7 g, 75%). Mp: 50-51° C. ¹H NMR (CDCl₃, 300 MHz) δ 3.91 (s, 2H), 7.68 (s, 2H), 7.84 (s, 1H), 9.86 (s, 1H).

3-Methylthiophenylacetaldehyde: (1.9 g, 56%). ¹H NMR (CDCl₃, 300 MHz) δ 2.48 (s, 3H), 3.66 (d, J=2.7 Hz, 2H), 6.96-7.03 (m, 1H) 7.07-7.23 (m, 2H), 7.25-7.34 (m, 1H), 9.74 (s, 1H).

3-Trifluoromethylthiophenylacetaldehyde: (2.29 g, 80%). ¹H NMR (CDCl₃, 300 MHz) δ 3.76 (d, J=2.4 Hz, 2H), 7.21-7.67 (m, 4H), 9.78 (s, 1H).

2-Methoxyphenylacetaldehyde: (2.04 g, 28%). ¹H NMR (CDCl₃, 300 MHz) δ 3.33 (s, 2H), 3.83 (s, 3H), 6.87-7.02 (m, 2H) 7.12-7.37 (m, 2H), 9.69 (s, 1H).

3-(3-Methyoxyphenyl)propionaldehyde: (2.4 g, 80%). ¹H NMR (CDCl₃, 300 MHz) δ 2.78 (t, J=6.6 Hz, 2H), 2.94 (t, J=6.6 Hz, 2H), 3.80 (s, 3H), 6.68-6.88 (m, 3H), 7.14-7.33 (m, 1H), 9.82 (s, 1H).

### Example 14

### Synthesis of Aldehyde Precursor to Compound II-4

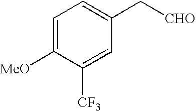

4-Methoxy-3-trifluoromethylphenylacetaldehyde: (1.44 g, 67%). To a cooled suspension of (methoxymethyl)triphenylphosphonium chloride (6.19 g, 18 mmol) in THF (40 mL) was added dropwise a solution of sodium bis(trimethylsilyl)amide in THF (1.0 M, 17.2 mL<17.2 mmol). The resulting mixture was stirred at −78° C. for one hour, and then a solution of 4-methoxy-3-trifluoromethylbenzaldehyde (2.0 g, 9.8 mmol) in THF (10 mL) was added. The resulting mixture was stirred at −78° C. for 4 hours. The reaction was quenched with water. The layers were separated. The aqueous layer was extracted with ether (2×30 mL). The combined organic layers were washed with brine (20 mL), dried (MgSO₄), filtered, and concentrated in vacuo. The crude product was used in the next step without any further purification. A solution of the crude product from previous step was dissolved in a mixture of THF (40 mL) and HCl solution (3 N, 40 mL). The resulting mixture was stirred at room temperature overnight and then refluxed for 2 hours, cooled to room temperature. The layers were separated. The aqueous layer was extracted with ether (2×20 mL). The combined organic layers were washed with brine (30 mL), filtered, and concentrated in vacuo. The residue was purified on flash column chromatography (silica gel, 5% EtOAc/hexane) to give an oil (0.74 g, 83%). ¹H NMR (CDCl₃, 300 MHz) δ 3.70 (s, 2H), 3.91 (s, 3H), 7.02 (d, J=8.4 Hz, 1H) 7.31-7.47 (m, 2H), 9.75 (s, 1H).

### Example 15

### Synthesis of 4Aldehyde Precursor to Compound II-11

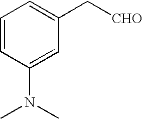

3-Dimethylaminophenylacetaldehyde: To a solution of 3-dimethylaminobenzyl alcohol (4.6 g, 304 mmol) in acetone (250 mL) was added MnO₂ (26.4 g, 30.4 mmol). The resulting mixture was heated at 60° C. for 4 hours, cooled, and filtered through a short Celite pad. The filtrate was concentrated in vacuo. The residue was purified on flash column chromatography (silica gel, 5% EtOAc/hexane) to give 3-dimethylamino-benzaldehyde (3.1 g, 64%). ¹H NMR (CDCl₃, 300 MHz) δ 3.02 (s, 6H), 6.94-7.04 (m, 1H), 7.16-7.25 (m, 2H), 7.34-7.46 (m, 1H), 9.96 (s, 1H). To a cooled suspension of (methoxymethyl)triphenylphosphonium chloride (14.1 g, 41 mmol) in THF (40 mL) was added a solution of sodium bis(trimethylsilyl) amide in THF (1.0 M, 42.2 mL, 42.2 mmol). The resulting mixture was stirred at −78° C. for 1 hour, and then a solution of 3-dimethylaminobenzylaldehyde (3.06 g, 20.5 mmol) in THF (15 mL) was added. The resulting mixture was stirred at −78° C. for 5 hours, and warmed gradually to room temperature overnight. The reaction was quenched with water. The layers were separated. The aqueous layer was extracted with ether (2×40 mL). The combined organic layers were washed with brine (20 mL), dried (MgSO₄), filtered, and concentrated. The residue (3.63 g) was used directly in the next step without any further purification. A solution of the residue (3.63 g) and HCl (3.0 N, 70 mL) in THF (70 mL) was heated at 80° C. for 1 hour, and then cooled to room temperature. The layers were separated. The aqueous layer was extracted with EtOAc (3×30 mL). The combined organic layers were washed with brine, dried (MgSO₄), filtered, and concentrated in vacuo. The residue was purified on flash column chromatography (silica gel, 5% EtOAc/hexane) to give an oil (1.34 g, 40%). ¹H NMR (CDCl₃, 300 MHz) δ 2.96 (s, 6H), 3.62 (d, J=3.0 Hz, 2H), 6.50-6.74 (m, 3H) 7.19-7.26 (m, 1H), 9.74 (s, 1H).

### Example 16

### Wittig Reaction to Form Olefin Precursor to Formula II

### (E)- and (Z)-2-Fluoro-4-(3-fluoro-5-trifluoromethylphenyl)-E-but-2-enoic acid ethyl ester

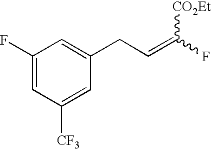

To a solution of 5.0 g (20.6 mmol) of triethyl-2-fluoro-2-phosphonoacetate (Aldrich Chemical Co.) in 94 mL of anhydrous THF cooled to 0° C. under Ar atmosphere in a 3 neck flask affixed with reflux condenser and addition funnel, was added 10.7 mL (21.4 mmol) of 2M isopropylmagnesium chloride solution in THF. The mixture was stirred for 20 min at 0° C., then at ambient temperature for 35 min. The mixture was place in a 80° C. oil bath and brought to reflux, and a solution of 4.04 g (19.6 mmol) of 3-Fluoro-5-trifluoromethyl-phenylacetaldehyde in 19.6 mL of anhydrous THF was added over 5 min. Heating was continued for 30 minutes, and the mixture was cooled to 0° C. and poured into 136 mL of saturated aqueous NH₄Cl and 45 mL of water. The mixture was extracted with two 100 mL portions of EtOAc. The combined EtOAc layers were washed with 100 mL of water and 50 mL of brine, dried (MgSO₄), filtered, and concentrated to give a brown oil. Purification by silica gel chromatography (2.5/97.5 EtOAc/hexane) provided 2.75 g (46%) of (E)-isomer and 2.11 g (35%) of (Z)-isomer. E-isomer: ¹H NMR (CDCl₃, 300 MHz) δ 1.37 (t, J=7.3 Hz, 3H), 3.96 (d, J=8.5 Hz, 2H), 4.35 (q, J=7.3 Hz, 2H), 6.03 (d of t, J=8.5, 19.5 Hz, 1H) 7.09-7.29 (m, 3H). ¹³C NMR (CDCl₃, 75 MHz) δ 15.3, 32.5, 63.2, 112.5 (d of q, J=3.7, 24.2 Hz), 120.2, 120.4, 120.8, 121.1, 122.4 (m), 144.1 (d of d, J=2.4, 7.3 Hz), 149.5 (d, J=258 Hz), 162.1 (d, J=35 Hz), 164.0 (d, J=249 Hz), MS (ESI) m/z calculated for C₁₃H₁₁F₅O₂ (M+1): 295. Found: 231 (apparent loss of 64). Z-isomer: ¹H NMR (CDCl₃, 300 MHz) δ 1.31 (t, J=7.3 Hz, 3H), 3.61 (d, J=7.9 Hz, 2H), 4.28 (q, J=7.3 Hz, 2H), 6.24 (d of t, J=7.9, 31.1 Hz, 1H) 7.08-7.26 (m, 3H). ¹³C NMR (CDCl₃, 75 MHz) δ 15.4, 31.3, 63.3, 112.6 (d of q, J=4.3, 24.4 Hz), 117.9, 118.0, 120.2, 120.5, 122.4 (m), 143.2 (d of d, J=2.4, 7.3 Hz), 150.2 (d, J=260 Hz), 161.7 (d, J=35 Hz), 164.1 (d, J=249 Hz), MS (ESI) m/z calculated for C₁₃H₁₁F₅O₂ (M+1): 295. Found: 231 (apparent loss of 64).

The compounds in the remainder of this example were synthesized according to the procedure for (E)- and (Z)-2-Fluoro-4-(3-fluoro-5-trifluoromethylphenyl)-E-but-2-enoic acid ethyl ester described above using the appropriate starting material prepared from Example 13, 14, or 15.

(E)-Ethyl-2-fluoro-4-(3-trifluoromethylphenyl)-2-butenoate: (0.65 g, 31%). ¹H NMR (CDCl₃, 300 MHz) δ 1.38 (t, J=7.2 Hz, 3H), 3.98 (d, J=8.7 Hz, 2H), 4.36 (q, J=7.2 Hz, 2H), 6.05 (dt, J=20.1, 8.4 Hz, 1H), 7.37-7.60 (m, 4H).

(E)-Ethyl-2-fluoro-4-(4-methoxyphenyl)-2-butenoate: (0.73 g, 35%). ¹H NMR (CDCl₃, 300 MHz) δ 1.37 (t, J=7.2 Hz, 3H), 3.80 (s, 3H), 3.83 (d, J=7.8 Hz, 2H), 4.35 (q, J=7.2 Hz, 2H), 6.06 (dt, J=21.6, 7.8 Hz, 1H), 6.85 (d, J=8.4 Hz, 2H), 7.14 (d, J=8.4 Hz, 2H).

(E)-Ethyl-2-fluoro-4-(4-methoxy-3-trifluoromethylphenyl)-2-butenoate: (0.54 g, 55%). ¹H NMR (CDCl₃, 300 MHz) δ 1.38 (t, J=7.2 Hz, 3H), 3.87 (d, J=8.4 Hz, 2H), 3.88 (s, 3H), 4.35 (q, J=7.2 Hz, 2H), 6.02 (dt, J=20.1, 8.7 Hz, 1H), 6.95 (d, J=8.4 Hz, 1H), 7.30-7.48 (m, 2H).

(E)-Ethyl-2-fluoro-4-(4-fluorophenyl)-2-butenoate: (1.1 g, 58%). ¹H NMR (CDCl₃, 300 MHz) δ 1.37 (t, J=7.2 Hz, 3H), 3.87 (d, J=7.8 Hz, 2H), 4.35 (q, J=7.2 Hz, 2H), 6.04 (dt, J=20.7, 8.7 Hz, 1H), 6.99 (d, J=8.7 Hz, 2H), 7.14-7.23 (m, 2H).

(E)-Ethyl-2-fluoro-4-(3-methylphenyl)-2-butenoate: (0.4 g, 24%). ¹H NMR (CDCl₃, 300 MHz) δ 1.38 (t, J=7.2 Hz, 3H), 2.34 (s, 3H), 3.86 (d, J=8.4 Hz, 2H), 4.35 (q, J=7.2 Hz, 2H), 6.08 (dt, J=21.3, 8.4 Hz, 1H), 6.94-7.25 (m, 4H).

(E)-Ethyl-2-fluoro-4-(3-fluorophenyl)-2-butenoate: (1.1 g, 48%). ¹H NMR (CDCl₃, 300 MHz) δ 1.37 (t, J=7.2 Hz, 3H), 3.90 (d, J=8.1 Hz, 2H), 4.35 (q, J=7.2 Hz, 2H), 6.05 (dt, J=21.0, 8.7 Hz, 1H), 6.87-7.07 (m, 3H), 7.22-7.35 (m, 1H).

(E)-Ethyl-2-fluoro-4-(3-methoxyphenyl)-2-butenoate: (0.78 g, 37%). ¹H NMR (CDCl₃, 300 MHz) δ 1.37 (t, J=7.2 Hz, 3H), 3.80 (s, 3H), 3.87 (d, J=7.8 Hz, 2H), 4.34 (q, J=7.8 Hz, 2H), 6.08 (dt, J=20.7, 7.8 Hz, 1H), 6.72-7.88 (m, 3H), 7.23 (t, J=7.8 Hz, 1H).

(E)-Ethyl-2-fluoro-4-phenyl-2-butenoate: (0.56 g, 28%). ¹H NMR (CDCl₃, 300 MHz) δ 1.38 (t, J=7.2 Hz, 3H), 3.90 (d, J=8.7 Hz, 2H), 4.35 (q, J=7.2 Hz, 2H), 6.09 (dt, J=20.7, 8.7 Hz, 1H), 7.02-7.60 (m, 5H).

(E)-Ethyl-2-fluoro-4-(3-trifluoromethoxyphenyl)-2-butenoate: (1.5 g, 72%). ¹H NMR (CDCl₃, 300 MHz) δ 1.37 (t, J=7.2 Hz, 3H), 3.92 (d, J=8.4 Hz, 2H), 4.35 (q, J=6.6 Hz, 2H), 6.05 (dt, J=20.4, 8.4 Hz, 1H), 7.04-7.21 (m, 3H), 7.34 (t, J=8.1 Hz, 1H).

(E)-Ethyl-2-fluoro-4-(3-dimethylaminophenyl)-2-butenoate: (0.39 g, 21%). ¹H NMR (CDCl₃, 300 MHz) δ 1.38 (t, J=6.6 Hz, 3H), 2.94 (s, 6H), 3.85 (d, J=8.1 Hz, 2H), 4.34 (q, J=6.6 Hz, 2H), 6.11 (dt, J=21.3, 8.1 Hz, 1H), 6.53-6.70 (m, 3H), 7.18 (t, J=7.8 Hz, 1H).

(E)-Ethyl-2-fluoro-4-(3,5-ditrifluoromethylphenyl)-2-butenoate: (1.12 g, 52%). ¹H NMR (CDCl₃, 300 MHz) δ 1.38 (t, J=7.2 Hz, 3H), 4.05 (d, J=8.4 Hz, 2H), 4.36 (q, J=7.2 Hz, 2H), 6.05 (dt, J=19.8, 8.4 Hz, 1H), 7.68 (s, 2H), 7.77 (s, 1H).

(E)-Ethyl-2-fluoro-4-(3-methylthiophenyl)-2-butenoate: (1.7 g, 59%). ¹H NMR (CDCl₃, 300 MHz) δ 1.35 (t, J=7.5 Hz, 3H), 2.48 (s, 3H), 3.87 (d, J=8.4 Hz, 2H), 4.35 (q, J=7.2 Hz, 2H), 6.06 (dt, J=20.7, 8.4 Hz, 1H), 6.95-7.03 (m, 1H), 7.09-7.17 (m, 2H), 7.19-7.25 (m, 1H).

(E)-Ethyl-2-fluoro-4-(3-trifluoromethylthiophenyl)-2-butenoate: (1.88 g, 61%). ¹H NMR (CDCl₃, 300 MHz) δ 1.38 (t, J=6.6 Hz, 3H), 3.93 (d, J=8.7 Hz, 2H), 4.35 (q, J=7.2 Hz, 2H), 6.06 (dt, J=20.4, 8.4 Hz, 1H), 7.31-7.43 (m, 2H), 7.47-7.60 (m, 2H).

(E)-Ethyl-2-fluoro-4-(2-methoxyphenyl)-2-butenoate: (0.92 g, 43%). ¹H NMR (CDCl₃, 300 MHz) δ 1.37 (t, J=7.2 Hz, 3H), 3.83 (s, 3H), 3.87 (d, J=8.4 Hz, 2H), 4.34 (q, J=7.2 Hz, 2H), 6.13 (dt, J=21.3, 7.8 Hz, 1H), 6.81-6.96 (m, 2H), 7.14-7.29 (m, 2H).

(E)-Ethyl-2-fluoro-5-(3-methoxyphenyl)-2-pentanoate: (1.3 g, 45%). ¹H NMR (CDCl₃, 300 MHz) δ 1.34 (t, J=7.5 Hz, 3H), 2.69-2.91 (m, 4H), 3.80 (s, 3H), 4.29 (q, J=7.2 Hz, 2H), 5.93 (dt, J=21.6, 7.2 Hz, 1H), 6.70-6.87 (m, 3H), 7.16-7.26 (m, 1H).

(E)-Ethyl-2-fluoro-4-(3-methoxyphenyl)-4-methyl-2-butenoate: (0.6 g, 27%). ¹H NMR (CDCl₃, 300 MHz) δ 1.35 (t, J=7.5 Hz, 3H), 1.78 (d, J=5.5 Hz, 3H), 3.79 (s, 3H), 3.91 (s, 2H), 4.32 (q, J=7.5 Hz, 2H), 6.72-7.86 (m, 3H), 7.16-7.26 (m, 1H).

### Example 17

### Synthesis of Alcohol Precursor to Formula II

### (E)-2-Fluoro-4-(3-fluoro-5-trifluoromethylphenyl)-but-2-en-1-ol

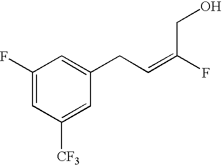

To a stirred solution of 1.77 g (6.02 mmol) of (E)-2-Fluoro-4-(3-fluoro-5-trifluoromethylphenyl)-E-but-2-enoic acid ethyl ester in 30 mL of hexane under Ar atmosphere and cooled to between −30° C. and −15° C. was added 12.04 mL (18.06 mmol) of a 1.5M solution of diisobutylaluminum hydride in toluene over 2 min. The mixture was stirred for 30 min with continued cooling, and 18 mL of MeOH was added followed by 22.5 mL of 6N HCl solution. The mixture was extracted with two 50 mL portions of EtOAc, and the combined EtOAc layers were washed sequentially with water and brine, dried (MgSO₄), filtered, and concentrated to an oil. Purification by silica gel chromatography (36/65 EtOAc/hexane) provided 1.39 g (91%) of product as a colorless oil: ¹H NMR (CDCl₃, 300 MHz) δ 1.99 (brd t, J=5.5 Hz, 1H), 3.45 (d, J=8.5 Hz, 2H), 4.33 (d of d, J=5.5, 10.8 Hz, 2H), 5.39 (d of t, J=8.5, 19.5 Hz, 1H,) 7.09-7.25 (m, 3H).

The compounds in the remainder of this example were synthesized according to the procedure for (E)-2-Fluoro-4-(3-fluoro-5-trifluoromethylphenyl)-but-2-en-1-ol described above using the appropriate starting material prepared from Example 16.

(Z)-2-Fluoro-4-(3-fluoro-5-trifluoromethylphenyl)-but-2-en-1-ol: 94% yield: ¹H NMR (CDCl₃, 300 MHz) δ 1.99 (brd t, 1H), 3.51 (d, J=8.0 Hz, 2H), 4.19 (d, J=14.6 Hz, 2H), 5.05 (d of t, J=7.9, 34.8 Hz, 1H) 7.09-7.25 (m, 3H).

(E)-2-Fluoro-4-(3-trifluoromethylphenyl)-2-butenol: (0.52 g, 94%). ¹H NMR (CDCl₃, 300 MHz) δ 3.46 (d, J=7.8 Hz, 2H), 3.35 (d, J=20.1 Hz, 2H), 5.56 (dt, J=20.1, 8.4 Hz, 1H), 7.35-7.59 (m, 4H),

(E)-2-Fluoro-4-(4-methoxyphenyl)-2-butenol: (0.41 g, 70%). ¹H NMR (CDCl₃, 300 MHz) δ 3.33 (d, J=8.4 Hz, 2H), 3.79 (s, 3H), 4.33 (d, J=20.7 Hz, 2H), 5.45 (dt, J=20.7, 8.4 Hz, 1H), 6.85 (d, J=8.4 Hz, 2H), 7.11 (d, J=8.4 Hz, 2H),

(E)-2-Fluoro-4-(4-methoxy-3-trifluoromethylphenyl)-2-butenol: (0.39 g, 83%). ¹H NMR (CDCl₃, 300 MHz) δ 3.36 (d, J=8.1 Hz, 2H), 3.89 (s, 3H), 4.33 (d, J=21.3 Hz, 2H), 5.38 (dt, J=20.1, 8.1 Hz, 1H), 6.95 (d, J=8.4 Hz, 1H), 7.29-7.43 (m, 2H),

(E)-2-Fluoro-4-(4-fluorophenyl)-2-butenol: (1.1 g, 90%). ¹H NMR (CDCl₃, 300 MHz) δ 3.35 (d, J=8.7 Hz, 2H), 4.33 (dd, J=20.7, 6.0 Hz, 2H), 5.39 (dt, J=21.0, 7.8 Hz, 1H), 6.99 (t, J=8.7 Hz, 2H), 7.09-7.21 (m, 2H),

(E)-2-Fluoro-4-(3-methylphenyl)-2-butenol: (0.24 g, 73%). ¹H NMR (CDCl₃, 300 MHz) δ 2.34 (s, 3H), 3.35 (d, J=8.7 Hz, 2H), 4.33 (d, J=19.8 Hz, 2H), 5.42 (dt, J=20.1, 8.4 Hz, 1H), 6.93-7.11 (m, 3H), 7.20 (t, J=8.1 Hz, 1H).

(E)-2-Fluoro-4-(3-fluorophenyl)-2-butenol: (0.94 g, 87%). ¹H NMR (CDCl₃, 300 MHz) δ 3.38 (d, J=7.8 Hz, 2H), 4.33 (d, J=21.3 Hz, 2H), 5.4o (dt, J=20.1, 8.7 Hz, 1H), 6.84-7.04 (m, 3H), 7.19-7.35 (m, 1H).

(E)-2-Fluoro-4-(3-methoxyphenyl)-2-butenol: (0.71 g, 93%). ¹H NMR (CDCl₃, 300 MHz) δ 3.36 (d, J=8.7 Hz, 2H), 3.80 (s, 3H), 4.33 (dd, J=20.7, 6.0 Hz, 2H), 5.42 (dt, J=20.7, 8.1 Hz, 1H), 6.68-6.87 (m, 3H), 7.22 (t, J=7.8 Hz, 1H).

(E)-2-Fluoro-4-phenyl-2-butenol: (0.34 g, 93%). ¹H NMR (CDCl₃, 300 MHz) δ 3.39 (d, J=7.8 Hz, 2H), 4.34 (dd, J=20.7, 6.0 Hz, 2H), 5.43 (dt, J=20.7, 8.4 Hz, 1H), 7.13-7.39 (m, 5H).

(E)-2-Fluoro-4-(3-trifluoromethoxyphenyl)-2-butenol: (0.91 g, 83%). ¹H NMR (CDCl₃, 300 MHz) δ 3.42 (d, J=8.7 Hz, 2H), 4.33 (dd, J=20.7, 6.0 Hz, 2H), 5.40 (dt, J=20.7, 8.4 Hz, 1H), 7.00-9.19 (m, 3H), 7.33 (t, J=8.1 Hz, 1H).

(E)-2-Fluoro-4-(3-dimethylaminophenyl)-2-butenol: (0.2 g, 60%). ¹H NMR (CDCl₃, 300 MHz) δ 2.94 (s, 6H), 3.34 (d, J=8.4 Hz, 2H), 4.33 (dd, J=21.3, 3.6 Hz, 2H), 5.45 (dt, J=20.1, 8.4 Hz, 1H), 6.50-6.71 (m, 3H), 7.18 (t, J=7.8 Hz, 1H),

(E)-2-Fluoro-4-(3,5-ditrifluoromethylphenyl)-2-butenol: (0.61 g, 64%). ¹H NMR (CDCl₃, 300 MHz) δ 3.55 (d, J=8.1 Hz, 2H), 4.37 (dd, J=20.1, 6.3 Hz, 2H), 5.41 (dt, J=19.5, 7.8 Hz, 1H), 7.66 (s, 2H), 7.75 (s, 1H).

(E)-2-Fluoro-4-(3-methylthiophenyl)-2-butenol: (1.1 g, 78%). ¹H NMR (CDCl₃, 300 MHz) δ 2.48 (s, 3H), 3.35 (d, J=8.1 Hz, 2H), 3.80 (s, 3H), 4.33 (dd, J=20.1, 3.0 Hz, 2H), 5.41 (dt, J=20.1, 8.4 Hz, 1H), 6.96 (d, J=7.2 Hz, 1H), 7.05-7.17 (m, 2H), 7.23 (t, J=8.1 Hz, 1H).

(E)-2-Fluoro-4-(3-trifluoromethylthiophenyl)-2-butenol: (1.3 g, 78%). ¹H NMR (CDCl₃, 300 MHz) δ 3.42 (d, J=8.1 Hz, 2H), 4.35 (d, J=21.3 Hz, 2H), 5.41 (dt, J=20.1, 7.8 Hz, 1H), 7.29-7.42 (m, 2H), 7.45-7.60 (m, 2H).

(E)-2-Fluoro-4-(2-methoxyphenyl)-2-butenol: (0.65 g, 89%). ¹H NMR (CDCl₃, 300 MHz) δ 3.36 (d, J=8.4 Hz, 2H), 3.84 (s, 3H), 4.35 (dd, J=21.6, 6.9 Hz, 2H), 5.37 (dt, J=20.1, 8.7 Hz, 1H), 6.80-6.99 (m, 2H), 7.09-7.33 (m, 2H),

(E)-2-Fluoro-5-(3-methoxyphenyl)-2-pentanol: (0.95 g, 90%). ¹H NMR (CDCl₃, 300 MHz) δ 2.33 (q, J=7.2 Hz, 2H), 2.67 (t, J=7.2 Hz, 2H), 3.80 (s, 3H), 4.03 (d, J=21.3 Hz, 2H), 5.22 (dt, J=20.7, 8.4 Hz, 1H), 6.68-6.87 (m, 3H), 7.22 (t, J=8.1 Hz, 1H).

(E)-2-Fluoro-4-(3-methoxyphenyl)-4-methyl-2-butenol: (0.26 g, 55%). ¹H NMR (CDCl₃, 300 MHz) δ 1.63 (d, J=3.9 Hz, 3H), 3.34 (s, 2H), 3.80 (s, 3H), 4.38 (dd, J=22.5, 6.0 Hz, 2H), 6.69-6.83 (m, 3H), 7.22 (t, J=7.8 Hz, 1H).

### Example 18

### Synthesis of N-Phthalyl-Protected Precursors to Formula II

### (E)-N-(2-Fluoro-4-(3-fluoro-5-trifluoromethylphenyl)-but-2-enyl)phthalimide

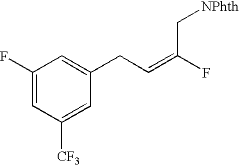

To a stirred partially dissolved solution of 849 mg (3.37 mmol) of (E)-2-Fluoro-4-(3-fluoro-5-trifluoromethylphenyl)-but-2-en-1-ol, 546 mg (3.71 mmol) of phthalimide, and
1.326 g (5.05 mmol) of triphenylphosphine in 16.35 mL of THF at 0° C. was added over 8 min a solution of 1.021 g (5.05 mmol) of diisopropylazodicarboxylate in 8 mL of THF. The cooling bath was removed, and the mixture was stirred at ambient temperature for 18 h. THF was removed under vacuum, and the residue was purified by silica gel chromatography (15/85 EtOAc/hexane) to provide 1.05 g (82%) of product as a white solid: ¹H NMR (CDCl₃, 300 MHz) δ 3.66 (d, J=8.5 Hz, 2H), 4.54 (d, J=19.5 Hz, 2H), 5.44 (d of t, J=7.9, 19.5 Hz, 1H,) 7.17-7.33 (m, 3H), 7.75 (m, 2H), 7.88 (m, 2H).

The compounds in the remainder of this example were synthesized according to the procedure for (E)-N-(2-Fluoro-4-(3-fluoro-5-trifluoromethylphenyl)-but-2-enyl)phthalimide described above using the appropriate starting material prepared from Example 17.

(Z)-N-(2-Fluoro-4-(3-fluoro-5-trifluoromethylphenyl)-but-2-enyl)phthalimide: 93% yield: ¹H NMR (CDCl₃, 300 MHz) δ 3.48 (d, J=13.4 Hz, 2H), 4.44 (d, J=7.9 Hz, 2H), 5.11 (d of t, J=7.9, 34.2 Hz, 1H) 7.07-7.23 (m, 3H), 7.75 (m, 2H), 7.89 (m, 2H).

(E)-N-(2-Fluoro-4-(3-trifluoromethylphenyl)-2-butenyl)phthalimide: (0.68 g, 86%). Mp: 75-76° C. ¹H NMR (CDCl₃, 300 MHz) δ 3.67 (d, J=8.7 Hz, 2H), 4.46 (d, J=19.5 Hz, 2H), 5.46 (dt, J=19.5, 8.1 Hz, 1H), 7.35-7.59 (m, 4H), 7.71-7.81 (m, 2H), 7.83-7.98 (m, 2H)

(E)-N-(2-Fluoro-4-(4-methoxyphenyl)-2-butenyl)phthalimide: (0.57 g, 85%). Mp: 43-44° C. ¹H NMR (CDCl₃, 300 MHz) δ 3.54 (d, J=8.1 Hz, 2H), 3.80 (s, 3H), 4.55 (d, J=18.9 Hz, 2H), 5.44 (dt, J=21, 8.7 Hz, 1H), 6.86 (d, J=8.4 Hz, 2H), 7.19 (d, J=8.4 Hz, 2H), 7.71-7.81 (m, 2H), 7.83-7.98 (m, 2H).

(E)-N-(2-Fluoro-4-(4-methoxy-3-trifluoromethylphenyl)-2-butenyl)phthalimide: (0.54 g, 94%). Mp: 107-109° C. ¹H NMR (CDCl₃, 300 MHz) δ 3.57 (d, J=7.2 Hz, 2H), 3.89 (s, 3H), 4.55 (d, J=18.9 Hz, 2H), 5.42 (dt, J=19.5, 8.7 Hz, 1H), 6.96 (d, J=7.8 Hz, 1H), 7.36-7.50 (m, 2H), 7.71-7.83 (m, 2H), 7.84-7.98 (m, 2H).

(E)-N-(2-Fluoro-4-(4-fluorophenyl)-2-butenyl)phthalimide: (0.91 g, 78%). Mp: 80-81° C. ¹H NMR (CDCl₃, 300 MHz) δ 3.57 (d, J=7.8 Hz, 2H), 4.55 (d, J=19.5 Hz, 2H), 5.43 (dt, J=19.5, 8.1 Hz, 1H), 7.00 (t, J=8.7 Hz, 2H), 7.18-7.36 (m, 2H), 7.71-7.81 (m, 2H), 7.83-7.98 (m, 2H).

(E)-N-(2-Fluoro-4-(3-methylphenyl)-2-butenyl)phthalimide: (0.33 g, 84%). Mp: 75-76° C. ¹H NMR (CDCl₃, 300 MHz) δ 2.34 (s, 3H), 3.56 (d, J=8.1 Hz, 2H), 4.55 (d, J=18.9 Hz, 2H), 5.47 (dt, J=19.5, 8.4 Hz, 1H), 6.96-7.14 (m, 3H), 7.21 (t, J=8.4 Hz, 1H), 7.67-7.82 (m, 2H), 7.83-7.98 (m, 2H).

(E)-N-(2-Fluoro-4-(3-fluorophenyl)-2-butenyl)phthalimide: (0.93 g, 85%). Mp: 62-63° C. ¹H NMR (CDCl₃, 300 MHz) δ 3.60 (d, J=8.1 Hz, 2H), 4.54 (d, J=19.8 Hz, 2H), 5.45 (dt, J=19.8, 8.1 Hz, 1H), 6.84-7.14 (m, 3H), 7.18-7.40 (m, 1H), 7.67-7.82 (m, 2H), 7.83-7.98 (m, 2H)

(E)-N-(2-Fluoro-4-(3-methoxyphenyl)-2-butenyl)phthalimide: (0.9 g, 84%). Mp: 74-75° C. ¹H NMR (CDCl₃, 300 MHz) δ 3.57 (d, J=7.8 Hz, 2H), 3.82 (s, 3H), 4.55 (d, J=18.9 Hz, 2H), 5.47 (dt, J=20.1, 7.8 Hz, 1H), 6.68-6.95 (m, 3H), 7.23 (t, J=8.1 Hz, 1H), 7.67-7.82 (m, 2H), 7.83-7.98 (m, 2H)

(E)-N-(2-Fluoro-4-phenyl-2-butenyl)phthalimide: (0.5 g, 84%). Mp: 73-74° C. ¹H NMR (CDCl₃, 300 MHz) δ 3.60 (d, J=8.7 Hz, 2H), 4.56 (d, J=18.9 Hz, 2H), 5.48 (dt, J=19.5, 8.4 Hz, 1H), 7.15-7.42 (m, 5H), 7.67-7.82 (m, 2H), 7.83-7.98 (m, 2H).

(E)-N-(2-Fluoro-4-(3-trifluoromethoxyphenyl)-2-butenyl)phthalimide: (1.1 g, 84%). ¹H NMR (CDCl₃, 300 MHz) δ 3.63 (d, J=8.4 Hz, 2H), 4.55 (d, J=19.5 Hz, 2H), 5.46 (dt, J=19.8, 7.8 Hz, 1H), 7.03-7.26 (m, 3H), 7.34 (t, J=8.7 Hz, 1H), 7.67-7.82 (m, 2H), 7.83-7.98 (m, 2H)

(E)-N-(2-Fluoro-4-(3-dimethylaminophenyl)-2-butenyl)phthalimide: (0.23 g, 75%). Mp: 96-97° C. ¹H NMR (CDCl₃, 300 MHz) δ 2.95 (s, 6H), 3.55 (d, J=7.2 Hz, 2H), 4.56 (d, J=19.5 Hz, 2H), 5.49 (dt, J=20.1, 8.7 Hz, 1H), 6.50-6.76 (m, 3H), 7.18 (t, J=7.2 Hz, 2H), 7.69-7.81 (m, 2H), 7.83-7.95 (m, 2H).

(E)-N-(2-Fluoro-4-(3,5-ditrifluoromethylphenyl)-2-butenyl)phthalimide: (0.84 g, 71%). Mp: 112-113° C. ¹H NMR (CDCl₃, 300 MHz) δ 3.75 (d, J=7.8 Hz, 2H), 4.56 (d, J=20.1 Hz, 2H), 5.44 (dt, J=19.5, 8.1 Hz, 1H), 7.71-7.83 (m, 5H), 7.84-7.96 (m, 2H).

(E)-N-(2-Fluoro-4-(3-methylthiophenyl)-2-butenyl)phthalimide: (1.33 g, 75%). Mp: 88-89° C. ¹H NMR (CDCl₃, 300 MHz) δ 2.50 (s, 3H), 3.57 (d, J=7.8 Hz, 2H), 4.55 (d, J=19.8 Hz, 2H), 5.45 (dt, J=20.4, 7.8 Hz, 1H), 7.00-7.32 (m, 4H), 7.69-7.80 (m, 2H), 7.83-7.95 (m, 2H).

(E)-N-(2-Fluoro-4-(3-trifluoromethylthiophenyl)-2-butenyl)phthalimide: (1.61 g, 90%). Mp: 54-55° C. ¹H NMR (CDCl₃, 300 MHz) δ 3.64 (d, J=7.8 Hz, 2H), 4.55 (d, J=20.1 Hz, 2H), 5.45 (dt, J=19.5, 7.8 Hz, 1H), 7.31-7.46 (m, 2H), 7.48-7.60 (m, 2H), 7.70-7.81 (m, 2H), 7.84-7.95 (m, 2H).

(E)-N-(2-Fluoro-4-(2-methoxyphenyl)-2-butenyl)phthalimide: (0.79 g, 75%). Mp: 88-89° C. ¹H NMR (CDCl₃, 300 MHz) δ 3.55 (d, J=8.1 Hz, 2H), 3.85 (s, 3H), 4.60 (d, J=19.8 Hz, 2H), 5.46 (dt, J=20.1, 8.7 Hz, 1H), 6.78-7.01 (m, 2H), 7.16-7.33 (m, 2H), 7.69-7.81 (m, 2H), 7.83-7.95 (m, 2H).

(E)-N-(2-Fluoro-5-(3-methoxyphenyl)-2-pentenyl)phthalimide: (1.14 g, 75%). Mp: 86-87° C. ¹H NMR (CDCl₃, 300 MHz) δ 2.54 (q, J=8.1 Hz, 2H), 2.74 (d, J=8.1 Hz, 2H), 3.82 (s, 3H), 4.33 (d, J=20.1 Hz, 2H), 5.29 (dt, J=20.7, 7.8 Hz, 1H), 6.67-6.91 (m, 3H), 7.22 (t, J=8.1 Hz, 1H), 7.67-7.80 (m, 2H), 7.81-7.96 (m, 2H).

(E)-N-(2-Fluoro-4-(3-methoxyphenyl)-4-methyl-2-butenyl)phthalimide: (0.56 g, 65%). Mp: 99-100° C. ¹H NMR (CDCl₃, 300 MHz) δ 1.60 (d, J=3.6 hz, 3H), 3.57 (s, 2H), 3.82 (s, 3H), 4.61 (d, J=20.1 Hz, 2H), 6.71-6.89 (m, 2H), 7.17-7.32 (m, 2H), 7.71-7.81 (m, 2H), 7.83-7.98 (m, 2H).

### Example 19

### Synthesis of N-Phthalyl-Protected Precursor to Compound II-14-E

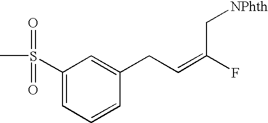

(E)-N-(2-Fluoro-4-(3-methanesulfonylphenyl)-2-butenyl)phthalimide: A solution of periodic acid (0.2 g, 0.87 mmol) in dry acetonitrile (2.5 mL) was stirred vigorously for one hour, and then CrO₃ (4.16 mg, 0.042 mmol) was added. The resulting mixture was stirred at room temperature for 5 min to give a clear orange solution. This periodic acid/CrO₃ complex solution was added dropwise over a period of 45 min to a solution of (E)-2-Fluoro-4-(3-methylthiophenyl)-2-butenolphthalimide (0.142 g, 0.42 mmol) in EtOAc (5.0 mL) at −35° C. The resulting mixture was stirred at −35° C. for one hour, and quenched by the addition of saturated Na₂SO₃ solution (10 mL). The mixture was filtered, and the solid was washed EtOAc. The filtrate was washed with saturated Na₂SO₃ solution (2×20 mL) and brine (20 mL), dried (MgSO₄), filtered, and concentrated in vacuo to give a solid (0.13 g, 83%). Mp: 124-126° C. ¹H NMR (CDCl₃, 300 MHz) δ 3.10 (s, 3H), 3.70 (d, J=8.4 Hz, 2H), 4.57 (d, J=19.8 Hz, 2H), 5.46 (dt, J=19.5, 8.7 Hz, 1H), 7.48-7.66 (m, 3H), 7.72-7.80 (m, 2H), 7.81-7.86 (m, 1H), 7.86-7.98 (m, 2H)

### Example 20

### N-Phthalyl Deprotection to Form Compounds of Formula II

### (E)-2-Fluoro-4-(3-fluoro-5-trifluoromethylphenyl)-but-2-enylamine (II-1-E)

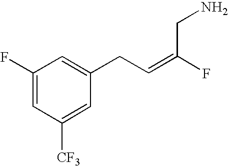

(E)-N-(2-Fluoro-4-(3-fluoro-5-trifluoromethylphenyl)-but-2-enyl)phthalimide (844 mg, 2.21 mmol) was dissolved in a mixture of 18.4 mL of EtOH and 36.7 mL of a 33% solution of MeNH₂ in EtOH. The resulting solution was refluxed under Ar atmosphere for 2.5 h. When cool the mixture was concentrated under vacuum, and the residue was purified by silica gel chromatography (50/50/0.1 CH₃CN/CH₂Cl₂/conc. NH₄OH) to provide 492 mg (89%) of desired product as an oil: ¹H NMR (CDCl₃) δ 3.45 (d, J=8.0 Hz, 2H), 3.50 (d, J=20.8 Hz, 2H), 5.25 (d of t, J=7.9, 20.1 Hz, 1H) 7.07-7.23 (m, 3H).

(Z)-2-Fluoro-4-(3-fluoro-5-trifluoromethylphenyl)-but-2-enylamine (II-1-Z): This compound was prepared from (Z)-N-(2-Fluoro-4-(3-fluoro-5-trifluoromethylphenyl)-but-2-enyl)phthalimide as described above for II-1-E. 90% yield: ¹H NMR (CDCl₃, 300 MHz) δ 3.39 (d, J=13.4 Hz, 2H), 3.47 (d, J=7.9 Hz, 2H), 4.90 (d of t, J=7.9, 35.4 Hz, 1H) 7.24-7.08 (m, 3H).

### Example 21

### HCl Salt Formation for Compounds of Formula II

### (E)-2-Fluoro-4-(3-fluoro-5-trifluoromethylphenyl)-but-2-enylamine HCl salt (II-1-E)

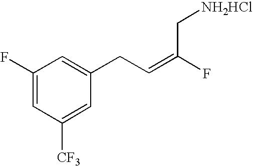

To a solution of 473 mg (1.88 mmol) of the free amine, compound II-1-E, in 9.4 mL of anhydrous Et₂O was added 3.76 mL (3.76 mmol) of a 1M solution of HCl in Et₂O. The resulting white precipitate was collected by filtration to provide 419 mg (77%) of hydrogen chloride salt as a white solid: m.p. 179-180° C.; Elemental analysis calculated for C₁₁H₁₁ClF₅N: C, 45.93; H, 3.85; N, 4.87. Found: C, 46.17; H, 3.89; N, 5.00.

(Z)-2-Fluoro-4-(3-fluoro-5-trifluoromethylphenyl)-but-2-enylamine HCl salt (II-1-Z): This compound was prepared from compound II-1-Z as described above for II-1-E. 88% yield: m.p. 89.5-91.5° C.; Elemental analysis calculated for C₁₁H₁₁ClF₅N: C, 45.93; H, 3.85; N, 4.87. Found: C, 45.76; H, 3.79; N, 5.05.

### Example 22

### Sequential N-Phthalyl Deprotection and HCl Salt Formation for Compounds of Formula II

The salt form of compounds of formula II described in this example were prepared in a sequential fashion by performing the deprotection described in Example 20 followed by the HCl salt formation described in Example 21.

(E)-2-Fluoro-4-(3-trifluoromethylphenyl)-2-butenylamine hydrochloride (II-2): (0.21 g, 93%). Mp: 145-147° C. ¹H NMR (MeOD-d₃, 300 MHz) δ 3.57 (d, J=7.8 Hz, 2H), 4.00 (d, J=19.5 Hz, 2H), 5.69 (dt, J=20.1, 7.8 Hz, 1H), 7.38-7.76 (m, 4H). ESMS m/z 234 (M+H)⁺. Calcd for C₁₁H₁₂ClF₄N: C, 48.99; H, 4.48; N, 5.19. Found: C, 49.17; H, 3.96; N, 5.27.

(E)-2-Fluoro-4-(4-methoxyphenyl)-2-butenylamine hydrochloride (II-3): (0.32 g, 86%). Mp: 232-233° C. ¹H NMR (MeOD-d₃, 300 MHz) δ 3.44 (d, J=8.7 Hz, 2H), 3.87 (s, 3H), 3.96 (d, J=18.9 Hz, 2H), 5.67 (dt, J=21.0, 7.8 Hz, 1H), 7.12 (d, J=7.8 Hz, 1H), 7.39-7.56 (m, 2H). ESMS m/z 264 (M+H)⁺. Calcd for C₁₂H₁₄ClF₄NO: C, 48.09; H, 4.71; N, 4.67. Found: C, 47.93; H, 4.27; N, 4.83.

(E)-2-Fluoro-4-(4-methoxy-3-trifluoromethylphenyl)-2-butenylamine hydrochloride (II-4): (0.3 g, 74%). Mp: 146-147° C. ¹H NMR (MeOD-d₃, 300 MHz) δ 3.36 (d, J=7.8 Hz, 2H), 3.75 (s, 3H), 3.92 (d, J=19.5 Hz, 2H), 5.65 (dt, J=20.7, 8.1 Hz, 1H), 6.86 (d, J=8.4 Hz, 2H), 7.13 (d, J=8.4 Hz, 2H). ESMS m/z 196 (M+H)⁺. Calcd for C₁₁H₁₅ClFNO: C, 57.02; H, 6.52; N, 6.04. Found: C, 56.88; H, 6.04; N, 6.37.

(E)-2-Fluoro-4-(4-fluorophenyl)-2-butenylamine hydrochloride (II-5): (0.51 g, 82%). Mp: 213-214° C. ¹H NMR (MeOD-d₃, 300 MHz) δ 3.42 (d, J=8.4 Hz, 2H), 3.95 (d, J=19.5 Hz, 2H), 5.66 (dt, J=21, 8.1 Hz, 1H), 7.02 (t, J=8.7 Hz, 2H), 7.18-7.33 (m, 2H). ESMS m/z 184 (M+H)⁺. Calcd for C₁₀H₁₂ClF₂N: C, 54.68; H, 5.51; N, 6.38. Found: C, 54.51; H, 5.59; N, 6.36.

(E)-2-Fluoro-4-(3-methylphenyl)-2-butenylamine hydrochloride (II-6): (0.16 g, 97%). Mp: 195-196° C. ¹H NMR (MeOD-d₃, 300 MHz) δ 2.31 (s, 3H), 3.39 (d, J=8.7 Hz, 2H), 3.94 (d, J=19.5 Hz, 2H), 5.66 (dt, J=21.0, 8.4 Hz, 1H), 6.69-7.10 (m, 3H), 7.17 (t, J=7.2 Hz, 1H). ESMS m/z 180 (M+H)⁺. Calcd for C₁₁H₁₅ClFN: C, 61.25; H, 7.01; N, 6.49. Found: C, 61.28; H, 6.78; N, 6.56.

(E)-2-Fluoro-4-(3-fluorophenyl)-2-butenylamine hydrochloride (II-7): (0.12 g, 78%). Mp: 175-176° C. ¹H NMR (MeOD-d₃, 300 MHz) δ 3.46 (d, J=8.7 Hz, 2H), 3.95 (d, J=19.5 Hz, 2H), 5.68 (dt, J=20.1, 8.4 Hz, 1H), 6.89-7.12 (m, 3H), 7.24-7.41 (m, 1H). ESMS m/z 184 (M+H)⁺. Calcd for C₁₀H₁₂ClF₂N: C, 54.68; H, 5.51; N, 6.38. Found: C, 54.73; H, 5.15; N, 6.66.

(E)-2-Fluoro-4-(3-methoxyphenyl)-2-butenylamine hydrochloride (II-8): (0.47 g, 75%). Mp: 175-176° C. ¹H NMR (MeOD-d₃, 300 MHz) δ 3.40 (d, J=8.7 Hz, 2H), 3.77 (s, 3H), 3.94 (d, J=18.9 Hz, 2H), 5.68 (dt, J=20.7, 7.8 Hz, 1H), 6.72-6.87 (m, 3H), 7.15-7.28 (m, 1H). ESMS m/z 196 (M+H)⁺. Calcd for C₁₁H₁₅ClFNO: C, 57.02; H, 6.52; N, 6.04. Found: C, 57.10; H, 6.13; N, 6.25.

(E)-2-Fluoro-4-phenyl-2-butenylamine hydrochloride (II-9): (0.11 g, 95%). Mp: 120-121° C. ¹H NMR (MeOD-d₃, 300 MHz) δ 3.44 (d, J=8.4 Hz, 2H), 3.95 (d, J=19.5 Hz, 2H), 5.68 (dt, J=20.7, 7.8 Hz, 1H), 7.17-7.39 (m, 5H). ESMS m/z 166 (M+H)⁺. Calcd for C₁₀H₁₃ClFN: C, 59.56; H, 6.50; N, 6.95. Found: C, 59.77; H, 6.30; N, 7.16.

(E)-2-Fluoro-4-(3-trifluoromethoxyphenyl)-2-butenylamine hydrochloride (II-10): (0.75 g, 98%). Mp: 174-175° C. ¹H NMR (MeOD-d₃, 300 MHz) δ 3.50 (d, J=8.7 Hz, 2H), 3.96 (d, J=19.5 Hz, 2H), 5.69 (dt, J=20.7, 8.1 Hz, 1H), 7.11-7.31 (m, 3H), 7.41 (t, J=7.2 Hz, 1H). ESMS m/z 250 (M+H)⁺. Calcd for C₁₁H₁₂ClF₄NO: C, 46.25; H, 4.23; N, 4.90. Found: C, 46.26; H, 4.39; N, 4.94.

(E)-2-Fluoro-4-(3-dimethylaminophenyl)-2-butenylamine hydrochloride (II-11): (0.16 g, 85%). ¹H NMR (MeOD-d₃, 300 MHz) δ 3.27 (s, 6H), 3.55 (d, J=7.8 Hz, 2H), 4.00 (d, J=19.5 Hz, 2H), 5.71 (dt, J=20.1, 8.7 Hz, 1H), 7.37-7.61 (m, 4H). ESMS m/z 209 (M+H)⁺.

(E)-2-Fluoro-4-(3,5-ditrifluoromethylphenyl)-2-butenylamine hydrochloride (II-12): (0.51 g, 79%). Mp: 197-198° C. ¹H NMR (MeOD-d₃, 300 MHz) δ 3.65 (d, J=7.8 Hz, 2H), 4.00 (d, J=19.5 Hz, 2H), 5.74 (dt, J=20.1, 7.8 Hz, 1H), 7.85 (s, 1H), 7.88 (s, 2H). ESMS m/z 302 (M+H)⁺. Calcd for C₁₂H₁₁ClF₇N: C, 42.68; H, 3.28; N, 4.15. Found: C, 42.89; H, 3.49; N, 4.51.

(E)-2-Fluoro-4-(3-methylthiophenyl)-2-butenylamine hydrochloride (II-13): (0.13 g, 96%). Mp: 192-193° C. ¹H NMR (MeOD-d₃, 300 MHz) δ 2.46 (s, 3H), 3.41 (d, J=7.8 Hz, 2H), 3.94 (d, J=18.9 Hz, 2H), 5.67 (dt, J=20.7, 7.8 Hz, 1H), 7.00 (d, J=7.8 Hz, 1H), 7.08-7.18 (m, 2H), 7.24 (t, J=7.8 Hz, 1H). ESMS m/z 212 (M+H)⁺. Calcd for C₁₁H₁₅ClFNS: C, 53.32; H, 6.10; N, 5.65. Found: C, 53.13; H, 5.77; N, 5.72.

(E)-2-Fluoro-4-(3-methanesulfonylphenyl)-2-butenylamine hydrochloride (II-14): (0.33 g, 66%). Mp: 196-197° C. ¹H NMR (MeOD-d₃, 300 MHz) 83.12 (s, 3H), 3.58 (d, J=8.7 Hz, 2H), 3.99 (d, J=19.5 Hz, 2H), 5.72 (dt, J=20.1, 8.1 Hz, 1H), 7.54-7.70 (m, 2H), 7.78-7.98 (m, 2H). ESMS m/z 244 (M+H)⁺. Calcd for C₁₁H₁₅ClFNO₂S: C, 47.23; H, 5.40; N, 5.01. Found: C, 46.92; H, 5.68; N, 5.15.

(E)-2-Fluoro-4-(3-trifluoromethylthiophenyl)-2-butenylamine hydrochloride (II-15): (0.26 g, 72%). Mp: 188-189° C. ¹H NMR (MeOD-d₃, 300 MHz) δ 3.51 (d, J=8.1 Hz, 2H), 3.97 (d, J=19.5 Hz, 2H), 5.69 (dt, J=20.1, 8.7 Hz, 1H), 7.42-7.52 (m, 2H), 7.54-7.70 (m, 2H). ESMS m/z 266 (M+H)⁺. Calcd for C₁₁H₁₂ClF₄NS: C, 43.79; H, 4.01; N, 4.64. Found: C, 43.64; H, 4.31; N, 4.81.

(E)-2-Fluoro-4-(2-methoxyphenyl)-2-butenylamine hydrochloride (II-17): (0.46 g, 83%). Mp: 133-134° C. ¹H NMR (MeOD-d₃, 300 MHz) δ 3.36 (d, J=8.4 Hz, 2H), 3.84 (s, 3H), 3.97 (d, J=19.5 Hz, 2H), 5.61 (dt, J=20.7, 8.4 Hz, 1H), 6.83-7.03 (m, 2H), 7.09-7.34 (m, 2H). ESMS m/z 196 (M+H)⁺. Calcd for C₁₁H₁₅ClFNO: C, 57.02; H, 6.52; N, 6.04. Found: C, 57.38; H, 6.78; N, 6.19.

(E)-2-Fluoro-4-(3-methoxyphenyl)-4-methyl-2-butenylamine hydrochloride (II-18): (0.27 g, 78%). Mp: 133-134° C. ¹H NMR (MeOD-d₃, 300 MHz) δ 1.65 (d, J=3.9 Hz, 3H), 3.40 (s, 2H), 3.77 (s, 3H), 3.98 (d, J=20.1 Hz, 2H), 6.69-7.84 (m, 2H), 7.22 (t, J=8.7 Hz, 1H). ESMS m/z 210 (M+H)⁺. Calcd for C₁₂H₁₇ClFNO: C, 58.66; H, 6.97; N, 5.70. Found: C, 58.38; H, 6.79; N, 6.03.

(E)-2-Fluoro-5-(3-methoxyphenyl)-2-pentenylamine hydrochloride (I′-24): (0.69 g, 86%). Mp: 112-113° C. ¹H NMR (MeOD-d₃, 300 MHz) δ 2.38 (q, J=7.2 Hz, 2H), 2.70 (t, J=7.2 Hz, 2H), 3.62 (d, J=19.5 Hz, 2H), 3.77 (s, 3H), 5.50 (dt, J=21.3, 8.1 Hz, 1H), 6.72-6.86 (m, 3H), 7.14-7.28 (m, 1H). ESMS m/z 210 (M+H)⁺. Calcd for Cl₂H₁₇ClFNO: C, 58.66; H, 6.97; N, 5.70. Found: C, 58.75; H, 7.26; N, 5.96.

### Example 23

### Synthesis of Compound II-16

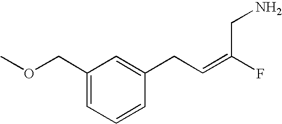

(E)-N-t-Butoxycarbonyl-2-fluoro-4-bromo-butenylamine: To a cooled solution of (E)-N-t-butoxycarbonyl-2-fluoro-4-hydroxyl-butenylamine (0.396 g, 1.93 mmol, see synthesis below) and CBr₄ (0.96 g, 2.89 mmol) in dichloromethane (15 mL) was added a solution of PPh₃ (0.76 g, 2.89 mmol) in dichloromethane (5 mL). The resulting mixture was stirred at room temperature overnight, and then concentrated in vacuo. The residue was purified on flash column chromatography (silica gel, 10% EtOAc/hexane) to give the desired product as a solid (0.36 g, 69%). Mp: 39-40° C. ¹H NMR (CDCl₃, 300 MHz) δ 1.45 (s, 9H), 3.98 (dd, J=20.7, 6.0 Hz, 2H), 4.09 (d, J=8.7 Hz, 2H), 5.54 (dt, J=17.1, 8.4 Hz, 1H).

(E)-N-t-Butoxycarbonyl-2-fluoro-4-(3-methoxymethylphenyl)-2-butenylamine: A mixture of 3-(methoxymethyl)phenylboronic acid (0.275 g, 1.66 mmol), (E)-N-t-Butoxycarbonyl-2-fluoro-4-bromo-2-butenylamine (0.0.21 g, 0.78 mmol), K2CO3 (0.95 g, 6.85 mmol), and bis(dibenzylideneacetone)palladium (0) (8.88 mg, 0.015 mmol) in benzene (10 mL) was heated under N₂ at 85° C. for 20 min, cooled to room temperature. EtOAc (20 mL) and brine (10 mL) were added. The layers were separated. The aqueous layer was extracted with EtOAc (2×10 mL). The combined organic layers were washed with brine (10 mL), dried (Na₂SO₄), filtered, and concentrated. The residue was purified on flash column chromatography (silica gel, 2% EtOAc/hexane) to give the desired product (0.17 g, 65%). ¹H NMR (CDCl₃, 300 MHz) δ 1.46 (s, 9H), 3.37-3.46 (m, 5H), 3.94-4.08 (m, 2H), 5.27-5.46 (m, 2H), 6.02-6.20 (m, 1H), 7.08-7.43 (m, 4H).

(E)-2-Fluoro-4-(3-methoxymethylphenyl)-2-butenylamine hydrochloride (II-16): This deprotection was carried out as described in Example 8. (0.13 g, 96%). Mp: 165-166° C. ¹H NMR (MeOD-d₃, 300 MHz) δ 3.37 (s, 3H), 3.58 (d, J=8.4 Hz, 2H), 3.96 (d, J=18.9 Hz, 2H), 4.44 (s, 2H), 5.69 (dt, J=20.7, 8.7 Hz, 1H), 7.13-7.25 (m, 3H), 7.30 (t, J=7.2 Hz, 1H). ESMS m/z 210 (M+H)⁺. Calcd for C₁₂H₁₇ClFNO: C, 58.66; H, 6.97; N, 5.70. Found: C, 58.43; H, 6.66; N, 5.70.

### Example 24

### Synthesis of Scheme 5 Intermediates

### Ethyl(tert-butyldiphenylsilanyloxy)acetate

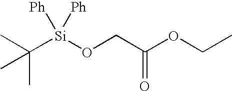

To a cooled solution of ethyl glycolate (5.31 g, 50 mmol) and imidazole (4.17 g, 61 mmol) in DMF (90 mL) was added tert-butyldiphenyl silane chloride (15.7 mL, 61.2 mmol). The reaction mixture was stirred at room temperature for 4 hours, concentrated in vacuo to give crude product. This crude product was used directly in the next step without any further purification. ¹H NMR (CDCl₃, 300 MHz) δ 1.09 (s, 9H), 1.22 (t, J=7.5 Hz, 3H), 4.14 (q, J=6.9 Hz, 2H), 4.23 (s, 2H), 7.34-7.47 (m, 6H), 7.64-7.79 (m, 4H).

### Ethyl(tert-butyldiphenylsilanyloxy)acetaldehyde

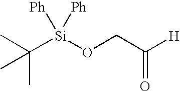

To a cooled mixture of ethyl(tert-butyl-diphenylsilanyloxy)acetate (4.98 g, 14.5 mmol) in dichloromethane (20 mL)/hexane (10 mL) was added dropwise a solution of DIBAL in toluene (1.5 M, 11.6 mL, 17.4 mmol). The resulting mixture was stirred under N₂ at −70° C. for 1 hour and quenched by adding citric acid solution (1 M, 20 mL) and EtOAc (20 mL). The layers were separated. The aqueous layer was extracted with EtOAc (2×20 mL). The combined organic layers were washed with brine (20 mL), dried (Na₂SO₄), filtered, and concentrated. The crude product was obtained (4.57 g). ¹H NMR (CDCl₃, 300 MHz) δ 1.07 (s, 9H), 4.23 (d, J=7.8 Hz, 2H), 7.29-7.50 (m, 6H), 7.58-7.85 (m, 4H), 9.73 (s, 1H).

### (E)-Ethyl 4-(tert-butyldiphenylsilanyloxy)-2-fluoro-2-butenoate

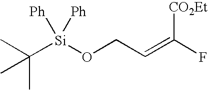

To a cooled suspension of triethyl 2-fluoro-2-phosphonoacetate (2.96 g, 12.2 mmol) in THF (40 mL) was added a solution of isopropylmagnesium chloride in THF (2.0 M, 7.13 mL, 14.3 mmol). The resulting mixture was stirred at 0° C. under N₂ for 2 hours, which resulted in a deep red solution. To this solution was added a solution of ethyl(tert-butyldiphenylsilanyloxy)-acetaldehyde (3.04 g, 10.2 mmol) in THF (10 mL). The resulting mixture was stirred at 0° C. for 5 hours and then at room temperature overnight. The reaction mixture was quenched with water (20 mL). The mixture was extracted with EtOAc (3×20 mL). The combined organic layers were washed with brine (30 mL), dried (MgSO₄), filtered, and concentrated. The residue was purified on flash column chromatography (silica gel, 1% MTBE/hexane) to give the desired product (2.35 g, 58%). ¹H NMR (CDCl₃, 300 MHz) δ 1.06 (s, 9H), 1.20 (t, J=7.2 Hz, 3H), 4.16 (q, J=7.2 Hz, 2H), 4.67 (dd, J=5.4, 3.6 Hz, 2H), 6.11 (dt, J=20.4, 5.4 Hz, 1H), 7.33-7.50 (m, 6H), 7.58-7.75 (m, 4H).

### (E)-Ethyl 4-(tert-butyldiphenylsilanyloxy)-2-fluoro-2-butenol

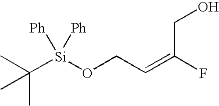

To a cooled solution of ethyl 4-(tert-butyldiphenylsilanyloxy)-2-fluoro-2-butenoate (2.8 g, 7.24 mmol) in hexane (20 mL) was added a solution of DIBAL in toluene (1.5 M, 14.5 mL, 21.7 mmol). The resulting mixture was stirred at −30° C. under N2 for 30 min and then at room temperature for 30 min. The reaction was quenched with a solution of citric acid (1.0 M, 20 mL). The mixture was extracted with EtOAc (3×20 mL). The combined organic layers were washed with brine (20 mL), dried (MgSO₄), filtered, and concentrated. The residue was purified on flash column chromatography (silica gel, 5% EtOAc/hexane) to give the desired product (0.43 g, 17%). ¹H NMR (CDCl₃, 300 MHz) δ 1.04 (s, 9H), 4.04 (dd, J=19.5, 6.6 Hz, 2H), 4.22 (dd, J=7.2, 1.8 Hz, 2H), 5.41 (dt, J=20.1, 7.2 Hz, 1H), 7.33-7.52 (m, 6H), 7.61-7.75 (m, 4H).

### (E)-N-t-Butoxycarbonyl-N-(ethoxyoxoacetyl)-4-(tert-butyldiphenylsilanyloxy)-2-fluoro-2-butenylamine

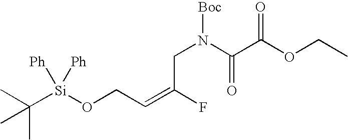

To a cooled solution of (E)-N-t-butoxycarbonyl-2-fluoro-4-(3-methoxyphenoxy)-2-butenylamine (0.42 g, 1.22 mmol), ethyl N-t-butoxycarbonylaminooxoacetate (0.318 g, 1.46 mmol), and PPh₃ (0.384 g, 1.46 mmol) in THF (20 mL) was added a solution of DIAD (0.32 g, 1.58 mmol) in THF (5 mL). The resulting mixture was stirred at room temperature overnight, and concentrated in vacuo. The residue was purified on flash column chromatography (silica gel, 2% EtOAc/hexane) to provide the desired product (0.535 g, 81%). ¹H NMR (CDCl₃, 300 MHz) δ 1.04 (s, 9H), 1.34 (t, J=6.9 Hz, 3H), 1.46 (s, 9H), 4.22-4.38 (m, 6H), 5.42 (dt, J=19.5, 7.2 Hz, 1H), 7.33-7.52 (m, 6H), 7.61-7.75 (m, 4H).

### (E)-N-t-Butoxycarbonyl-4-(tert-butyldiphenylsilanyloxy)-2-fluoro-2-butenylamine

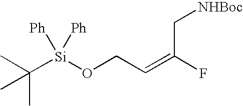

To a solution of (E)-N-t-Butoxycarbonyl-N-(ethoxyoxoacetyl)-4-(tert-butyldiphenylsilanyloxy)-2-fluoro-2-butenylamine (0.54 g, 0.98 mmol) in THF (10 mL) was added an aqueous solution of LiOH (2.0 M, 2.48 mL, 4.96 mmol). The resulting mixture was stirred at room temperature for 3 hours. The layers were separated. The aqueous layer was extracted with EtOAc (2×10 mL). The combined organic layers were washed with brine (20 mL), dried (Na₂SO₄), filtered, and concentrated to give crude product (0.44 g, 100%). ¹H NMR (CDCl₃, 300 MHz) δ 1.04 (s, 9H), 1.46 (s, 9H), 3.64-3.79 (m, 2H), 4.17-4.30 (m, 2H), 5.31-5.42 (m, 1H), 7.34-7.52 (m, 6H), 7.63-7.75 (m, 4H).

### (E)-N-t-Butoxycarbonyl-2-fluoro-4-hydroxyl-butenylamine

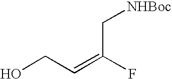

To a solution of (E)-N-t-butoxycarbonyl-4-(tert-butyldiphenylsilanyloxy)-2-fluoro-2-butenylamine 0.43 g, 0.97 mmol) in THF (5 mL) was added tetrabutylammonium fluoride trihydrate (0.61 g, 1.94 mmol). The mixture was stirred at room temperature for 2 hours. The mixture was diluted with water (10 mL) and EtOAc (10 mL). The layers were separated. The aqueous layer was extracted with EtOAc (2×10 mL). The combined organic layers were washed with brine (20 mL), dried (Na₂SO₄), filtered, and concentrated. The residue was purified on flash column chromatography (silica gel, 30% EtOAc/hexane) to give the desired product (0.18 g, 91%). ¹H NMR (CDCl₃, 300 MHz) δ 1.44 (s, 9H), 3.89 (dd, J=21.9, 6.0 Hz, 2H), 4.09-4.20 (m, 2H), 5.54 (dt, J=20.1, 7.5 Hz, 1H).

### Example 25

### Synthesis of Precursors to Compounds of Formula II as Described in Scheme 5

General procedure for the preparation of (E)-N-t-Butoxycarbonyl-2-fluoro-4-(substituted-phenoxy)-2-butenylamine: To a cooled mixture of substituted phenol (1.2 eq.), PPh₃ (1.2 eq.), and (E)-N-t-Butoxycarbonyl-2-fluoro-4-hydroxyl-butenylamine (1.0 eq) in THF (20 mL) was added dropwise a solution of DIAD (1.2 eq.) in THF (5.0 mL). The resulting mixture was stirred under N₂ at room temperature overnight, concentrated in vacuo. The residue was purified on flash column chromatography (silica gel, 5% EtOAc/hexane) to give desired product.

(E)-N-t-Butoxycarbonyl-2-fluoro-4-(3-methoxyphenoxy)-2-butenylamine: (0.22 g, 81%). ¹H NMR (CDCl₃, 300 MHz) δ 1.45 (s, 9H), 3.79 (s, 3H), 3.98 (dd, J=20.1, 5.4 Hz, 2H), 4.59 (d, J=6.6 Hz, 2H), 5.52 (dt, J=18.3, 7.8 Hz, 1H), 6.42-6.62 (m, 3H), 7.19 (t, J=7.8 Hz, 1H).

(E)-N-t-Butoxycarbonyl-2-fluoro-4-(3-methoxyphenylsulfanyl)-2-butenylamine: (0.1 g, 40%). ¹H NMR (CDCl₃, 300 MHz) δ 1.44 (s, 9H), 3.54 (d, J=8.7 Hz, 2H), 3.70 (dd, J=21.9, 7.5 Hz, 2H), 3.81 (s, 3H), 5.32 (dt, J=18.9, 8.4 Hz, 1H), 6.76-6.88 (m, 1H), 6.90-7.07 (m, 2H), 7.18-7.26 (m, 1H).

(E)-N-t-Butoxycarbonyl-2-fluoro-4-(3-trifluoromethylphenoxy)-2-butenylamine: (0.31 g, 91%). ¹H NMR (CDCl₃, 300 MHz) δ 1.45 (s, 9H), 4.00 (dd, J=20.4, 6.3 Hz, 2H), 4.66 (d, J=7.2 Hz, 2H), 5.52 (dt, J=18.6, 8.1 Hz, 1H), 7.04-7.26 (m, 3H), 7.34-7.47 (m, 1H).

(E)-N-t-Butoxycarbonyl-2-fluoro-4-(3-pyridyloxy)-2-butenylamine: (0.13 g, 62%). ¹H NMR (CDCl₃, 300 MHz) δ 1.44 (s, 9H), 3.97 (dd, J=20.1, 6.0 Hz, 2H), 4.68 (d, J=7.5 Hz, 2H), 5.55 (dt, J=18.9, 8.7 Hz, 1H), 7.18-7.26 (m, 2H), 8.20-8.28 (m, 1H), 8.30-8.39 (m, 1H).

### Example 26

### Synthesis of Compounds of Formula II as Described in Scheme 5

The compounds of Formula II described in this example where prepared from Boc-protected products as described in Example 25 using acidic deprotections as described in Examples 7 and 8.

(E)-2-Fluoro-4-(3-methoxyphenoxy)-2-butenylamine hydrochloride (II-20): (0.13 g, 84%). ¹H NMR (MeOD-d₃, 300 MHz) δ 3.77 (s, 3H), 3.97 (d, J=19.5 Hz, 2H), 4.58-4.66 (m, 2H), 5.85 (dt, J=19.5, 6.9 Hz, 1H), 6.47-6.63 (m, 3H), 7.19 (t, J=7.8 Hz, 1H). ESMS m/z 212 (M+H)⁺.

(E)-2-Fluoro-4-(3-methoxyphenylsulfanyl)-2-butenylamine hydrochloride (I′-21): (0.07 g, 93%). Mp: 88-89° C. ¹H NMR (MeOD-d₃, 300 MHz) δ 3.54-3.65 (m, 4H), 3.79 (s, 3H), 5.62 (dt, J=18.9, 8.7 Hz, 1H), 6.80-7.05 (m, 3H), 7.25 (t, J=8.1 Hz, 1H). ESMS m/z 228 (M+H)⁺.

(E)-2-Fluoro-4-(3-trifluoromethylphenoxy)-2-butenylamine hydrochloride (I′-22): (0.22 g, 86%). ¹H NMR (MeOD-d₃, 300 MHz) δ 3.99 (d, J=19.5 Hz, 2H), 4.71 (dd, J=7.2, 1.8 Hz, 2H), 5.89 (dt, J=19.5, 7.2 Hz, 1H), 7.19-7.34 (m, 3H), 7.50 (t, J=8.4 Hz, 1H). ESMS m/z 250 (M+H)⁺.

(E)-2-Fluoro-4-(3-pyridyloxy)-2-butenylamine hydrochloride (II-23): (0.13 g, 96%). ¹H NMR (MeOD-d₃, 300 MHz) δ 4.04 (d, J=19.5 Hz, 2H), 4.91-5.02 (m, 2H), 5.93 (dt, J=18.3, 7.5 Hz, 1H), 7.93-8.06 (m, 1H), 8.16-8.28 (m, 1H), 8.42-8.54 (m, 1H), 8.60-8.71 (m, 1H). ESMS m/z 183 (M+H)⁺. HRMS Calcd for Cl₂H₁₈ClFNO: 183.0928. Found: 183.0929.

### Example 27

### In Vitro Inhibition of SSAO Activity

SSAO activity was measured as described (Lizcano J M. Et al. (1998) Biochem J. 331:69). Briefly, rat lung or human umbilical cord homogenates were prepared by chopping the freshly removed tissue into small pieces and washing them thoroughly in PBS. The tissue was then homogenized 1:10 (w/v) for lung or 1:5 (w/v) for umbilical cord, in 10 mM potassium phosphate buffer (pH 7.8). Homogenates were then centrifuged at 1000 G at 4° C. for 10 min (lung) or 25,000 g for 30 min (umbilical cord); the supernatants were kept frozen until ready to use. Lung or umbilical cord homogenate was preincubated with clorgyline and pargyline at 1 μM to inhibit MAO-A and -B activity, respectively, and SSAO inhibitors were generally present at 1 nM-10M. The reaction was initiated by addition of 20 μM ¹⁴C— benzylamine as substrate. The reaction was carried out at 37° C. in a final volume of 400 μL of 100 mM potassium phosphate buffer (pH 7.2) and stopped with 100 μl of 2M citric acid. Radioactively labeled products were extracted into toluene/ethyl acetate (1:1, v/v) containing 0.6% (w/v) 2,5-diphenyloxazole (PPO) before liquid scintillation counting. Results are shown in Table 1 in the next example.

### Example 28

### Comparison of Inhibition of the Activity of SSAO/VAP-1 Versus MAO-A and MAO-B activities

The specificities of the different SSAO inhibitors was tested by determining their abilities to inhibit MAO-A and MAO-B activities in vitro. Recombinant human MAO-A and human MAO-B enzymes were obtained from BD Biosciences (MA, USA). MAO activity was measured using the colorimetric method essentially as described (Holt, A. et al. (1997) Anal. Biochem. 244: 384). A pre-determined amount of inhibitor diluted in 0.2M potassium phosphate buffer, pH 7.6, was added to each well, if required. The amount of inhibitor varied in each assay but was generally at a final concentration of between 1 nM and 1 mM. Controls lacked inhibitor. The following agents were then added to a final reaction volume of 200 μL in 0.2M potassium phosphate buffer, pH 7.6: 0.04 mg/ml of MAO-A or 0.07 mg/ml MAO-B enzyme, 15 μL of 10 mM tyramine substrate (for MAO-A), or 15 μL 100 mM benzylamine substrate (for MAO-B), and 50 μL of freshly made chromogenic solution. The chromogenic solution contained 750 μM vanillic acid (Sigma#V-2250), 400 μM 4-aminoantipyrine (Sigma # A-4328) and 12 U/mL horseradish peroxidase (Sigma # P-8250) in order to cause a change of 0.5 OD A490 nm/h. This was within the linear response range for the assay. The plates were incubated for 60 min at 37° C. The increase in absorbance, reflecting MAO activity, was measured at 490 nm using microplate spectrophotometer (Power Wave 40, Bio-Tek Inst.). Inhibition was presented as percent inhibition compared to control after correcting for background absorbance and IC₅₀ values calculated using GraphPad Prism software. Clorgyline and pargyline (inhibitors of MAO-A and -B, respectively) at 1 μM, were added to some wells as positive controls for MAO inhibition. The ability of compounds of the previous Examples to inhibit SSAO activity versus MAO activity is shown in Table 1. The results show that the compounds described in the present invention are specific inhibitors of SSAO activity. The compounds described in the present invention are therefore expected to have therapeutic utility in the treatment of diseases and conditions in which the activity of SSAO/VAP-1 plays a role, that is, in SSAO/VAP-1 mediated diseases and conditions.

TABLE 1

| | Rat | Human | Human | Specificity | Human | Specificity |
|---|---|---|---|---|---|---|
| Compound | SSAO | SSAO | MAO-A | of SSAO | MAO-B | of SSAO |
| No: | IC50 (μM) | IC50 (μM) | IC50 (μM) | on MAO-A | IC50 (μM) | on MAO-B |
| I-1-Z | 0.013 | 0.018 | 85 | 4700 | 1.6 | 88 |
| I-2-E | 0.008 | 0.009 | 85 | 9400 | 1.7 | 189 |
| I-2-Z | 0.005 | 0.01 | 79 | 7900 | 12 | 1200 |
| I-3-Z | | 0.028 | 91 | 3250 | 0.084 | 3 |
| I-5-Z | | 0.028 | 74 | 2643 | 1.8 | 64.3 |
| I-8-E | | 0.0114 | 48 | 4211 | 35 | 3070 |
| I-8-Z | | 0.005 | 35 | 7000 | 27 | 5400 |
| I-9-Z | | 0.012 | 109 | 8385 | 2.4 | 200 |
| I-14-E | | 0.013 | 77 | 5923 | 0.033 | 2.54 |
| I-14-Z | | 0.008 | 90 | 11250 | 9.6 | 1250 |
| I-15-Z | | 0.007 | 119 | 17000 | 38 | 5429 |
| I-19-Z | | 0.025 | 34 | 1360 | 0.69 | 27.6 |
| I-39-Z | | 1.2 | | | | |
| I-40-Z | | 0.92 | 632 | 687 | 170 | 185 |
| I-42-Z | | 73%@8 mM | | | | |
| I-43-Z | | 1.4 | | | | |
| I-44-Z | | 0.72 | 105 | 146 | 16 | 22 |
| I-45-Z | | 0.14 | 20 | 143 | 1.2 | 8.6 |
| I-47-E | | 0.17 | 135 | 794 | 0.42 | 2.5 |
| I-48-E | | 10 | | | | |
| I-49-E | | 5.5 | | | | |
| I-50-E | | 1 | | | | |
| I-51-E | | 33%@40 mM | | | | |
| I-99-Z | | 0.13 | 23 | 177 | 0.27 | 2.1 |
| I-102-Z | | 0.03 | 0.62 | 21 | 0.56 | 19 |
| I-109-Z | | 0.27 | 21 | 78 | 1.6 | 5.9 |
| II-1-E | 0.038 | 0.036 | >87 | >2400 | >87 | >2400 |
| II-3-E | | 0.45 | 1000 | 2222 | 1000 | 2222 |
| II-4-E | | 0.51 | | | | |
| II-5-E | | 0.3 | >1000 | >3333 | >1000 | >3333 |
| II-6-E | | 0.13 | 1000 | 7692 | 1000 | 7692 |
| II-7-E | | 0.22 | 1000 | 4545 | 1000 | 4545 |
| II-8-E | | 0.022 | 1100 | 50000 | 1100 | 50000 |
| II-9-E | | 0.099 | 1200 | 12120 | 1200 | 12120 |
| II-10-E | | 0.018 | 870 | 48330 | 966 | 53670 |
| II-11-E | | 0.11 | 286 | 2600 | 800 | 7272 |
| II-12-E | | 55%@7 mM | | | | |
| II-13-E | | 0.01 | 1000 | 100000 | 1000 | 100000 |
| II-14-E | | 0.07 | 1000 | 14290 | 1000 | 14286 |
| II-15-E | | 0.023 | >800 | >34780 | >800 | >34780 |
| II-17-E | | 4.5 | | | | |
| II-18-E | | 0.29 | >1000 | >3448 | >1000 | >3448 |
| II-19-E | | 0.16 | >1000 | >6250 | 850 | 5312 |
| II-20-E | | 0.75 | >1000 | >1333 | 1000 | 1333 |
| II-21-E | | 0.088 | | | | |
| II-23-E | | >1000 | | | | |
| IV-1 | | 45%@8 mM | | | | |
| IV-3 | | 3 | | | | |
| IV-8 | | 3.7 | | | | |
| IV-10 | | 1.5 | | | | |

### Example 29

### Inhibition of Collagen-Induced Arthritis in Mice

Collagen-induced arthritis (CIA) in mice is widely used as an experimental model for rheumatoid arthritis (RA) in humans. CIA is mediated by autoantibodies to a particular region of type II collagen and complement. The murine CIA model used in this study is called antibody-mediated CIA, and can be induced by i.v. injection of a combination of different anti-type II collagen monoclonal antibodies (Terato K., et al. (1995). Autoimmunity. 22:137). Several compounds have been used to successfully block inflammation in this model, including anti-α1β1 and anti-α2β2 integrins monoclonal antibodies (de Fougerolles A. R. (2000) J. Clin. Invest. 105: 721).

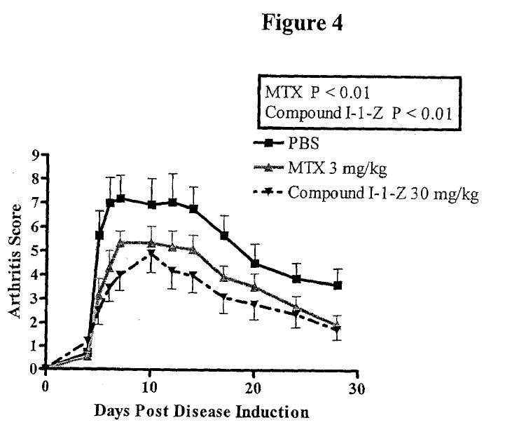

In this example, arthrogen-collagen-induced arthritis antibody kits were purchased from Chemicon International (Temecula, Calif.) and arthritis was induced using the manufacturer's protocol. Mice were injected i.v. with a cocktail of 4 anti-collagen Type II monoclonal antibodies (1.5 mg each) on day 0, followed by i.p. injection of 25 μg lipopolysaccharide (LPS) on day 2. Mice develop swollen wrists, ankles, and digits 3-4 days after LPS injection, with disease incidence of 90% by day 7. Severity of arthritis in each limb was scored for 3-4 weeks as follows: 0=normal; 1=mild redness, slight swelling of ankle or wrist; 2=moderate redness and swelling of ankle or wrist; 3=severe redness and swelling of some digits, ankle and paw; 4=maximally inflamed limb, with a maximum score of 16 per animal. In the experiment shown in FIG. 4, animals were divided in 3 groups of 10 animals: vehicle, methotrexate (MTX)-treated, and compound-treated. All treatments were between days 1 and 10, and were delivered i.p. Animals in the vehicle group were injected with phosphate buffer saline (PBS), once daily for 10 days (starting on day 1). MTX (3 mg/kg) was administered starting on day 1 and continuing every other day (Mon., Weds., Fri.) between days 1 and 10. Administration of compound I-1-Z (30 mg/kg/dose, i.p., one dose daily) was initiated at day 1 and continued until day 10. The results are shown in FIG. 4. The administration of 30 mg/kg of compound I-1-Z, once daily, on days 1-10 clearly reduced the final arthritis score and paw swelling in this model. Statistical analyses were performed by repeated measures ANOVA followed by Dunnett's test for multiple comparisons.

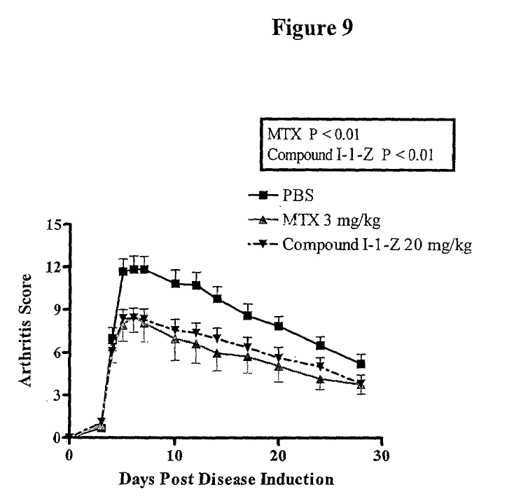

The effect of therapeutic dosing with compound I-1-Z is shown in FIG. 9. Arthritis was induced using the manufacturer's protocol, as described above. The Animals were treated with PBS, or with compound I-1-Z at 20 mg/kg once daily between day 4 and 13, or with methotrexate at 3 mg/kg on days 4, 6, 8, 10 and 12. All treatments were delivered i.p. Mice were monitored for clinical signs of arthritis from mild swelling to maximal inflammation of the paw and scored on a pre-defined scale of 1-4 per paw giving a maximal score of 16 per animal. Statistical analyses were performed by repeated measures ANOVA followed by Dunnett's test for multiple comparisons.

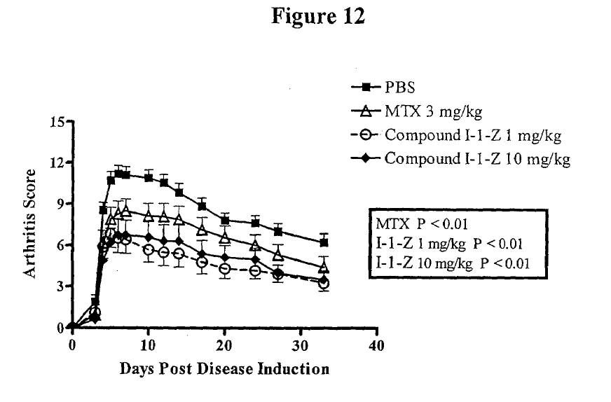

The effects of low doses of compound I-1-Z are shown in FIG. 12. Arthritis was induced using the manufacturer's protocol, as described above. LPS at 25 μg was given i.p. on day 2. Animals were treated with PBS, or with compound I-1-Z at 1 or 10 mg/kg once daily between days 1 and 10, or with methotrexate at 3 mg/kg on days 1, 3, 5, 7 and 9. All treatments were delivered i.p. Mice were monitored for clinical signs of arthritis from mild swelling to maximal inflammation of the paw and scored on a pre-defined scale of 1-4 per paw giving a maximal score of 16 per animal. Statistical analyses were performed with repeated measures ANOVA followed by Dunnett's test for multiple comparisons.

### Alternate Collagen-Induced Arthritis Model in Mice

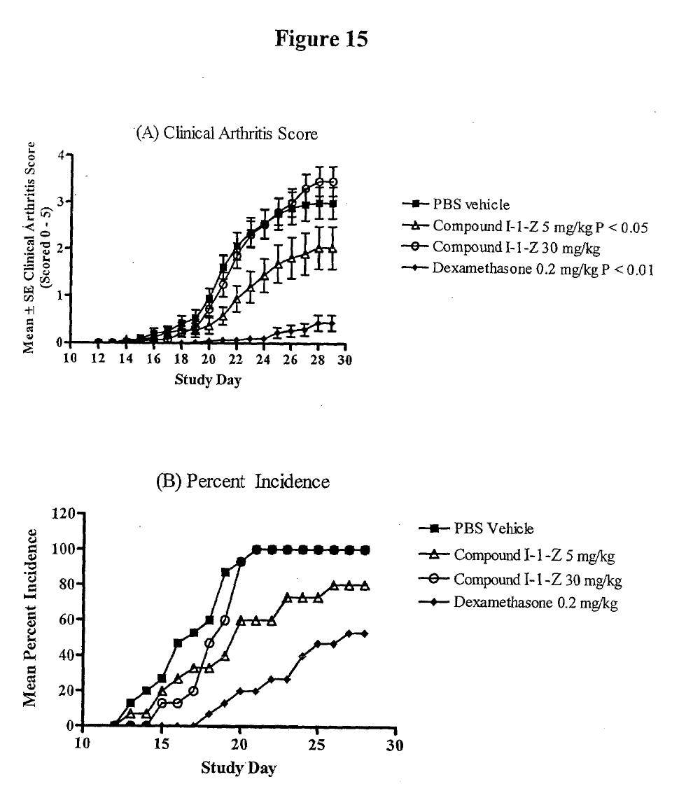

Collagen-induced arthritis can also be induced by direct injection of collagen. Groups of 15 male B10RIII mice were injected intradermally with Type II collagen in Complete Freund Adjuvant (CFA) on days 0 and 15. Mice were dosed p.o. starting from day 0 to day 28 with phosphate buffered saline (PBS), 5 mg/kg compound I-1-Z, 30 mg/kg compound I-1-Z or 0.2 mg/kg dexamethasone. The results are shown in FIG. 15. Compound I-1-Z at 5 mg/kg reduced both the incidence of arthritis and the clinical scores as compared to PBS; however, no clinical efficacy was observed when animals were dosed with compound I-1-Z at 30 mg/kg. Clinical scores were determined daily for each paw based on a scale between 0-5 based on joint erythema and swelling. Statistical analysis was by Student's t-test versus the PBS-treated disease control group.

### Example 30

### Inhibition of Experimental Autoimmune Encephalomyelitis in Mice by SSAO Inhibitors

SSAO/VAP-1 is expressed on the endothelium of inflamed tissues/organs including brain and spinal cord. Its ability to support lymphocyte transendothelial migration may be an important systemic function of SSAO/VAP-1 in inflammatory diseases such as multiple sclerosis and Alzheimer's disease. An analysis of the use of SSAO inhibitors to treat inflammatory disease of the central nervous system (CNS) was performed through the use of an experimental autoimmune encephalomyelitis model (EAE) in C57BL/6 mice. EAE in rodents is a well-characterized and reproducible animal model of multiple sclerosis in human (Benson J. M. et al. (2000) J. Clin. Invest. 106:1031). Multiple sclerosis is a chronic immune-mediated disease of the CNS characterized by patchy perivenular inflammatory infiltrates in areas of demyelination and axonal loss. As an animal model, EAE can be induced in mice by immunization with encephalitogenic myelin antigens in the presence of adjuvant. The pathogenesis of EAE comprises presentation of myelin antigens to T cells, migration of activated T cells to the CNS, and development of inflammation and/or demyelination upon recognition of the same antigens.

To examine the role of SSAO/VAP-1 as a major regulator of the lymphocyte recruitment to the CNS, compound II-1-E, an SSAO inhibitor, was evaluated in an EAE model.

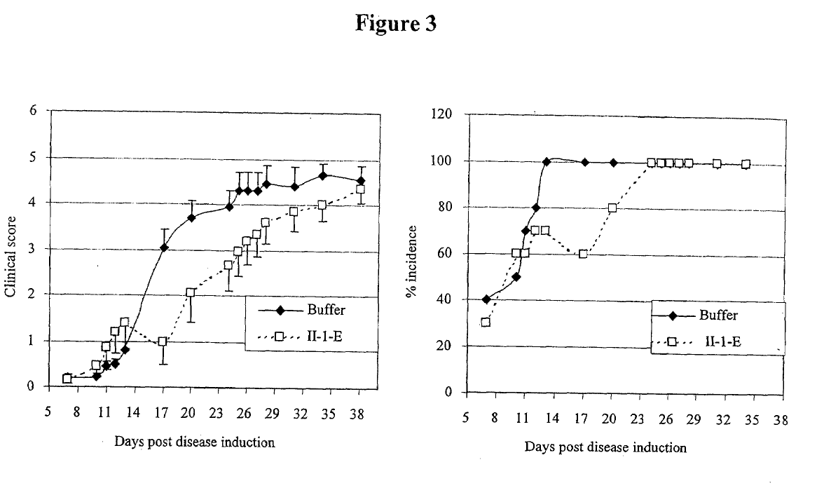

Twenty female C57BL/6 mice were immunized subcutaneously (s.c). with myelin oligodendrocyte glycoprotein 35-55 (MOG peptide 35-55) in Complete Freund Adjuvant (CFA) on day 0, followed by i.p. injections of 500 ng pertussis toxin (one pertussis toxin injection on day 0, a second pertussis toxin injection on day 2). Groups of 10 mice received either compound II-1-E 40 mg/kg/dose, once daily i.p. for 30 consecutive days), or vehicle control (once/day for 30 consecutive days) all starting from one day after the immunization and all administered i.p. The animals were monitored for body weight, signs of paralysis and death according to a 0-5 scale of scoring system as follows: 1=limp tail or waddling gait with tail tonicity; 2=waddling gait with limp tail (ataxia); 2.5=ataxia with partial limb paralysis; 3=full paralysis of one limb; 3.5=full paralysis of one limb with partial paralysis of second limb; 4=full paralysis of two limbs; 4.5=moribund; 5=death. Results are shown in FIG. 3. In the control group, disease reached 100% incidence at day 13, compared with day 24 for the compound II-1-E-treated group. In addition, from day 14 onward the mean clinical score of the treated group was well below that in the control group, and similar clinical scores between the two groups was not reached until day 38.

### Example 31

### Inhibition of Carrageenan-Induced Rat Paw Edema

Carrageenan-induced paw edema has been extensively used in the evaluation of anti-inflammatory effects of various therapeutic agents and is a useful experimental system for assessing the efficacy of compounds to alleviate acute inflammation (Whiteley P E and Dalrymple S A, 1998. Models of inflammation: carrageenan-induced paw edema in the rat, in Current Protocols in Pharmacology. Enna S J, Williams M, Ferkany J W, Kenaki T, Porsolt R E and Sullivan J P, eds., pp 5.4.1-5.4.3, John Wiley & Sons, New York). The full development of the edema is neutrophil-dependent (Salvemini D. et al. (1996) Br. J. Pharmacol. 118: 829).

Female Sprague Dawley rats were used in groups of 8-12 and compounds of the invention were administered orally at up to 50 mg/kg 60 min prior to carrageenan exposure. The control group was administered orally an equal volume of vehicle (PBS). Edema in the paws was induced as previously described by injecting 50 μL of a 0.5% solution of carrageenan (Type IV Lambda, Sigma) in saline with a 27-G needle s.c. in the right foot pad. (See Whiteley P. E. and Dalrymple S. A. (1998), Models of inflammation: carrageenan-induced paw edema in the rat, in Current Protocols in Pharmacology, Enna S J, Williams M, Ferkany J W, Kenaki T, Porsolt R E and Sullivan J P, eds., pp 5.4.1-5.4.3, John Wiley & Sons, New York) The size of the tested foot of each animal was measured volumetrically with a plethysmometer immediately after injection of carrageenan solution and at various times up to 360 min after carrageenan induction.

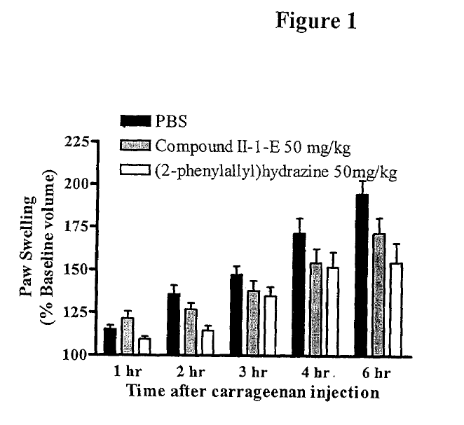

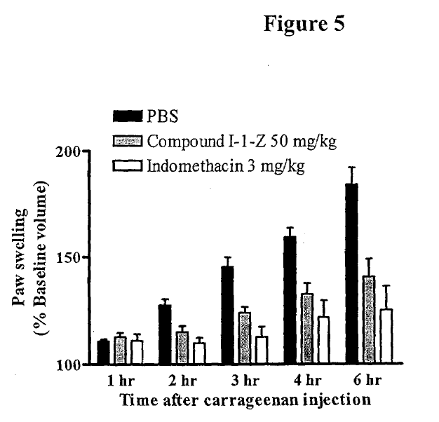

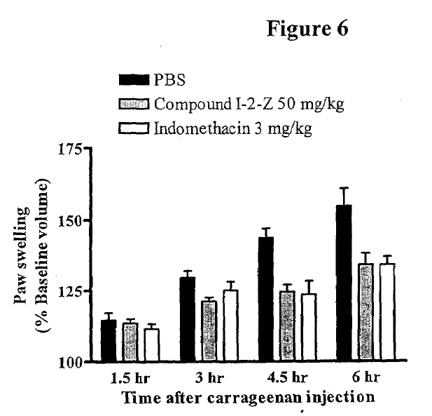

Results of experiments with the compounds II-1-E, I-1-Z, and I-2-Z are shown in FIG. 1, FIG. 5, and FIG. 6, respectively. In each case the 50 mg/kg dose clearly reduced the paw swelling between 2-6 h (Compound II-1-E and Compound I-1-Z), and between 3-6 h for Compound I-2-Z. FIG. 1 also shows comparison to (2-phenylallyl)hydrazine at 50 mg/kg, while FIG. 5 and FIG. 6 show comparison to indomethacin (3 mg/kg).

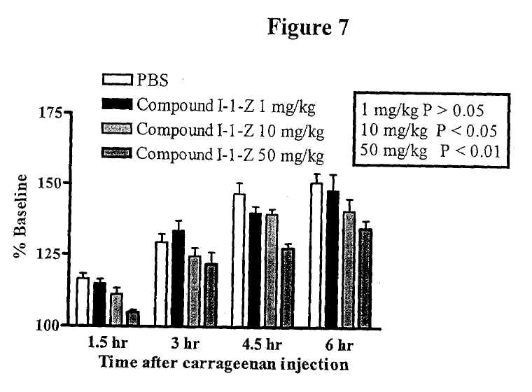

FIG. 7 shows a dose response effect of compound I-1-Z on carrageenan-induced paw edema in the rats. Using the same procedure discussed above, the size of the foot of each animal was measured volumetrically before induction of edema, and at 1.5, 3, 4.5 and 6 h after the carrageenan injection. The absolute decreases in paw swelling at 6 h with 1, 10 and 50 mg/kg were 5±11%, 20±9% and 32±6% respectively. Statistical analyses were performed with repeated measures ANOVA followed by Dunnett's test for multiple comparisons.

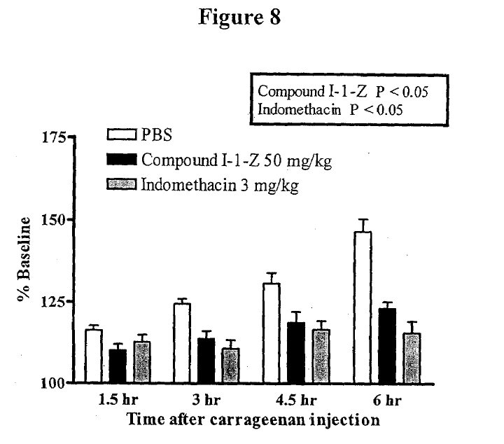

FIG. 8 shows the effect of therapeutic dosing with compound I-1-Z on carrageenan-induced paw edema in the rats. Paw edema was induced in groups of eight Sprague Dawley rats by injecting 50 μL of a 0.375% solution of carrageenan λ in saline, subcutaneously in the foot. After one hour animals were orally dosed once with PBS, compound I-1-Z at 50 mg/kg or with indomethacin at 3 mg/kg. The size of the paws were measured before induction of edema (baseline), and at 1.5, 3, 4.5 and 6 h after carrageenan injection. The magnitude of inhibition steadily increased up to 6 h, to give 50±4% inhibition by compound I-1-Z and 67±8% by indomethacin at 6 h. Statistical analyses were performed with repeated measures ANOVA followed by Dunnett's test for multiple comparisons.

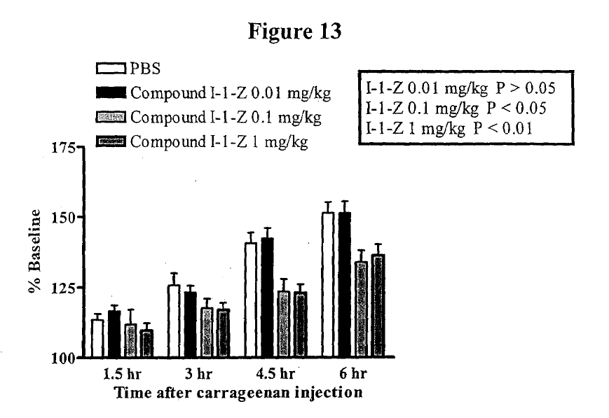

FIG. 13 shows the effect of low doses of compound I-1-Z on carrageenan-induced paw edema in the rats. Groups of eight Sprague Dawley rats were orally administered PBS, or 0.01, 0.1 or 1 mg/kg of compound I-1-Z. One hour later paw edema was induced in all animals by injecting 50 μL of a 0.5% solution of carrageenan λ in saline, subcutaneously into the footpad. The size of the foot of each animal was measured volumetrically before induction of edema, and at 1.5, 3, 4.5 and 6 h after the carrageenan injection. The absolute decreases in paw swelling at 6 h with 0.1 and 1 mg/kg were 34±8 and 29±8% respectively. Statistical analyses were performed with repeated measures ANOVA followed by Dunnett's test for multiple comparisons.

### Example 32

### Inhibition of Oxazolone-Induced Colitis

Oxazolone-induced colitis is a TH2-mediated process that closely resembles ulcerative colitis and is responsive to anti-IL4 therapy ((Strober W. et al (2002) Annu. Rev. Immunol. 20: 495; Boirivant M. et al. (1998) J. Ex. Med. 188: 1929). Oxazolone colitis is induced as described (Fuss I. J. et al. (2002) J. Immunol. 168: 900). Briefly, mice are pre-sensitized by epicutaneous application of 1% oxazolone (4-ethoxymethylene-2-phenyl-2oxazolin-5-one, Sigma) in 100% EtOH (200 μL) on day 0, followed by intrarectal administration of 0.75% oxazolone in 50% EtOH (100 μL) to anesthetized SJL/J male mice on day 5 through a 3.5 F catheter inserted 4 cm proximal to the anal verge. Mice are divided in two treatment groups and injected i.p. twice a day with either PBS or a compound of the invention. Injections are initiated at day 0 and are continued through day 12. Disease progression is evaluated by monitoring body weight and survival.

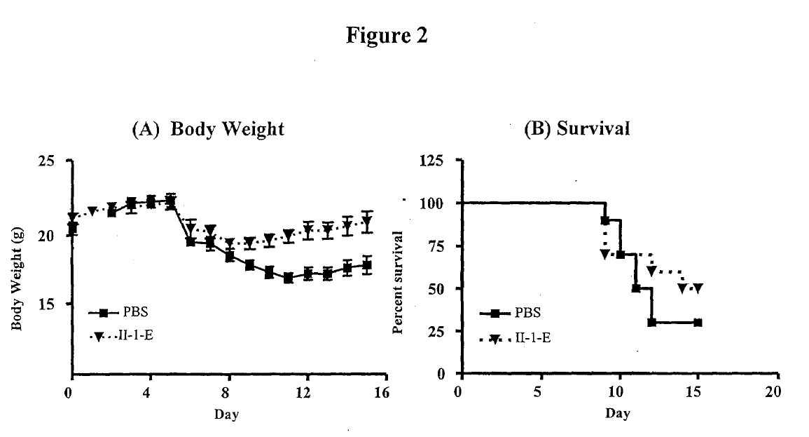

A study was carried out using the protocol described above for oxazolone-induced colitis. Compound II-1-E at 30 mg/kg or buffer injections, administered intraperitoneally were initiated at day 0 and were continued until day 12. Disease progression was evaluated until day 15 (10 days after intrarectal administration) by monitoring survival rates and body weight. When following the above-described protocol, disease severity as measured by body weight drop was maximal at day 11 (6 days after intrarectal challenge), although animals started dying on day 9. Results showed that compound II-1-E improved survival rates and body weight loss when compared with the vehicle group; see FIG. 2A and FIG. 2B.

### Example 33

### Acute Toxicity Studies

Oral (p.o.) and intravenous (i.v.) LD₅₀ values for the compounds of the invention are determined in mice. Six-week old C57Bl/6 female mice are divided in groups of five and administered a single i.v., p.o. or i.p. injection of compound dissolved in PBS (10-100 mg/kg in 100 μL i.v.; 30-1000 mg/kg p.o.; 30-500 mg/kg in 200 μL i.p.). Control groups are administered the same volume of PBS i.p., p.o. or i.v. Appearance and overt behavior are noted daily, and body weight is measured before compound administration (Day 1) and on Days, 3, 5 and 7. After seven days, animals are euthanized and their liver, spleen, and kidneys are weighed.

### Example 34

### Inhibition of Concanavalin A-Induced Liver Injury

Prevention of inflammation by administration of compounds of the invention is assessed in the concanavalin A (Con A) murine model of liver injury. Con A activates T lymphocytes and causes T cell-mediated hepatic injury in mice. Tumor necrosis factor alpha is a critical mediator in this experimental model. T-cell-mediated liver injury involves the migration of immune cells, notably CD4+ T lymphocytes, into liver tissue. Balb/c mice are inoculated with 10 mg/kg concanavalin A administered i.v. in 200 μL pyrogen-free saline as described (Willuweit A. et al. (2001) J Immunol. 167:3944). Previous to Con A administration, animals are separated into treatment groups and injected i.p with PBS, or with different concentrations of compound of the invention (e.g., 20 mg/kg). Liver damage is evaluated by determining serum levels of liver enzymes such as transaminase and alkaline phosphatase, hepatic histopathology, and levels of different inflammatory cytokines in plasma and liver tissue.

This procedure is used to screen for compounds which inhibit the development of liver damage as compared to control animals.

### Example 35

### Effect of Compounds of the Invention in a Mouse Model of Alzheimer's Disease

Alzheimer's disease (AD) is characterized clinically by a dementia of insidious onset and pathologically by the presence of numerous neuritic plaques and neurofibrillary tangles. The plaques are composed mainly of β-amyloid (Aβ) peptide fragments, derived from processing of the amyloid precursor protein (APP). Tangles consist of paired helical filaments composed of the microtubule-associated protein, tau. Transgenic mice carrying a pathogenic mutation in APP show marked elevation of Aβ-protein level and Aβ deposition in the cerebral cortex and hippocampus from approximately 1 year of age (Hsiao K. et al. (1996) Science 274:99). Mutant PS-1 transgenic mice do not show abnormal pathological changes, but do show subtly elevated levels of the Aβ42/43 peptide (Duff K, et al. (1996) Nature 383:710). Transgenic mice derived from a cross between these mice (PS/APP) show markedly accelerated accumulation of Aβ into visible deposits compared with APP singly transgenic mice (Holcomb L. et al. (1998) Nat Med 4:97). Further, a recent study indicates that in these mice, inflammatory responses may be involved in the Aβ depositions (Matsuoka Y. et al. (2001) Am J Pathol. 158(4):1345).

The PS/APP mouse, therefore, has considerable utility in the study of the amyloid phenotype of AD and is used in studies to assess efficacy of the compounds of the invention to treat Alzheimer's patients. Mice are injected with vehicle (e.g., PBS) or a compound of the invention (at, e.g., 10-20 mg/kg), and are evaluated by analysis of memory deficits, histological characteristics of sample tissues, and other indicators of disease progression.

### Alternate Alzheimer's Model: Assessing Efficacy in Amyloid-B-Induced Auto Immune Encephalitis

The abnormal processing and extracellular deposition of amyloid-B (Aβ) peptide, is a defining characteristic of Alzheimer's disease (AD). Recent evidence suggests that vaccination of transgenic mouse models of AD with Aβ causes a marked reduction in brain amyloid burden (e.g. Schenk D et al. (1999) Nature 400:173). Moreover, a recently published report suggests that vaccination with Aβ can, in certain circumstances, determine an aberrant autoimmune reaction to Aβ within the CNS, resulting in a perivenular inflammatory encephalomyelitis (Furlan R et al. (2003) Brain 126:285).

Evaluation of the efficacy of compounds of the invention is carried out in the Aβ-induced autoimmune encephalomyelitis model. Thirty female C57BL/6 mice are immunized subcutaneously (s.c). with 100 μg of Aβ1-42 peptide in Complete Freund Adjuvant (CFA) on day 0, followed by i.p. injections of pertussis toxin (one pertussis toxin injection on day 0, a second pertussis toxin injection on day 2). Groups of 10 mice receive either a compound of the invention (10 mg/kg/dose, twice daily for 18 consecutive days), methotrexate (2.5 mg/kg/day, three times a week, till day 18) or vehicle control (twice/day for 18 consecutive days), all starting from one day after the immunization and all administered i.p. Then animals are monitored for body weight, signs of paralysis and death according to a 0-5 scale of scoring system as follows: 1=limp tail or waddling gait with tail tonicity; 2=waddling gait with limp tail (ataxia); 2.5=ataxia with partial limb paralysis; 3=full paralysis of one limb; 3.5=full paralysis of one limb with partial paralysis of second limb; 4=full paralysis of two limbs; 4.5=moribund; 5=death.

### Example 36

### Effect of Compounds of the Invention in Murine Models of Type I Diabetes Mellitus

It is widely accepted that proinflammatory cytokines play an important role in the development of Type I diabetes. Thus, compounds of the invention can be used to treat patients suffering from this disease. A mouse with diabetes induced by multiple low doses of streptozotocin (STZ) can be used as an animal model for Type I diabetes. STZ is used to induce diabetes in C57BL/6J mice. Briefly, STZ (40 mg/kg) or citrate buffer (vehicle) is given i.p. once daily for 5 consecutive days as described (Carlsson P. O. et al. (2000) Endocrinology. 141(8):2752). Compound administration (i.p. 10 mg/kg, twice a day) is started 5 days before STZ injections and continues for 2 weeks. Another widely used model is the NOD mouse model of autoimmune Type I diabetes (Wong F. S. and Janeway C. A. Jr. (1999) Curr Opin Immunol. 11(6):643. Female NOD mice are treated with daily injections of a compound of the invention (20 mg/kg/day) from week 10 through week 25. The effect of the compounds of the invention in preventing the development of insulitis and diabetes in NOD-scid/scid females after adoptive transfer of splenocytes from diabetic NOD females is also assessed. For both the STZ and NOD models, the incidence of diabetes is monitored in several ways, including monitoring of blood glucose levels. Insulin secretion is assessed in pancreatic islets isolated from experimental mice. Cytokine production is measured in mouse sera. Islet apoptosis is assessed quantitatively.

This procedure is used to screen for compounds which inhibit development of diabetes as compared to control animals.

### Example 37

### Effect of Compounds of the Invention in Models of Airway Inflammation

Anti-inflammatory compounds such as SSAO inhibitors can have beneficial effects in airway inflammatory conditions such as asthma and chronic obstructive pulmonary disease. The rodent model here described has been extensively used in efficacy studies. Other murine models of acute lung inflammation can also be used to test the compounds of the invention.

For the evaluation of the effects of SSAO inhibitors in preventing airway inflammation, three groups of sensitized rats are studied. Animals are challenged with aerosolized OVA (ovalbumin) after intraperitoneal administration of the vehicle saline, a compound of the invention, or a positive control (e.g. prednisone) twice daily for a period of seven days. At the end of the week animals are anesthetized for measurements of allergen-induced airway responses as described (Martin J. G. et al. (2002) J Immunol. 169(7):3963). Animals are intubated endotracheally with polyethylene tubing and placed on a heating pad to maintain a rectal temperature of 36° C. Airflow is measured by placing the tip of the endotracheal tube inside a Plexiglas box (˜250 mL). A pneumotachograph coupled to a differential transducer is connected to the other end of the box to measure airflow. Animals are challenged for 5 min with an aerosol of OVA (5% w/v). A disposable nebulizer will be used with an output of 0.15 mL/min. Airflow is measured every 5 min for 30 min after challenge and subsequently at 15 min intervals for a total period of 8 h. Animals are then sacrificed for bronchoalveolar lavage (BAL). BAL is performed 8 h after challenge with five instillations of 5 mL of saline. The total cell count and cell viability is estimated using a hemacytometer and trypan blue stain. Slides are prepared using a Cytospin and the differential cell count is assessed with May-Grunwald-Giemsa staining, and eosinophil counts by immunocytochemistry.

Alternate Model of Airway Inflammation: Assessing the Effect of Compounds of the Invention

LPS-induced pulmonary inflammation in rats is a widely used model of airway inflammation (e.g. Billah M et al. (2002) J. Pharmacol. Exp. Ther. 302:127). For the experiment described here, groups of Sprague-Dawley female rats (180-200 g) were orally dosed with either a compound of the invention (1 or 10 mg/kg), or with vehicle 1 h before the LPS challenge. Animals were placed in a plexiglass chamber and LPS at 100 μg/ml was nebulized for ten minutes. The nebulizer was turned off for ten minutes, then the chamber was evacuated for a further ten minutes. The animals remained in the chamber throughout this 30 minute cycle. Animals were then returned to their cages for the remainder of the experiment. Four hours after exposure to LPS animals were sacrificed and lungs excised. Lungs were washed with three cycles of 3 ml RPMI 1640 medium and the lavage fluid combined. Cells were pelleted by centrifugation and washed in the same medium.

Total cell counts were performed using a hemacytometer. Differential cell counts were conducted on Cytospin-prepared slides stained with Diff-Quick stain. Standard morphological criteria were used to define mononuclear and neutrophilic cells.

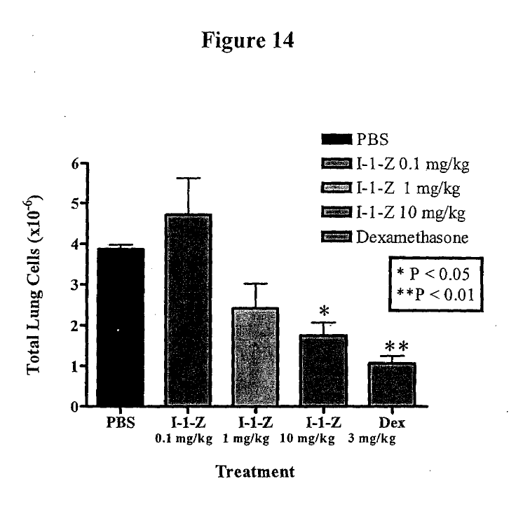

Using the above protocol, an experiment to assess the effects of compound I-1-Z was carried out. Groups of four female Sprague Dawley rats were orally administered PBS, compound I-1-Z at the indicated doses, or dexamethasone at 3 mg/kg. Rats were then exposed to nebulized LPS for 30 minutes. Four hours later rats were sacrificed, and cells harvested from lungs by bronchioalveolar lavage. Approximately 5×10⁵ alveolar macrophages were harvested from lungs of animals not exposed to LPS, and 90-95% of LPS-induced cells were neutrophils. Statistical analysis was performed by one way ANOVA followed by Dunnett's test for multiple comparisons. The results are shown in FIG. 14, and indicate that doses of 1 mg/kg or 10 mg/kg of compound I-1-Z are effective in reducing LPS-induced lung inflammation.

### Example 38

### Efficacy in Model of Systemic Inflammation

Evaluation of the efficacy of compounds of the invention is carried out in a model of endotoxemia (Pawlinski R et al. (2003) Blood 103:1342). Sixteen female C57Bl/6 mice (eight to ten weeks old) are divided in two treatment groups: group A animals are administered 500 μL of PBS orally; group B animals are administered 100 mg/kg of a compound of the invention in 500 μL of PBS orally. 30 min after oral administration of compound, inflammation is induced in all animals by administering i.p. 5 mg/kg of LPS (O111:B4, Sigma) in PBS. Blood samples (˜50 μL) are collected from the retro-orbital sinus at 0 (before oral administration of compound), 1, 2, 4, and 8 h after LPS injection. Each sample is immediately diluted ½ in PBS. Half of the diluted sample is used to prepare blood smear and the other 50 μL is centrifuged and serum is collected. Sera samples are used to determine IL1, IL6 and TNFa levels by ELISA. Animal survival rates are recorded for the next 3 days.

### Example 39

### Inhibition of Cutaneous Inflammation in the SCID Mouse Model of Psoriasis

Recent establishment of the SCID-human skin chimeras with transplanted psoriasis plaques has opened new vistas to study the molecular complexities involved in psoriasis. This model also offers a unique opportunity to investigate various key biological events such as cell proliferation, homing in of T cells in target tissues, inflammation and cytokine/chemokine cascades involved in an inflammatory reaction. The SCID mouse model has been used to evaluate the efficacy of several compounds for psoriasis and other inflammatory diseases (Boehncke W. H. et al. (1999) Arch Dermatol Res. 291(2-3):104).

Transplantations are to be done as described previously (Boehncke, W. H. et al. (1994) Arch. Dermatol. Res. 286:325). Human full-thickness xenografts are transplanted onto the backs of 6- to 8-week-old C.B17 SCID mice (Charles River). For the surgical procedure, mice are anesthetized by intraperitoneal injection of 100 mg/kg ketamine and 5 mg/kg xylazine. Spindle-shaped pieces of full-thickness skin measuring 1 cm in diameter are grafted onto corresponding excisional full-thickness defects of the shaved central dorsum of the mice and fixed by 6-0 atraumatic monofilament sutures. After applying a sterile petroleum jelly-impregnated gauze, the grafts are protected from injury by suturing a skin pouch over the transplanted area using the adjacent lateral skin. The sutures and over-tied pouches are left in place until they resolve spontaneously after 2-3 weeks. Grafts are allowed 2 weeks for acceptance and healing. Thereafter, daily intraperitoneal injections are performed between days 15 and 42 after transplantation. Mice are injected with either vehicle (PBS), dexamethasone (0.2 mg/kg body weight), or a compound of the invention (at, e.g., 20 mg/kg body weight) in a final volume of 200 μL. Mice are sacrificed at day 42, and after excision with surrounding mouse skin the grafts are formalin-embedded. Subsequently, routine hematoxylin-and-eosin staining is performed, and the grafts are analyzed with regard to their pathological changes both qualitatively (epidermal differentiation, inflammatory infiltrate) and quantitatively (epidermal thickness).

### Example 40

### Oral Bioavailability Studies in Rodents

Oral bioavailability studies in mice and rats are to be performed using the following procedure. Briefly, C57B1/6 female mice and Sprague Dawley female rats are administered 50 mg/kg of different compounds of the invention by oral gavage. Animals are bled at different time intervals after compound administration and the levels of inhibitor in plasma are determined using the calorimetric assay described in Example 28 above.

### Example 41

### Dose-Response Effect from In Vivo Administration of SSAO/VAP-1 Inhibitors

In vivo inhibition of SSAO is assessed in rat aorta and lungs, two of the tissues where SSAO activity is highest. Six week old female Sprague Dawley rats are to be administered 0, 0.1, 1, 10 and 50 mg/kg of a compound of the invention in 2.5 mL/kg PBS by oral gavage. Four hours after compound administration the animals are euthanized and their aortas and lungs are removed and frozen in liquid nitrogen. Tissues are homogenized in 0.01M potassium phosphate pH 7.8, and used to measure SSAO activity in the radioactive assay following the protocol described by Lizcano J. M. et al. (1998) Biochem. J. 331:69. Details of the assay are given in Example 27 above.

### Example 42

### Blocking of In Vitro Adhesion by SSAO/VAP-1 Inhibitors

These studies are carried out in order to determine whether SSAO/VAP-1 transfected into endothelial cells will retain the adhesion function and whether it plays any role in the adhesion of freshly isolated human PBMCs to these cells. Moreover, the studies are also designed to determine whether blocking of SSAO/VAP-1 will have an impact on the level of adhesion between these two cell types. Adhesion assays are performed using cells labeled with the fluorescent dye Calcein-AM (Molecular Probes, OR, USA) as per the manufacturer's instructions. Briefly, rat lymph node high endothelial cells (HEC; isolation and culture is described in Ager, A. (1987) J. Cell Sci. 87: 133) are plated overnight in 96-well plates (2,000 cells/well). PBMCs (peripheral blood mononuclear cells) (1×10⁷) are labeled with 1 mL of 10 M Calcein-AM for 1 h at 37° C., washed three times with RPMI, and added to the 96 well plates containing monolayers of HEC cells mock-transfected or transfected with full-length human SSAO/VAP-1 (60,000 PBMCs were plated per well containing 2,000 HEC cells). Adhesion is carried out for 3 h at 37° C. Non-adherent cells are removed by washing three times with RPMI and fluorescence is measured in a fluorescence plate reader at an excitation wavelength of 485 nm and emission wavelength of 530 nm. Several controls are to be included, such as HEC cells and PBMCs (labeled and unlabeled) alone.

The next experiments are designed in order to investigate whether blocking the enzymatic catalytic site will have any effect on the adhesion function of SSAO/VAP-1, and whether or not inhibitors according to the invention will mediate an adhesion-inhibiting effect. Published results suggest that blocking SSAO enzymatic activity with semicarbazide inhibited lymphocyte rolling under laminar sheer on cardiac endothelial monolayers (Salmi et al. Immunity (2001) 14:265). These studies can thus be repeated using the adhesion assay as described above to evaluate the inhibitors of the invention. Adhesion blockers can include an anti-human VAP-1 monoclonal antibody (Serotec, Oxford, UK), neuramidase (a sialidase, because SSAO/VAP-1 is a sialoglycoprotein; Sigma), and several function-blocking antibodies to rat adhesion molecules (CD31-PECAM, CD54-ICAM-1, CD92P-P Selectin). Controls can include the SSAO inhibitor semicarbazide (Sigma), MAO-A and MAO-B inhibitors (clorgyline and pargyline, respectively; Sigma), and mouse IgG1 and IgG2 isotype controls (BD, USA). Antibodies (10 μg/ml) and neuramidase (5 mU) are incubated with the HECs for 30 min at 37° C.; excess antibody is washed away prior to the addition of the labeled PBMCs. Small-molecule inhibitors are pre-incubated the same way at IC₁₀₀ concentrations, but the amounts present in the supernatant are not washed away to preserve the IC₁₀₀ concentration during the adhesion step.

### Example 43

### Effect of Therapeutically Administered I-1-Z on Cell Trafficking

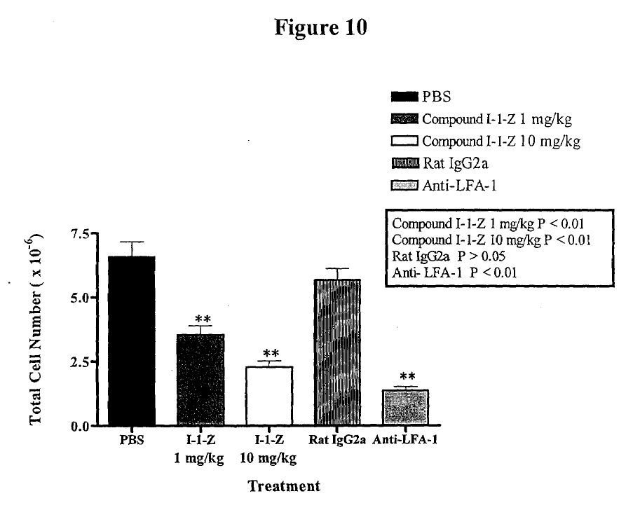

Air pouches were introduced, on day 1, into the back of groups of eight C57BL/6 mice by subcutaneous injection of filtered air. On day 2 the air pouches were injected with 0.5 ml of 1% carrageenan. Four hours later mice were administered: PBS, LJP 1586 at 1 or 10 mg/kg orally, or rat IgG2a antibody or anti-LFA-1 at 100 μg intravenously. Sixteen hours later the mice were sacrificed, cells removed by lavage and counted. Statistical analyses were performed using one-way ANOVA followed by Dunnett's test for multiple comparisons. The results are shown in FIG. 10.

### Example 44

### Effect of Route of Administration of I-1-Z on Cell Trafficking

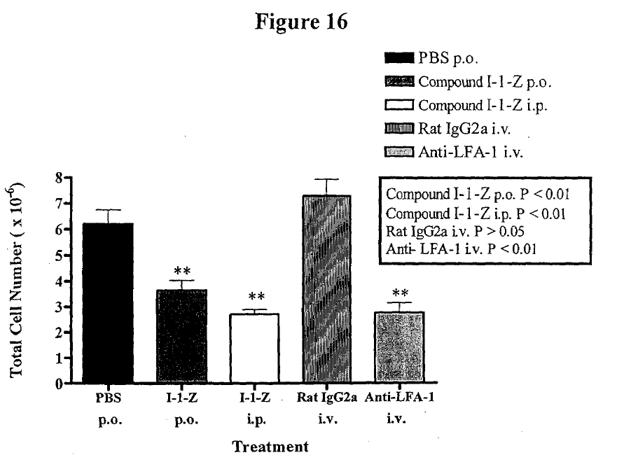

Air pouches were introduced onto mice as described in Example 43 (n=8). Twenty-four hours later mice were administered: PBS p.o. or 10 mg/kg LJP 1586, either p.o. or i.p., or 100 μg rat IgG2a antibody or anti-LFA-1, both intravenously. One hour later the air pouches were injected with 0.5 ml of 1% carrageenan and 24 hours later the mice were sacrificed, cells removed by lavage and counted. Statistical analyses were performed using one-way ANOVA followed by Dunnett's test for multiple comparisons. The results are shown in FIG. 16.

### Example 45

ED50 of Compound I-1-Z for Rat Lung SSAO

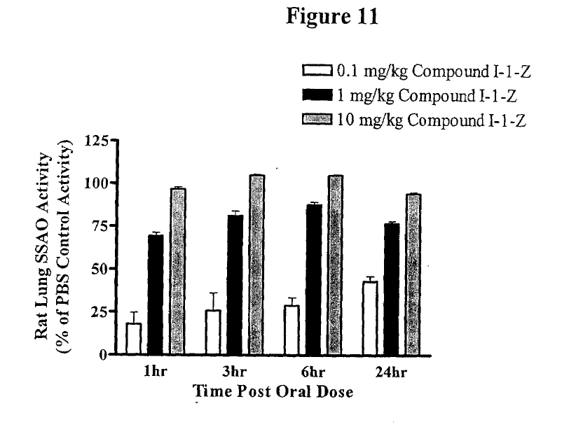

Groups of five Sprague Dawley rats were orally administered the indicated doses of compound I-1-Z. At 1, 3, 6 or 24 h later, the animals were sacrificed, lungs excised and SSAO activity in lung homogenates determined. Results are presented as percent inhibition of SSAO activity relative to animals that were administered PBS. The results are shown in FIG. 11. The ED50 for inhibition of rat lung SSAO activity by compound I-1-Z is between 0.1 mg/kg and 1 mg/kg.

### Example 46

### Comparison of Inhibition of the SSAO Activity of SSAO/VAP-1 Versus DAO

Compounds of the invention can be tested for their inhibitory activity against diamine oxidase (DAO). Recombinant DAO was expressed in CHO cells using standard molecular biology methods as described herein.

The DNA sequence for DAO (AOC1, NM-001091) was synthetically produced by Genscript Corp. A codon-optimized DAO sequence was subcloned into the mammalian expression vector pcDNA5/FRT. CHO cells with a stable genome integrated FLP recombination site allow Frt recombinase mediated integration into a stable transcriptionally active site in the genome. The pcDNA5/FRT containing DAO was cotransfected with pOGG44, a vector that encodes the Frt recombinase at a ration of 1:50 using a total of ˜1 ug of DNA to transfect ˜1E4 cells by the Fugene method. Cells were cultured in F-12K media with 10% FBS containing media containing 0.5 mg/ml Hygromycin until single colonies emerged. Single colonies were picked and grown individually in a 24 well culture plate until confluent. Samples of the media were taken and screened for enzymatic activity using 1,4-diaminobutane as substrate in an AMPLEX Red-based peroxide detection assay. Positive expression clones, compared to media from mock transfected cells which show no activity above background, were expanded and grown in larger quantities in serum containing media until ˜80% confluent. The serum containing media was removed and the cells were switched to CHO SFM serum free media for 48 hours. The media containing enzymatic activity displayed a prominent single band of approximately 78 kD on denaturing SDS-PAGE. Naive CHO SFM media and mock transfection incubated media did not display this signal. The media was collected and used directly as a source of diamine oxidase for enzyme analysis. The enzyme as prepared by this method was inhibited at an IC₅₀ of approximately 12.5 nM by aminoguanidine, similar to values reported in the literature for aminoguanidine inhibition of diamine oxidase (Bieganski et al., Biochim. Biophys. Acta. 756:196 (1983); Holt & Baker, Prog. Brain Res. 106:187 (1995)).

Inhibition of diamine oxidase was determined by using the AMPLEX Red Monoamine Oxidase assay sold by Invitrogen (Carlsbad, Calif.) (AMPLEX is a registered trademark of Molecular Probes, Inc., Eugene Oreg., for fluorogenic chemicals and enzyme-coupled assays for use in scientific research.) The assay protocol was used, with the substitution of diamine oxidase for monoamine oxidase and the substitution of putrescine (1,4-diaminobutane) for the benzylamine/tyramine substrate. (See Nicotra et al., Biogenic Amines 15, 307 (1999); Zhou et al., Anal. Biochem. 253, 162-168 (1997); Zhou et al., Anal. Biochem. 253, 169-174 (1997); Holt et al., Anal. Biochem. 244:384-92 (1997); Hall et al., Biochem. Pharmacol. 18:1447-54 (1969); and Youdim et al., Methods Enzymol. 142:617-27 (1987).)

The specificity of compound I-1-Z for human SSAO versus human diamine oxidase (DAO) was tested. Compound I-1-Z has an IC₅₀ for human SSAO of 0.018 uM, as reported above. The IC₅₀ of compound I-1-Z for human DAO is 92 uM. The IC₅₀ for DAO divided by the IC₅₀ for SSAO of compound I-1-Z is about 5,111, indicating that compound I-1-Z is about 5,000 times more specific for SSAO than for DAO.

### Example 47

### Screening for Receptors Affected by SSAO Inhibitor

A screening procedure was employed to determine whether compound I-1-Z affects specific ligand binding to various receptors. Ligand binding to the following receptors was not affected by a 10 μM concentration of compound I-1-Z:

Non-Peptide Receptors: Adenosine, Adrenergic, Benzodiazepine, Cannabinoid, Dopamine, GABA, Glutamate, Histamine, Melatonin, Muscarinic, Prostanoid, Purinergic, Serotonin, Sigma;

Peptide Receptors: Angiotensin II, Bombesin, Bradykinin, Calcitonin-related peptide, Chemokine, Cholecystokinin, Cytokine, Endothein, Galanin, Growth factor, Melanocortin, Neurokinin, Neuropeptide Y, Neurotensin, Opioid, Somatostatin, VIP, Vasopressin;

Ion Channels: Calcium, Potassium, Sodium.

Ligand binding to the following receptors was affected by a 10 μM concentration of compound I-1-Z:

5-HT_{1A} (34% inhibition);

Amine transporters: Dopamine (38% inhibition), Norepinephrine (68% inhibition), Serotonin (47% inhibition).

Table 2, below, shows receptors that were tested for their potential interaction., and includes the reference compound and short bibliographic reference for the assay. Table 3, below, provides additional information about the assay conditions. Table 4, below, reports the summary data in terms of the percentage by which compound I-1-Z inhibited binding of the reference compound to the receptor. The abbreviation (h) in the tables below indicates that the human receptor was used. Table 2-A, Table 2-B, Table 2-C, and Table 2-D provide additional information about the receptors.

TABLE 2

| Receptor Assay | Origin | Reference Compound | Bibliography |
|---|---|---|---|
| A₁ (h) | human recombinant | DPCPX | Townsend-Nicholson and |
| | (CHO cells) | | Schofield (1994) |
| A_{2A} (h) | human recombinant | NECA | Luthin et al. (1995) |
| | (HEK-293 cells) | | |
| A₃ (h) | human recombinant | IB-MECA | Salvatore et al. (1993) |
| | (HEK-293 cells) | | |
| α₁ (non-selective) | rat cerebral cortex | prazosin | Greengrass and Bremner (1979) |
| α₂ (non-selective) | rat cerebral cortex | yohimbine | Uhlen and Wikberg (1991) |
| β₁ (h) | human recombinant | atenolol | Levin et al. (2002) |
| | (HEK-293 cells) | | |
| β₂ (h) | human recombinant | ICI 118551 | Smith and Teitler (1999) |
| | (Sf9 cells) | | |
| AT₁ (h) | human recombinant | saralasin | Bergsma et al. (1992) |
| | (CHO cells) | | |
| AT₂ (h) | human recombinant | saralasin | Tsuzuki et al. (1994) |
| | (Hela cells) | | |
| BZD (central) | rat cerebral cortex | diazepam | Speth et al. (1979) |
| BZD (peripheral) | rat heart | PK 11195 | Le Fur et al. (1983) |
| BB (non-selective) | rat cerebral cortex | bombesin | Guard et al. (1993) |
| B₂ (h) | human recombinant | NPC 567 | Pruneau et al. (1998) |
| | (CHO cells) | | |
| CGRP (h) | human recombinant | hCGRPα | Aiyar et al. (1996) |
| | (CHO cells) | | |
| CB₁ (h) | human recombinant | CP 55940 | Rinaldi-Carmona et al. (1996) |
| | (CHO cells) | | |
| CCK_{A} (h) (CCK₁) | human recombinant | CCK-8 | Bignon et al. (1999) |
| | (CHO cells) | | |
| CCK_{B} (h) (CCK₂) | human recombinant | CCK-8 | Lee et al. (1993) |
| | (CHO cells) | | |
| D₁ (h) | human recombinant | SCH 23390 | Zhou et al. (1990) |
| | (CHO cells) | | |
| D_{2S} (h) | human recombinant | (+)butaclamol | Grandy et al. (1989) |
| | (HEK-293 cells) | | |
| D₃ (h) | human recombinant | (+)butaclamol | Mackenzie et al. (1994) |
| | (CHO cells) | | |
| D_{4.4} (h) | human recombinant | clozapine | Van Tol et al. (1992) |
| | (CHO cells) | | |
| D₅ (h) | human recombinant | SCH 23390 | Sunahara et al. (1991) |
| | (GH4 cells) | | |
| ET_{A} (h) | human recombinant | endothelin-1 | Buchan et al. (1994) |
| | (CHO cells) | | |
| ET_{B} (h) | human recombinant | endothelin-3 | Fuchs et al. (2001) |
| | (CHO cells) | | |
| GABA (non-selective) | rat cerebral cortex | GABA | Tsuji et al. (1988) |
| GAL1 (h) | human recombinant | galanin | Sullivan et al. (1997) |
| | (HEK-293 cells) | | |
| GAL2 (h) | human recombinant | galanin | Bloomquist et al. (1998) |
| | (CHO cells) | | |
| PDGF | Balb/c 3T3 cells | PDGF BB | Williams et al. (1984) |
| CXCR2 (h) (IL-8B) | human recombinant | IL-8 | White et al. (1998) |
| | (HEK-293 cells) | | |
| TNF-α (h) | U-937 cells | TNF-α | Brockhaus et al. (1990) |
| CCR1 (h) | human recombinant | MIP-1α | Neote et al. (1993) |
| | (HEK-293 cells) | | |
| H₁ (h) | human recombinant | pyrilamine | Smit et al. (1996) |
| | (HEK-293 cells) | | |
| H₂ (h) | human recombinant | cimetidine | Leurs et al. (1994) |
| | (CHO cells) | | |
| MC₄ (h) | human recombinant | NDP-α-MSH | Schioth et al. (1997) |
| | (CHO cells) | | |
| MT₁ (h) | human recombinant | melatonin | Witt-Enderby and Dubocovich |
| | (CHO cells) | | (1996) |
| M₁ (h) | human recombinant | pirenzepine | Dorje et al. (1991) |
| | (CHO cells) | | |
| M₂ (h) | human recombinant | methoctramine | Dorje et al. (1991) |
| | (CHO cells) | | |
| M₃ (h) | human recombinant | 4-DAMP | Peralta et al. (1987) |
| | (CHO cells) | | |
| M₄ (h) | human recombinant | 4-DAMP | Dorje et al. (1991) |
| | (CHO cells) | | |
| M₅ (h) | human recombinant | 4-DAMP | Dorje et al. (1991) |
| | (CHO cells) | | |
| NK₁ (h) | U-373MG cells | [Sar⁹,Met(O₂)¹¹]-SP | Heuillet et al. (1993) |
| NK₂ (h) | human recombinant | [Nle¹⁰]-NKA(4-10) | Aharony et al. (1993) |
| | (CHO cells) | | |
| NK₃ (h) | human recombinant | SB 222200 | Sarau et al. (1997) |
| | (CHO cells) | | |
| Y₁ (h) | SK-N-MC cells | NPY | Wieland et al. (1995) |
| Y₂ (h) | KAN-TS cells | NPY | Fuhlendorff et al. (1990) |
| NT₁ (h) (NTS1) | human recombinant | neurotensin | Vita et al. (1993) |
| | (CHO cells) | | |
| δ₂ (h) (DOP) | human recombinant | DPDPE | Simonin et al. (1994) |
| | (CHO cells) | | |
| κ (KOP) | guinea-pig | U 50488 | Kinouchi and Pasternak (1991) |
| | cerebellum | | |
| μ (h) (MOP) (agonist site) | human recombinant | DAMGO | Wang et al. (1994) |
| | (HEK-293 cells) | | |
| ORL1 (h) (NOP) | human recombinant | nociceptin | Ardati et al. (1997) |
| | (HEK-293 cells) | | |
| PACAP (PAC₁) (h) | human recombinant | PACAP₁₋₃₈ | Ohtaki et al. (1998) |
| | (CHO cells) | | |
| PCP | rat cerebral cortex | MK 801 | Vignon et al. (1986) |
| TXA₂/PGH₂ (h) (TP) | human platelets | U 44069 | Hedberg et al. (1988) |
| P2X | rat urinary bladder | α,β-MeATP | Bo and Burnstock (1990) |
| P2Y | rat cerebral cortex | dATPαS | Simon et al. (1995) |
| 5-HT_{1A} (h) | human recombinant | 8-OH-DPAT | Mulheron et al. (1994) |
| | (HEK-293 cells) | | |
| 5-HT_{1B} | rat cerebral cortex | serotonin | Hoyer et al. (1985) |
| 5-HT_{2A} (h) | human recombinant | ketanserin | Bonhaus et al. (1995) |
| | (HEK-293 cells) | | |
| 5-HT_{2C} (h) | human recombinant | RS-102221 | Stam et al. (1994) |
| | (CHO cells) | | |
| 5-HT₃ (h) | human recombinant | MDL 72222 | Hope et al. (1996) |
| | (CHO cells) | | |
| 5-HT_{5A} (h) | human recombinant | serotonin | Rees et al. (1994) |
| | (CHO cells) | | |
| 5-HT₆ (h) | human recombinant | serotonin | Monsma et al. (1993) |
| | (CHO cells) | | |
| 5-HT₇ (h) | human recombinant | serotonin | Shen et al. (1993) |
| | (CHO cells) | | |
| σ (non-selective) | rat cerebral cortex | haloperidol | Shirayama et al. (1993) |
| sst (non-selective) | AtT-20 cells | somatostatin | Brown et al. (1990) |
| VIP₁ (h) (VPAC₁) | human recombinant | VIP | Couvineau et al. (1985) |
| | (CHO cells) | | |
| V₁ₐ (h) | human recombinant | [d(CH₂)₅¹,Tyr(Me)₂]-AVP | Tahara et al. (1998) |
| | (CHO cells) | | |
| Ca²⁺ channel (L, verapamil site) | rat cerebral cortex | D 600 | Reynolds et al. (1986) |
| (phenylalkylamines) | | | |
| K⁺_{V} channel | rat cerebral cortex | α-dendrotoxin | Sorensen and Blaustein (1989) |
| SK⁺_{Ca} channel | rat cerebral cortex | apamin | Hugues et al. (1982) |
| Na⁺ channel (site 2) | rat cerebral cortex | veratridine | Brown (1986) |
| Cl⁻ channel | rat cerebral cortex | picrotoxinin | Lewin et al. (1989) |
| NE transporter (h) | human recombinant | protriptyline | Pacholczyk et al. (1991) |
| | (CHO cells) | | |
| DA transporter (h) | human recombinant | BTCP | Pristupa et al. (1994) |
| | (CHO cells) | | |
| 5-HT transporter (h) | human recombinant | imipramine | Tatsumi et al. (1999) |
| | (CHO cells) | | |

TABLE 2-A
Non-Peptide Receptors

| Class | Family | Receptor |
|---|---|---|
| Non-Peptide | Adenosine | A₁ |
| | | A_{2A} |
| | | A₃ |
| | Adrenergic | α₁ |
| | | α₂ |
| | | β₁ |
| | | β₂ |
| | Benzodiazepine | BZD (central) |
| | | BZD (peripheral) |
| | Cannabinoid | CB₁ |
| | Dopamine | D₁ |
| | | D_{2S} |
| | | D₃ |
| | | D_{4.4} |
| | | D₅ |
| | γ-aminobutyric acid | GABA (non-selective) |
| | Histamine | H₁ |
| | | H₂ |
| | Melatonin | MT₁ |
| | Muscarinic | M₁ |
| | | M₂ |
| | | M₃ |
| | | M₄ |
| | | M₅ |
| | Glutamate | PCP |
| | Prostanoid | TXA₂/PGH₂ |
| | Purinergic | P2X |
| | | P2Y |
| | Serotonin | 5-HT_{1A} |
| | | 5-HT_{1B} |
| | | 5-HT_{2A} |
| | | 5-HT_{2C} |
| | | 5-HT₃ |
| | | 5-HT_{5A} |
| | | 5-HT₆ |
| | | 5-HT₇ |
| | Sigma | σ (non-selective) |
| | Somatostatin | sst (non-selective) |

TABLE 2-B
Peptide Receptors

| Class | Family | Receptor |
|---|---|---|
| Peptide | Angiotensin | AT1 |
| | | AT2 |
| | Bombesin | BB |
| | Bradykinin | B2 |
| | Calcitonin gene-related peptide | CGRP |
| | Cholecystokinin | CCK_{A} (CCK₁) |
| | | CCK_{B} (CCK₂) |
| | Endothelin | ET_{A} |
| | | ET_{B} |
| | Galanin | GAL1 |
| | | GAL2 |
| | Platelet-derived growth factor | PDGF |
| | Chemokine Receptor | CXCR2 |
| | | CCR1 |
| | Cytokine | TNFα |
| | Melanocortin | MC₄ |
| | Neurokinin | NK₁ |
| | | NK₂ |
| | | NK₃ |
| | Neuropeptide Y | Y₁ |
| | | Y₂ |
| | Neurotensin | NT₁ |
| | Opioid & opioid-like | δ₂ |
| | | κ |
| | | μ |
| | | ORL1 |
| | Vasoactive intestinal peptide | PACAP |
| | | VIP₁ |
| | Vasopressin | V₁ₐ |

TABLE 2-C
Ion Channels

| Class | Family | Receptor |
|---|---|---|
| Ion Channel | Calcium | Ca²⁺ (L, verapamil site) |
| | Potassium | K⁺V |
| | | SK⁺_{Ca} |
| | Sodium | Na⁺ (site 2) |
| | Chloride | Cl⁻ |

TABLE 2-D
Amine Transporters

| Class | Family | Receptor |
|---|---|---|
| Transporter | Norepinephrine | NE transporter |
| | Dopamine | DA transporter |
| | Serotonin | 5-HT transporter |

TABLE 3

| | | | | | | Method of |
|---|---|---|---|---|---|---|
| Assay | Ligand | Conc. | | Non Specific | Incubation | Detection |
| A₁ (h) | [³H]DPCPX | 1 | nM | DPCPX | 60 min./22° C. | Scintillation |
| | | | | (1 μM) | | counting |
| A_{2A} (h) | [³H]CGS 21680 | 6 | nM | NECA | 120 min./22° C. | Scintillation |
| | | | | (10 μM) | | counting |
| A₃ (h) | [¹²⁵I]AB-MECA | 0.15 | nM | IB-MECA | 120 min./22° C. | Scintillation |
| | | | | (1 μM) | | counting |
| α₁ (non-selective) | [³H]prazosin | 0.25 | nM | prazosin | 60 min./22° C. | Scintillation |
| | | | | (0.5 μM) | | counting |
| α₂ (non-selective) | [³H]RX 821002 | 0.5 | nM | (−)epinephrine | 60 min./22° C. | Scintillation |
| | | | | (100 μM) | | counting |
| β₁ (h) | [³H](−)CGP 12177 | 0.15 | nM | alprenolol | 60 min./22° C. | Scintillation |
| | | | | (50 μM) | | counting |
| β₂ (h) | [³H](−)CGP 12177 | 0.15 | nM | alprenolol | 60 min./22° C. | Scintillation |
| | | | | (50 μM) | | counting |
| AT₁ (h) | [¹²⁵I][Sar¹,Ile⁸]-ATII | 0.05 | nM | angiotensin II | 60 min./37° C. | Scintillation |
| | | | | (10 μM) | | counting |
| AT₂ (h) | [¹²⁵I]CGP 42112A | 0.05 | nM | angiotensin II | 180 min./37° C. | Scintillation |
| | | | | (1 μM) | | counting |
| BZD (central) | [³H]flunitrazepam | 0.4 | nM | diazepam | 60 min./4° C. | Scintillation |
| | | | | (3 μM) | | counting |
| BZD (peripheral) | [³H]PK 11195 | 0.2 | nM | PK 11195 | 15 min./22° C. | Scintillation |
| | | | | (10 μM) | | counting |
| BB (non-selective) | [¹²⁵I][Tyr⁴]bombesin | 0.01 | nM | bombesin | 60 min./22° C. | Scintillation |
| | | | | (1 μM) | | counting |
| B₂ (h) | [³H]bradykinin | 0.2 | nM | bradykinin | 60 min./22° C. | Scintillation |
| | | | | (1 μM) | | counting |
| CGRP (h) | [¹²⁵I]hCGRPα | 0.03 | nM | hCGRPα | 90 min./22° C. | Scintillation |
| | | | | (1 μM) | | counting |
| CB₁ (h) | [³H]CP 55940 | 0.5 | nM | WIN 55212-2 | 120 min./37° C. | Scintillation |
| | | | | (10 μM) | | counting |
| CCK_{A} (h) (CCK₁) | [¹²⁵I]CCK-8 | 0.08 | nM | CCK-8 | 60 min./22° C. | Scintillation |
| | | | | (1 μM) | | counting |
| CCK_{B} (h) (CCK₂) | [¹²⁵I]CCK-8 | 0.04 | nM | CCK-8 | 60 min./22° C. | Scintillation |
| | | | | (1 μM) | | counting |
| D₁ (h) | [³H]SCH 23390 | 0.3 | nM | SCH 23390 | 60 min./22° C. | Scintillation |
| | | | | (1 μM) | | counting |
| D_{2S} (h) | [³H]spiperone | 0.3 | nM | (+)butaclamol | 60 min./22° C. | Scintillation |
| | | | | (10 μM) | | counting |
| D₃ (h) | [³H]spiperone | 0.3 | nM | (+)butaclamol | 60 min./22° C. | Scintillation |
| | | | | (10 μM) | | counting |
| D_{4.4} (h) | [³H]spiperone | 0.3 | nM | (+)butaclamol | 60 min./22° C. | Scintillation |
| | | | | (10 μM) | | counting |
| D₅ (h) | [³H]SCH 23390 | 0.3 | nM | SCH 23390 | 60 min./22° C. | Scintillation |
| | | | | (10 μM) | | counting |
| ET_{A} (h) | [¹²⁵I]endothelin-1 | 0.03 | nM | endothelin-1 | 120 min./37° C. | Scintillation |
| | | | | (0.1 μM) | | counting |
| ET_{B} (h) | [¹²⁵I]endothelin-1 | 0.03 | nM | endothelin-1 | 120 min./37° C. | Scintillation |
| | | | | (0.1 μM) | | counting |
| GABA (non-selective) | [³H]GABA | 10 | nM | GABA | 60 min./22° C. | Scintillation |
| | | | | (100 μM) | | counting |
| GAL1 (h) | [¹²⁵I]galanin | 0.1 | nM | galanin | 60 min./22° C. | Scintillation |
| | | | | (1 μM) | | counting |
| GAL2 (h) | [¹²⁵I]galanin | 0.05 | nM | galanin | 120 min./22° C. | Scintillation |
| | | | | (1 μM) | | counting |
| PDGF | [¹²⁵I]PDGF BB | 0.03 | nM | PDGF BB | 180 min./4° C. | Scintillation |
| | | | | (10 nM) | | counting |
| CXCR2 (h) (IL-8B) | [¹²⁵I]IL-8 | 0.025 | nM | IL-8 | 60 min./22° C. | Scintillation |
| | | | | (0.3 μM) | | counting |
| TNF-α (h) | [¹²⁵I]TNF-α | 0.1 | nM | TNF-α | 120 min./4° C. | Scintillation |
| | | | | (10 nM) | | counting |
| CCR1 (h) | [¹²⁵I]MIP-1α | 0.03 | nM | MIP-1α | 120 min./22° C. | Scintillation |
| | | | | (0.1 μM) | | counting |
| H₁ (h) | [³H]pyrilamine | 3 | nM | pyrilamine | 60 min./22° C. | Scintillation |
| | | | | (1 μM) | | counting |
| H₂ (h) | [¹²⁵I]APT | 0.2 | nM | tiotidine | 120 min./22° C. | Scintillation |
| | | | | (100 μM) | | counting |
| MC₄ (h) | [¹²⁵I]NDP-α-MSH | 0.05 | nM | NDP-α-MSH | 120 min./37° C. | Scintillation |
| | | | | (1 μM) | | counting |
| MT₁ (h) | [¹²⁵I]iodomelatonin | 0.025 | nM | melatonin | 60 min./22° C. | Scintillation |
| | | | | (1 μM) | | counting |
| M₁ (h) | [³H]pirenzepine | 2 | nM | atropine | 60 min./22° C. | Scintillation |
| | | | | (1 μM) | | counting |
| M₂ (h) | [³H]AF-DX 384 | 2 | nM | atropine | 60 min./22° C. | Scintillation |
| | | | | (1 μM) | | counting |
| M₃ (h) | [³H]4-DAMP | 0.2 | nM | atropine | 60 min./22° C. | Scintillation |
| | | | | (1 μM) | | counting |
| M₄ (h) | [³H]4-DAMP | 0.2 | nM | atropine | 60 min./22° C. | Scintillation |
| | | | | (1 μM) | | counting |
| M₅ (h) | [³H]4-DAMP | 0.3 | nM | atropine | 60 min./22° C. | Scintillation |
| | | | | (1 μM) | | counting |
| NK₁ (h) | [¹²⁵I]BH-SP | 0.15 | nM | [Sar⁹,Met(O₂)¹¹]-SP | 60 min./22° C. | Scintillation |
| | | | | (1 μM) | | counting |
| NK₂ (h) | [¹²⁵I]NKA | 0.1 | nM | [Nle¹⁰]-NKA(4-10) | 60 min./22° C. | Scintillation |
| | | | | (10 μM) | | counting |
| NK₃ (h) | [³H]SR 142801 | 0.4 | nM | SB 222200 | 120 min./22° C. | Scintillation |
| | | | | (10 μM) | | counting |
| Y₁ (h) | [¹²⁵I]peptide YY | 0.025 | nM | NPY | 120 min./37° C. | Scintillation |
| | | | | (1 μM) | | counting |
| Y₂ (h) | [¹²⁵I]peptide YY | 0.015 | nM | NPY | 60 min./37° C. | Scintillation |
| | | | | (1 μM) | | counting |
| NT₁ (h) (NTS1) | [¹²⁵I]Tyr³-neurotensin | 0.05 | nM | neurotensin | 60 min./4° C. | Scintillation |
| | | | | (1 μM) | | counting |
| δ₂ (h) (DOP) | [³H]DADLE | 0.5 | nM | naltrexone | 120 min./22° C. | Scintillation |
| | | | | (10 μM) | | counting |
| κ (KOP) | [³H]U 69593 | 0.7 | nM | naloxone | 80 min./22° C. | Scintillation |
| | | | | (10 μM) | | counting |
| μ (h) (MOP) (agonist site) | [³H]DAMGO | 0.5 | nM | naloxone | 120 min./22° C. | Scintillation |
| | | | | (10 μM) | | counting |
| ORL1 (h) (NOP) | [³H]nociceptin | 0.2 | nM | nociceptin | 60 min./22° C. | Scintillation |
| | | | | (1 μM) | | counting |
| PACAP (PAC₁) (h) | [¹²⁵I]PACAP₁₋₂₇ | 0.015 | nM | PACAP₁₋₂₇ | 120 min./22° C. | Scintillation |
| | | | | (0.1 μM) | | counting |
| PCP | [³H]TCP | 5 | nM | MK 801 | 60 min./22° C. | Scintillation |
| | | | | (10 μM) | | counting |
| TXA₂/PGH₂ (h) (TP) | [³H]SQ 29548 | 5 | nM | U 44069 | 60 min./22° C. | Scintillation |
| | | | | (50 nM) | | counting |
| P2X | [³H]α,β-MeATP | 3 | nM | α,β-MeATP | 120 min./4° C. | Scintillation |
| | | | | (10 μM) | | counting |
| P2Y | [³⁵S]dATPαS | 10 | nM | dATPαS | 60 min./22° C. | Scintillation |
| | | | | (10 μM) | | counting |
| 5-HT_{1A} (h) | [³H]8-OH-DPAT | 0.3 | nM | 8-OH-DPAT | 60 min./22° C. | Scintillation |
| | | | | (10 μM) | | counting |
| 5-HT_{1B} | [¹²⁵I]CYP (+30 μM | 0.1 | nM | serotonin | 120 min./37° C. | Scintillation |
| | (−)propranolol) | | | (10 μM) | | counting |
| 5-HT_{2A} (h) | [³H]ketanserin | 0.5 | nM | ketanserin | 60 min./22° C. | Scintillation |
| | | | | (1 μM) | | counting |
| 5-HT_{2C} (h) | [³H]mesulergine | 1 | nM | RS-102221 | 60 min./37° C. | Scintillation |
| | | | | (10 μM) | | counting |
| 5-HT₃ (h) | [³H]BRL 43694 | 0.5 | nM | MDL 72222 | 120 min./22° C. | Scintillation |
| | | | | (10 μM) | | counting |
| 5-HT_{5A} (h) | [³H]LSD | 1 | nM | serotonin | 60 min./37° C. | Scintillation |
| | | | | (100 μM) | | counting |
| 5-HT₆ (h) | [³H]LSD | 2 | nM | serotonin | 120 min./37° C. | Scintillation |
| | | | | (100 μM) | | counting |
| 5-HT₇ (h) | [³H]LSD | 4 | nM | serotonin | 120 min./22° C. | Scintillation |
| | | | | (10 μM) | | counting |
| σ (non-selective) | [³H]DTG | 8 | nM | haloperidol | 120 min./22° C. | Scintillation |
| | | | | (10 μM) | | counting |
| sst (non-selective) | [¹²⁵I]Tyr¹¹-somatostatin | 0.05 | nM | somatostatin | 60 min./37° C. | Scintillation |
| | | | | (0.3 μM) | | counting |
| VIP₁ (h) (VPAC₁) | [¹²⁵I]VIP | 0.04 | nM | VIP | 60 min./22° C. | Scintillation |
| | | | | (0.3 μM) | | counting |
| V₁ₐ (h) | [³H]AVP | 0.3 | nM | AVP | 60 min./22° C. | Scintillation |
| | | | | (1 μM) | | counting |
| Ca²⁺ channel (L, verapamil | [³H](−)D 888 | 3 | nM | D 600 | 120 min./22° C. | Scintillation |
| site) (phenylalkyl amines) | | | | (10 μM) | | counting |
| K⁺_{V} channel | [¹²⁵I]α-dendrotoxin | 0.01 | nM | α-dendrotoxin | 60 min./22° C. | Scintillation |
| | | | | (50 μM) | | counting |
| SK⁺_{Ca} channel | [¹²⁵I]apamin | 0.007 | nM | apamin | 60 min./4° C. | Scintillation |
| | | | | (0.1 μM) | | counting |
| Na⁺ channel (site 2) | [³H]batrachotoxinin | 10 | nM | veratridine | 60 min./22° C. | Scintillation |
| | | | | (300 μM) | | counting |
| Cl⁻ channel | [³⁵S]TBPS | 3 | nM | picrotoxinin | 120 min./22° C. | Scintillation |
| | | | | (20 μM) | | counting |
| NE transporter (h) | [³H]nisoxetine | 1 | nM | desipramine | 120 min./4° C. | Scintillation |
| | | | | (1 μM) | | counting |
| DA transporter (h) | [³H]BTCP | 4 | nM | BTCP | 120 min./4° C. | Scintillation |
| | | | | (10 μM) | | counting |
| 5-HT transporter (h) | [³H]imipramine | 2 | nM | imipramine | 60 min./22° C. | Scintillation |
| | | | | (10 μM) | | counting |

For Table 4 below, the specific ligand binding to the receptors is defined as the difference between the total binding and the nonspecific binding determined in the presence of an excess of unlabelled ligand. The results are expressed as a percent inhibition of control specific binding obtained in the presence of Compound I-1-Z. The IC₅₀ values (concentration causing a half-maximal inhibition of control specific binding) and Hill coefficients (n_{H}) were determined by non-linear regression analysis of the competition curves using Hill equation curve fitting. The inhibition constants (Kᵢ) were calculated from the Cheng Prusoff equation (Kᵢ=IC₅₀/(1+(L/K_{D})), where L=concentration of radioligand in the assay, and K_{D}=affinity of the radioligand for the receptor). In each experiment, the respective reference compound was tested concurrently with Compound I-1-Z in order to assess the assay suitability. The reference compounds were tested at several concentrations to confirm that their IC₅₀ values were comparable with historical values, in order to confirm the validity of the assay (data not shown).

The threshold for significance of the effect of the test compound (i.e., the threshold for a true positive for receptor interaction) is taken as greater than or equal to about 30% here, although other thresholds can be used (such as greater than or equal to about 15%, or greater than or equal to about 20%, or greater than or equal to about 25%). Low to moderate negative values for inhibition have no real meaning and are attributable to variability of the signal around the control level.

TABLE 4
Summary Results
10 uM Concentration of Compound I-1-Z

| Receptor | Mean Value |
|---|---|
| A₁ (h) | −3 |
| A_{2A} (h) | 13 |
| A₃ (h) | 1 |
| α₁ (non-selective) | 8 |
| α₂ (non-selective) | 23 |
| β₁ (h) | 5 |
| β₂ (h) | 6 |
| AT₁ (h) | 10 |
| AT₂ (h) | −19 |
| BZD (central) | 14 |
| BZD (peripheral) | −6 |
| BB (non-selective) | 21 |
| B₂ (h) | 1 |
| CGRP (h) | −7 |
| CB₁ (h) | 14 |
| CCK_{A} (h) (CCK₁) | −13 |
| CCK_{B} (h) (CCK₂) | −5 |
| D₁ (h) | −2 |
| D_{2S} (h) | −4 |
| D₃ (h) | −2 |
| D_{4.4} (h) | 2 |
| D₅ (h) | −10 |
| ET_{A} (h) | 8 |
| ET_{B} (h) | −5 |
| GABA (non-selective) | −19 |
| GAL1 (h) | 0 |
| GAL2 (h) | 12 |
| PDGF | −14 |
| CXCR2 (h) (IL-8B) | −8 |
| TNF-α (h) | −6 |
| CCR1 (h) | 3 |
| H₁ (h) | 3 |
| H₂ (h) | 16 |
| MC₄ (h) | 8 |
| MT₁ (h) | 0 |
| M₁ (h) | 28 |
| M₂ (h) | 3 |
| M₃ (h) | 16 |
| M₄ (h) | 22 |
| M₅ (h) | 27 |
| NK₁ (h) | −6 |
| NK₂ (h) | 3 |
| NK₃ (h) | 1 |
| Y₁ (h) | −2 |
| Y₂ (h) | 11 |
| NT₁ (h) (NTS1) | −1 |
| δ₂ (h) (DOP) | 0 |
| κ (KOP) | −18 |
| μ (h) (MOP) (agonist site) | 14 |
| ORL1 (h) (NOP) | −9 |
| PACAP (PAC₁) (h) | 5 |
| PCP | −12 |
| TXA₂/PGH₂ (h) (TP) | 4 |
| P2X | −2 |
| P2Y | −1 |
| 5-HT_{1A} (h) | 34 |
| 5-HT_{1B} | 16 |
| 5-HT_{2A} (h) | −4 |
| 5-HT_{2C} (h) | 21 |
| 5-HT₃ (h) | 4 |
| 5-HT_{5A} (h) | −3 |
| 5-HT₆ (h) | 1 |
| 5-HT₇ (h) | 13 |
| σ (non-selective) | 24 |
| sst (non-selective) | −4 |
| VIP₁ (h) (VPAC₁) | −13 |
| V₁ₐ (h) | 9 |
| Ca²⁺ channel (L, verapamil site) | 14 |
| (phenylalkylamines) | |
| K⁺_{V} channel | −5 |
| SK⁺_{Ca} channel | 4 |
| Na⁺ channel (site 2) | 9 |
| Cl⁻ channel | −6 |
| NE transporter (h) | 68 |
| DA transporter (h) | 38 |
| 5-HT transporter (h) | 47 |

The following references describe the receptors and assays used in these screens: Aharony, D., et al. (1993) Mol. Pharmacol., 44: 356-363; Aiyar, N., et al., (1996) J. Biol. Chem., 271: 11325-11329; Ardati, A., et al., (1997) Mol. Pharmacol., 51: 816-824; Bergsma, D. J., et al. (1992) Biochem. Biophys. Res. Commun., 183: 989-995; Bignon, E., et al. (1999) J. Pharmacol. Exp. Ther. 289: 742-751; Bloomquist, B. T. et al. (1998) Biochem. Biophys. Res. Commun., 243: 474-479; Bo, X. and Burnstock, G. (1990); Brit. J. Pharmacol., 101: 291-296; Bonhaus, D. W., et al. (1995) Brit. J. Pharmacol., 115: 622-628; Brockhaus, M., et al. (1990) Proc. Natl. Acad. Sci. U.S.A., 87: 3127-3131; Brown, G. B. (1986) J. Neurosci., 6: 2064-2070; Brown, P. J., et al. (1990) J. Biol. Chem., 265: 17995-18004; Buchan, K. W., et al. (1994) Brit. J. Pharmacol., 112: 1251-1257; Couvineau, A., et al. (1985) Biochem. J., 231: 139-143; Dorje, F., et al. (1991) J. Pharmacol. Exp. Ther., 256: 727-733; Fuchs, S., et al. (2001) Mol. Med., 7: 115-124; Fuhlendorff, J., et al. (1990) Proc. Natl. Acad. Sci. U.S.A., 87: 182-186; Grandy, D. K., et al. (1989) Proc. Natl. Acad. Sci. U.S.A., 86: 9762-9766; Greengrass, P. and Bremner, R. (1979) Eur. J. Pharmacol., 55: 323-326; Guard, S., et al. (1993) Eur. J. Pharmacol., 240: 177-184; Hedberg, A., et al. (1988) J. Pharmacol. Exp. Ther., 245: 786-792; Heuillet, E., et al. (1993) J. Neurochem., 60: 868-876; Hope, A. G., et al. (1996) Brit. J. Pharmacol., 118: 1237-1245; Hoyer, D., et al. (1985) Eur. J. Pharmacol., 118: 1-12; Hughes, M., et al. (1982) J. Biol. Chem., 257: 2762-2769; Kinouchi, K. and Pasternak, G. W. (1991) Eur. J. Pharmacol., 2077: 135-141; Le Fur, G., et al. (1983) Life Sci., 33: 449-457; Lee, Y.-M., et al. (1993) J. Biol. Chem., 268: 8164-8169; Leurs, R., et al. (1994) Brit. J. Pharmacol., 112: 847-854; Levin, M. C., et al. (2002) J. Biol. Chem., 277: 30429-30435; Lewin, A. H., et al. (1989) Mol. Pharmacol., 35: 189-194; Luthin, D. R., et al. (1995) Mol. Pharmacol., 47: 307-313; Mackenzie, R. G., et al. (1994) Eur. J. Pharmacol., 266: 79-85; Monsma, F. J., et al. (1993) Mol. Pharmacol., 43: 320-327; Mulheron, J. G., et al. (1994) J. Biol. Chem., 269: 12954-12962; Neote, K., et al. (1993) Cell, 72: 415-425; Ohtaki, T., et al. (1998) J. Biol. Chem., 273: 15464-15473; Pacholczyk, T., et al. (1991) Nature, 350: 350-354; Peralta, E. G., et al. (1987) EMBO. J., 6: 3923-3929; Pristupa, Z. B., et al. (1994) Mol. Pharmacol., 45: 125-135; Pruneau, D., et al. (1998) Brit. J. Pharmacol., 125: 365-372; Rees, S., et al. (1994) FEBS Lett., 355: 242-246; Reynolds, I. J., et al. (1986) J. Pharmacol. Exp. Ther., 237: 731-738; Rinaldi-Carmona, M., et al. (1996) J. Pharmacol. Exp. Ther., 278: 871-878; Salvatore, C. A., et al. (1993) Proc. Natl. Acad. Sci. U.S.A., 90: 10365-10369; Sarau, H. M., et al. (1997) J. Pharmacol. Exp. Ther., 281: 1303-13111; Schioth, H. B., et al. (1997) Neuropeptides, 31: 565-571; Shen, Y., et al. (1993) J. Biol. Chem., 268: 18200-18204; Shirayama, Y., et al. (1993) Eur. J. Pharmacol., 2377: 117-126; Simon, J., et al. (1995) Pharmacol. Toxicol., 76: 302-307; Simonin, F., et al. (1994) Mol. Pharmacol., 46: 1015-1021; Smit, M. J., et al. (1996) Brit. J. Pharmacol., 1177: 1071-1080; Smith, C. and Teitler, M. (1999) Cardiovasc. Drugs Ther., 13: 123-126; Sorensen, R. G. and Blaustein, M. P. (1989) Mol. Pharmacol., 36: 689-698; Speth, R. C., et al. (1979) Life Sci., 24: 351-358; Stain, N. J., et al. (1994) Eur. J. Pharmacol., 269: 339-348; Sullivan, K. A., et al. (1997) Biochem. Biophys. Res. Commun., 233: 823-828; Sunahara, R. K., et al. (1991) Nature, 350: 614-619; Tahara, A., et al. (1998) Brit. J. Pharmacol., 125: 1463-1470; Tatsumi, M., et al. (1999) Eur. J. Pharmacol., 368: 277-283; Townsend-Nicholson, A. and Schofield, P. R. (1994) J. Biol. Chem., 269: 2373-2376; Tsuji, A., et al. (1988) Antimicrob. Agents Chemother., 32: 190-194; Tsuzuki, S., et al. (1994) Biochem. Biophys. Res. Commun., 200: 1449-1454; Uhlen, S. and Wikberg, J. E. (1991) Pharmacol. Toxicol., 69: 341-350; Van Tol, H. H. M., et al. (1992) Nature, 358: 149-152; Vignon, J., et al. (1986) Brain Res., 378: 133-141; Vita, N., et al. (1993) FEBS Lett., 317: 139-142; Wang, J.-B., et al. (1994) FEBS Lett., 338: 217-222; White, J. R., et al. (1998) J. Biol. Chem., 273: 10095-10098; Wieland, H. A., et al. (1995) J. Pharmacol. Exp. Ther., 275: 143-149; Williams, L. T., et al. (1984) J. Biol. Chem., 259: 5287-5294; Witt-Enderby, P. A. and Dubocovich, M. L. (1996) Mol. Pharmacol., 50: 166-174; Zhou, Q.-Y., et al. (1990) Nature, 347: 76-80.

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

1. A method of treating or preventing inflammation, an inflammatory disease, an immune disease, or an autoimmune disease, comprising administering a therapeutically effective amount of a compound of the formula: 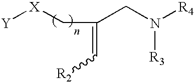
wherein Y is aryl or heteroaryl optionally substituted with one or more groups of the form R₁, wherein each R₁ is independently selected from C₁-C₈ alkyl, C₃-C₈ cycloalkyl, —O—C₁-C₈ alkyl, —O—C₃-C₈ cycloalkyl, —C₆-C₁₀ aryl, —O—C₁-C₄ alkyl-C₆-C₁₀ aryl, —S—C₁-C₈ alkyl, —CF₃, —S—CF₃, —OCF₃, —OCH₂CF₃, F, Cl, Br, I, —NO₂, —OH, —CN, —NR₅R₆, —NHR₇, and —S(O₂)—(C₁-C₈ alkyl); R₂ is selected from H, F, Cl, C₁-C₄ alkyl, and —CF₃; R₃ and R₄ are independently selected from H, —C₁-C₈ alkyl, —C₁-C₄ alkyl-C₆-C₁₀ aryl, or R₃ and R₄ together with the nitrogen to which they are attached form a nitrogen-containing ring; R₅ and R₆ are independently selected from H, —C₁-C₈ alkyl, —C₁-C₄ alkyl-C₆-C₁₀ aryl, or R₅ and R₆ together with the nitrogen to which they are attached form a nitrogen-containing ring; R₇ is selected from —C(═O)—(C₁-C₈ alkyl) and —C(═O)—(C₆-C₁₀ aryl); X is —CH₂—, —O—, or —S—; n is 0, 1, 2, or 3;
and any stereoisomer, mixture of stereoisomers, prodrug, metabolite, crystalline form, non-crystalline form, hydrate, solvate, or salt thereof.

2. The method of claim 1, wherein X is CH₂ and n is 0.

3. The method of claim 1, wherein X is CH₂ and n is 1.

4. The method of claim 1, wherein Y is phenyl, optionally substituted with one or more groups of the form R₁.

5. The method of claim 1, wherein R₃ and R₄ are both H.

6. The method of claim 1, wherein the double bond is in the Z configuration.

7. The method of claim 1, wherein the double bond is in the E configuration.

8. The method of claim 1, wherein R₂ is F.

9. The method of claim 1, wherein R₂ is Cl.

10. The method of claim 1, wherein X is O and n is 0.

11. The method of claim 1, wherein the inflammation or inflammatory disease or immune or autoimmune disease is multiple sclerosis, chronic multiple sclerosis, synovitis, systemic inflammatory sepsis, inflammatory bowel diseases, Crohn's disease, ulcerative colitis, Alzheimer's disease, vascular dementia, atherosclerosis, rheumatoid arthritis, juvenile rheumatoid arthritis, pulmonary inflammation, asthma, skin inflammation, contact dermatitis, liver inflammation, liver autoimmune diseases, autoimmune hepatitis, primary biliary cirrhosis, sclerosing cholangitis, autoimmune cholangitis, alcoholic liver disease, Type I diabetes and/or complications thereof, Type II diabetes and/or complications thereof, atherosclerosis, chronic heart failure, congestive heart failure, ischemic diseases, stroke and/or complications thereof, or myocardial infarction and/or complications thereof.

12. The method of claim 11, wherein the inflammation or inflammatory disease or immune or autoimmune disease is multiple sclerosis, chronic multiple sclerosis, stroke, or complications of stroke.

13. The method of claim 1, wherein the compound is:
(Z)-2-(4-ethoxybenzyl)-3-fluoroprop-2-en-1-amine,
(Z)-2-(4-chlorobenzyl)-3-fluoroprop-2-en-1-amine,
(Z)-2-(3-chlorobenzyl)-3-fluoroprop-2-en-1-amine,
(Z)-3-fluoro-2-(4-isopropoxybenzyl)prop-2-en-1-amine,
(Z)-3-fluoro-2-(4-(methylthio)benzyl)prop-2-en-1-amine,
(Z)-3-fluoro-2-(3-(methylthio)benzyl)prop-2-en-1-amine,
(Z)-3-fluoro-2-(4-(methylsulfonyl)benzyl)prop-2-en-1-amine,
(Z)-3-fluoro-2-(4-methylbenzyl)prop-2-en-1-amine,
(Z)-3-fluoro-2-(3-methylbenzyl)prop-2-en-1-amine,
(Z)-3-fluoro-2-(4-isopropylbenzyl)prop-2-en-1-amine,
(Z)-2-(4-tert-butylbenzyl)-3-fluoroprop-2-en-1-amine,
(Z)-2-(biphenyl-4-ylmethyl)-3-fluoroprop-2-en-1-amine,
(Z)-3-fluoro-2-(4-fluorobenzyl)prop-2-en-1-amine,
(Z)-3-fluoro-2-(3-(trifluoromethyl)benzyl)prop-2-en-1-amine,
(Z)-3-fluoro-2-(3-fluorobenzyl)prop-2-en-1-amine,
(Z)-3-fluoro-2-(3-fluoro-4-methylbenzyl)prop-2-en-1-amine,
(Z)-3-fluoro-2-(3-fluoro-4-methoxybenzyl)prop-2-en-1-amine,
(Z)-3-fluoro-2-(4-fluoro-3-methylbenzyl)prop-2-en-1-amine,
(Z)-2-(3-chloro-4-fluorobenzyl)-3-fluoroprop-2-en-1-amine,
(Z)-2-(2,5-difluorobenzyl)-3-fluoroprop-2-en-1-amine,
(Z)-2-(3-chloro-5-fluorobenzyl)-3-fluoroprop-2-en-1-amine,
(Z)-2-(2,4-difluorobenzyl)-3-fluoroprop-2-en-1-amine,
(Z)-2-(3,5-dichlorobenzyl)-3-fluoroprop-2-en-1-amine,
(Z)-2-(3,4-difluorobenzyl)-3-fluoroprop-2-en-1-amine,
(Z)-2-(chloromethylene)-4-(4-fluorophenyl)butan-1-amine,
(Z)-2-(chloromethylene)-4-(4-chlorophenyl)butan-1-amine,
(Z)-2-(chloromethylene)-4-(4-methoxyphenyl)butan-1-amine,
(Z)-2-(chloromethylene)-4-(4-ethoxyphenyl)butan-1-amine,
(Z)-2-(chloromethylene)-4-(4-(trifluoromethyl)phenyl)butan-1-amine,
(Z)-4-(4-butoxyphenyl)-2-(chloromethylene)butan-1-amine,
(Z)-2-(chloromethylene)-4-m-tolylbutan-1-amine,
(Z)-2-(chloromethylene)-4-(3-methoxyphenyl)butan-1-amine,
(Z)-2-(chloromethylene)-4-p-tolylbutan-1-amine,
(Z)-2-(chloromethylene)-4-(3-fluoro-5-(trifluoromethyl)phenyl)butan-1-amine,
(Z)-2-(chloromethylene)-4-(4-fluorophenyl)butan-1-amine,
(Z)-2-(chloromethylene)-4-(4-chlorophenyl)butan-1-amine,
(Z)-2-(chloromethylene)-4-(4-methoxyphenyl)butan-1-amine,
(Z)-2-(chloromethylene)-4-(4-ethoxyphenyl)butan-1-amine,
(Z)-2-(chloromethylene)-4-(4-(trifluoromethyl)phenyl)butan-1-amine,
(Z)-4-(4-butoxyphenyl)-2-(chloromethylene)butan-1-amine,
(Z)-2-(chloromethylene)-4-m-tolylbutan-1-amine,
(Z)-2-(chloromethylene)-4-(3-methoxyphenyl)butan-1-amine,
(Z)-2-(chloromethylene)-4-p-tolylbutan-1-amine,
(Z)-2-(chloromethylene)-4-(3-fluoro-5-(trifluoromethyl)phenyl)butan-1-amine,
(Z)-2-(4-(cyclopropylmethoxy)benzyl)-3-fluoroprop-2-en-1-amine,
(Z)-2-(4-(cyclopropylmethoxy)-3-fluorobenzyl)-3-fluoroprop-2-en-1-amine,
(Z)-3-fluoro-2-(3-fluoro-4-(pentyloxy)benzyl)prop-2-en-1-amine,
(Z)-3-fluoro-2-(2,3,5,6-tetrafluoro-4-methoxybenzyl)prop-2-en-1-amine,
(Z)-2-(4-ethoxy-3-fluorobenzyl)-3-fluoroprop-2-en-1-amine,
(Z)-2-(2,3-difluoro-4-methoxybenzyl)-3-fluoroprop-2-en-1-amine,
(Z)-2-(4-(benzyloxy)-3-fluorobenzyl)-3-fluoroprop-2-en-1-amine,
(Z)-3-fluoro-2-(4-fluoro-3-(trifluoromethoxy)benzyl)prop-2-en-1-amine,
(Z)-3-fluoro-2-(4-(2,2,2-trifluoroethoxy)benzyl)prop-2-en-1-amine,
(Z)-2-(3,5-difluoro-4-methoxybenzyl)-3-fluoroprop-2-en-1-amine,
(Z)-2-(3-ethoxybenzyl)-3-fluoroprop-2-en-1-amine,
(Z)-3-fluoro-2-(3-isopropoxybenzyl)prop-2-en-1-amine,
(Z)-3-fluoro-2-(3-(trifluoromethoxy)benzyl)prop-2-en-1-amine,
(Z)-2-(3-(cyclopropylmethoxy)benzyl)-3-fluoroprop-2-en-1-amine,
(Z)-2-(3-(benzyloxy)benzyl)-3-fluoroprop-2-en-1-amine,
(Z)-3-fluoro-2-(2-fluoro-5-(trifluoromethoxy)benzyl)prop-2-en-1-amine,
(Z)-2-(3-chloro-5-(trifluoromethoxy)benzyl)-3-fluoroprop-2-en-1-amine,
(Z)-3-fluoro-2-(2,3,5-trifluorobenzyl)prop-2-en-1-amine,
(Z)-3-fluoro-2-(3,4,5-trifluorobenzyl)prop-2-en-1-amine,
(Z)-3-(2-(aminomethyl)-3-fluoroallyl)benzonitrile,
(Z)-2-(4-bromobenzyl)-3-fluoroprop-2-en-1-amine,
(Z)-2-(3-bromobenzyl)-3-fluoroprop-2-en-1-amine,
(Z)-3-fluoro-2-(pyridin-2-ylmethyl)prop-2-en-1-amine,
(Z)-3-fluoro-2-(pyridin-3-ylmethyl)prop-2-en-1-amine,
(Z)-3-fluoro-2-(naphthalen-2-ylmethyl)prop-2-en-1-amine,
(Z)-2-(benzofuran-2-ylmethyl)-3-fluoroprop-2-en-1-amine,
(Z)-2-(benzofuran-3-ylmethyl)-3-fluoroprop-2-en-1-amine,
(Z)-3-fluoro-2-(quinolin-3-ylmethyl)prop-2-en-1-amine,
(Z)-2-(benzo[b]thiophen-3-ylmethyl)-3-fluoroprop-2-en-1-amine,
(Z)-2-(benzo[b]thiophen-2-ylmethyl)-3-fluoroprop-2-en-1-amine,
(E)-2-((4-ethoxyphenoxy)methyl)-3-fluoroprop-2-en-1-amine,
(E)-2-((4-butoxyphenoxy)methyl)-3-fluoroprop-2-en-1-amine,
(E)-3-fluoro-2-(m-tolyloxymethyl)prop-2-en-1-amine,
(E)-3-fluoro-2-(p-tolyloxymethyl)prop-2-en-1-amine,
(E)-3-fluoro-2-((3-fluoro-5-(trifluoromethyl)phenoxy)methyl)prop-2-en-1-amine,
(Z)-3-fluoro-2-(furan-2-ylmethyl)prop-2-en-1-amine,
(Z)-3-fluoro-2-(thiophen-2-ylmethyl)prop-2-en-1-amine,
(Z)-2-((5-chlorothiophen-2-yl)methyl)-3-fluoroprop-2-en-1-amine,
(Z)-3-fluoro-2-((5-methylthiophen-2-yl)methyl)prop-2-en-1-amine,
(Z)-3-fluoro-2-(furan-3-ylmethyl)prop-2-en-1-amine,
(Z)-3-fluoro-2-(thiophen-3-ylmethyl)prop-2-en-1-amine,
(Z)-2-((5-chlorothiophen-3-yl)methyl)-3-fluoroprop-2-en-1-amine,
(Z)-2-(fluoromethylene)-4-(3-fluorophenyl)butan-1-amine,
(Z)-4-(4-chlorophenyl)-2-(fluoromethylene)butan-1-amine,
(Z)-2-(fluoromethylene)-4-(4-methoxyphenyl)butan-1-amine,
(Z)-4-(4-ethoxyphenyl)-2-(fluoromethylene)butan-1-amine,
(Z)-2-(fluoromethylene)-4-(4-(trifluoromethyl)phenyl)butan-1-amine,
(Z)-4-(4-butoxyphenyl)-2-(fluoromethylene)butan-1-amine,
(Z)-2-(fluoromethylene)-4-m-tolylbutan-1-amine,
(Z)-2-(fluoromethylene)-4-(3-methoxyphenyl)butan-1-amine,
(Z)-2-(fluoromethylene)-4-p-tolylbutan-1-amine,
(Z)-4-(3-fluoro-5-(trifluoromethyl)phenyl)-2-(fluoromethylene)butan-1-amine,
(E)-2-(4-ethoxybenzyl)-3-fluoroprop-2-en-1-amine,
(E)-2-(4-chlorobenzyl)-3-fluoroprop-2-en-1-amine,
(E)-2-(3-chlorobenzyl)-3-fluoroprop-2-en-1-amine,
(E)-3-fluoro-2-(4-isopropoxybenzyl)prop-2-en-1-amine,
(E)-3-fluoro-2-(4-(methylthio)benzyl)prop-2-en-1-amine,
(E)-3-fluoro-2-(3-(methylthio)benzyl)prop-2-en-1-amine,
(E)-3-fluoro-2-(4-(methylsulfonyl)benzyl)prop-2-en-1-amine,
(E)-3-fluoro-2-(4-methylbenzyl)prop-2-en-1-amine,
(E)-3-fluoro-2-(3-methylbenzyl)prop-2-en-1-amine,
(E)-3-fluoro-2-(4-isopropylbenzyl)prop-2-en-1-amine,
(E)-2-(4-tert-butylbenzyl)-3-fluoroprop-2-en-1-amine,
(E)-2-(biphenyl-4-ylmethyl)-3-fluoroprop-2-en-1-amine,
(E)-3-fluoro-2-(4-fluorobenzyl)prop-2-en-1-amine,
(E)-3-fluoro-2-(3-(trifluoromethyl)benzyl)prop-2-en-1-amine,
(E)-3-fluoro-2-(3-fluorobenzyl)prop-2-en-1-amine,
(E)-3-fluoro-2-(3-fluoro-4-methylbenzyl)prop-2-en-1-amine,
(E)-3-fluoro-2-(3-fluoro-4-methoxybenzyl)prop-2-en-1-amine,
(E)-3-fluoro-2-(4-fluoro-3-methylbenzyl)prop-2-en-1-amine,
(E)-2-(3-chloro-4-fluorobenzyl)-3-fluoroprop-2-en-1-amine,
(E)-2-(2,5-difluorobenzyl)-3-fluoroprop-2-en-1-amine,
(E)-2-(3-chloro-5-fluorobenzyl)-3-fluoroprop-2-en-1-amine,
(E)-2-(2,4-difluorobenzyl)-3-fluoroprop-2-en-1-amine,
(E)-2-(3,5-dichlorobenzyl)-3-fluoroprop-2-en-1-amine,
(E)-2-(3,4-difluorobenzyl)-3-fluoroprop-2-en-1-amine,
(E)-2-(chloromethylene)-4-(4-fluorophenyl)butan-1-amine,
(E)-2-(chloromethylene)-4-(4-chlorophenyl)butan-1-amine,
(E)-2-(chloromethylene)-4-(4-methoxyphenyl)butan-1-amine,
(E)-2-(chloromethylene)-4-(4-ethoxyphenyl)butan-1-amine,
(E)-2-(chloromethylene)-4-(4-(trifluoromethyl)phenyl)butan-1-amine,
(E)-4-(4-butoxyphenyl)-2-(chloromethylene)butan-1-amine,
(E)-2-(chloromethylene)-4-m-tolylbutan-1-amine,
(E)-2-(chloromethylene)-4-(3-methoxyphenyl)butan-1-amine,
(E)-2-(chloromethylene)-4-p-tolylbutan-1-amine,
(E)-2-(chloromethylene)-4-(3-fluoro-5-(trifluoromethyl)phenyl)butan-1-amine,
(E)-2-(chloromethylene)-4-(4-fluorophenyl)butan-1-amine,
(E)-2-(chloromethylene)-4-(4-chlorophenyl)butan-1-amine,
(E)-2-(chloromethylene)-4-(4-methoxyphenyl)butan-1-amine,
(E)-2-(chloromethylene)-4-(4-ethoxyphenyl)butan-1-amine,
(E)-2-(chloromethylene)-4-(4-(trifluoromethyl)phenyl)butan-1-amine,
(E)-4-(4-butoxyphenyl)-2-(chloromethylene)butan-1-amine,
(E)-2-(chloromethylene)-4-m-tolylbutan-1-amine,
(E)-2-(chloromethylene)-4-(3-methoxyphenyl)butan-1-amine,
(E)-2-(chloromethylene)-4-p-tolylbutan-1-amine,
(E)-2-(chloromethylene)-4-(3-fluoro-5-(trifluoromethyl)phenyl)butan-1-amine,
(E)-2-(4-(cyclopropylmethoxy)benzyl)-3-fluoroprop-2-en-1-amine,
(E)-2-(4-(cyclopropylmethoxy)-3-fluorobenzyl)-3-fluoroprop-2-en-1-amine,
(E)-3-fluoro-2-(3-fluoro-4-(pentyloxy)benzyl)prop-2-en-1-amine,
(E)-3-fluoro-2-(2,3,5,6-tetrafluoro-4-methoxybenzyl)prop-2-en-1-amine,
(E)-2-(4-ethoxy-3-fluorobenzyl)-3-fluoroprop-2-en-1-amine,
(E)-2-(2,3-difluoro-4-methoxybenzyl)-3-fluoroprop-2-en-1-amine,
(E)-2-(4-(benzyloxy)-3-fluorobenzyl)-3-fluoroprop-2-en-1-amine,
(E)-3-fluoro-2-(4-fluoro-3-(trifluoromethoxy)benzyl)prop-2-en-1-amine,
(E)-3-fluoro-2-(4-(2,2,2-trifluoroethoxy)benzyl)prop-2-en-1-amine,
(E)-2-(3,5-difluoro-4-methoxybenzyl)-3-fluoroprop-2-en-1-amine,
(E)-2-(3-ethoxybenzyl)-3-fluoroprop-2-en-1-amine,
(E)-3-fluoro-2-(3-isopropoxybenzyl)prop-2-en-1-amine,
(E)-3-fluoro-2-(3-(trifluoromethoxy)benzyl)prop-2-en-1-amine,
(E)-2-(3-(cyclopropylmethoxy)benzyl)-3-fluoroprop-2-en-1-amine,
(E)-2-(3-(benzyloxy)benzyl)-3-fluoroprop-2-en-1-amine,
(E)-3-fluoro-2-(2-fluoro-5-(trifluoromethoxy)benzyl)prop-2-en-1-amine,
(E)-2-(3-chloro-5-(trifluoromethoxy)benzyl)-3-fluoroprop-2-en-1-amine,
(E)-3-fluoro-2-(2,3,5-trifluorobenzyl)prop-2-en-1-amine,
(E)-3-fluoro-2-(3,4,5-trifluorobenzyl)prop-2-en-1-amine,
(E)-3-(2-(aminomethyl)-3-fluoroallyl)benzonitrile,
(E)-2-(4-bromobenzyl)-3-fluoroprop-2-en-1-amine,
(E)-2-(3-bromobenzyl)-3-fluoroprop-2-en-1-amine,
(E)-3-fluoro-2-(pyridin-2-ylmethyl)prop-2-en-1-amine,
(E)-3-fluoro-2-(pyridin-3-ylmethyl)prop-2-en-1-amine,
(E)-3-fluoro-2-(naphthalen-2-ylmethyl)prop-2-en-1-amine,
(E)-2-(benzofuran-2-ylmethyl)-3-fluoroprop-2-en-1-amine,
(E)-2-(benzofuran-3-ylmethyl)-3-fluoroprop-2-en-1-amine,
(E)-3-fluoro-2-(quinolin-3-ylmethyl)prop-2-en-1-amine,
(E)-2-(benzo[b]thiophen-3-ylmethyl)-3-fluoroprop-2-en-1-amine,
(E)-2-(benzo[b]thiophen-2-ylmethyl)-3-fluoroprop-2-en-1-amine,
(Z)-2-((4-ethoxyphenoxy)methyl)-3-fluoroprop-2-en-1-amine,
(Z)-2-((4-butoxyphenoxy)methyl)-3-fluoroprop-2-en-1-amine,
(Z)-3-fluoro-2-(m-tolyloxymethyl)prop-2-en-1-amine,
(Z)-3-fluoro-2-(p-tolyloxymethyl)prop-2-en-1-amine,
(Z)-3-fluoro-2-((3-fluoro-5-(trifluoromethyl)phenoxy)methyl)prop-2-en-1-amine,
(E)-3-fluoro-2-(furan-2-ylmethyl)prop-2-en-1-amine,
(E)-3-fluoro-2-(thiophen-2-ylmethyl)prop-2-en-1-amine,
(E)-2-((5-chlorothiophen-2-yl)methyl)-3-fluoroprop-2-en-1-amine,
(E)-3-fluoro-2-((5-methylthiophen-2-yl)methyl)prop-2-en-1-amine,
(E)-3-fluoro-2-(furan-3-ylmethyl)prop-2-en-1-amine,
(E)-3-fluoro-2-(thiophen-3-ylmethyl)prop-2-en-1-amine,
(E)-2-((5-chlorothiophen-3-yl)methyl)-3-fluoroprop-2-en-1-amine,
(E)-2-(fluoromethylene)-4-(3-fluorophenyl)butan-1-amine,
(E)-4-(4-chlorophenyl)-2-(fluoromethylene)butan-1-amine,
(E)-2-(fluoromethylene)-4-(4-methoxyphenyl)butan-1-amine,
(E)-4-(4-ethoxyphenyl)-2-(fluoromethylene)butan-1-amine,
(E)-2-(fluoromethylene)-4-(4-(trifluoromethyl)phenyl)butan-1-amine,
(E)-4-(4-butoxyphenyl)-2-(fluoromethylene)butan-1-amine,
(E)-2-(fluoromethylene)-4-m-tolylbutan-1-amine,
(E)-2-(fluoromethylene)-4-(3-methoxyphenyl)butan-1-amine,
(E)-2-(fluoromethylene)-4-p-tolylbutan-1-amine, or
(E)-4-(3-fluoro-5-(trifluoromethyl)phenyl)-2-(fluoromethylene)butan-1-amine,
or any stereoisomer, mixture of stereoisomers, prodrug, metabolite, crystalline form, non-crystalline form, hydrate, solvate, or salt thereof.

14. The method of claim 1, wherein the compound is of the formula: 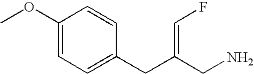
or any solvate, hydrate, crystalline form, non-crystalline form, or salt thereof, in a therapeutically effective amount.

15. A method of inhibiting SSAO activity in a subject, comprising administering (Z)-2-(4′-methoxybenzyl)-3-fluoroallylamine: 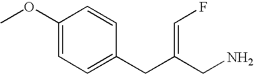
to the subject in an amount sufficient to inhibit SSAO activity by at least about 75% while inhibiting MAO by no more than about 10%.

16. (canceled)

17. A method of treating or preventing inflammation or an inflammatory disease, immune or autoimmune disease, multiple sclerosis or chronic multiple sclerosis, or ischemic disease or the sequelae of an ischemic disease, comprising administering a therapeutically effective amount of a compound of the formula: 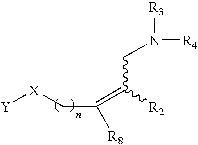
wherein Y is a phenyl, naphthyl, or pyridyl group optionally substituted with one or more groups of the form R₁, wherein each R₁ is independently selected from H, C₁-C₈ alkyl, C₃-C₈ cycloalkyl, O—C₁-C₈ alkyl, —O—C₃-C₈ cycloalkyl, —C₆-C₁₀ aryl, —O—C₁-C₄ alkyl-C₆-C₁₀ aryl, —S—C₁-C₈ alkyl, —CF₃, —OCF₃, F, Cl, Br, I, —NO₂, —OH, —NR₅R₆, —NHR₇, and —S(O₂)—(C₁-C₈ alkyl); R₂ is selected from H, F, Cl, C₁-C₄ alkyl, and CF₃; R₃ and R₄ are independently selected from H, —C₁-C₈ alkyl, —C₁-C₄ alkyl-C₆-C₁₀ aryl, or R₃ and R₄ together with the nitrogen to which they are attached form a nitrogen-containing ring; R₅ and R₆ are independently selected from H, —C₁-C₈ alkyl, —C₁-C₄ alkyl-C₆-C₁₀ aryl, or R₅ and R₆ together with the nitrogen to which they are attached form a nitrogen-containing ring; R₇ is selected from —C(═O)—(C₁-C₈ alkyl) and —C(═O)—(C₆-C₁₀ aryl); R₈ is selected from H, C₁-C₈ alkyl, C₆-C₁₀ aryl, —C₁-C₄ alkyl-C₆-C₁₀ aryl, and —CF₃; X is —CH₂—, —O—, or —S—; n is 0, 1, 2, or 3; and p is 0, 1, 2, or 3; and any stereoisomer, mixture of stereoisomers, prodrug, metabolite, crystalline form, non-crystalline form, hydrate, solvate, or salt thereof.

18. The method of claim 17, wherein X is CH₂ and n is 1.

19. The method of claim 17, wherein X is CH₂ and n is 0.

20. The method of claim 17, wherein Y is phenyl, optionally substituted with one or more groups of the form R₁.

21. The method of claim 17, wherein R₃ and R₄ are both H.

22. The method of claim 17, wherein X is O or S and n is 1.

23. The method of claim 17, wherein the double bond is in the E configuration.

24. The method of claim 17, wherein the double bond is in the Z configuration.

25. The method of claim 17, wherein the compound is:
(E)-2-fluoro-4-(3-fluoro-5-(trifluoromethyl)phenyl)but-2-en-1-amine,
(E)-2-fluoro-4-(3-(trifluoromethyl)phenyl)but-2-en-1-amine,
(E)-2-fluoro-4-(4-methoxyphenyl)but-2-en-1-amine,
(E)-2-fluoro-4-(4-methoxy-3-(trifluoromethyl)phenyl)but-2-en-1-amine,
(E)-2-fluoro-4-(4-fluorophenyl)but-2-en-1-amine,
(E)-2-fluoro-4-m-tolylbut-2-en-1-amine,
(E)-2-fluoro-4-(3-fluorophenyl)but-2-en-1-amine,
(E)-2-fluoro-4-(3-methoxyphenyl)but-2-en-1-amine,
(E)-2-fluoro-4-phenylbut-2-en-1-amine,
(E)-2-fluoro-4-(3-(trifluoromethoxy)phenyl)but-2-en-1-amine,
(E)-3-(4-amino-3-fluorobut-2-enyl)-N,N-dimethylbenzenamine,
(E)-4-(3,5-bis(trifluoromethyl)phenyl)-2-fluorobut-2-en-1-amine,
(E)-2-fluoro-4-(3-(methylthio)phenyl)but-2-en-1-amine,
(E)-2-fluoro-4-(3-(methylsulfonyl)phenyl)but-2-en-1-amine,
(E)-2-fluoro-4-(3-(trifluoromethylthio)phenyl)but-2-en-1-amine,
(E)-2-fluoro-4-(3-(methoxymethyl)phenyl)but-2-en-1-amine,
(E)-2-fluoro-4-(2-methoxyphenyl)but-2-en-1-amine,
(E)-2-fluoro-4-(3-methoxyphenyl)-3-methylbut-2-en-1-amine,
(E)-2-fluoro-4-(3-methoxyphenoxy)but-2-en-1-amine,
(E)-2-fluoro-4-(3-methoxyphenylthio)but-2-en-1-amine,
(E)-2-fluoro-4-(3-(trifluoromethyl)phenoxy)but-2-en-1-amine,
(E)-2-fluoro-4-(pyridin-3-yloxy)but-2-en-1-amine,
(E)-2-fluoro-5-(3-methoxyphenyl)pent-2-en-1-amine,
(Z)-2-fluoro-4-(3-fluoro-5-(trifluoromethyl)phenyl)but-2-en-1-amine,
(Z)-2-fluoro-4-(3-(trifluoromethyl)phenyl)but-2-en-1-amine,
(Z)-2-fluoro-4-(4-methoxyphenyl)but-2-en-1-amine,
(Z)-2-fluoro-4-(4-methoxy-3-(trifluoromethyl)phenyl)but-2-en-1-amine,
(Z)-2-fluoro-4-(4-fluorophenyl)but-2-en-1-amine,
(Z)-2-fluoro-4-m-tolylbut-2-en-1-amine,
(Z)-2-fluoro-4-(3-fluorophenyl)but-2-en-1-amine,
(Z)-2-fluoro-4-(3-methoxyphenyl)but-2-en-1-amine,
(Z)-2-fluoro-4-phenylbut-2-en-1-amine,
(Z)-2-fluoro-4-(3-(trifluoromethoxy)phenyl)but-2-en-1-amine,
(Z)-3-(4-amino-3-fluorobut-2-enyl)-N,N-dimethylbenzenamine,
(Z)-4-(3,5-bis(trifluoromethyl)phenyl)-2-fluorobut-2-en-1-amine,
(Z)-2-fluoro-4-(3-(methylthio)phenyl)but-2-en-1-amine,
(Z)-2-fluoro-4-(3-(methylsulfonyl)phenyl)but-2-en-1-amine,
(Z)-2-fluoro-4-(3-(trifluoromethylthio)phenyl)but-2-en-1-amine,
(Z)-2-fluoro-4-(3-(methoxymethyl)phenyl)but-2-en-1-amine,
(Z)-2-fluoro-4-(2-methoxyphenyl)but-2-en-1-amine,
(Z)-2-fluoro-4-(3-methoxyphenyl)-3-methylbut-2-en-1-amine,
(Z)-2-fluoro-4-(3-methoxyphenoxy)but-2-en-1-amine,
(Z)-2-fluoro-4-(3-methoxyphenylthio)but-2-en-1-amine,
(Z)-2-fluoro-4-(3-(trifluoromethyl)phenoxy)but-2-en-1-amine,
(Z)-2-fluoro-4-(pyridin-3-yloxy)but-2-en-1-amine, or
(Z)-2-fluoro-5-(3-methoxyphenyl)pent-2-en-1-amine,
or any stereoisomer, mixture of stereoisomers, prodrug, metabolite, crystalline form, non-crystalline form, hydrate, solvate, or salt thereof.

26. The method of claim 17, wherein the inflammation or inflammatory disease or immune or autoimmune disease is multiple sclerosis, chronic multiple sclerosis, synovitis, systemic inflammatory sepsis, inflammatory bowel diseases, Crohn's disease, ulcerative colitis, Alzheimer's disease, vascular dementia, atherosclerosis, rheumatoid arthritis, juvenile rheumatoid arthritis, pulmonary inflammation, asthma, skin inflammation, contact dermatitis, liver inflammation, liver autoimmune diseases, autoimmune hepatitis, primary biliary cirrhosis, sclerosing cholangitis, autoimmune cholangitis, alcoholic liver disease, Type I diabetes and/or complications thereof, Type II diabetes and/or complications thereof, atherosclerosis, chronic heart failure, congestive heart failure, ischemic diseases, stroke and/or complications thereof, or myocardial infarction and/or complications thereof.

27. The method of claim 26, wherein the inflammation or inflammatory disease or immune or autoimmune disease is multiple sclerosis, chronic multiple sclerosis, stroke, or complications of stroke.

28. The method of claim 17, wherein the compound is of the formula: 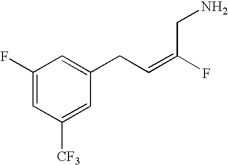
or any solvate, hydrate, crystalline form, non-crystalline form, or salt thereof, in a therapeutically effective amount.

29-53. (canceled)
